(19) United States
(12) Patent Application Publication
(10) Pub. No.: US 2025/0154170 A1
(43) Pub. Date: May 15, 2025

(54) FUSED PYRIMIDINES AS KRAS INHIBITORS

(71) Applicant: Bayer Aktiengesellschaft, Leverkusen (DE)

(72) Inventors: Daniel Hillebrand O'DONOVAN, Cambridge (GB); Wolfgang SCHWEDE, Glienicke (DE); Jeremie Xavier G MORTIER, Berlin (DE); Volker SCHULZE, Hohen Neuendorf, OT Bergf (DE); Sebastian ESSIG, Sinsheim (DE); Lisa CANDISH, Wuppertal (DE); Franziska SIEGEL, Berlin (DE); Lukas Patrik BRAUN, Berlin (DE); Gary Norbert HERMANN, Düsseldorf (DE); Benjamin BADER, Berlin (DE); Yung-Ning CHANG, Berlin (DE); Roman HILLIG, Highton, VIC (AU)

(21) Appl. No.: 18/836,180

(22) PCT Filed: Feb. 9, 2023
(86) PCT No.: PCT/EP2023/053248

§ 371 Date: Aug. 6, 2024

Related U.S. Application Data
(60) Provisional application No. 63/308,915, filed on Feb. 10, 2022.

(51) Int. Cl. C07D 519/00; A61K 31/52; A61K 31/551; A61K 45/06

(52) U.S. Cl. CPC C07D 519/00; A61K 31/52; A61K 31/551; A61K 45/06

(57) ABSTRACT

The present invention covers fused pyrimidine compounds of general formula (I):

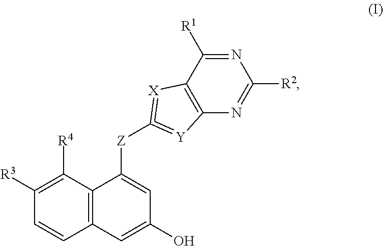

in which R¹, R², R³, R⁴, X, Y and Z are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of neoplastic disorders, respectively cancer or conditions with dysregulated immune responses or other disorders associated with aberrant KRAS signaling, as a sole agent or in combination with other active ingredients.

The present invention covers fused pyrimidine compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular for neoplastic disorders, respectively cancer or conditions with dysregulated immune responses or other disorders associated with aberrant KRAS signaling, as a sole agent or in combination with other active ingredients.

### BACKGROUND

Mutant KRAS is a well-understood oncogenic driver and has a wide-spread prevalence in various human cancer indications (Bos, 1989). In 1982, mutationally activated RAS genes were detected in human cancer, marking the first discovery of mutated genes in this disease (Cox, 2010). The frequent mutation of RAS in three of the four most lethal cancers (lung, colon and pancreatic cancers) in the United States has spurred intense interest and effort in developing RAS inhibitors (Cox, 2014). Overall, RAS mutations have been detected in 9-30% of all tumor samples sequenced. In pancreatic ductal adenocarcinoma (PDAC; ˜90% of all pancreatic cancers) and lung adenocarcinoma (LAC; 30-35% of all lung cancers) KRAS mutations display a frequency of 97% and 32% respectively. Other indications with frequently mutated KRAS include colorectal carcinoma (CRC) (52%), and multiple myeloma (43%) (Cox, 2014).

RAS proteins act as molecular switches that cycle between an active, GTPbound state and an inactive, GDP-bound state. Activated by guanine nucleotide exchange factors (GEFs), RAS in its GTPbound state interacts with a number of effectors (Hillig, 2019). Return to the inactive state is driven by GTPase-activating proteins (GAPs), which down-regulate active RAS by accelerating the weak intrinsic GTPase activity by up to 5 orders of magnitude. For oncogenic RAS mutants, however, the GAP activity is impaired or greatly reduced, resulting in permanent activation, which is the basis of oncogenic RAS signaling (Haigis, 2017); for example, through the RAS-RAF-MEK-ERK and RAS-PI3K-PDK1-AKT pathways, both essential to cell survival and proliferation (Downward 2003). For decades, mutant KRAS has been considered “undruggable” with classical pharmacological small molecule inhibitors. However, KRASG12C was recently identified to be potentially druggable by allele-specific covalent targeting of Cys-12 in vicinity to an inducible allosteric switch II pocket (S—IIP) (Oestrem, 2013; Janes, 2018).

Covalent KRASG12C inhibitors as described by Shokat et al. (Ostrem J M, Shokat K M (2016) Direct small-molecule inhibitors of KRAS: From structural insights to mechanism-based design. Nat Rev Drug Discov 15:771-785.) occupy the so-called switch-II pocket and bind with their Michael acceptor system covalently to the cysteine mutation at G12 in this specific KRAS mutant. Occupation of this pocket with the covalent inhibitor results in a locked inactive GDP-bound protein conformation. Captured in this conformation, cycling of the mutated protein into the active GTP-bound state is prevented and thereby activity of the mutant KRASG12C is shut down.

For decades, mutant KRAS has been considered “undruggable” via classical pharmacological small molecule inhibitors. However, in 2013 the G12C mutant of KRAS was found to be potentially druggable by covalent targeting of Cys-12 in vicinity to an inducible so-called “switch II pocket” (S—IIP) of KRAS G12C (Oestrem, 2013; Janes, 2018). There have since been significant efforts by the pharmaceutical industry to develop KRas inhibitors targeting the SII—P for cancer therapy and several agents have entered clinical trials. However, no such therapies have yet won regulatory approval (McCormick, 2015). Besides G12C, other oncogenic mutants of KRAS include G12D, G12V and G12R, all of which represent attractive drug targets, with the G12D mutation being most prevalent across tumor types (Kashofer, 2020). There is therefore a clear and continued desire for therapies targeting KRas mutants and especially G12D for the treatment of cancer.

### STATE OF THE ART

Covalent inhibitors of KRAS G12C have been described in literatures and patent applications. Biaryl derivatives were mentioned as KRAS G12C covalent inhibitors (WO2014152588, WO2016049524 and WO 2016044772). WO2016164675, WO2015054572, WO2016044772, WO2016049568, WO2016168540, WO20170070256, WO2017087528, WO2017100546, WO2017172979, WO2018064510, WO2018145012, WO2018145014 disclosed quinazoline, quinoline, dihydrobenzo-naphthyridinone, quinazolinone, dihydropyrimidoquinolinone, isoquinoline derivatives. Further disclosures include anilinoacetamide and biaryl derivatives (WO2016049565, WO 2017058768, WO 2017058792), naphthalene or hexahydrofurofurane derivatives (WO 2014143659), quinazolinone (WO2017015562), phenylpyrazine derivatives (WO 2017058728). bezoimidazolsulfone, dihydroquinoxaline or dihydroquinoxalinone (WO 2017058805), phenylpiperazine-1-carbohydrazide (WO 2017058807), tetrahydronaphthyridine (WO 2017058902), imidazolopyridine (WO 2017058915), various chemical entities (WO2018068017), bicyclic 6,5-aryl, hetaryl rings containing compounds (WO2018140600).

Benzimidazol, (aza)indole, imidazopyridine derivatives were disclosed as KRAS covalent inhibitors in WO2018145013, benzothiazole, benzothiophene, benzisoxazole derivatives in WO2018140599, pyridopyrimidone, benzothiazole in WO2018119183 and tetrahydropyridopyrimidine in WO2017201161.

### Compounds of the Following General Formula

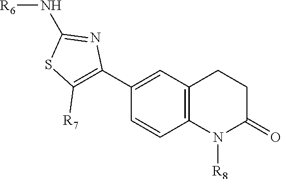

are described in US 2018/0201610 (NantBio) which selectively inhibit mutant K-Ras, especially G12V and/or G12D over wild type K-Ras or other mutant K-Ras forms.

### Substituted Quinazoline Compounds of the Following General Formula

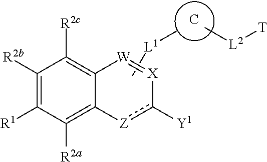

are described as inhibitors of Ras-protein in WO 2017/172979 (Araxes).

### Compounds of General Formula

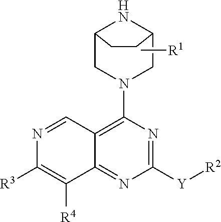

are described as inhibitors of KRAS G12D in WO 2021/041671 (Mirati).

### Compounds of General Formula

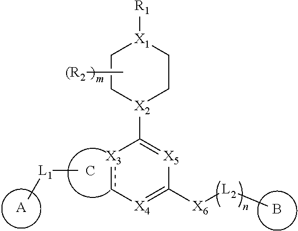

are described as to inhibit mutant KRAS in CN 112047948 (Xuanzhu).

Reversible, non-covalent inhibitors of KRAS G12D have been described in patent applications (WO2021041671 and WO2017172979A1). However, so far compounds of general formula (I) have not been disclosed as reversible, non-covalent KRAS G12D inhibitors.

It has now been found, and this constitutes the basis of the present invention, that the compounds of the present invention have surprising and advantageous properties.

In particular, the compounds of the present invention have surprisingly been found to effectively inhibit KRAS, especially KRAS G12D, and may therefore be used for the treatment or prophylaxis of neoplastic disorders, repectively cancer or conditions with dysregulated immune responses or other disorders associated with aberrant KRAS signaling, for example.

### DESCRIPTION OF THE INVENTION

In accordance with a first aspect, the present invention covers compounds of general formula (I):

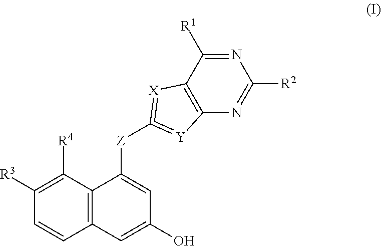

in which
X is selected from the group consisting of ═N—, —NR^{a}—, —CR^{b}═ and —S—;
   R^{a} is H or C₁₋₃-alkyl, optionally substituted by one or more F;
   R^{b} is selected from the group consisting of H, F, Cl, and C₁₋₃-alkyl, optionally substituted by one or more F;
Y is selected from the group consisting of ═N—, —NR^{c}—, —CR^{d}═ and —S—;
   R^{c} is H or C₁₋₃-alkyl, optionally substituted by one or more F;
   R^{d} is selected from the group consisting of H, F, Cl, and C₁₋₃-alkyl, optionally substituted by one or more F;
   with the condition, that X and Y are not simultaneous —CH═ or simultaneous —S—;
   It is understood that the fragment —X═C(—Z)═Y— in general formula (I) has the meaning of either —X—C(—Z)═Y— or —X═C(—Z)—Y—;
Z is selected from the group consisting of —NH—, —N(CH₃)—, —CH₂—, —C(CH₃)—, —C(OH)—, —O—, —S—, —S(═O)—, —S(═O)₂— and —S(═O)(═NH)—;
R¹ is selected from the group consisting of

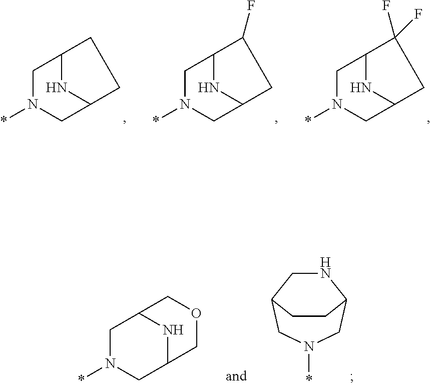 [SMILES: *N1CC2CCC(C1)N2 ; *N1CC2CC(F)C(C1)N2 ; *N1CC2CC(F)(F)C(C1)N2 ; *N1CC2COCC(C1)N2 ; *N1CC2CCC(C1)NC2]

R² is selected from the group consisting of

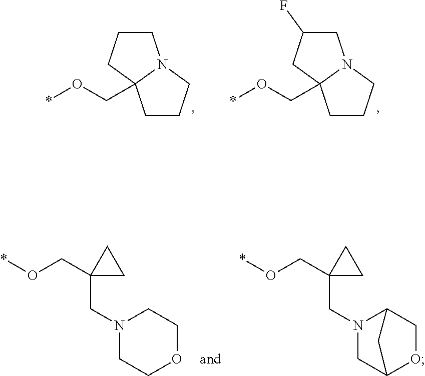 [SMILES: *OCC12CCCN1CCC2 ; *OCC12CCCN1CC(F)C2 ; *OCC1(CN2CCOCC2)CC1 ; *OCC1(CN2CC3CC2CO3)CC1]

R³ is selected from the group consisting of H, F, Cl, Br, I, and —CH₃;
R⁴ is selected from the group consisting of H, F, Cl, Br, I, —CH₃, —CH₂—CH₃, —CH═CH₂, —C≡CH, —C≡C—CH₃, and —C≡C—CH₂—CH₃;
   or a stereoisomer, a tautomer, an N-oxide, a hydrate, a solvate, or a salt thereof, or a mixture of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “comprising” when used in the specification includes “consisting of”.

If within the present text any item is referred to as “as mentioned herein”, it means that it may be mentioned anywhere in the present text.

The terms as mentioned in the present text have the following meanings: The term “C₁-C₃-alkyl” means a linear or branched, saturated, monovalent hydrocarbon group having 1, 2, or 3 carbon atoms, e.g. a methyl, ethyl, propyl, or isopropyl group.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely

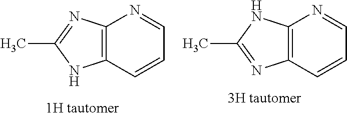

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexyl-carbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (I), supra, in which:
X is selected from the group consisting of ═N—, —NH—, —N(CH₃)—, —CH═, —C(CH₃)═ and —S—;
Y is selected from the group consisting of ═N—, —NH—, —N(CH₃)—, —CH═, —C(CH₃)═ and —S—, with the condition, that X and Y are not simultaneous —CH═ or simultaneous —S—;
   It is understood that the fragment —X═C(—Z)═Y— in general formula (I) has the meaning of either —X—C(—Z)═Y— or —X═C(—Z)—Y—;
Z is selected from the group consisting of —NH—, —N(CH₃)—, —CH₂—, —CH(CH₃)—, —CH(OH)— and —O—;
R¹ is selected from the group consisting of

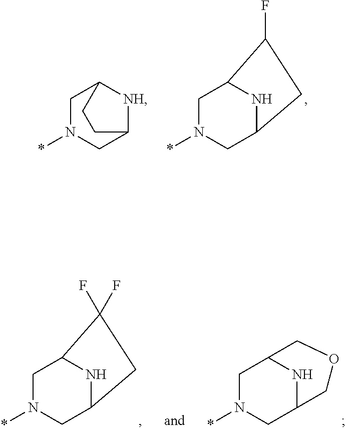

R² is selected from the group consisting of

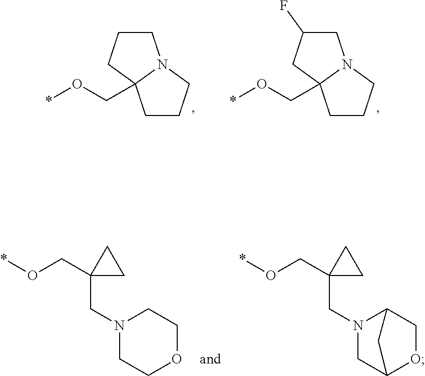 [SMILES: *OCC12CCCN1CCC2 ; *OCC12CCCN1CC(F)C2 ; *OCC1(CN2CCOCC2)CC1 ; *OCC1(CN2CC3CC2CO3)CC1]

R³ is selected from the group consisting of H, F, Cl, and —CH₃;
R⁴ is selected from the group consisting of H, F, Cl, —CH₃, and —C≡CH;
   or a stereoisomer, a tautomer, an N-oxide, a hydrate, a solvate, or a salt thereof, or a mixture of same.

The present invention also covers the following compounds of general formula (I), supra,
4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol,
4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-5-ethynyl-2-naphthol,
4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-fluoronaphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}oxy)-5-methylnaphthalen-2-ol,
4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-ethynyl-6-fluoronaphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynyl-6-fluoronaphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}amino)naphthalen-2-ol,
4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)-5-fluoronaphthalen-2-ol,
4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)naphthalen-2-ol,
4-({6-(3,8-diazabicyclo[3.2.1]octan-3-yl)-2-[(tetrahydro-1H-pyrrolizin-7a(5H)-yl)methoxy]-9H-purin-8-yl}oxy)naphthalen-2-ol,
4-({6-(3,8-diazabicyclo[3.2.1]octan-3-yl)-2-[(tetrahydro-1H-pyrrolizin-7a(5H)-yl)methoxy]-9H-purin-8-yl}oxy)-5-fluoronaphthalen-2-ol,
5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol,
5-ethynyl-6-fluoro-4-({7-(3-oxa-7,9-diazabicyclo[3.3.1]nonan-7-yl)-5-[(tetrahydro-1H-pyrrolizin-7a(5H)-yl)methoxy][1,3]thiazolo[5,4-d]pyrimidin-2-yl}oxy)naphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}methyl)-5-fluoronaphthalen-2-ol,
5-Chlor-4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol,
4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethenyl-6-fluoronaphthalen-2-ol,
4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)naphthalen-2-ol,
5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol,
4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-[(7-[(1S*,5S*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)-5-ethynyl-6-fluoronaphthalen-2-ol
6-chloro-4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynylnaphthalen-2-ol
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]amino}naphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}naphthalen-2-ol,
4-{[5-{1[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]amino}-2-naphthol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}-5-ethynylnaphthalen-2-ol, and
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]methyl}-5-ethynyl-6-fluoronaphthalen-2-ol formic acid (1/1)
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is selected from the group consisting of ═N—, —NR^{a}—, —CR^{b}═ and —S—;
   R^{a} is H or C₁₋₃-alkyl, optionally substituted by one or more F;
   R^{b} is selected from the group consisting of H, F, Cl, and C₁₋₃-alkyl, optionally substituted by one or more F;
      or a stereoisomer, a tautomer, an N-oxide, a hydrate, a solvate, or a salt thereof, or a mixture of same.

In a further embodiment of the first aspect, the present invention covers compounds of formula (I), supra, in which:
X is selected from the group consisting of ═N—, —NH—, —N(CH₃)—, —CH═, —C(CH₃)═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is selected from the group consisting of ═N—, —CH═, —C(CH₃)═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is selected from the group consisting of ═N—, —CH═, and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is ═N—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is selected from the group consisting of —CH═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is —CH═;
   or a stereoisomer, a tautomer, an N-oxide, a hydrate, a solvate, or a salt thereof, or a mixture of same.

In a further embodiment of the first aspect, the present invention covers compounds of formula (I), supra, in which:
X is —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is selected from the group consisting of ═N—, —NR^{c}—, —CR^{d}═ and —S—;
   R^{c} is H or C₁₋₃-alkyl, optionally substituted by one or more F;
R^{d} is selected from the group consisting of H, F, Cl, and C₁₋₃-alkyl, optionally substituted by one or more F;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is selected from the group consisting of ═N—, —NH—, —N(CH₃)—, —CH═, —C(CH₃)═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is selected from the group consisting of ═N—, —CH═, —C(CH₃)═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is selected from the group consisting of —CH═ and —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is —CH═;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Y is —S—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Z is selected from the group consisting of —NH—, —N(CH₃)—, —CH₂—, —C(CH₃)—, —C(OH)—, —O—, —S—, —S(═O)—, —S(═O)₂— and —S(═O)(═NH)—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Z is selected from the group consisting of —NH—, —N(CH₃)—, —CH₂—, —CH(CH₃)—,
   —CH(OH)— and —O—;
      or a stereoisomer, a tautomer, an N-oxide, a hydrate, a solvate, or a salt thereof, or a mixture of same.

In a further embodiment of the first aspect, the present invention covers compounds of formula (I), supra, in which:
Z is selected from the group consisting of —NH—, —CH₂—, and —O—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Z is selected from the group consisting of —CH₂—, and —O—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Z is —CH₂—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
Z is —O—;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R¹ is selected from the group consisting of

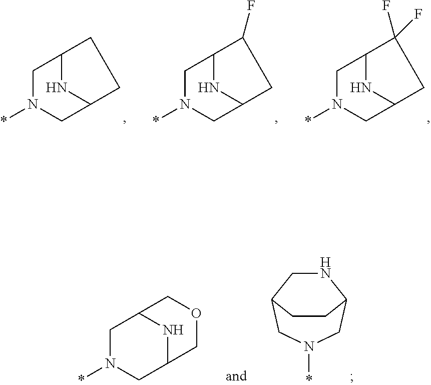 [SMILES: *N1CC2CCC(C1)N2 ; *N1CC2CC(F)C(C1)N2 ; *N1CC2CC(F)(F)C(C1)N2 ; *N1CC2COCC(C1)N2 ; *N1CC2CCC(C1)NC2]

or a stereoisomer, a tautomer, an N-oxide, a hydrate, a solvate, or a salt thereof, or a mixture of same.

In a further embodiment of the first aspect, the present invention covers compounds of formula (I), supra, in which:
R¹ is selected from the group consisting of

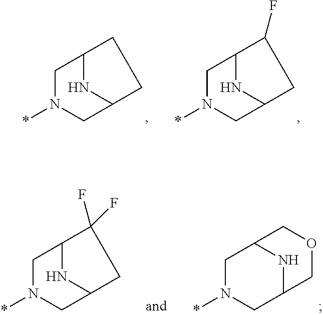 [SMILES: *N1CC2CCC(C1)N2 ; *N1CC2CC(F)C(C1)N2 ; *N1CC2CC(F)(F)C(C1)N2 ; *N1CC2COCC(C1)N2]

or a stereoisomer, a tautomer, an N-oxide, a hydrate, a solvate, or a salt thereof, or a mixture of same.

In a further embodiment of the first aspect, the present invention covers compounds of formula (I), supra, in which:
R¹ is

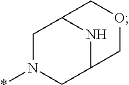 [SMILES: *N1CC2COCC(C1)N2]

or a stereoisomer, a tautomer, an N-oxide, a hydrate, a solvate, or a salt thereof, or a mixture of same.

In a further embodiment of the first aspect, the present invention covers compounds of formula (I), supra, in which:
R² is selected from the group consisting of

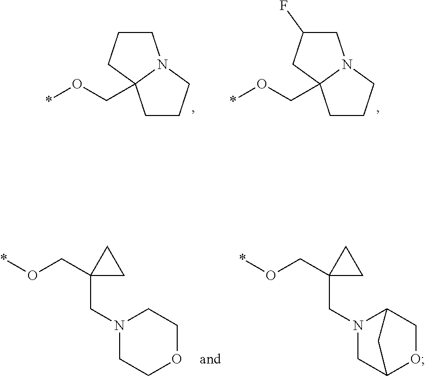 [SMILES: *OCC12CCCN1CCC2 ; *OCC12CCCN1CC(F)C2 ; *OCC1(CN2CCOCC2)CC1 ; *OCC1(CN2CC3CC2CO3)CC1]

or a stereoisomer, a tautomer, an N-oxide, a hydrate, a solvate, or a salt thereof, or a mixture of same.

In a further embodiment of the first aspect, the present invention covers compounds of formula (I), supra, in which:
R² is selected from the group consisting of

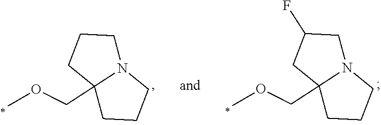

or a stereoisomer, a tautomer, an N-oxide, a hydrate, a solvate, or a salt thereof, or a mixture of same.

In a further embodiment of the first aspect, the present invention covers compounds of formula (I), supra, in which:
R³ is selected from the group consisting of H, F, Cl, Br, I, and —CH₃;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R³ is selected from the group consisting of H, F, Cl, and —CH₃;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R³ is selected from the group consisting of H, F, and Cl;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R⁴ is selected from the group consisting of H, F, Cl, Br, I, —CH₃, —CH₂—CH₃, —CH═CH₂, —C≡CH, —C≡C—CH₃, and —C≡C—CH₂—CH₃;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R⁴ is selected from the group consisting of H, F, Cl, Br, I, —CH₃, —CH₂—CH₃, —CH═CH₂, —C≡CH, and —C≡C—CH₃;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R⁴ is selected from the group consisting of H, F, Cl, —CH₃, and —C≡C—CH₃;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R⁴ is selected from the group consisting of H, F, Cl, —CH₃, and —C≡CH;
   or a stereoisomer, a tautomer, an N-oxide, a hydrate, a solvate, or a salt thereof, or a mixture of same.

In a further embodiment of the first aspect, the present invention covers compounds of formula (I), supra, in which:
R⁴ is selected from the group consisting of F, and —C≡CH;
   or a stereoisomer, a tautomer, an N-oxide, a hydrate, a solvate, or a salt thereof, or a mixture of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention.

The present invention covers the compounds of general formula (I) which are disclosed in the Example Section of this text, infra.

The compounds according to the invention of general formula (I) can be prepared according to the following schemes 1 and 2. The schemes and procedures described below illustrate synthetic routes to the compounds of general formula (I) of the invention and are not intended to be limiting. It is clear to the person skilled in the art that the order of transformations as exemplified in schemes 1 and 2 can be modified in various ways. The order of transformations exemplified in these schemes is therefore not intended to be limiting. In addition, interconversion of any of the substituents, R¹, R² or R³, can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. Compounds of the present invention have surprisingly been found to effectively inhibit KRAS and it is possible therefore that said compounds be used for the treatment or prophylaxis of diseases, preferably neoplasic disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular neoplastic disorders.

The pharmaceutical activity of the compounds according to the invention can be explained by their activity as KRAS inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular neoplastic disorders, respectively cancer or conditions with dysregulated immune responses or other disorders associated with aberrant KRAS signaling.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular neoplastic disorders, respectively cancer or conditions with dysregulated immune responses or other disorders associated with aberrant KRAS signaling.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular neoplastic disorders, respectively cancer or conditions with dysregulated immune responses or other disorders associated with aberrant KRAS signaling.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular neoplastic disorders, respectively cancer or conditions with dysregulated immune responses or other disorders associated with aberrant KRAS signaling.

In accordance with a further aspect, the present invention covers a method of treatment or prophylaxis of diseases, in particular neoplastic disorders, respectively cancer or conditions with dysregulated immune responses or other disorders associated with aberrant KRAS signaling, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides, fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab©), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a neoplastic disorder, respectively cancer or conditions with dysregulated immune responses or other disorders associated with aberrant KRAS signaling.

Particularly, the present invention covers a pharmaceutical combination, which comprises:
one or more first active ingredients, in particular compounds of general formula (I) as defined supra, and
one or more further active ingredients, in particular cancer agents.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known cancer agents.

### Examples of Cancer Agents Include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, darolutamide, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of neoplastic disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ(intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

Optical rotations were measured using a JASCO P2000 Polarimeter. Typical, a solution of the compound with a concentration of 1 mg/mL to 15 mg/mL was used for the measurement. The specific rotation [α]_{D} was calculated according to the following formula:

$$[ α ] D = \frac{∝}{β \times d}$$

In this equation, α is the measured rotation in degrees; d is the path length in decimetres and β is the concentration in g/mL.

The following table 1 lists the abbreviations used in this paragraph and in the Examples section as far as they are not explained within the text body. Other abbreviations have their meanings customary per se to the skilled person.

### Table 1: Abbreviations

Table 1 lists the abbreviations used in this paragraph and in the Intermediates and Examples sections as far as they are not explained within the text body.

TABLE 1

| Abbreviation | Meaning |
|---|---|
| ACN | acetonitrile |
| AcOH | acetic acid (ethanoic acid) |
| aq. | Aqueous |
| BINAP | 2,2′-bis(diphenylphosphino)-1,1′-binaphthyl |
| Boc | t-butoxycarbonyl |
| BOP | (benzotriazol-1-yloxy)tris(dimethylamino)phos- |
| | phonium hexafluorophosphate |
| br | Broad |
| CI | chemical ionization |
| Cs2CO3 | cesium carbonate |
| d | Doublet |
| DAD | diode array detector |
| DBU | 1,8-diazabicyclo(5.4.0)undec-7-ene |
| DCC | N,N′-dicyclohexylcarbodiimide |
| DCM | Dichloromethane |
| dd | double-doublet |
| DIBALH | diisobutylaluminium hydride |
| DIC | N,N′-diisopropylcarbodiimide |
| DIPEA | Diisopropylethylamine |
| DMA | Dimethylacetamide |
| DMF | N,N-dimethylformamide |
| DMSO | dimethyl sulfoxide |
| dt | double-triplet |
| EDC | 1-ethyl-3-(3-dimethylaminopropyl)carbodiimide |
| ELSD | Evaporative Light Scattering Detector |
| EtOAc | ethyl acetate |
| EtOH | Ethanol |
| eq. | Equivalent |
| ESI | electrospray (ES) ionization |
| h | Hour |
| HATU | 1-[bis(dimethylamino)methylene]-1H-1,2,3- |
| | triazolo[4,5-b]pyridinium 3-oxid |
| | hexafluorophosphate |
| HBTU | (o-benzotriazole-10yl)-N,N,N′,N,- |
| | tetramethyluronium hexafluorophosphate |
| HCl | hydrochloric acid |
| HPLC | high performance liquid chromatography |
| K2CO3 | potassium carbonate |
| LC-MS | liquid chromatography mass spectrometry |
| LiHMDS | lithium bis(trimethylsilyl)amide |
| m | Multiplet |
| mCPBA | meta-Chloroperbenzoic acid |
| min | Minute |
| MeCN | Acetonitrile |
| MeOH | Methanol |
| MS | mass spectrometry |
| MTBE | methyl-tert-butyl ether |
| n-BuLi | n-butyllithium |
| NaCl | sodium chloride |
| NaH | sodium hydride |
| NH3 | ammonia |
| NH4Cl | ammonium chloride |
| NBS | 1-bromopyrrolidine-2,5-dione; N-Bromsuccinimid |
| NaHCO3 | sodium hydrogen carbonate or sodium bicarbonate |
| NMR | nuclear magnetic resonance spectroscopy: chemical |
| | shifts (δ) are given in ppm. The chemical shifts were |
| | corrected by setting the DMSO signal to 2.50 ppm |
| | unless otherwise stated. |
| Na2SO4 | sodium sulfate |
| PDA | Photo Diode Array |
| Pd/C | palladium on activated charcoal |
| Pd2(dba)3 | tris(dibenzylideneacetone)dipalladium(0) |
| Pd(OAc)2 | Palladium(II) acetate |
| PyBOP | (benzotriazol-1-yloxy)tripyrrolidinophosphonium |
| | hexafluorophosphate |
| q | Quartet |
| r.t. or rt or RT | room temperature |
| Rt | retention time (as measured either with HPLC or |
| | UPLC) in minutes |
| s | Singlet |
| sat. | Saturated |
| SEM | (2-methoxyethyl)(trimethyl)silyl group |
| SEM-Cl | [2-(chloromethoxy)ethyl](trimethyl)silane |
| SFC | supercritical fluid chromatography |
| SIBX | stabilized 2-iodoxybenzoic acid |
| SM | starting material |
| SQD | Single-Quadrupole-Detector |
| T3P | propylphosphonic anhydride |
| t | Triplet |
| tBuBrettPhos | di-tert-butyl(2′,4′,6′-triisopropyl-3,6- |
| | dimethoxy[biphenyl]-2-yl)phosphine (CAS |
| | 1160861-53-9) |
| tBuBrettPhos Pd G3 | (2′-amino[biphenyl]-2-yl)(methanesulfonato- |
| | kappaO)palladium-di-tert-butyl(2′,4′,6′-triisopropyl- |
| | 3,6-dimethoxy[biphenyl]-2-yl)phosphine (1:1) |
| | (CAS: 1536473-72-9) |
| td | triple-doublet |
| TEA | Triethylamine |
| TFA | trifluoroacetic acid |
| TFAA | trifluoroacetic anhydride |
| THF | Tetrahydrofuran |
| UPLC | ultra performance liquid chromatography |
| XPhos or | Dicyclohexyl[2′,4′,6′-tris(propan-2-yl)[1,1′- |
| X-Phos | biphenyl]-2-yl]phosphane; |
| | Dicyclohexyl(2′,4′,6′-triisopropyl[biphenyl]-2- |
| | yl)phosphine; |
| | CAS-RN: [564483-18-7] |
| XPhos Pd G3; or | (2-Dicyclohexylphosphino-2′,4′,6′-triisopropyl-1,1′- |
| XPhos-Pd-G3 | biphenyl)[2-(2′-amino-1,1′-biphenyl)]palladium(II) |
| | methanesulfonate |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP—NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

NMR peak forms in the following specific experimental descriptions are stated as they appear in the spectra, possible higher order effects have not been considered.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ(intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Reactions employing microwave irradiation may be run with a Biotage Initiator® microwave oven optionally equipped with a robotic unit. The reported reaction times employing microwave heating are intended to be understood as fixed reaction times after reaching the indicated reaction temperature.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example pre-packed silica gel cartridges, e.g. from Separtis such as Isolute® Flash silica gel or Isolute® Flash NH₂ silica gel in combination with a Isolera® autopurifier (Biotage) and eluents such as gradients of e.g. hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Air and moisture sensitive liquids and solutions were transferred via syringe or cannula and introduced into reaction vessels through rubber septa. Commercial grade reagents and solvents were used without further purification. The term “concentrated in vacuo” refers to the use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

### UPLC-MS Standard Procedures

UPLC-MS-data given in the subsequent specific experimental descriptions refer (unless otherwise noted) to the following conditions:

### Method 1:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Acquity BEH C18 1.7 50 × 2.1 mm |
| Solvent: | A = water + 0.1% vol. formic acid (99%) |
| | B = acetonitrile |
| Gradient: | 0-1.6 min 1-99% B, 1.6-2.0 min 99% B |
| Flow: | 0.8 mL/min |
| Temperature: | 60° C. |
| Injection: | 2.0 μL |
| Detection: | DAD scan range 210-400 nm |
| | MS ESI+, ESI−, scan range 160-1000 m/z |
| | ELSD |

### Method 2:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Acquity BEH C18 1.7 50 × 2.1 mm |
| Solvent: | A = water + 0.2% vol. ammonia (32%) |
| | B = acetonitrile |
| Gradient: | 0-1.6 min 1-99% B, 1.6-2.0 min 99% B |
| Flow: | 0.8 mL/min |
| Temperature: | 60° C. |
| Injection: | 2.0 μL |
| Detection: | DAD scan range 210-400 nm |
| | MS ESI+, ESI−, scan range 160-1000 m/z |
| | ELSD |

### Method 3:

| | |
|---|---|
| System: | MS: Thermo Scientific FT-MS, UHPLC+: Thermo |
| | Scientific Vanquish |
| Column: | Waters, HSST3, 2.1 × 75 mm, C18 1.8 μm |
| Solvent: | A = water + 0.01% vol. formic acid (99%) |
| | B = acetonitrile + 0.01% vol. formic acid (99%) |
| Gradient: | 0-2.5 min 10-95% B, 2.5-3.5 min 95% B |
| Flow: | 0.9 mL/min |
| Temperature: | 50° C. |
| Injection: | 2.0 μL |
| Detection: | 210 nm |

### Method 4:

| | |
|---|---|
| System: | MS: Thermo Scientific FT-MS, UHPLC+: Thermo |
| | Scientific UltiMate 3000 |
| Column: | Waters, HSST3, 2.1 × 75 mm, C18 1.8 μm |
| Solvent: | A = water + 0.01% vol. formic acid (99%) |
| | B = acetonitrile + 0.01% vol. formic acid (99%) |
| Gradient: | 0-2.5 min 10-95% B, 2.5-3.5 min 95% B |
| Flow: | 0.9 mL/min |
| Temperature: | 50° C. |
| Injection: | 2.0 μL |
| Detection: | 210 nm, Optimum Integration Path 210-300 nm |

### Method 5:

| | |
|---|---|
| System: | MS: Thermo Scientific FT-MS, UHPLC+: Thermo |
| | Scientific Vanquish |
| Column: | Waters, HSST3, 2.1 × 75 mm, C18 1.8 μm |
| Solvent: | A = water + 0.01% vol. formic acid (99%) |
| | B = acetonitrile + 0.01% vol. formic acid (99%) |
| Gradient: | 0-0.75 min 60-95% B, 0.75-3.5 min 95% B |
| Flow: | 1.0 mL/min |
| Temperature: | 50° C. |
| Injection: | 2.0 μL |
| Detection: | 210 nm, Optimum Integration Path 210-300 nm |

### Method 6:

| | |
|---|---|
| System: | MS: Waters TOF instrument, UPLC: Waters Acquity |
| | I-CLASS |
| Column: | Waters, HSST3, 2.1 × 50 mm, C18 1.8 μm |
| Solvent: | A = water + 0.01% vol. formic acid (99%) |
| | B = acetonitrile + 0.01% vol. formic acid (99%) |
| Gradient: | 0-0.5 min 2% B, 0.5-7.5 min 2-95% B, 7.5-10.0 min |
| | 95% B |
| Flow: | 1.0 mL/min |
| Temperature: | 50° C. |
| Injection: | 2.0 μL |
| Detection: | 210 nm |

### Preparative HPLC Conditions

“Purification by preparative HPLC” in the subsequent experimental descriptions refers to the following conditions (unless otherwise noted):

### Method A:

Instrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: water+0.1% formic acid; Eluent B: acetonitrile; gradient: given for intermediates and examples, rate 150 mL/min, temperature 25° C.; UV 220 nm

### Method B:

Istrument: Labomatic HD-5000, pump head HDK-280, gradient module NDB-1000, fraction collector Labomatic Labocol Vario 2000, Knauer UV detector Azura UVD 2.1S, Prepcon 5 software. Column: Chromatorex C18 10 μM 120×30 mm; Eluent A: 0.1% ammonia in water; Eluent B: acetonitrile; gradient: given for intermediates and examples, rate 150 mL/min, temperature 25° C.; UV 250 nm

### Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

Chemical names were generated using the ACD/Name software from ACD/Labs. In some cases generally accepted names of commercially available reagents were used in place of ACD/Name generated names.

Optical rotations were measured using a JASCO P2000 Polarimeter. Typical, a solution of the compound with a concentration of 1 mg/mL to 15 mg/mL was used for the measurement. The specific rotation [α]_{D} was calculated according to the following formula:

$$[ α ] D = \frac{∝}{β \times d}$$

In this equation, α is the measured rotation in degrees; d is the path length in decimetres and R is the concentration in g/mL.

### EXAMPLES AND INTERMEDIATES

### Example 1

4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol

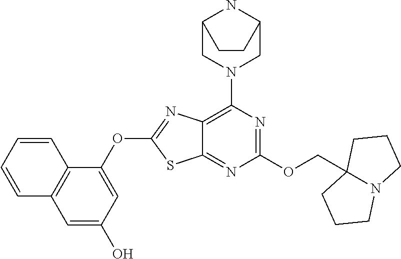 [SMILES: Oc1cc(Oc2nc3c(N4CC5CCC(C4)N5)nc(OCC45CCCN4CCC5)nc3s2)c2ccccc2c1]

tert-butyl 3-{2-[(3-methoxynaphthalen-1-yl)oxy]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (25.0 mg, 37.9 μmol) was dissolved in dichloromethane (180 μl). It was cooled to 0° C. and tribromborane (280 μl, 1.0 M in dichloromethane, 280 μmol; CAS-RN: [10294-33-4]) was added dropwise. After complete addition the ice-bath was removed and the mixture was stirred for 1.5 h. Then, it was diluted with dichloromethane and carefully quenched by addition of 25% MeOH in dichloromethane. Saturated aqueous NaHCO3 solution was added. It was extracted with a mixture of dichloromethane/isopropanol (7:3). The combined organic layers were dried using a water-resistant filter and concentrated under reduced pressure. The crude product was purified by flash chromatography using a 5 g silica column, gradient DCM/MeOH (with 5% NH3) 0-70% to give the title compound (9.50 mg, 85% purity, 39% yield).

LC-MS (method 2) Rₜ=1.15 min; MS (ESIneg): m/z=543 [M−H]⁻

### Example 1, Intermediate 1

### tert-butyl 3-(5-chloro[1,3]thiazolo[5,4-d]pyrimidin-7-yl)-3,8-diazabicyclo[3.2.1]octane-8-carboxylate

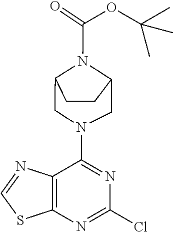 [SMILES: CC(C)(C)OC(=O)N1C2CCC1CN(c1nc(Cl)nc3scnc13)C2]

tert-butyl 3,8-diazabicyclo[3.2.1]octane-8-carboxylate (3.09 g, 14.6 mmol) and 5,7-dichloro[1,3]thiazolo[5,4-d]pyrimidine (3.00 g, 14.6 mmol) were suspended in dioxane. Triethylamine (5.1 ml, 36 mmol; CAS-RN: [121-44-8]) was added and the mixture was stirred at room temperature overnight. Afterwards, dioxane was removed by rotary evaporation. The residue was diluted with dichloromethane and water. The layers were separated and the aqueous layer was extracted with dichloromethane twice. The combined organic layers were dried using a water-resistant filter and concentrated under reduced pressure. The crude product (5.48 g) was used without purification.

LC-MS (method 2) Rₜ=1.40 min; MS (ESIpos): m/z=382 [M+H]⁺

¹H NMR (400 MHz, DMSO-d6) delta [ppm]: 1.437 (16.00), 2.518 (0.41), 3.565 (8.74), 4.294 (0.49), 9.253 (2.63)

### Example 1, Intermediate 2

### tert-butyl 3-[5-(tetrahydro-1H-pyrrolizin-7a(5H)-ylmethoxy)[1,3]thiazolo[5,4-d]pyrimidin-7-yl]-3,8-diazabicyclo[3.2.1]octane-8-carboxylate

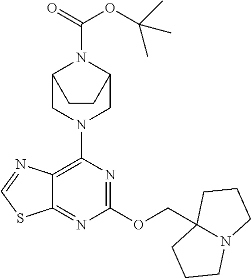 [SMILES: CC(C)(C)OC(=O)N1C2CCC1CN(c1nc(OCC34CCCN3CCC4)nc3scnc13)C2]

tert-butyl 3-(5-chloro[1,3]thiazolo[5,4-d]pyrimidin-7-yl)-3,8-diazabicyclo[3.2.1]octane-8-carboxylate (3.52 g, 9.20 mmol) and (tetrahydro-1H-pyrrolizin-7a(5H)-yl)methanol (3.90 g, 27.6 mmol) were dissolved in THF (60 ml). Sodium hydride (1.10 g, 60% purity, 27.6 mmol; CAS-RN:[7646-69-7]) was added portion wise. The reaction mixture was stirred at room temperature under Ar atmosphere for 5 minutes. Then N,N-dimethylacetamide (17 ml) was added and the reaction was heated to 75° C. for 1 h. Afterwards, the reaction mixture was carefully quenched with water and diluted with ethyl acetate. Then, the phases were separated and the organic layer was filtered through a silicone coated filter and concentrated under reduced pressure. The crude product was purified by flash chromatography (110 g column, aminophase; hexane/ethyl acetate 15%-75%) to give the title compound 3.51 g (97% purity, 76% yield)

LC-MS (method 2) Rₜ=1.48 min; MS (ESIpos): m/z=486 [M+H]⁺

1H-NMR (400 MHz, DMSO-d6) delta [ppm]: 1.153 (0.49), 1.171 (1.07), 1.189 (0.54), 1.435 (16.00), 1.519 (0.48), 1.538 (0.41), 1.549 (0.57), 1.567 (0.43), 1.612 (0.46), 1.631 (0.53), 1.713 (0.40), 1.729 (0.53), 1.746 (0.46), 1.750 (0.48), 1.766 (0.55), 1.781 (0.53), 1.796 (0.42), 1.819 (0.71), 1.835 (0.74), 1.847 (0.92), 1.861 (0.58), 1.986 (1.75), 2.523 (0.73), 2.891 (0.62), 2.905 (0.50), 2.915 (0.59), 2.941 (0.60), 3.930 (1.93), 4.275 (0.56), 8.985 (3.78).

### Example 1, Intermediate 3

### tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

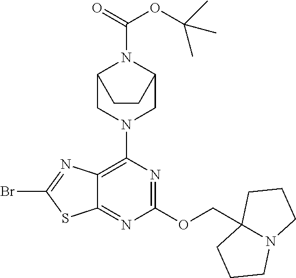 [SMILES: CC(C)(C)OC(=O)N1C2CCC1CN(c1nc(OCC34CCCN3CCC4)nc3sc(Br)nc13)C2]

To a solution of tert-butyl 3-{5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (1.00 g, 2.05 mmol) in tetrahydrofuran (THF, 8.3 ml) at −78° C. lithium bis(trimethylsilyl)amide (2.8 ml, 1.0 M in THF, 2.8 mmol; CAS-RN: [4039-32-1]) was added. It was stirred at −78° C. for 30 minutes. Then, a solution of bromine ((1.4 eq, 150 μl, 2.9 mmol; CAS-RN: [7726-95-6]) in 2.5 ml THF was added. Stirring was continued at −78° C. for 30 minutes. Afterwards, the reaction mixture was quenched with water. Then, it was diluted with 10% sodium thiosulfate solution and extracted twice with dichloromethane. The layers were separated and the organic layer was filtered through silicone filter paper and solvents were removed in vacuo. The crude product was purified by chromatography over amino phase silica with 5 to 50% ethyl acetate in hexanes to yield 395 mg (97% purity, 33% yield) of the title compound.

LC-MS (method 2) Rₜ=1.75 min; MS (ESIpos): m/z=565 [M+H]⁺

¹H-NMR (400 MHz, CHLOROFORM-d) delta [ppm]: 0.088 (0.52), 0.133 (1.50), 1.174 (0.93), 1.191 (2.08), 1.209 (1.03), 1.415 (0.75), 1.431 (16.00), 1.548 (0.42), 1.566 (0.60), 1.578 (0.47), 1.585 (0.46), 1.597 (0.48), 1.774 (0.64), 1.790 (0.65), 1.805 (0.45), 1.978 (3.58), 4.044 (0.71), 4.062 (0.72).

### Example 1, Intermediate 4

### tert-butyl 3-{2-[(3-methoxynaphthalen-1-yl)oxy]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

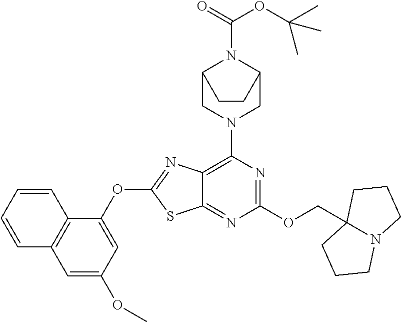 [SMILES: COc1cc(Oc2nc3c(N4CC5CCC(C4)N5C(=O)OC(C)(C)C)nc(OCC45CCCN4CCC5)nc3s2)c2ccccc2c1]

3-methoxynaphthalen-1-ol (37.0 mg, 60% purity, 127 μmol), tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (30.0 mg, 80% purity, 42.4 μmol) and potassium carbonate (29.3 mg, 212 μmol; CAS-RN: [584-08-7]) were added to N,N-dimethylacetamide (450 μl). The reaction mixture was heated to 95° C. in a microwave oven for 15 h. Afterwards, the reaction was cooled to room temperature. Water was added and it was and extracted with dichloromethane twice. The layers were separated and the organic layer was filtered through silicone paper and solvents were removed in vacuo. The crude product was purified by chromatography over amino phase silica with a gradient of 0 to 70% ethyl acetate in hexane to give 25 mg (84% purity, 25% yield) of the title compound.

LC-MS (method 1) Rₜ=1.27 min; MS (ESIpos): m/z=659 [M+H]⁺

1H-NMR (400 MHz, CHLOROFORM-d) delta [ppm]: 0.805 (0.74), 0.824 (0.47), 0.847 (0.45), 1.066 (0.61), 1.088 (0.75), 1.136 (0.45), 1.138 (1.68), 1.319 (3.09), 1.414 (16.00), 1.431 (2.94), 1.545 (0.61), 1.564 (0.44), 1.577 (0.65), 1.597 (0.63), 1.618 (0.42), 1.737 (0.47), 1.753 (0.92), 1.769 (0.95), 1.786 (0.73), 1.800 (0.63), 1.809 (0.43), 1.813 (0.46), 1.817 (0.44), 1.956 (0.42), 1.973 (0.53), 1.989 (0.41), 2.012 (2.59), 2.320 (0.41), 2.533 (0.44), 2.559 (0.47), 2.870 (2.46), 2.941 (4.15), 3.002 (0.52), 3.016 (0.45), 3.028 (0.48), 3.869 (6.40), 3.943 (0.74), 7.017 (0.60), 7.023 (0.69), 7.088 (1.22), 7.094 (1.03), 7.296 (0.49), 7.426 (0.54), 7.701 (0.57), 7.722 (0.48), 8.422 (0.44).

### Example 1 Building Block 1

### 3-methoxynaphthalen-1-ol

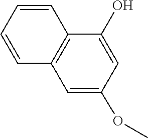

A mixture of 1-bromo-3-methoxynaphthalene (190 mg, 801 μmol), pd2dba3 (51.4 mg, 56.1 μmol; CAS-RN: [52409-22-0]), tBuXPhos (51.0 mg, 120 μmol; CAS-RN: [564483-19-8]) and KOH (225 mg, 4 mmol) was dissolved in dioxane (2.1 ml) and H₂O (870 μl). The mixture was degassed with N₂ for 5 min and heated in a microwave oven to 95° C. for 75 min. Then, the reaction was cooled to room temperature. 1N hydrochloric acid was added and it was extracted twice with dichloromethane. The combined organic layers were filtered through silicone paper and solvents were removed in vacuo. The residue was purified by chromatography over silica gel using a gradient of 10 to 45% EtOAc in hexane to afford the product as an orange residue 42.0 mg (60% purity, 18% yield).

LC-MS (method 2) Rₜ=0.73 min; MS (ESIneg): m/z=173 [M−H]⁻

¹H-NMR (400 MHz, CHLOROFORM-d) delta [ppm]: 0.952 (1.14), 0.969 (1.15), 1.125 (1.48), 1.143 (1.45), 1.278 (1.60), 1.282 (4.10), 1.295 (1.21), 1.315 (3.91), 1.624 (1.24), 2.410 (3.62), 3.829 (0.40), 3.900 (16.00), 3.917 (0.93), 5.757 (0.60), 6.506 (2.41), 6.512 (2.46), 6.722 (0.48), 6.762 (1.88), 6.766 (1.49), 6.977 (0.82), 7.310 (0.68), 7.313 (0.66), 7.327 (0.86), 7.331 (1.30), 7.334 (0.72), 7.348 (0.90), 7.351 (0.90), 7.407 (0.67), 7.413 (0.42), 7.416 (0.72), 7.419 (0.40), 7.424 (0.79), 7.428 (0.94), 7.431 (0.84), 7.445 (0.62), 7.448 (1.34), 7.451 (0.88), 7.465 (0.72), 7.468 (0.64), 7.518 (0.41), 7.558 (0.60), 7.685 (1.21), 7.705 (1.02), 8.058 (0.98), 8.079 (0.93).

### Example 2

### 4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-5-ethynyl-2-naphthol

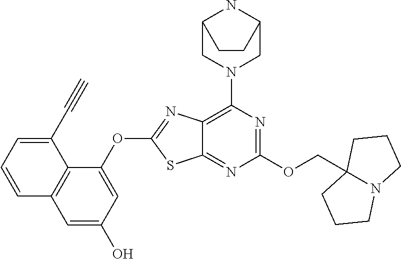 [SMILES: C#Cc1cccc2cc(O)cc(Oc3nc4c(N5CC6CCC(C5)N6)nc(OCC56CCCN5CCC6)nc4s3)c12]

4-[7-(3,8-diazabicyclo[3.2.1]octan-3-yl)-5-(1,2,3,5,6,7-hexahydropyrrolizin-8-ylmethoxy)thiazolo[5,4-d]pyrimidin-2-yl]oxy-5-(2-triisopropylsilylethynyl)naphthalen-2-ol (61 mg, 76 μmol) was dissolved in tetrahydrofuran (512 μL) to which was added a 1M solution of tetra-n-butylammonium fluoride in tetrahydrofuran (168 μL, 168 μmol; CAS-RN: 429-41-4) and the reaction was heated to 55° C. in a sealed vessel under N₂ for 16 hours. Then, the reaction was cooled to room temperature and diluted with water. The mixture was extracted three times with methylene chloride and the combined organic layers were washed with water and brine, dried by passage through a water-resistant filter and concentrated in vacuo. The crude product was purified by reverse phase HPLC using acidic conditions to afford the product 6.0 mg (99% purity, 12% yield).

LC-MS (method 1) Rₜ=0.73 min; MS (ESIpos): m/z=570 [M+H]⁺

1H-NMR (400 MHz, DMSO-d6) delta [ppm]: 0.847 (1.71), 0.865 (4.45), 0.884 (2.29), 1.026 (1.78), 1.035 (6.94), 1.042 (1.82), 1.052 (16.00), 1.070 (7.00), 1.232 (0.88), 1.251 (0.76), 1.270 (0.84), 1.288 (0.61), 1.333 (0.63), 1.351 (0.75), 1.371 (0.47), 1.487 (0.88), 1.505 (1.00), 1.520 (0.67), 1.539 (1.04), 1.551 (0.82), 1.557 (0.96), 1.569 (1.04), 1.587 (1.10), 1.607 (0.71), 1.705 (0.53), 1.719 (0.82), 1.735 (1.02), 1.751 (0.76), 1.769 (0.75), 1.783 (1.00), 1.798 (1.06), 1.813 (0.86), 1.825 (1.27), 1.838 (0.88), 1.851 (1.10), 1.865 (0.76), 1.881 (0.41), 1.904 (0.45), 2.194 (0.76), 2.323 (0.86), 2.327 (1.22), 2.331 (0.90), 2.337 (0.53), 2.343 (0.82), 2.361 (1.10), 2.378 (0.71), 2.518 (4.16), 2.523 (2.90), 2.560 (0.78), 2.565 (0.67), 2.575 (0.73), 2.580 (0.82), 2.600 (0.53), 2.660 (0.41), 2.665 (0.86), 2.669 (1.18), 2.673 (0.82), 2.915 (0.57), 2.929 (1.10), 2.942 (0.94), 2.954 (0.98), 2.968 (0.57), 3.021 (0.63), 3.051 (0.65), 3.243 (0.63), 3.350 (1.57), 3.410 (2.98), 3.428 (5.86), 3.446 (5.20), 3.463 (2.14), 3.504 (0.55), 3.522 (0.45), 3.911 (3.82), 4.233 (4.18), 7.229 (0.90), 7.235 (4.53), 7.243 (0.65), 7.421 (0.76), 7.439 (1.20), 7.460 (1.12), 7.514 (1.29), 7.517 (1.37), 7.532 (0.92), 7.535 (0.84), 7.852 (1.00), 7.856 (1.00), 7.873 (0.94), 7.877 (0.84), 8.224 (0.55).

### Example 2, Intermediate 1

### tert-butyl 3-{2-[(3-[(2-methoxyethoxy)methoxy]-8-{[tri(propan-2-yl)silyl]ethynyl}naphthalen-1-yl)oxy]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

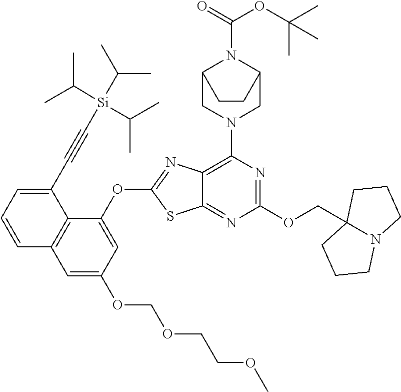 [SMILES: COCCOCOc1cc(Oc2nc3c(N4CC5CCC(C4)N5C(=O)OC(C)(C)C)nc(OCC45CCCN4CCC5)nc3s2)c2c(C#C[Si](C(C)C)(C(C)C)C(C)C)cccc2c1]

A mixture of tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (80.0 mg, 141 μmol), 3-[(2-methoxyethoxy)methoxy]-8-{[tri(propan-2-yl)silyl]ethynyl}naphthalen-1-ol (121 mg, 283 μmol), potassium carbonate (98 mg, 707 μmol; CAS-RN: [584-08-7]) in N,N-dimethylacetamide (1.5 ml) was stirred at 95° C. for 15 hours. Afterwards, the mixture was heated to 130° C. for 2 hours in the microwave reactor, then heated to 110° C. in the microwave reactor for a further 15 hours. Then, the reaction mixture was diluted with water and methylene chloride. It was extracted three times with methylene chloride, the combined organic layers were washed once with water and brine, filtered through a silicone coated filter and concentrated under reduced pressure. The crude product was purified by flash chromatography 11 g column, aminophase; hexane/ethyl acetate 0%-25% ethyl acetate to give the title compound 76.0 mg (84% purity, 49% yield).

LC-MS (methode 1) Rₜ=1.54 min; MS (ESIpos): m/z=914 [M+H]⁺

¹H-NMR (400 MHz, DMSO-d6) delta [ppm]: 0.794 (0.84), 0.813 (0.53), 0.819 (0.41), 0.836 (0.47), 0.844 (0.40), 0.862 (0.59), 0.924 (0.61), 0.930 (0.47), 0.939 (0.97), 0.960 (5.56), 0.972 (13.41), 0.990 (3.22), 1.006 (1.32), 1.026 (0.43), 1.034 (0.85), 1.074 (0.46), 1.083 (1.79), 1.100 (0.60), 1.113 (0.82), 1.120 (0.62), 1.134 (1.19), 1.139 (2.58), 1.154 (4.36), 1.172 (8.89), 1.190 (4.37), 1.259 (2.02), 1.416 (16.00), 1.436 (3.35), 1.498 (0.58), 1.510 (0.53), 1.517 (0.64), 1.530 (0.82), 1.546 (0.95), 1.565 (0.64), 1.693 (0.53), 1.709 (0.65), 1.726 (0.50), 1.739 (0.54), 1.755 (0.80), 1.769 (0.86), 1.783 (1.07), 1.791 (1.23), 1.800 (0.95), 1.811 (1.07), 1.824 (0.90), 1.840 (0.48), 1.955 (9.51), 1.987 (13.95), 2.331 (0.49), 2.518 (3.28), 2.523 (2.43), 2.673 (0.50), 2.781 (9.18), 2.878 (0.60), 2.892 (0.56), 2.903 (0.60), 2.917 (0.40), 2.941 (13.71), 3.050 (0.43), 3.068 (0.42), 3.181 (11.40), 3.195 (0.84), 3.223 (1.13), 3.444 (1.07), 3.452 (1.03), 3.455 (1.23), 3.460 (1.03), 3.468 (1.35), 3.747 (1.21), 3.755 (0.96), 3.759 (1.27), 3.763 (1.01), 3.771 (1.06), 3.902 (1.51), 3.999 (1.15), 4.017 (3.38), 4.035 (3.34), 4.053 (1.17), 5.111 (0.68), 5.413 (2.95), 5.759 (3.41), 7.436 (1.41), 7.443 (1.44), 7.533 (0.53), 7.552 (0.78), 7.573 (0.68), 7.594 (1.14), 7.600 (1.12), 7.637 (0.88), 7.640 (0.95), 7.655 (0.64), 7.658 (0.60), 7.989 (0.65), 7.992 (0.70), 8.010 (0.65), 8.013 (0.60), 8.991 (0.43), 10.851 (0.80).

### Example 2, Intermediate 2

### 4-[7-(3,8-diazabicyclo[3.2.1]octan-3-yl)-5-(1,2,3,5,6,7-hexahydropyrrolizin-8-ylmethoxy)thiazolo[5,4-d]pyrimidin-2-yl]oxy-5-(2-triisopropylsilylethynyl)naphthalen-2-ol

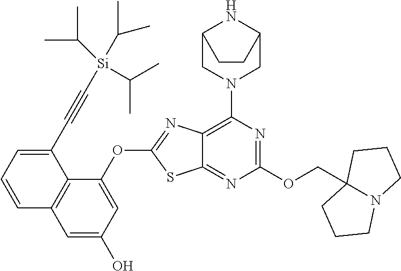 [SMILES: CC(C)[Si](C#Cc1cccc2cc(O)cc(Oc3nc4c(N5CC6CCC(C5)N6)nc(OCC56CCCN5CCC6)nc4s3)c12)(C(C)C)C(C)C]

A solution of tert-butyl 3-[5-(1,2,3,5,6,7-hexahydropyrrolizin-8-ylmethoxy)-2-[[13-(2-methoxyethoxymethoxy)-8-(2-triisopropylsilylethynyl)-1-naphthyl]oxy]thiazolo[5,4-d]pyrimidin-7-yl]-3,8-diazabicyclo[3.2.1]octane-8-carboxylate (59 mg, 78 μmol) in acetonitrile (620 μL) was cooled to 0° C. to which 4 M HCl in dioxane (624 μL, 2.49 mmol) was added. After 90 min, the reaction mixture was concentrated under reduced pressure to afford the product 57 mg (82% purity, 99% yield) which was used directly in the next reaction.

LC-MS (method 2) Rr 1.19 min; MS (ESIneg): m/z=725 [M+H]⁺

### Example 2 Building Block 1, 1. Step

### 8-{[tri(propan-2-yl)silyl]ethynyl}naphthalene-1,3-diol

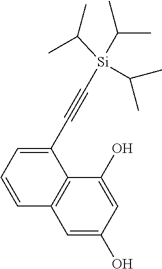 [SMILES: CC(C)[Si](C#Cc1cccc2cc(O)cc(O)c12)(C(C)C)C(C)C]

A mixture of naphthalene-1,3-diol (490 mg, 3.06 mmol, CAS: 132-86-5), (bromoethynyl)tri(propan-2-yl)silane (959 mg, 3.67 mmol, CAS: 111409-79-1), dichloro(P-cymene)ruthenium(II) dimer (187 mg, 306 μmol, CAS: 52462-29-0) and potassium acetate (600 mg, 6.12 mmol; CAS-RN: [127-08-2]) in 1,4-dioxane (5.9 ml) was stirred at 110° C. under Ar atmosphere overnight. Afterwards, the reaction mixture was diluted with ethyl acetate and water. It was extracted three times with ethyl acetate, the combined organic layers were washed once with water and brine, filtered through a silicone coated filter and concentrated under reduced pressure. The crude product was purified by flash chromatography. 50 g column, silica ULTRA; methylene chloride/ethanol 0%-3% to give the title compound (456 mg, 88% purity, 39% yield).

¹H-NMR (400 MHz, DMSO-d6) delta [ppm]: 0.959 (0.54), 1.110 (16.00), 1.132 (1.14), 6.510 (0.53), 6.516 (0.60), 6.577 (0.55), 6.583 (0.46), 7.294 (0.43), 7.297 (0.45), 9.586 (1.32), 9.945 (1.23).

### Example 2 Building Block 1, 2. Step

### 3-[(2-methoxyethoxy)methoxy]-8-{[tri(propan-2-yl)silyl]ethynyl}naphthalen-1-ol

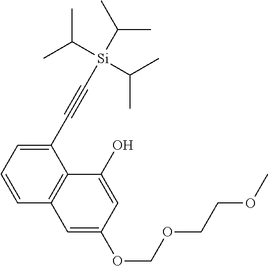 [SMILES: COCCOCOc1cc(O)c2c(C#C[Si](C(C)C)(C(C)C)C(C)C)cccc2c1]

8-{[tri(propan-2-yl)silyl]ethynyl}naphthalene-1,3-diol (456 mg, 88% purity, 1.18 mmol) was dissolved in dichloromethane 5.7 ml). N,N-diisopropylethylamine (510 μl, 2.9 mmol; CAS-RN: [7087-68-5]) was added. The mixture was cooled to 0° C. and 1-(chloromethoxy)-2-methoxyethane (MEM-Cl, 240 μl, 2.1 mmol, CAS: 3970-21-6) was added carefully. It was stirred for 30 min at 0° C. under Argon atmosphere. Afterwards, the reaction mixture was diluted with water and methylene chloride. It was extracted three times, washed once with water and brine, filtered through a silicone coated filter and concentrated under reduced pressure. The crude product was purified by flash chromatographie. 25 g column, silica sfär; hexane/ethyl acetate 0%-25% to yield the title compound (261 mg, 99% purity, 51% yield).

¹H-NMR (400 MHz, DMSO-d6) delta [ppm]: 1.116 (16.00), 3.205 (5.30), 3.444 (0.41), 3.456 (0.48), 3.468 (0.52), 3.714 (0.51), 3.726 (0.49), 3.737 (0.41), 5.290 (1.38), 6.622 (0.49), 6.629 (0.51), 6.900 (0.43), 6.905 (0.41), 7.410 (0.40), 10.171 (1.14).

### Example 3

### 4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-fluoronaphthalen-2-ol

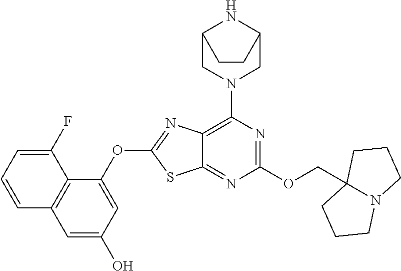 [SMILES: Oc1cc(Oc2nc3c(N4CC5CCC(C4)N5)nc(OCC45CCCN4CCC5)nc3s2)c2c(F)cccc2c1]

A solution of [tert-butyl (1R,5S)-3-{2-[(8-fluoro-3-methoxynaphthalen-1-yl)oxy]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (30.0 mg, 44.3 μmol) in dichloromethane (210 μl) was cooled to 0° C. Under cooling a solution of tribromboran, (220 μl, 1M in dichloromethane, 220 μmol; CAS-RN: [10294-33-4]) was added dropwise. After complete addition the ice-bath was removed and the mixture was stirred for 2 hours at room temperature.

Afterwards, it was evaporated in vacuo and the residue was suspended in water and basified with saturated NaHCO₃ to pH 10. The formed precipitate was filtered off and dried overnight in a vacuum oven at 50° C. The title compound was isolated as pale brown solid (16 mg, 95% purity, 61% yield).

LC-MS (method 1) Rₜ=0.77 min; MS (ESIpos): m/z=564 [M+H]⁺

¹H-NMR (400 MHz, DMSO-d6) delta [ppm]: 0.828 (0.86), 0.845 (2.16), 0.861 (1.30), 0.878 (0.65), 0.899 (0.86), 0.918 (0.43), 1.224 (16.00), 1.273 (0.86), 1.290 (0.65), 1.347 (0.86), 1.380 (0.86), 1.417 (3.03), 1.433 (3.46), 1.490 (1.51), 1.510 (3.68), 1.522 (3.24), 1.529 (3.68), 1.541 (5.41), 1.559 (4.97), 1.647 (0.65), 1.663 (1.51), 1.679 (1.95), 1.696 (3.24), 1.712 (3.89), 1.728 (2.38), 1.746 (2.81), 1.762 (3.89), 1.776 (4.11), 1.791 (3.89), 1.801 (4.97), 1.812 (3.89), 1.825 (4.32), 1.839 (3.03), 1.855 (1.73), 1.897 (0.65), 2.148 (0.65), 2.167 (1.08), 2.185 (0.65), 2.331 (2.38), 2.336 (1.08), 2.518 (15.57), 2.522 (11.46), 2.561 (1.95), 2.673 (2.38), 2.678 (1.08), 2.879 (2.16), 2.893 (3.89), 2.906 (3.46), 2.917 (3.46), 2.932 (2.16), 2.938 (1.30), 2.970 (2.59), 3.001 (2.59), 3.880 (14.05), 3.926 (0.65), 4.614 (0.65), 6.565 (1.95), 7.029 (2.38), 7.046 (2.59), 7.062 (2.38), 7.079 (2.59), 7.191 (8.00), 7.196 (10.38), 7.234 (4.54), 7.240 (6.70), 7.245 (3.46), 7.401 (1.95), 7.414 (1.95), 7.421 (3.03), 7.434 (3.03), 7.441 (2.16), 7.454 (1.95), 7.633 (5.19), 7.653 (4.32), 8.083 (1.30).

### Example 3, Intermediate 1

### tert-butyl (1R,5S)-3-{2-[(8-fluoro-3-methoxynaphthalen-1-yl)oxy]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

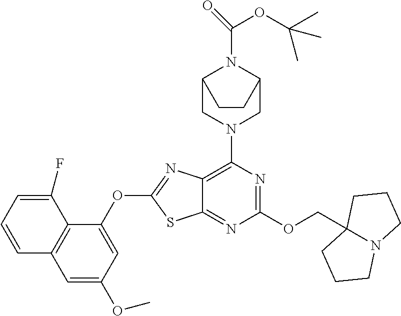 [SMILES: COc1cc(Oc2nc3c(N4CC5CCC(C4)N5C(=O)OC(C)(C)C)nc(OCC45CCCN4CCC5)nc3s2)c2c(F)cccc2c1]

A mixture of [8-fluoro-3-methoxynaphthalen-1-ol (50.0 mg, 80% purity, 208 μmol), tert-butyl (1R,5S)-3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (example 1 intermediate 3, 39.2 mg, 69.4 μmol) and potassium carbonate: (47.9 mg, 347 μmol; CAS-RN: [584-08-7]) in N,N-dimethylacetamide (740 μl) was stirred for 3.5 h at room temperature. Afterwards, it was heated to 60° C. and stirring was continued for 1 hour. Then, the reaction mixture was stirred at room temperature overnight before it was heated to 60° C. and stirred for 8 hours. Afterwards, the reaction mixture was poured into water and it was extracted with dichloromethane twice. The combined organic layers were filtered through silicone paper and solvents were removed in vacuo. The crude product was purified by flash-chromatography, amino phase, Hex/EtOAc to yield 35 mg (90% purity, 22% yield) of the title compound.

LC-MS (method 1) Rₜ=1.25 min; MS (ESIpos): m/z=677 [M+H]⁺

¹H-NMR (400 MHz, DMSO-d6) delta [ppm]: 0.794 (0.45), 1.083 (0.97), 1.154 (0.45), 1.172 (0.98), 1.190 (0.52), 1.259 (1.23), 1.402 (12.40), 1.424 (0.49), 1.434 (0.73), 1.498 (0.47), 1.518 (0.41), 1.530 (0.47), 1.548 (0.42), 1.692 (0.47), 1.708 (0.63), 1.724 (0.58), 1.737 (0.45), 1.753 (0.52), 1.767 (0.50), 1.781 (0.41), 1.794 (0.66), 1.807 (0.43), 1.819 (0.53), 1.896 (0.61), 1.955 (11.00), 1.987 (1.79), 2.518 (1.92), 2.523 (1.28), 2.781 (10.35), 2.873 (0.49), 2.886 (0.42), 2.898 (0.46), 2.942 (16.00), 3.874 (1.51), 3.934 (4.55), 4.017 (0.50), 4.034 (0.49), 7.406 (0.97), 7.412 (1.09), 7.508 (0.61), 7.513 (0.82), 7.780 (0.61), 7.799 (0.52).

### Example 3 Building Block 1

### 8-fluoro-3-methoxynaphthalen-1-ol

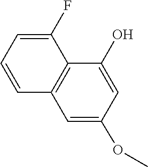

A mixture of 1-bromo-8-fluoro-3-methoxynaphthalene (122 mg, 478 μmol), pd2dba3, tBuXPhos (30.7 mg, 33.5 μmol; CAS-RN: [52409-22-0]) and KOH (158 mg, 85% purity, 2.39 mmol; CAS-RN:[1310-58-3]) in 1,4-dioxane (1.2 ml) and water (520 μl) was degassed with nitrogen for 5 min and afterwards heated to 95° C. in a microwave oven for 90 minutes. Afterwards, the reaction mixture was quenched by addition of 1M HCl and was extracted twice with dichloromethane. The combined organic layers were filtered through silicone paper and the solvents were removed in vacuo. The crude product was purified by flash chromatography; 10 g Si-column, Hex/EtOAc: 0-60% EtOAc to give the title compound 94.0 mg (70% purity, 72% yield).

LC-MS (method 2) Rₜ=1.02 min; MS (ESIpos): m/z=193 [M+H]⁺

¹H-NMR (400 MHz, DMSO-d6) delta [ppm]: 0.865 (1.25), 0.882 (1.23), 1.139 (1.22), 1.156 (1.78), 1.161 (4.28), 1.193 (4.09), 1.210 (0.45), 1.229 (0.62), 1.238 (2.31), 1.255 (2.35), 2.338 (1.51), 2.518 (0.68), 2.523 (0.49), 3.160 (0.56), 3.172 (0.55), 3.437 (0.47), 3.809 (16.00), 3.920 (0.65), 6.526 (2.75), 6.532 (2.97), 6.825 (1.12), 6.830 (2.27), 6.836 (1.08), 6.894 (0.79), 6.896 (0.82), 6.913 (1.03), 6.916 (1.79), 6.927 (0.82), 6.929 (0.86), 6.946 (0.90), 6.948 (0.89), 7.295 (0.70), 7.308 (0.73), 7.315 (0.98), 7.328 (1.01), 7.335 (0.79), 7.347 (0.97), 7.428 (0.46), 7.459 (0.44), 7.464 (0.45), 7.483 (1.51), 7.486 (1.59), 7.504 (1.31), 7.507 (1.20), 10.164 (1.01).

### Example 4

### 4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}oxy)-5-methylnaphthalen-2-ol

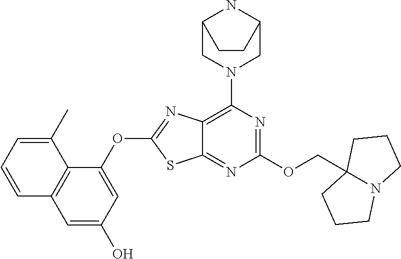 [SMILES: Cc1cccc2cc(O)cc(Oc3nc4c(N5CC6CCC(C5)N6)nc(OCC56CCCN5CCC6)nc4s3)c12]

tert-butyl 3-{2-({3-[(2-methoxyethoxy)methoxy]-8-methylnaphthalen-1-yl}oxy)-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (171 mg, 71% purity, 163 μmol) was dissolved in acetonitrile (1.3 mL). It was cooled to 0° C. and hydrochloric acid (410 μl, 4.0 M in dioxane, 1.6 mmol; CAS-RN: [7647-01-0]) was added. The reaction mixture was stirred at 0° C. for 30 minutes. Then, it was allowed to warm up to room temperature and stirred for another 30 minutes. For work up: The mixture was carefully diluted with saturated, aqueous NaHCO₃-solution till pH-value 8. A red precipitate was formed which was filtered off under vacuo. The filter cake was transferred into a flask and dried by rotary evaporation. The filter cake was dissolved in ethyl acetate and the aqueous filtrate was added. After separation of the layer the aqueous layer was extracted with ethyl acetate twice. The combined organic layers were dried using a water-resistant filter. The clear filtrate was concentrated under reduced pressure. Red particles in the aqueous layer and in the drying filter were extracted/dissolved with a mixture of dichloromethane/isopropanole (7:3). The combined organic layers were dried using a water-resistant filter. The clear filtrate was concentrated under reduced pressure. The crude products were combined (127 mg). The material was purified by preparative HPLC to give the title compound (20.0 mg (100% purity, 19%)

LC-MS: (method 2) Rₜ=0.75 min; MS (ESIpos): m/z=559 [M+H]⁺

¹H-NMR (400 MHz, DMSO-d6) δ[ppm]: 1.228 (0.63), 1.513 (0.74), 1.530 (2.54), 1.543 (2.24), 1.549 (2.61), 1.562 (4.69), 1.580 (4.25), 1.594 (1.30), 1.658 (1.96), 1.681 (2.42), 1.695 (1.75), 1.712 (2.36), 1.729 (2.71), 1.744 (1.94), 1.761 (1.89), 1.777 (2.63), 1.791 (2.78), 1.806 (2.35), 1.818 (3.41), 1.831 (2.44), 1.843 (3.20), 1.857 (2.10), 1.873 (1.09), 2.318 (0.42), 2.322 (0.95), 2.327 (1.33), 2.332 (0.93), 2.336 (0.44), 2.518 (4.36), 2.523 (3.37), 2.564 (2.06), 2.570 (2.24), 2.589 (1.67), 2.647 (16.00), 2.660 (0.87), 2.665 (1.18), 2.669 (1.52), 2.673 (1.08), 2.678 (0.59), 2.909 (1.47), 2.922 (3.02), 2.936 (2.52), 2.947 (2.73), 2.962 (1.48), 3.152 (1.11), 3.911 (11.90), 7.100 (2.55), 7.117 (3.01), 7.128 (5.90), 7.134 (7.13), 7.173 (5.93), 7.179 (4.47), 7.309 (2.55), 7.326 (2.49), 7.329 (3.08), 7.347 (2.30), 7.625 (2.85), 7.646 (2.50), 8.261 (4.72).

### Example 4, Intermediate 1

### tert-butyl 3-[2-({3-[(2-methoxyethoxy)methoxy]-8-methyl-1-naphthyl}oxy)-5-(tetrahydro-1H-pyrrolizin-7a(5H)-ylmethoxy)[1,3]thiazolo[5,4-d]pyrimidin-7-yl]-3,8-diazabicyclo[3.2.1]octane-8-carboxylate

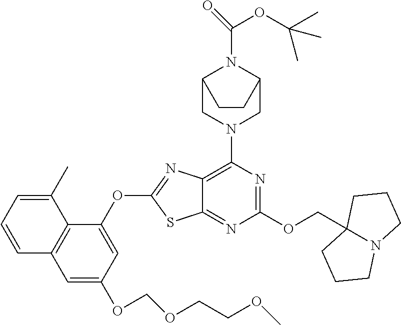 [SMILES: COCCOCOc1cc(Oc2nc3c(N4CC5CCC(C4)N5C(=O)OC(C)(C)C)nc(OCC45CCCN4CCC5)nc3s2)c2c(C)cccc2c1]

A mixture of tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (97.1 mg, 173 μmol), 3-[(2-methoxyethoxy)methoxy]-8-methylnaphthalen-1-ol (85.0 mg, 80% purity, 259 μmol), potassium carbonate: (119 mg, 864 μmol; CAS-RN: [584-08-7]) in N,N-dimethylacetamide (1.8 ml) was heated at 95° C. under microwave irradiation for 15 h. Afterwards, the mixture was diluted with water and dichloromethane. The aqueous layer was extracted with dichloromethane once again. The combined organic layers were washed with half saturated brine and dried using a water-resistant filter. The clear filtrate was concentrated under reduced pressure. The crude product 171 mg (71% purity, 94% yield) was used without further purification.

LC-MS (method 2) Rₜ=1.30 min; MS (ESIpos): m/z=747 [M+H]⁺

### Example 4 Building Block 1, 1. Step

### 2-(5-bromo-4-methyl-2-naphthyl)-4,4,5,5-tetramethyl-1,3,2-dioxaborolane

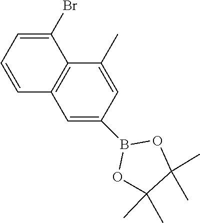 [SMILES: Cc1cc(B2OC(C)(C)C(C)(C)O2)cc2cccc(Br)c12]

1-bromo-8-methylnaphthalene (999 mg, 4.52 mmol), 4,4,4′,4′,5,5,5′,5′-octamethyl-2,2′-bi-1,3,2-dioxaborolane (1.26 g, 4.97 mmol; CAS-RN: [73183-34-3]), bis(1,5-yclooctadiene)dimethoxydiiridium (150 mg, 226 μmol; CAS-RN: [12148-71-9]) and 4,4′-di-tert-butyl-2,2′-bipyridine (121 mg, 452 μmol; CAS-RN: [72914-19-3]) were dissolved in cyclohexane (17 mL). Nitrogen was bubbled through the mixture for 30 seconds and then the reaction was stirred at 80° C. for two hours in a sealed vessel (microwave), The reaction was concentrated in vacuo and the crude product, still containing reagents and some solvent was used directly in the following step.

### Example 4 Building Block 1, 2. Step

### 4-bromo-5-methyl-naphthalen-2-ol

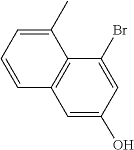

The crude material of building block 1, step 1 was dissolved in 1:1 THF/H₂O (7 ml+7 ml) to which was added sodium perborate tetrahydrate (6.74 g, 43.8 mmol). The mixture was stirred at room temperature for 2 hours. Then reaction mixture was extracted with dichloromethane. The organic layer was washed with 25% brine and then filtered through silicone paper. Solvents were removed in vacuo and the residue was purified by chromatography over SiO₂ using a gradient of 15 to 60% ethyl acetate in hexane to yield a mixture of isomers. The major isomer was isolated by repeated chromatography and assigned by HNMR as tittle structure (296 mg, 27%). HNMR structural assignment was based on literature values for the Methyl CH3 signal, which should appear at about 3.1 ppm for the title compound and 2.6 ppm for 5-bromo-4-methyl-naphthalen-2-ol (see literature Journal of the American Chemical Society, 2021, vol. 143, #28, p. 10686-10694).

¹H-NMR (400 MHz, CHLOROFORM-d) delta [ppm]: 1.273 (0.78), 1.584 (6.94), 2.063 (1.13), 3.085 (16.00), 4.851 (2.34), 4.865 (1.74), 7.123 (4.13), 7.130 (4.27), 7.184 (1.09), 7.188 (1.49), 7.190 (1.18), 7.202 (1.66), 7.204 (2.14), 7.207 (1.68), 7.287 (2.64), 7.305 (1.70), 7.504 (5.05), 7.510 (5.06), 7.537 (1.85), 7.539 (2.01), 7.559 (1.71), 7.561 (1.58).

### Example 4 Building Block 1, 3. Step

### 1-bromo-3-[(2-methoxyethoxy)methoxy]-8-methylnaphthalene

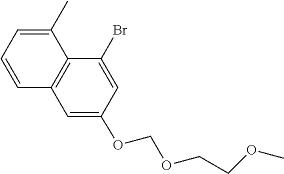 [SMILES: COCCOCOc1cc(Br)c2c(C)cccc2c1]

4-bromo-5-methylnaphthalen-2-ol (296 mg, 1.25 mmol) was dissolved in dichloromethane (6.0 ml). N,N-diisopropylethylamine (540 μl, 3.1 mmol; CAS-RN: [7087-68-5]) was added. The reaction mixture was put under nitrogen atmosphere and cooled to 0° C. At this temperature 1-(chloromethoxy)-2-methoxyethane (250 μl, 2.2 mmol) was carefully added. It was stirred for 30 min at 0° C. and at room temperature overnight. Afterwards, the reaction mixture was diluted with water and dichloromethane. It was extracted with dichloromethane twice. The combined organic layers were dried using a water-resistant filter and concentrated under reduced pressure to give ethe title compounds (407 mg, 90% purity, 90% yield).

¹H-NMR (400 MHz, DMSO-d6) δ[ppm]: 2.518 (0.60), 2.523 (0.42), 3.006 (9.26), 3.224 (0.57), 3.240 (4.45), 3.242 (2.65), 3.333 (16.00), 3.433 (0.48), 3.442 (0.46), 3.445 (0.71), 3.449 (0.60), 3.459 (3.08), 3.467 (2.20), 3.470 (3.00), 3.474 (2.19), 3.482 (3.21), 3.582 (0.60), 3.595 (0.55), 3.597 (0.46), 3.606 (0.42), 3.747 (3.11), 3.756 (2.05), 3.759 (2.99), 3.763 (2.06), 3.771 (2.72), 4.695 (2.31), 4.710 (0.41), 5.373 (11.02), 7.274 (0.98), 7.276 (0.76), 7.288 (1.11), 7.291 (1.45), 7.346 (1.50), 7.367 (1.71), 7.384 (1.08), 7.510 (2.42), 7.517 (2.70), 7.621 (3.44), 7.627 (2.93), 7.733 (1.25), 7.753 (1.11).

### Example 4 Building Block 1, 4. Step

### 3-[(2-methoxyethoxy)methoxy]-8-methylnaphthalen-1-ol

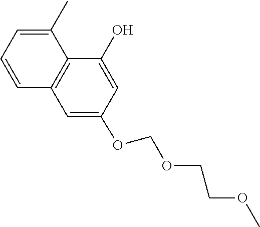 [SMILES: COCCOCOc1cc(O)c2c(C)cccc2c1]

To a solution of 1-bromo-3-[(2-methoxyethoxy)methoxy]-8-methylnaphthalene (400 mg, 1.23 mmol) in dioxane (3.2 mL) and water (1.3 mL) potassium hydroxide (345 mg, 6.15 mmol; CAS-RN: [1310-58-3]), Pd2(dba)3 (78.8 mg, 86.1 μmol; CAS-RN: [52409-22-0]) and di-tert-butylphosphino-2′,4′,6′-triisopropylbiphenyl (78.3 mg, 184 μmol; CAS-RN: [564483-19-8]) were added. It was degased with N₂ for 5 minutes. Then, the mixture was heated for 75 min. at 95° C. in a microwave reactor.

Afterwards, the mixture was diluted with dichloromethane and remaining catalyst. was filtered off over celite. The clear filtrate was diluted with 1-molar aqueous HCl-solution. The layers were separated and the aqueous layer was extracted once again. The combined organic layers were dried using a water-resistant filter. The clear filtrate was concentrated under reduced pressure. The crude product was purified using a 10 g spheric silica column, gradient hexanes/EtOAc 0-75% to yield the title compound (178 mg, 80% purity, 44% yield).

LC-MS (method 2) Rₜ=1.11 min; MS (ESIpos): m/z=263 [M+H]⁺

¹H-NMR (400 MHz, DMSO-d6) δ[ppm]: 0.866 (0.45), 0.882 (0.44), 1.140 (0.44), 1.161 (1.55), 1.194 (1.49), 1.238 (0.84), 1.255 (0.78), 1.988 (0.44), 2.339 (0.48), 2.518 (0.53), 2.797 (5.45), 3.146 (0.58), 3.181 (0.40), 3.217 (1.11), 3.224 (16.00), 3.240 (1.08), 3.242 (0.56), 3.460 (1.64), 3.469 (1.37), 3.472 (1.84), 3.476 (1.36), 3.484 (1.93), 3.724 (1.88), 3.732 (1.24), 3.736 (1.81), 3.739 (1.26), 3.748 (1.56), 4.695 (0.53), 5.283 (5.62), 6.563 (1.73), 6.569 (1.81), 6.836 (1.62), 6.842 (1.52), 6.917 (0.43), 6.958 (0.78), 6.973 (0.64), 6.975 (0.89), 6.978 (0.61), 7.181 (0.82), 7.198 (0.87), 7.201 (1.04), 7.218 (0.86), 7.451 (0.90), 7.472 (0.77), 10.032 (1.10).

### Example 5, Intermediate 1

### tert-butyl (1R,5S)-3-(2-{[7-fluoro-3-(methoxymethoxy)-8-{[tri(propan-2-yl)silyl]ethynyl}naphthalen-1-yl]oxy}-5-[(tetrahydro-1H-pyrrolizin-7a(5H)-yl)methoxy][1,3]thiazolo[5,4-d]pyrimidin-7-yl)-3,8-diazabicyclo[3.2.1]octane-8-carboxylate

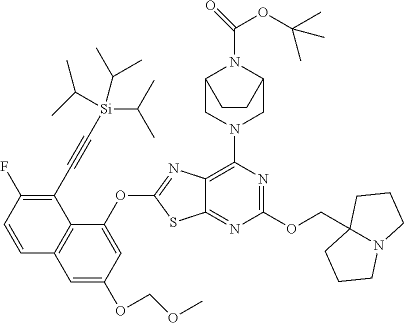 [SMILES: COCOc1cc(Oc2nc3c(N4CC5CCC(C4)N5C(=O)OC(C)(C)C)nc(OCC45CCCN4CCC5)nc3s2)c2c(C#C[Si](C(C)C)(C(C)C)C(C)C)c(F)ccc2c1]

tert-butyl (1R,5S)-3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (60.0 mg, 106 μmol, example 1, intermediate 3), 7-fluoro-3-(methoxymethoxy)-8-{[tri(propan-2-yl)silyl]ethynyl}naphthalen-1-ol (85.4 mg, 212 μmol, see WO 2021/041671, intermediate 15, page 96), potassium carbonate: (73.3 mg, 530 μmol; CAS-RN: [584-08-7]) were added to N,N-dimethylacetamide (1.1 ml). The reaction mixture was stirred at 110° C. for 1 hour in a microwave reactor. Afterwards, the reaction was poured into water and it was extracted with dichloromethane twice. The combined organic layers were filtered through silicone paper and solvents removed in vacuo. The crude product was purified by flash-chromatography (silica phase, hexane/ethyl acetate) to yield the title compound (48.0 mg 90% purity, 46% yield).

LC-MS (method 1) Rₜ=1.61 min; MS (ESIpos): m/z=888 [M+H]⁺

¹H-NMR (400 MHz, DMSO-d6) δ[ppm]: 0.958 (5.32), 0.971 (11.31), 0.986 (2.02), 0.997 (1.43), 1.015 (0.70), 1.033 (0.49), 1.052 (0.59), 1.070 (0.82), 1.088 (0.46), 1.135 (0.94), 1.154 (0.51), 1.158 (0.65), 1.172 (0.74), 1.231 (0.72), 1.413 (16.00), 1.436 (1.25), 1.505 (0.61), 1.524 (0.98), 1.536 (0.94), 1.545 (0.78), 1.552 (0.75), 1.697 (0.51), 1.713 (0.63), 1.730 (0.48), 1.743 (0.51), 1.758 (0.74), 1.772 (0.96), 1.787 (1.10), 1.795 (1.19), 1.803 (0.85), 1.815 (0.88), 1.829 (0.59), 1.903 (1.14), 1.956 (11.01), 1.987 (1.32), 2.331 (0.47), 2.518 (2.76), 2.523 (2.19), 2.673 (0.47), 2.781 (10.57), 2.884 (0.63), 2.898 (0.57), 2.909 (0.60), 2.941 (16.00), 3.362 (0.63), 3.413 (9.27), 3.910 (1.61), 5.346 (3.78), 7.519 (1.00), 7.524 (1.08), 7.566 (0.52), 7.588 (1.00), 7.610 (0.53), 7.643 (1.23), 7.649 (1.10), 8.072 (0.48), 8.087 (0.54), 8.096 (0.51), 8.109 (0.47).

### Example 5

### 4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-ethynyl-6-fluoronaphthalen-2-ol

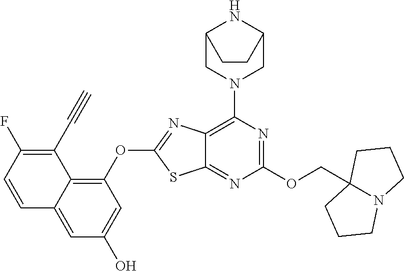 [SMILES: C#Cc1c(F)ccc2cc(O)cc(Oc3nc4c(N5CC6CCC(C5)N6)nc(OCC56CCCN5CCC6)nc4s3)c12]

tert-butyl (1R,5S)-3-(2-{[8-ethynyl-7-fluoro-3-(methoxymethoxy)naphthalen-1-yl]oxy}-5-[(tetrahydro-1H-pyrrolizin-7a(5H)-yl)methoxy][1,3]thiazolo[5,4-d]pyrimidin-7-yl)-3,8-diazabicyclo[3.2.1]octane-8-carboxylate (40.0 mg, 54.7 μmol) was dissolved in acetonitrile (430 μl). The mixture was cooled to 0° C. and hydrochloric acid (440 μl, 4.0 M in dioxane, 1.8 mmol; CAS-RN:[7647-01-0]) was added. The reaction mixture was stirred for 2.5 hours while the temperature was allowed to warm-up die room temperature over this time. Afterwards, the reaction mixture was evaporated under vacuo. The crude product was purified by HPLC to give the title compound 1 (2.0 mg, 90% purity, 34% yield)

LC-MS (method 1) Rₜ=0.77 min; MS (ESIpos): m/z=587 [M+H]⁺

¹H-NMR (400 MHz, DMSO-d6) δ[ppm]: 1.230 (0.79), 1.465 (2.46), 1.483 (2.81), 1.521 (1.20), 1.537 (3.07), 1.549 (2.46), 1.556 (2.85), 1.568 (3.65), 1.586 (4.03), 1.670 (0.63), 1.687 (1.09), 1.702 (1.51), 1.718 (2.46), 1.734 (2.93), 1.751 (2.28), 1.768 (2.17), 1.783 (3.02), 1.798 (3.15), 1.812 (2.53), 1.824 (3.86), 1.838 (2.89), 1.842 (2.72), 1.850 (3.33), 1.864 (2.32), 1.880 (1.20), 1.903 (0.61), 2.327 (3.27), 2.332 (2.41), 2.336 (1.09), 2.365 (1.34), 2.518 (16.00), 2.523 (10.94), 2.563 (4.13), 2.571 (2.58), 2.577 (2.76), 2.596 (1.76), 2.669 (3.48), 2.673 (2.53), 2.678 (1.17), 2.710 (1.43), 2.912 (1.73), 2.927 (3.42), 2.940 (2.97), 2.952 (3.17), 2.966 (1.91), 2.994 (14.92), 3.032 (2.12), 3.165 (1.24), 3.386 (6.79), 3.910 (11.97), 3.937 (1.22), 4.569 (6.96), 4.572 (7.20), 4.605 (0.83), 4.654 (0.89), 5.758 (1.91), 7.261 (0.41), 7.267 (0.48), 7.283 (2.75), 7.289 (7.89), 7.294 (7.55), 7.300 (2.81), 7.381 (0.51), 7.466 (2.39), 7.489 (4.83), 7.511 (2.49), 7.941 (2.18), 7.955 (2.32), 7.964 (2.31), 7.978 (2.11), 8.233 (12.17).

### Example 5, Intermediate 2

### tert-butyl (1R,5S)-3-(2-{[8-ethynyl-7-fluoro-3-(methoxymethoxy)naphthalen-1-yl]oxy}-5-[(tetrahydro-1H-pyrrolizin-7a(5H)-yl)methoxy][1,3]thiazolo[5,4-d]pyrimidin-7-yl)-3,8-diazabicyclo[3.2.1]octane-8-carboxylate

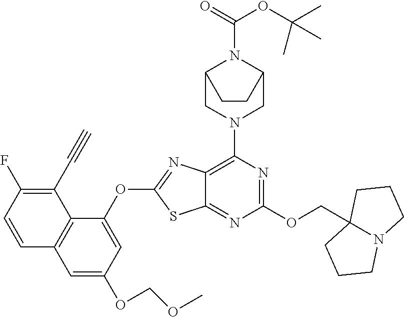 [SMILES: C#Cc1c(F)ccc2cc(OCOC)cc(Oc3nc4c(N5CC6CCC(C5)N6C(=O)OC(C)(C)C)nc(OCC56CCCN5CCC6)nc4s3)c12]

tert-butyl (1R,5S)-3-(2-{[7-fluoro-3-(methoxymethoxy)-8-{[tri(propan-2-yl)silyl]ethynyl}naphthalen-1-yl]oxy}-5-[(tetrahydro-1H-pyrrolizin-7a(5H)-yl)methoxy][1,3]thiazolo[5,4-d]pyrimidin-7-yl)-3,8-diazabicyclo[3.2.1]octane-8-carboxylate (47.0 mg, 53.0 μmol) was dissolved in tetrahydrofuran (320 μl). Tetrabutylammonium fluoride (110 μl, 1.0 M in tetrahydrofuran, 110 μmol; CAS-RN: [429-41-4]) was added and the reaction mixture was stirred under N₂ atmosphere for 1.5 hours at room temperature and for another 2 hours at 55° C. Afterwards, the reaction mixture was quenched with saturated NH₄Cl solution and was extracted with ethyl acetate. The combined organic layers were filtered through a silicone coated filter and concentrated under reduced pressure. The crude product (37 mg, 90% purity, 86% yield) was used without further purification.

LC-MS (method 2) Rₜ=1.32 min; MS (ESIpos): m/z=732 [M+H]⁺

¹H-NMR (400 MHz, DMSO-d6) δ[ppm]: 0.887 (0.69), 0.894 (0.41), 0.903 (0.98), 0.908 (0.72), 0.913 (0.56), 0.924 (1.72), 0.939 (1.48), 0.951 (0.61), 0.975 (16.00), 0.989 (7.71), 0.997 (2.14), 1.004 (0.56), 1.033 (1.30), 1.039 (0.49), 1.154 (1.84), 1.167 (0.56), 1.172 (4.04), 1.190 (2.12), 1.231 (0.80), 1.398 (13.11), 1.906 (0.81), 1.987 (7.25), 2.084 (1.89), 2.331 (0.43), 2.518 (2.42), 2.523 (1.62), 2.674 (0.50), 3.433 (8.36), 3.999 (0.68), 4.017 (1.87), 4.035 (1.97), 4.053 (0.72), 4.669 (1.17), 4.672 (1.22), 5.113 (1.43), 5.364 (3.06), 5.758 (0.60), 7.561 (0.42), 7.583 (0.82), 7.606 (0.43), 7.627 (0.60), 7.633 (0.90), 7.652 (1.13), 7.658 (0.68), 8.089 (0.41).

### Example 6

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynyl-6-fluoronaphthalen-2-ol

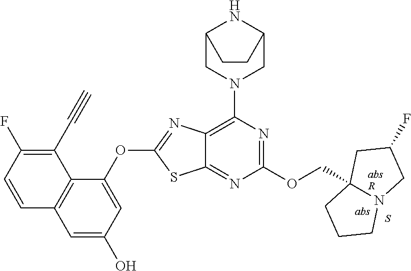

tert-butyl 3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (170 mg, 227 μmol) was dissolved in acetonitrile (1.8 ml). It was cooled to 0° C. Then HCl (1.8 ml, 4.0 M in dioxane, 7.3 mmol; CAS-RN: [7647-01-0]) was added and it was stirred at 0° C. for 30 minutes under Argon atmosphere. Afterwards, the reaction mixture was concentrated under reduced pressure. The remaining mixture was diluted with ethyl acetate and quenched with saturated NaHCO₃ solution. It was stirred for a few minutes and the organic layer was filtered through a silicone coated filter and concentrated under reduced pressure. The crude product (131 mg) was purified by HPLC chromatography with acidic conditions to give the title compound (69 mg, 100% purity, 50% yield).

LC-MS (method 1) Rₜ=0.76 min; MS (ESIpos): m/z=605 [M+H]⁺

¹H-NMR (400 MHz, DMSO-d6) δ[ppm]: 1.026 (1.28), 1.035 (7.74), 1.042 (1.37), 1.052 (16.00), 1.070 (7.09), 1.533 (1.54), 1.551 (1.75), 1.565 (0.62), 1.667 (1.24), 1.680 (1.20), 1.689 (1.21), 1.724 (1.51), 1.734 (1.11), 1.741 (1.14), 1.752 (1.07), 1.776 (0.69), 1.795 (0.43), 1.804 (0.41), 1.822 (1.04), 1.826 (0.93), 1.904 (0.58), 1.946 (0.82), 1.956 (0.63), 1.991 (1.51), 1.999 (1.70), 2.073 (1.32), 2.083 (0.93), 2.331 (0.91), 2.337 (0.41), 2.518 (4.84), 2.523 (3.38), 2.673 (0.94), 2.678 (0.45), 2.774 (0.40), 2.798 (0.79), 2.813 (0.96), 2.835 (0.47), 2.975 (1.11), 2.982 (1.46), 3.022 (0.51), 3.059 (2.31), 3.065 (1.93), 3.100 (1.47), 3.132 (1.36), 3.410 (2.65), 3.428 (7.68), 3.445 (6.45), 3.463 (2.68), 3.850 (1.95), 3.875 (2.75), 3.947 (3.04), 3.972 (2.04), 4.576 (4.50), 4.578 (4.63), 4.718 (0.48), 5.183 (0.73), 5.316 (0.70), 7.288 (2.26), 7.294 (4.70), 7.303 (4.23), 7.308 (1.92), 7.469 (1.53), 7.492 (3.03), 7.515 (1.53), 7.944 (1.35), 7.959 (1.41), 7.968 (1.41), 7.981 (1.29), 8.199 (15.36).

### Example 6, Intermediate 1

### tert-butyl 3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

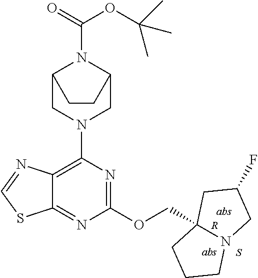

tert-butyl 3-(5-chloro[1,3]thiazolo[5,4-d]pyrimidin-7-yl)-3,8-diazabicyclo[3.2.1]octane-8-carboxylate (500 mg, 1.31 mmol, example 1, intermediate 1) and [(2R,7aS)-2-fluorotetrahydro-1H-pyrrolizin-7a(5H)-yl]methanol (625 mg, 3.93 mmol, see WO 2021/041671, intermediate 16, step E, page 100) were dissolved in tetrahydrofuran (8.5 ml). Sodium hydride (157 mg, 60% purity, 3.93 mmol; CAS-RN: [7646-69-7]) was added portion wise. It was stirred at room temperature under Argon atmosphere for 5 minutes. Then N,N-dimethylacetamide (2.4 ml) and stirring was continued for 1 hour. Afterwards, the reaction mixture was carefully quenched with water and diluted with ethyl acetate. It was stirred for a few minutes and then the organic layer was filtered through a silicone coated filter and concentrated under reduced pressure. The crude product was purified by flash chromatography (11 g colummn, aminophase; hexane/ethyl acetate 0%-75%). The obtained product was dissolved in dichloromethane and treated with half saturated NaCl solution. It was stirred for a few minutes and the organic layer was filtered through a silicone coated filter and concentrated under reduced pressure to give the title compound (584 mg, 88% yield).

¹H-NMR (400 MHz, DMSO-d6) delta [ppm]: 1.422 (0.44), 1.436 (16.00), 1.609 (0.50), 1.628 (0.59), 1.833 (0.63), 1.955 (3.93), 2.010 (0.49), 2.017 (0.50), 2.518 (0.47), 2.781 (3.67), 2.941 (5.69), 2.998 (0.69), 3.063 (0.50), 3.077 (0.48), 3.084 (0.46), 3.936 (0.61), 4.004 (0.63), 4.276 (0.57), 5.758 (0.76), 8.993 (3.42).

### Example 6, Intermediate 2

### tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

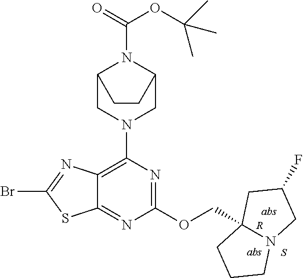

tert-butyl 3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (894 mg, 1.77 mmol) was dissolved in THF (7.2 ml) and cooled down to −78° C. Then LiHMDS, (2.4 ml, 1.0 M in THF, 2.4 mmol; CAS-RN:[4039-32-1]) was added and it was stirred at this temperature for 30 minutes. Afterwards, a solution of bromine (130 μl, 2.5 mmol) in THF (2 ml) was added drop wise. Stirring was continued at −78° C. under Argon atmosphere for 30 minutes. Then, the reaction mixture was carefully quenched with water and saturated Na₂S₂O₃ solution and diluted with ethyl acetate. It was extracted three times with ethyl acetate. The combined organic layers were washed once with water and brine, filtered through a silicone coated filter and concentrated under reduced pressure. The crude product was purified by flash chromatography 25 g column, silica ULTRA; ethy acetate/ethanol 0%-15% to give the title compound (181 mg, 97% purity, 17% yield).

LC-MS (method 1) Rₜ=1.61 min; MS (ESIpos): m/z=583 [M+H]⁺

1H-NMR (400 MHz, DMSO-d6) delta [ppm]: 1.065 (5.25), 1.156 (0.66), 1.437 (16.00), 1.630 (0.43), 1.831 (0.56), 2.000 (0.50), 2.007 (0.52), 2.518 (0.53), 2.990 (0.53), 3.058 (0.52), 3.067 (0.53), 3.074 (0.47), 3.924 (0.60), 3.936 (0.83), 3.989 (0.63), 4.283 (0.62), 5.758 (0.48).

### Example 6, Intermediate 3

### tert-butyl 3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

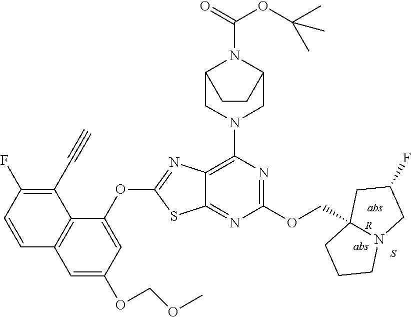

tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (175 mg, 300 μmol), 7-fluoro-3-(methoxymethoxy)-8-{[tri(propan-2-yl)silyl]ethynyl}naphthalen-1-ol (241 mg, 600 μmol, see WO 2021/041671, intermediate 15, page 96) and potassium carbonate (207 mg, 1.50 mmol; CAS-RN: [584-08-7]) were dissolved in N,N-dimethylacetamide (3.2 ml). The reaction mixture was stirred in a microwave reactor at 110° C. for 15 hours. Afterwards, the reaction mixture was diluted with water and dichloromethane. It was extracted with dichloromethane twice. The combined organic layers were dried using a water-resistant filter and concentrated under reduced pressure. The crude material was purified by flash chromatography using a 11 g spheric amino column, gradient hexanes/EtOAc 0-50%. To give the title compound (174 mg, 77.7%).

1H-NMR (400 MHz, DMSO-d6) delta [ppm]: 1.065 (0.95), 1.396 (8.39), 1.436 (0.68), 1.955 (10.77), 2.518 (0.54), 2.781 (10.08), 2.941 (16.00), 2.981 (0.45), 3.431 (5.27), 4.670 (0.76), 4.672 (0.80), 5.363 (1.94), 7.579 (0.54), 7.631 (0.59), 7.646 (0.77), 7.653 (0.44).

### Example 7

### 4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}amino)naphthalen-2-ol

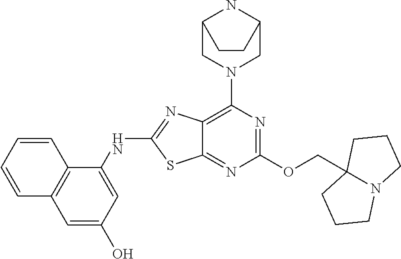 [SMILES: Oc1cc(Nc2nc3c(N4CC5CCC(C4)N5)nc(OCC45CCCN4CCC5)nc3s2)c2ccccc2c1]

tert-butyl 3-{2-[(3-methoxynaphthalen-1-yl)amino]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (31.0 mg, 47.1 μmol) was dissolved in dichloromethane. The solution was cooled to 0° C. and tribromborane (710 μl, 1.0 M in dichloromethane, 710 μmol; CAS-RN: [10294-33-4]) was added dropwise. It was stirred at 0° C. for 30 minutes under Argon atmosphere. Afterwards, the reaction mixture was poured into saturated NaHCO₃ solution. Dichloromethane/isopropanole 7:3 was added and it was stirred for 5 minutes. The organic layer was filtered through a silicone coated filter and concentrated under reduced pressure. The crude product was purified by HPLC chromatography with acidic conditions to give the title compound (11.0 mg, 100% purity, 37% yield).

LC-MS (method 1) Rₜ=0.67 min; MS (ESIpos): m/z=544 [M+H]⁺

¹H-NMR (400 MHz, DMSO-d6) δ[ppm]: 1.026 (9.62), 1.035 (4.88), 1.042 (10.00), 1.052 (11.76), 1.070 (5.91), 1.159 (0.46), 1.230 (1.79), 1.397 (0.48), 1.414 (0.94), 1.432 (0.49), 1.574 (1.16), 1.591 (3.13), 1.603 (2.30), 1.610 (2.64), 1.622 (3.38), 1.639 (2.70), 1.699 (2.85), 1.730 (3.52), 1.748 (3.29), 1.762 (3.66), 1.779 (3.61), 1.796 (3.20), 1.809 (2.90), 1.824 (3.63), 1.839 (3.62), 1.854 (2.64), 1.870 (3.75), 1.888 (2.96), 1.901 (3.82), 1.915 (2.90), 1.931 (1.53), 2.084 (0.50), 2.193 (2.88), 2.318 (1.35), 2.322 (2.92), 2.327 (4.19), 2.332 (3.05), 2.336 (1.32), 2.518 (16.00), 2.523 (11.46), 2.611 (1.37), 2.628 (2.49), 2.635 (2.29), 2.644 (2.27), 2.651 (2.94), 2.660 (2.36), 2.665 (3.93), 2.669 (5.92), 2.673 (3.79), 2.678 (1.72), 2.709 (0.54), 2.983 (1.86), 2.997 (3.37), 3.009 (3.08), 3.022 (3.22), 3.036 (1.94), 3.085 (0.90), 3.213 (2.39), 3.239 (2.52), 3.411 (3.40), 3.428 (6.23), 3.445 (6.85), 3.463 (3.18), 3.504 (2.20), 3.522 (2.00), 3.645 (3.58), 3.738 (1.20), 3.753 (1.53), 3.769 (1.77), 3.784 (1.60), 3.800 (1.25), 3.965 (10.45), 4.031 (1.21), 4.159 (0.65), 4.809 (0.62), 4.964 (0.67), 6.754 (0.60), 6.908 (4.90), 6.913 (5.03), 7.126 (0.80), 7.131 (0.63), 7.281 (1.72), 7.284 (1.79), 7.298 (2.36), 7.301 (3.09), 7.305 (2.05), 7.319 (2.29), 7.322 (2.24), 7.397 (2.18), 7.400 (2.29), 7.417 (3.35), 7.435 (1.96), 7.437 (1.93), 7.564 (0.55), 7.579 (0.53), 7.681 (3.75), 7.701 (3.23), 7.806 (5.76), 7.811 (5.79), 8.113 (3.36), 8.134 (3.24), 8.222 (2.34), 8.271 (0.65), 8.292 (0.48), 10.258 (3.30).

### Example 7, Intermediate 1

### tert-butyl 3-{2-[(3-methoxynaphthalen-1-yl)amino]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

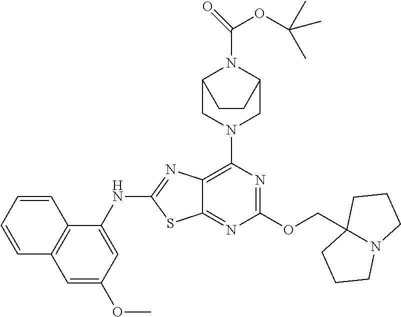 [SMILES: COc1cc(Nc2nc3c(N4CC5CCC(C4)N5C(=O)OC(C)(C)C)nc(OCC45CCCN4CCC5)nc3s2)c2ccccc2c1]

tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (100 mg, 17.7 μmol, example 1, intermediate 3), 3-methoxynaphthalen-1-amine (30.6 mg, 177 μmol), palladium(II) acetate (3.97 mg, 17.7 μmol; CAS-RN: [3375-31-3]), 4,5-bis(diphenylphosphino)-9,9-dimethylxanthene (10.2 mg, 17.7 μmol; CAS-RN: [161265-03-8]) and cesium carbonate (28.8 mg, 88.4 μmol; CAS-RN: [534-17-8]) were dissolved in 1,4-dioxane (380 μl). The reaction mixture was stirred at 100° C. in a sealed tube und N₂ atmosphere overnight. Then, the reaction mixture was diluted with ethyl acetate and water. It was extracted three times with ethyl acetate, the combined organic layers were washed with water and brine, filtered through a silicone coated filter and concentrated under reduced pressure. The crude product was purified by flash chromatography to yield the title compound (31.0 mg, 80% purity, 21% yield).

### Example 7, Building Block 1

### 3-methoxynaphthalen-1-amine

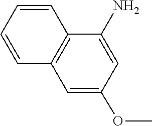

1-bromo-3-methoxynaphthalene (1.00 g, 4.22 mmol, CAS-RN: [5111-34-2]), benzophenone imine (803 mg, 4.43 mmol; CAS-RN: [1013-88-3]), racemic 2,2′-bis(diphenylphosphino)-1,1′-binaphthyl (131 mg, 211 μmol, CAS-RN: [98327-87-8]) were suspended in toluene (18 ml). Nitrogen was bubbled through this mixture for 5 minutes. Then, sodium tert-butoxide (608 mg, 6.33 mmol; CAS-RN:[865-48-5]) and tris(dibenzylideneacetone)di-palladium(0) (96.6 mg, 105 μmol; CAS-RN:[52409-22-0]) were added. The reaction mixture was stirred at 90° C. und N₂ atmosphere for 2 hours. Afterwards, the reaction mixture was quenched with ammonium chloride solution and extracted with ethyl acetate. The crude product was then treated with ethyl acetate and HCl (2N) and it was stirred vigorously at room temperature for 4 hours. The phases were separated and the pH of the aqueous layer was adjusted to 8 by addition of NaOH (2N). It was extracted with ethyl acetate to obtain the product which was purified by flash chromatography to yield the title compound (488 mg, 100% purity, 67% yield).

LC-MS (method 1) Rₜ=0.97 min; MS (ESIpos): m/z=174 [M+H]⁺

¹H-NMR (400 MHz, DMSO-d6) δ[ppm]: 2.518 (0.80), 2.523 (0.53), 3.334 (16.00), 5.737 (3.65), 6.305 (3.75), 6.311 (4.16), 6.520 (2.53), 6.525 (2.40), 7.153 (0.97), 7.157 (1.03), 7.170 (1.29), 7.174 (1.83), 7.177 (1.12), 7.191 (1.30), 7.195 (1.27), 7.310 (1.09), 7.313 (1.13), 7.327 (0.99), 7.331 (1.92), 7.334 (1.37), 7.348 (1.05), 7.351 (1.00), 7.596 (1.75), 7.617 (1.52), 7.619 (1.47), 7.925 (1.63), 7.946 (1.54).

### Example 8

### 4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)-5-fluoronaphthalen-2-ol—Hydrogen Chloride Salt

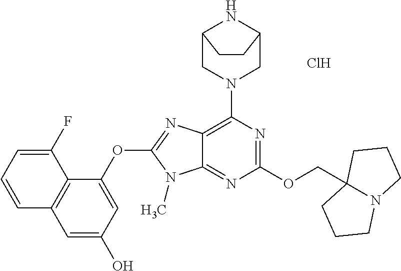 [SMILES: Cn1c(Oc2cc(O)cc3cccc(F)c23)nc2c(N3CC4CCC(C3)N4)nc(OCC34CCCN3CCC4)nc21 ; Cl]

To a stirred solution of tert-butyl 3-{8-({8-fluoro-3-[(oxan-2-yl)oxy]naphthalen-1-yl}oxy)-9-methyl-2-[(tetrahydro-1H-pyrrolizin-7a(5H)-yl)methoxy]-9H-purin-6-yl}-3,8-diazabicyclo[3.2.1]octane-8-carboxylate (45.0 mg, 60.5 μmol) in dichloromethane (0.45 mL) and methanol (0.9 mL) was added HCl in dioxane (450 μl, 4.0 M, 1.8 mmol). The mixture was stirred at 40° C. for 2 h. The solvent was removed in vacuum and the residue was triturated with dichloromethane to give 28.0 mg (73% yield) of the title compound as a solid as a hydrogen chloride salt.

LC-MS (Method 2): Rₜ=1.01 min; MS (ESIneg): m/z=558 [M−H]⁻

¹H-NMR (400 MHz, DMSO-d₆): δ[ppm]=10.57 (br s, 2H), 9.69 (br d, 1H), 9.55-9.40 (m, 1H), 7.61 (d, 1H), 7.40 (td, 1H), 7.28 (d, 1H), 7.18 (t, 1H), 7.02 (ddd, 1H), 4.92-4.68 (m, 2H), 4.45 (s, 2H), 4.03 (br s, 2H), 3.64 (s, 3H), 3.55-3.37 (m, 4H), 3.28-3.14 (m, 2H), 2.21-2.06 (m, 4H), 2.05-1.94 (m, 4H), 1.92-1.84 (m, 2H), 1.62 (br d, 2H)

### Intermediate 8-1

### 2-[(4-bromo-5-fluoronaphthalen-2-yl)oxy]oxane

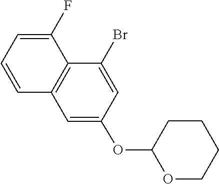 [SMILES: Fc1cccc2cc(OC3CCCCO3)cc(Br)c12]

To a stirred solution of 3,4-dihydro-2H-pyran (560 μl, 6.2 mmol) in dichloromethane (10 mL) was added pyridine 4-methylbenzenesulfonate (52.1 mg, 207 μmol) and then a solution of 4-bromo-5-fluoronaphthalen-2-ol (500 mg, 2.07 mmol) in dichloromethane (10 mL) was slowly added at room temperature. The mixture was stirred at room temperature for 1 h. A solution of sodium hydroxide (c=1N) was added, the organic phase was separated, washed with half-saturated sodium chloride solution, dried (sodium sulfate), filtered and the solvent was removed in vacuum. Silicagel chromatography (Gradient: hexanes/ethyl acetate 10-20%) gave 602 mg (89% yield) of the title compound.

¹H-NMR (400 MHz, DMSO-d₆): δ[ppm]=7.77-7.71 (m, 1H), 7.66 (d, 1H), 7.60 (t, 1H), 7.50 (td, 1H), 7.26 (ddd, 1H), 5.70 (t, 1H), 3.76 (ddd, 1H), 3.66-3.56 (m, 1H), 1.98-1.73 (m, 3H), 1.71-1.51 (m, 3H).

### Intermediate 8-2

### 8-fluoro-3-[(oxan-2-yl)oxy]naphthalen-1-ol

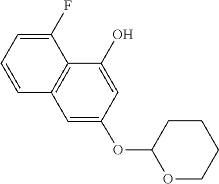 [SMILES: Oc1cc(OC2CCCCO2)cc2cccc(F)c12]

To a stirred solution of 2-[(4-bromo-5-fluoronaphthalen-2-yl)oxy]oxane (550 mg, 1.69 mmol) in dioxane (10 mL) in a microwave tube was added 5-(di-tert-butylphosphanyl)-1′,3′,5′-triphenyl-1′H-1,4′-bipyrazole, (85.7 mg, 169 μmol, CAS-RN: [894086-00-1]), Pd₂(dba)₃ (38.7 mg, 42.3 μmol), cesium hydroxide (761 mg, 5.07 mmol) and the flask was twice degased and backfilled with argon. The mixture was heated to 100° C. for 2 h. The cooled reaction mixture was directly used for silicagel chromatography (hexane, ethyl acetate 20-100%) to give 162 mg (37% yield) of the title compound as well as a second batch of 131 mg (30% yield) of the title compound.

### Batch 1 (162 mg):

LC-MS (Method 1): Rₜ=1.22 min; MS (ESIpos): m/z=263 [M+H]⁺

¹H NMR (DMSO-d₆, 400 MHz) δ10.20 (d, 1H, J=1.5 Hz), 7.47 (dd, 1H, J=0.8, 8.4 Hz), 7.31 (dt, 1H, J=4.9, 7.9 Hz), 6.9-7.0 (m, 2H), 6.64 (d, 1H, J=2.0 Hz), 5.54 (t, 1H, J=3.0 Hz), 3.7-3.8 (m, 1H), 3.6-3.6 (m, 1H), 1.7-2.0 (m, 3H), 1.5-1.7 (m, 3H).

### Batch 2 (131 mg):

LC-MS (Method 1): Rₜ=1.22 min; MS (ESIpos): m/z=263 [M+H]⁺

### Intermediate 8-3

### tert-butyl 3-(2-chloro-9-methyl-9H-purin-6-yl)-3,8-diazabicyclo[3.2.1]octane-8-carboxylate

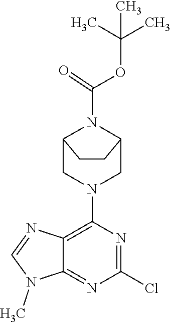 [SMILES: Cn1cnc2c(N3CC4CCC(C3)N4C(=O)OC(C)(C)C)nc(Cl)nc21]

To a stirred solution of 2,6-dichloro-9-methyl-9H-purine (3.00 g, 14.8 mmol; CAS-RN: [2382-10-7]) and tert-butyl 3,8-diazabicyclo[3.2.1]octane-8-carboxylate (3.45 g, 16.3 mmol) in THF (200 ml) was added N,N-diisopropylethylamine (7.7 ml, 44 mmol; CAS-RN: [7087-68-5]) at 0° C. and the mixture was stirred at room temperature for 24 h. The reaction mixture was combined with a second identical reaction starting with 300 mg (1.48 mM) of 2,6-dichloro-9-methyl-9H-purine (CAS-RN: [2382-10-7]), ethyl acetate was added and the mixture was washed with half-saturated sodium chloride solution, dried (sodium sulfate), filtered and the solvent was removed in vacuum to give a solid that was triturated with ethyl acetate to give 5.72 g (93% yield) of the title compound.

LC-MS (Method 1): Rₜ=1.28 min; MS (ESIpos): m/z=379 [M+H]⁺

¹H-NMR (400 MHz, DMSO-d₆): δ[ppm]=8.17 (s, 1H), 5.78-5.39 (m, 1H), 4.78-4.37 (m, 1H), 4.27 (br s, 2H), 3.70 (s, 3H), 3.48-3.35 (m, 1H), 3.21-2.90 (m, 1H), 1.83 (br s, 2H), 1.56 (br d, 2H), 1.44 (s, 9H).

### Intermediate 8-4

### tert-butyl 3-{9-methyl-2-[(tetrahydro-1H-pyrrolizin-7a(5H)-yl)methoxy]-9H-purin-6-yl}-3,8-diazabicyclo[3.2.1]octane-8-carboxylate

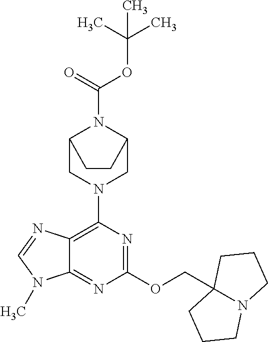 [SMILES: Cn1cnc2c(N3CC4CCC(C3)N4C(=O)OC(C)(C)C)nc(OCC34CCCN3CCC4)nc21]

To a stirred solution of sodium hydride (633 mg, 60% purity, 15.8 mmol; CAS-RN: [7646-69-7]) in THF (20 ml) distributed equaly to two microwave vials, was added (tetrahydro-1H-pyrrolizin-7a(5H)-yl)methanol (1.68 g, 11.9 mmol) at 0° C. After stirring for 30 minutes, tert-butyl 3-(2-chloro-9-methyl-9H-purin-6-yl)-3,8-diazabicyclo[3.2.1]octane-8-carboxylate (3.00 g, 7.92 mmol) dissolved in THF (10 ml) was added and the reaction mixture was stirred at 60° C. for 27 h. The reaction mixture was combined with a second identical reaction starting with 300 mg (0.79 mM) of 3-(2-chloro-9-methyl-9H-purin-6-yl)-3,8-diazabicyclo[3.2.1]octane-8-carboxylate, water and a saturated sodium bicarbonate solution were added until pH 12 was reached and the mixture was extracted with ethyl acetate. The organic phase was dried (sodium sulfate), filtered and the solvent was removed in vacuum. Aminophase-silicagel chromatography (Gradient: hexane/ethyl acetate 10-45%) gave 3.51 g (79% yield) of the title compound.

LC-MS (Method 2): Rₜ=1.34 min; MS (ESIpos): m/z=484 [M+H]⁺

¹H-NMR (400 MHz, DMSO-d₆): δ[ppm]=7.94 (s, 1H), 5.74-5.38 (m, 1H), 4.80-4.48 (m, 1H), 4.24 (br s, 2H), 3.91 (s, 2H), 3.63 (s, 3H), 3.32-3.20 (m, 1H), 3.20-2.99 (m, 1H), 2.91 (dt, 2H), 2.56-2.52 (m, 2H), 1.90-1.68 (m, 8H), 1.61-1.49 (m, 4H), 1.43 (s, 9H).

### Intermediate 8-5

### tert-butyl 3-{8-bromo-9-methyl-2-[(tetrahydro-1H-pyrrolizin-7a(5H)-yl)methoxy]-9H-purin-6-yl}-3,8-diazabicyclo[3.2.1]octane-8-carboxylate

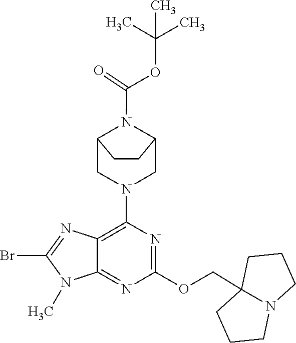 [SMILES: Cn1c(Br)nc2c(N3CC4CCC(C3)N4C(=O)OC(C)(C)C)nc(OCC34CCCN3CCC4)nc21]

To a stirred solution of tert-butyl 3-{9-methyl-2-[(tetrahydro-1H-pyrrolizin-7a(5H)-yl)methoxy]-9H-purin-6-yl}-3,8-diazabicyclo[3.2.1]octane-8-carboxylate (100 mg, 207 μmol) in tetrahydrofurane (4 mL) was added a solution of LiHMDS (620 μl, 1.0 M in THF, 620 μmol) at room temperature. The solution was stirred at room temperature for 1 h. The solution was cooled to 0° C. and Bromine-1,4-dioxane complex (61.5 mg, 248 μmol; CAS-RN: [15481-39-7]) was added. The solution was allowed to warm up to room temperature and was stirred at room temperature for 3 h. An aqueous solution of disodium sulfurothioate and a saturated solution of potassium carbonate were added and the mixture was extracted with chloroform and methanol (10:1 mixture). The organic phase was dried (sodium sulfate), filtered and the solvent was removed in vacuum. Silicagel chromatography (Gradient: dichloromethane/0-50% ethanol containing 1% conc. ammonium hydroxide solution) gave 86.0 mg (88% purity, 65% yield) of the title compound containing approx. 12% of the starting material tert-butyl 3-{9-methyl-2-[(tetrahydro-1H-pyrrolizin-7a(5H)-yl)methoxy]-9H-purin-6-yl}-3,8-diazabicyclo[3.2.1]octane-8-carboxylate.

LC-MS (Method 1): Rₜ=1.04 min; MS (ESIpos): m/z=562 [M+H]⁺

¹H-NMR (400 MHz, DMSO-d₆): δ[ppm]=5.50-5.06 (m, 1H), 4.80-4.43 (m, 1H), 4.26 (br s, 2H), 3.91 (s, 2H), 3.57 (s, 3H), 3.43-3.34 (m, 1H), 3.21-2.97 (m, 1H), 2.96-2.85 (m, 2H), 2.56-2.51 (m, 2H), 1.93-1.65 (m, 8H), 1.63-1.48 (m, 4H), 1.43 (s, 9H).

### Intermediate 8-6

### tert-butyl 3-{8-({8-fluoro-3-[(oxan-2-yl)oxy]naphthalen-1-yl}oxy)-9-methyl-2-[(tetrahydro-1H-pyrrolizin-7a(5H)-yl)methoxy]-9H-purin-6-yl}-3,8-diazabicyclo[3.2.1]octane-8-carboxylate

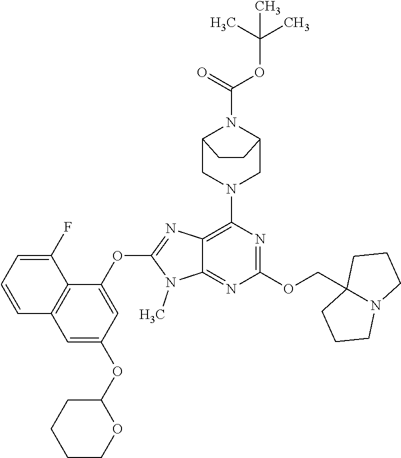 [SMILES: Cn1c(Oc2cc(OC3CCCCO3)cc3cccc(F)c23)nc2c(N3CC4CCC(C3)N4C(=O)OC(C)(C)C)nc(OCC34CCCN3CCC4)nc21]

To a stirred solution of tert-butyl 3-{8-bromo-9-methyl-2-[(tetrahydro-1H-pyrrolizin-7a(5H)-yl)methoxy]-9H-purin-6-yl}-3,8-diazabicyclo[3.2.1]octane-8-carboxylate (75.0 mg, 133 μmol) and 8-fluoro-3-[(oxan-2-yl)oxy]naphthalen-1-ol (105 mg, 400 μmol) in N,N-dimethylacetamide (1.2 ml) was added potassium carbonate (92.1 mg, 667 μmol) and the mixture was stirred at 100° C. for 48 h. Water was added and the mixture was extracted with ethyl acetate. The organic phase was washed with half-saturated sodium chloride solution, dried (sodium sulfate), filtered and the solvent was removed in vacuum. Aminophase-silicagel chromatography (Gradient: hexanes/ethyl acetate 10-60%) followed by silicagel chromatography (Gradient: dichloromethane/ethanol 0-50%) gave 48.0 mg (48% yield) of the title compound as a crude product that was used without further purification.

LC-MS (Method 1): Rₜ=1.33 min; MS (ESIpos): m/z=744 [M−H]⁺

¹H-NMR (400 MHz, DMSO-d6) δ[ppm]: 1.385 (16.00), 1.433 (1.51), 1.506 (0.71), 1.523 (0.79), 1.536 (0.59), 1.543 (0.73), 1.555 (0.86), 1.573 (0.70), 1.691 (0.40), 1.710 (0.46), 1.724 (0.77), 1.740 (0.98), 1.756 (0.81), 1.765 (0.64), 1.782 (0.71), 1.796 (0.70), 1.810 (0.59), 1.826 (0.71), 1.839 (1.16), 1.852 (0.92), 1.856 (0.87), 1.869 (0.86), 1.883 (0.72), 1.899 (0.43), 2.518 (1.21), 2.523 (1.10), 2.893 (0.48), 2.906 (0.81), 2.919 (0.68), 2.930 (0.72), 2.945 (0.42), 3.571 (0.64), 3.618 (6.64), 3.906 (2.28), 5.728 (0.78), 5.758 (0.42), 7.150 (0.41), 7.478 (0.51), 7.486 (0.70), 7.491 (1.55), 7.497 (0.80), 7.669 (0.48), 7.714 (0.88), 7.733 (0.74).

### Example 9

### 4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)naphthalen-2-ol—Hydrogen Chloride Salt

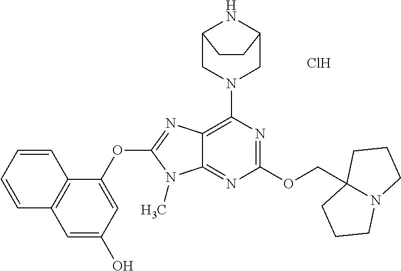 [SMILES: Cn1c(Oc2cc(O)cc3ccccc23)nc2c(N3CC4CCC(C3)N4)nc(OCC34CCCN3CCC4)nc21 ; Cl]

To a stirred solution of tert-butyl 3-{9-methyl-8-({3-[(oxan-2-yl)oxy]naphthalen-1-yl}oxy)-2-[(tetrahydro-1H-pyrrolizin-7a(5H)-yl)methoxy]-9H-purin-6-yl}-3,8-diazabicyclo[3.2.1]octane-8-carboxylate (28.0 mg, 38.6 μmol) in dichloromethane (0.57 mL) and methanol (0.28 mL) was added HCl in dioxane (290 μl, 4.0 M, 1.2 mmol). The mixture was stirred at 40° C. for 2 h. The solvent was mainly removed in vacuum and the residue was triturated with dichloromethane to give 12.0 mg (50% yield) of the title compound as a solid as a hydrogen chloride salt.

LC-MS (Method 2): Rₜ=1.17 min; MS (ESIneg): m/z=540 [M−H]⁻

1H-NMR (400 MHz, DMSO-d6): δ[ppm]=10.56 (br s, 1H), 10.34-9.95 (m, 1H), 9.74 (br d, 1H), 9.53-9.41 (m, 1H), 8.06-7.99 (m, 1H), 7.77 (d, 1H), 7.49 (ddd, 1H), 7.35 (ddd, 1H), 7.27 (d, 1H), 7.06 (d, 1H), 5.09-4.70 (m, 2H), 4.47 (s, 2H), 4.11 (br s, 2H), 3.67 (s, 3H), 3.59-3.43 (m, 4H), 3.21 (br dd, 2H), 2.20-2.06 (m, 4H), 2.05-1.88 (m, 6H), 1.74-1.66 (m, 2H).

### Intermediate 9-1

### 2-[(4-bromonaphthalen-2-yl)oxy]oxane

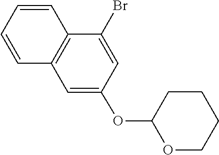 [SMILES: Brc1cc(OC2CCCCO2)cc2ccccc12]

To a stirred solution of 3,4-dihydro-2H-pyran (2.5 ml, 27 mmol) in dichloromethane (40 mL) was added pyridine 4-methylbenzenesulfonate (225 mg, 897 μmol) and then a solution of 4-bromonaphthalen-2-ol (2.00 g, 8.97 mmol) in dichloromethane (40 mL) was slowly added at room temperature. The mixture was stirred at room temperature for 1 h. A solution of sodium hydroxide (c=1N) was added, the organic phase was separated, washed with half-saturated sodium chloride solution, dried (sodium sulfate), filtered and the solvent was removed in vacuum. Silicagel chromatography (Gradient: hexanes/ethyl acetate 10-20%) gave 2.66 g (97% yield) of the title compound.

¹H-NMR (400 MHz, DMSO-d₆): δ[ppm]=8.06-7.99 (m, 1H), 7.91-7.85 (m, 1H), 7.67 (d, 1H), 7.59-7.48 (m, 3H), 5.68 (t, 1H), 3.78 (ddd, 1H), 3.65-3.56 (m, 1H), 1.98-1.75 (m, 3H), 1.71-1.50 (m, 3H).

### Intermediate 9-2

### 3-[(oxan-2-yl)oxy]naphthalen-1-ol

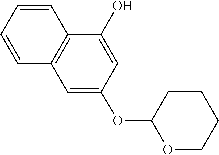 [SMILES: Oc1cc(OC2CCCCO2)cc2ccccc12]

To a stirred solution of 2-[(4-bromonaphthalen-2-yl)oxy]oxane (1.00 g, 3.26 mmol) in dioxane (20 mL) in a microwave tube was added 5-(di-tert-butylphosphanyl)-1′,3′,5′-triphenyl-1′H-1,4′-bipyrazole (165 mg, 326 μmol, CAS-RN: [894086-00-1]), Pd₂(dba)₃ (74.5 mg, 81.4 μmol) and cesium hydroxide (1.46 g, 9.77 mmol) and the flask was twice degased and backfilled with argon. The mixture was heated to 100° C. for 2 h. The cooled reaction mixture was directly used for silicagel chromatography (hexane, ethyl acetate 20-100%) to give 398 mg (50% yield) of the title compound.

LC-MS (Method 1): Rₜ=1.18 min; MS (ESIpos): m/z=245 [M+H]⁺

¹H-NMR (400 MHz, DMSO-d₆): δ[ppm]=10.23 (s, 1H), 8.00 (d, 1H), 7.66 (d, 1H), 7.40 (ddd, 1H), 7.31-7.25 (m, 1H), 6.90 (d, 1H), 6.62 (d, 1H), 5.53 (t, 1H), 3.86-3.74 (m, 1H), 3.61-3.54 (m, 1H), 1.96-1.71 (m, 3H), 1.70-1.48 (m, 3H).

### Intermediate 9-3

### tert-butyl 3-{9-methyl-8-({3-[(oxan-2-yl)oxy]naphthalen-1-yl}oxy)-2-[(tetrahydro-1H-pyrrolizin-7a(5H)-yl)methoxy]-9H-purin-6-yl}-3,8-diazabicyclo[3.2.1]octane-8-carboxylate

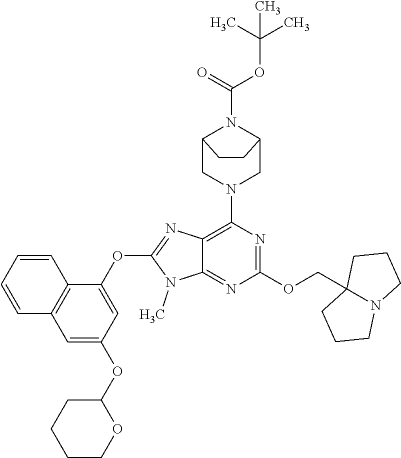 [SMILES: Cn1c(Oc2cc(OC3CCCCO3)cc3ccccc23)nc2c(N3CC4CCC(C3)N4C(=O)OC(C)(C)C)nc(OCC34CCCN3CCC4)nc21]

To a stirred solution of tert-butyl 3-{8-bromo-9-methyl-2-[(tetrahydro-1H-pyrrolizin-7a(5H)-yl)methoxy]-9H-purin-6-yl}-3,8-diazabicyclo[3.2.1]octane-8-carboxylate (55.3 mg, 98.3 μmol) and 3-[(oxan-2-yl)oxy]naphthalen-1-ol (72.0 mg, 295 μmol) in N,N-dimethylacetamide (890 μl) was added potassium carbonate (67.9 mg, 492 μmol) and the mixture was stirred at 100° C. for 48 h. Water was added and the mixture was extracted with ethyl acetate. The organic phase was washed with half-saturated sodium chloride solution, dried (sodium sulfate), filtered and the solvent was removed in vacuum. Aminophase-silicagel chromatography (Gradient: hexanes/ethyl acetate 10-40%) gave 30.0 mg (42% yield) of the title compound as a crude product that was used without further purification.

LC-MS (Method 1): Rₜ=1.43 min; MS (ESIpos): m/z=726 [M+H]⁺

¹H-NMR (400 MHz, DMSO-d6) δ[ppm]: 1.172 (0.41), 1.405 (16.00), 1.530 (0.68), 1.542 (0.61), 1.549 (0.72), 1.560 (1.21), 1.579 (1.15), 1.729 (0.50), 1.746 (0.66), 1.763 (0.63), 1.770 (0.69), 1.785 (1.00), 1.802 (1.04), 1.814 (1.05), 1.828 (0.82), 1.836 (0.63), 1.846 (0.97), 1.862 (0.66), 1.876 (0.86), 1.889 (0.63), 1.955 (2.54), 1.987 (0.84), 2.518 (1.43), 2.523 (1.03), 2.560 (0.41), 2.782 (2.39), 2.898 (0.45), 2.911 (0.74), 2.924 (0.61), 2.936 (0.78), 2.941 (4.00), 2.950 (0.43), 3.658 (6.19), 3.922 (2.13), 5.681 (0.74), 5.759 (2.78), 7.380 (0.96), 7.385 (0.98), 7.462 (0.51), 7.466 (0.68), 7.469 (0.41), 7.483 (0.51), 7.486 (0.48), 7.540 (0.42), 7.543 (0.45), 7.561 (0.69), 7.564 (0.52), 7.700 (0.41), 7.876 (0.73), 7.896 (0.66), 8.134 (0.64), 8.155 (0.60).

### Example 10

### 4-({6-(3,8-diazabicyclo[3.2.1]octan-3-yl)-2-[(tetrahydro-1H-pyrrolizin-7a(5H)-yl)methoxy]-9H-purin-8-yl}oxy)naphthalen-2-ol—Hydrogen Chloride Salt

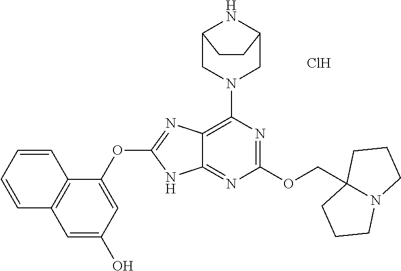 [SMILES: Oc1cc(Oc2nc3c(N4CC5CCC(C4)N5)nc(OCC45CCCN4CCC5)nc3[nH]2)c2ccccc2c1 ; Cl]

To a stirred solution of tert-butyl 3-[8-({3-[(oxan-2-yl)oxy]naphthalen-1-yl}oxy)-2-[(tetrahydro-1H-pyrrolizin-7a(5H)-yl)methoxy]-9-{[2-(trimethylsilyl)ethoxy]methyl}-9H-purin-6-yl]-3,8-diazabicyclo[3.2.1]octane-8-carboxylate (8.00 mg, 9.50 μmol) in dichloromethane (140 μL) and methanol (70 μL) was added HCl in dioxane (71 μl, 4.0 M, 280 μmol). The mixture was stirred at 40° C. for 2 h. The solvent was removed in vacuum and the residue was triturated with ethyl acetate to give 5.60 mg (83% yield) of the title compound as a solid as a hydrogen chloride salt.

LC-MS (Method 2): Rₜ=0.71 min; MS (ESIpos): m/z=528.6 [M+H]⁺

¹H-NMR (400 MHz, DMSO-d₆): δ[ppm]=13.50-13.20 (m, 1H), 10.53-10.26 (m, 1H), 10.17-10.02 (m, 1H), 9.62-9.50 (m, 1H), 9.36-9.22 (m, 1H), 7.94 (d, 1H), 7.77 (d, 1H), 7.48 (ddd, 1H), 7.34 (ddd, 1H), 7.10 (d, 1H), 7.05 (d, 1H), 5.12-4.66 (m, 2H), 4.41 (s, 2H), 4.12 (br s, 2H), 3.53-3.43 (m, 4H), 3.25-3.15 (m, 2H), 2.16-2.07 (m, 4H), 2.02-1.91 (m, 7H), 1.80-1.69 (m, 2H).

### Intermediate 10-1

### tert-butyl 3-(2-chloro-7H-purin-6-yl)-3,8-diazabicyclo[3.2.1]octane-8-carboxylate

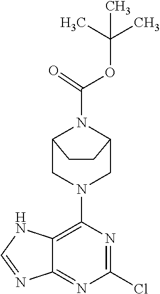 [SMILES: CC(C)(C)OC(=O)N1C2CCC1CN(c1nc(Cl)nc3nc[nH]c13)C2]

To a stirred solution of 2,6-dichloro-7H-purine (300 mg, 1.59 mmol) and tert-butyl 3,8-diazabicyclo[3.2.1]octane-8-carboxylate (371 mg, 1.75 mmol) in 1,4-dioxane (23 ml) was added N,N-diisopropylethylamine (830 μl, 4.8 mmol; CAS-RN: [7087-68-5]) at room temperature and the mixture was stirred at room temperature for 2 h. Ethyl acetate was added and the mixture was washed with half-saturated sodium chloride solution, dried (sodium sulfate), filtered and the solvent was removed in vacuum. Silicagel chromatography (hexane, ethyl acetate 20-40%) gave 441 mg (76% yield) of the title compound.

LC-MS (Method 1): Rₜ=1.16 min; MS (ESIpos): m/z=365 [M+H]⁺

¹H-NMR (400 MHz, DMSO-d₆): δ[ppm]=13.25 (br s, 1H), 8.16 (s, 1H), 5.73-5.46 (m, 1H), 4.76-4.38 (m, 1H), 4.27 (br s, 2H), 3.50-3.35 (m, 1H), 3.21-2.93 (m, 1H), 1.84 (br s, 2H), 1.58 (br d, 2H), 1.44 (s, 9H).

### Intermediate 10-2

### tert-butyl 3-(2-chloro-9-{[2-(trimethylsilyl)ethoxy]methyl}-9H-purin-6-yl)-3,8-diazabicyclo[3.2.1]octane-8-carboxylate

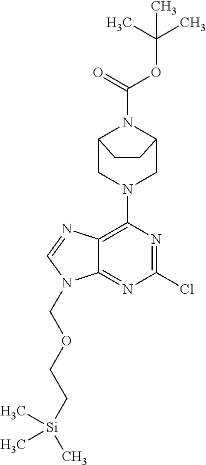 [SMILES: CC(C)(C)OC(=O)N1C2CCC1CN(c1nc(Cl)nc3c1ncn3COCC[Si](C)(C)C)C2]

To a solution of tert-butyl 3-(2-chloro-7H-purin-6-yl)-3,8-diazabicyclo[3.2.1]octane-8-carboxylate (747 mg, 2.05 mmol) in N,N-dimethylformamide (7.0 ml) was added sodium hydride (90.1 mg, 60% purity, in mineral oil, 2.25 mmol; CAS-RN: [7646-69-7]) at 0° C. After stirring for 10 minutes, [2-(chloromethoxy)ethyl](trimethyl)silane (420 μl, 2.4 mmol) was added and the reaction mixture was stirred at room temperature for 1 h. A half-saturated sodium bicarbonate solution was added and the mixture was extracted with ethyl acetate. The organic phase was washed with a half-saturated sodium chloride solution, dried (sodium sulfate), filtered and the solvent was removed in vacuum. Silicagel chromatography (Gradient: hexane/ethyl acetate 10-45%) gave 734 mg (72% yield) of the title compound.

LC-MS (Method 2): Rₜ=1.64 min; MS (ESIpos): m/z=495, 497 [M+H]⁺

¹H-NMR (400 MHz, DMSO-d₆): δ[ppm]=8.37 (s, 1H), 5.64-5.51 (m, 1H), 5.50 (s, 2H), 4.64-4.45 (m, 1H), 4.27 (br s, 2H), 3.61-3.52 (m, 2H), 3.49-3.35 (m, 1H), 3.16-2.98 (m, 1H), 1.84 (br s, 2H), 1.65-1.50 (m, 2H), 1.44 (s, 9H), 0.88-0.79 (m, 2H), −0.05-0.10 (m, 9H).

### Intermediate 10-3

### tert-butyl 3-(2-[(tetrahydro-1H-pyrrolizin-7a(5H)-yl)methoxy]-9-{[2-(trimethylsilyl)ethoxy]methyl}-9H-purin-6-yl)-3,8-diazabicyclo[3.2.1]octane-8-carboxylate

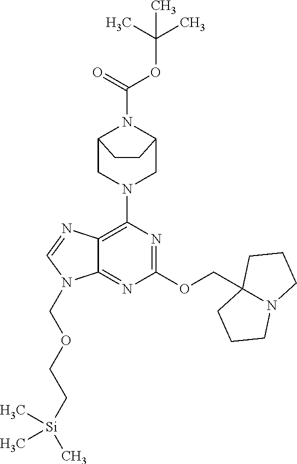 [SMILES: CC(C)(C)OC(=O)N1C2CCC1CN(c1nc(OCC34CCCN3CCC4)nc3c1ncn3COCC[Si](C)(C)C)C2]

To a stirred solution of sodium hydride (297 mg, 60% purity, 7.43 mmol; CAS-RN: [7646-69-7]) in THF (20 ml) distributed equaly to two microwave vials, was added (tetrahydro-1H-pyrrolizin-7a(5H)-yl)methanol (1.05 g, 7.43 mmol) at 0° C. After stirring for 15 minutes, tert-butyl 3-(2-chloro-9-{[2-(trimethylsilyl)ethoxy]methyl}-9H-purin-6-yl)-3,8-diazabicyclo[3.2.1]octane-8-carboxylate (1.84 g, 3.72 mmol) dissolved in THF (5 ml) was added and the reaction mixture was stirred at 70° C. for 5 h. A half-saturated sodium bicarbonate solution was added and the mixture was extracted with ethyl acetate. The organic phase was dried (sodium sulfate), filtered and the solvent was removed in vacuum. Aminophase-silicagel chromatography (Gradient: hexane/ethyl acetate 20-60%) followed by silicagel chromatography (Gradient: dichloromethane/ethanol 0-50%) gave 1.72 g (77% yield) of the title compound.

LC-MS (Method 1): Rₜ=1.18 min; MS (ESIpos): m/z=600 [M+H]⁺

¹H-NMR (400 MHz, DMSO-d₆): δ[ppm]=8.17-8.06 (m, 1H), 5.70-5.50 (m, 1H), 5.44 (s, 2H), 4.74-4.45 (m, 1H), 4.25 (br s, 2H), 3.90 (s, 2H), 3.61-3.52 (m, 2H), 3.46-3.35 (m, 1H), 3.20-2.97 (m, 1H), 2.95-2.85 (m, 2H), 2.57-2.51 (m, 2H), 1.90-1.64 (m, 8H), 1.62-1.48 (m, 4H), 1.44 (s, 9H), 0.89-0.80 (m, 2H), −0.08 (s, 9H).

### Intermediate 10-4

### tert-butyl 3-(8-bromo-2-[(tetrahydro-1H-pyrrolizin-7a(5H)-yl)methoxy]-9-{[2-(trimethylsilyl)ethoxy]methyl}-9H-purin-6-yl)-3,8-diazabicyclo[3.2.1]octane-8-carboxylate

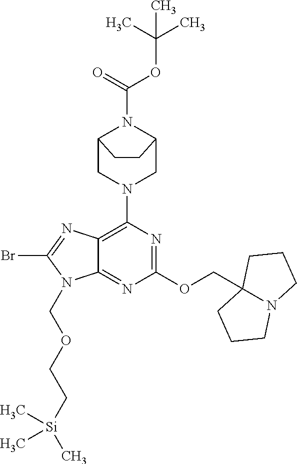 [SMILES: CC(C)(C)OC(=O)N1C2CCC1CN(c1nc(OCC34CCCN3CCC4)nc3c1nc(Br)n3COCC[Si](C)(C)C)C2]

To a stirred solution of tert-butyl 3-(2-[(tetrahydro-1H-pyrrolizin-7a(5H)-yl)methoxy]-9-{[2-(trimethylsilyl)ethoxy]methyl}-9H-purin-6-yl)-3,8-diazabicyclo[3.2.1]octane-8-carboxylate (743 mg, 1.24 mmol) in tetrahydrofurane (25 mL) was added a solution of LiHMDS (3.6 ml, 1.0 M in THF, 3.6 mmol) at room temperature. The solution was stirred at room temperature for 3 h. Bromine-1,4-dioxane complex (366 mg, 1.5 mmol; CAS-RN: [15481-39-7]), was added and the solution was stirred at room temperature for 16 h. Further solution of LiHMDS (3.6 ml, 1.0 M in THF, 3.6 mmol) was added at room temperature and the solution was stirred at room temperature for 2 h. Then, further Bromine-1,4-dioxane complex (366 mg, 1.5 mmol; CAS-RN: [15481-39-7]), was added and the solution was stirred at room temperature for 15 min. A third time a solution of LiHMDS (3.6 ml, 1.0 M in THF, 3.6 mmol) was added at room temperature and the solution was stirred at room temperature for 2 h. Then, further Bromine-1,4-dioxane complex (366 mg, 1.5 mmol; CAS-RN: [15481-39-7]), was added and the solution was stirred at room temperature for 15 min, and LC-MS indicated complete consumption of the starting material. An aqueous solution of disodium sulfurothioate and a saturated solution of sodium bicarbonate were added and the mixture was extracted with ethyl acetate. The organic phase was dried (sodium sulfate), filtered and the solvent was removed in vacuum. Aminophase-silicagel chromatography (Gradient: hexane/ethyl acetate 10-20%) gave 424 mg (50% yield) of the title compound.

LC-MS (Method 1): Rₜ=1.28 min; MS (ESIpos): m/z=679 [M+H]⁺

¹H-NMR (400 MHz, DMSO-d₆): δ[ppm]=5.40 (s, 2H), 5.35-5.10 (m, 1H), 4.74-4.45 (m, 1H), 4.27 (br s, 2H), 3.90 (s, 2H), 3.65-3.51 (m, 2H), 3.47-3.35 (m, 1H), 3.13-2.95 (m, 1H), 2.94-2.85 (m, 2H), 2.56-2.51 (m, 2H), 1.89-1.81 (m, 4H), 1.80-1.66 (m, 4H), 1.60-1.48 (m, 4H), 1.44 (s, 9H), 0.88-0.82 (m, 2H), −0.09 (s, 9H).

### Intermediate 10-5

### tert-butyl 3-[8-({3-[(oxan-2-yl)oxy]naphthalen-1-yl}oxy)-2-[(tetrahydro-1H-pyrrolizin-7a(5H)-yl)methoxy]-9-{[2-(trimethylsilyl)ethoxy]methyl}-9H-purin-6-yl]-3,8-diazabicyclo[3.2.1]octane-8-carboxylate

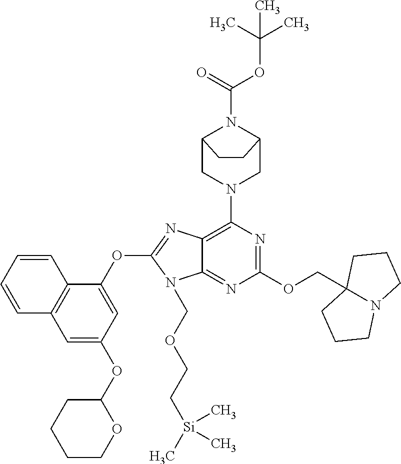 [SMILES: CC(C)(C)OC(=O)N1C2CCC1CN(c1nc(OCC34CCCN3CCC4)nc3c1nc(Oc1cc(OC4CCCCO4)cc4ccccc14)n3COCC[Si](C)(C)C)C2]

To a stirred solution of tert-butyl 3-(8-bromo-2-[(tetrahydro-1H-pyrrolizin-7a(5H)-yl)methoxy]-9-{[2-(trimethylsilyl)ethoxy]methyl}-9H-purin-6-yl)-3,8-diazabicyclo[3.2.1]octane-8-carboxylate (39.0 mg, 57.5 μmol) and 3-[(oxan-2-yl)oxy]naphthalen-1-ol (42.1 mg, 172 μmol) in N,N-dimethylacetamide (1.2 ml) was added potassium carbonate (39.7 mg, 287 μmol) and the mixture was stirred at 100° C. for 48 h. The solvent was removed in vacuum. Silicagel chromatography (Gradient: dichloromethane/ethanol 0-50%) followed by aminophase-silicagel chromatography (Gradient: hexanes/ethyl acetate 10-20%) gave 10.0 mg (90% purity, 19% yield) of the title compound as a crude product that was used without further purification.

LC-MS (Method 1): Rₜ=1.47 min; MS (ESIpos): m/z=842 [M+H]⁺

¹H-NMR (400 MHz, DMSO-d₆): δ[ppm]=8.13 (d, 1H), 7.89 (d, 1H), 7.74 (br s, 1H), 7.56 (ddd, 1H), 7.45 (ddd, 1H), 7.39 (d, 1H), 5.72-5.65 (m, 1H), 5.54 (s, 2H), 5.23-4.44 (m, 2H), 4.18 (br s, 2H), 3.92 (s, 2H), 3.77 (td, 1H), 3.71-3.63 (m, 2H), 3.62-3.54 (m, 1H), 3.28-2.99 (m, 2H), 2.96-2.88 (m, 2H), 2.57-2.52 (m, 2H), 1.93-1.70 (m, 12H), 1.62-1.51 (m, 6H), 1.41 (s, 9H), 0.88 (dd, 2H), −0.12 (s, 9H).

### Example 11

### 4-({6-(3,8-diazabicyclo[3.2.1]octan-3-yl)-2-[(tetrahydro-1H-pyrrolizin-7a(5H)-yl)methoxy]-9H-purin-8-yl}oxy)-5-fluoronaphthalen-2-ol—Hydrogen Chloride Salt

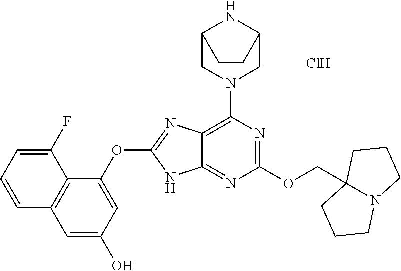 [SMILES: Oc1cc(Oc2nc3c(N4CC5CCC(C4)N5)nc(OCC45CCCN4CCC5)nc3[nH]2)c2c(F)cccc2c1 ; Cl]

To a stirred solution of tert-butyl 3-[8-({8-fluoro-3-[(oxan-2-yl)oxy]naphthalen-1-yl}oxy)-2-[(tetrahydro-1H-pyrrolizin-7a(5H)-yl)methoxy]-9-{[2-(trimethylsilyl)ethoxy]methyl}-9H-purin-6-yl]-3,8-diazabicyclo[3.2.1]octane-8-carboxylate (50.0 mg, 58.1 μmol) in dichloromethane (860 μL) and methanol (430 μL) was added HCl in dioxane (440 μl, 4.0 M, 1.7 mmol). The mixture was stirred at 40° C. for 2 h. The solvent was mainly removed in vacuum and the residue was triturated with dichloromethane to give 23.0 mg (60% yield) of the title compound as a solid as a hydrogen chloride salt.

LC-MS (Method 2): Rₜ=0.65 min; MS (ESIneg): m/z=544 [M−H]⁻

¹H-NMR (400 MHz, DMSO-d₆): δ[ppm]=13.72-12.57 (m, 1H), 10.58 (br s, 2H), 9.66 (br d, 1H), 9.55-9.38 (m, 1H), 7.60 (d, 1H), 7.40 (td, 1H), 7.17 (t, 1H), 7.10 (d, 1H), 7.01 (ddd, 1H), 4.84-4.73 (m, 2H), 4.40 (s, 2H), 4.03 (br s, 2H), 3.54-3.37 (m, 4H), 3.25-3.13 (m, 2H), 2.16-2.04 (m, 4H), 2.04-1.93 (m, 4H), 1.93-1.84 (m, 2H), 1.65 (br d, 2H).

### Intermediate 11-1

### tert-butyl 3-[8-({8-fluoro-3-[(oxan-2-yl)oxy]naphthalen-1-yl}oxy)-2-[(tetrahydro-1H-pyrrolizin-7a(5H)-yl)methoxy]-9-{[2-(trimethylsilyl)ethoxy]methyl}-9H-purin-6-yl]-3,8-diazabicyclo[3.2.1]octane-8-carboxylate

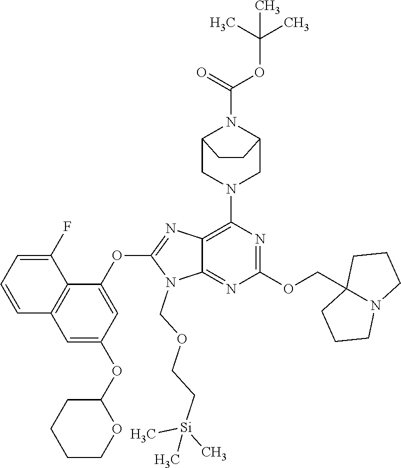 [SMILES: CC(C)(C)OC(=O)N1C2CCC1CN(c1nc(OCC34CCCN3CCC4)nc3c1nc(Oc1cc(OC4CCCCO4)cc4cccc(F)c14)n3COCC[Si](C)(C)C)C2]

To a stirred solution of tert-butyl 3-(8-bromo-2-[(tetrahydro-1H-pyrrolizin-7a(5H)-yl)methoxy]-9-{[2-(trimethylsilyl)ethoxy]methyl}-9H-purin-6-yl)-3,8-diazabicyclo[3.2.1]octane-8-carboxylate (75.0 mg, 110 μmol) and 8-fluoro-3-[(oxan-2-yl)oxy]naphthalen-1-ol (86.9 mg, 331 μmol) in N,N-dimethylacetamide (1.2 ml) was added potassium carbonate (76.4 mg, 552 μmol) and the mixture was stirred at 100° C. for 48 h. Water was added and the mixture was extracted with ethyl acetate. The organic phase was washed with half-saturated sodium chloride solution, dried (sodium sulfate), filtered and the solvent was removed in vacuum. Aminophase-silicagel chromatography (Gradient: hexanes/ethyl acetate 10-60%) gave 52.0 mg (55% yield) of the title compound as a crude product that was used without further purification.

LC-MS (Method 1): Rₜ=1.50 min; MS (ESIpos): m/z=861 [M+H]⁺

¹H-NMR (400 MHz, DMSO-d₆): δ[ppm]=7.77-7.68 (m, 1H), 7.63 (br s, 1H), 7.54-7.44 (m, 2H), 7.21-7.11 (m, 1H), 5.76-5.70 (m, 1H), 5.47 (s, 2H), 4.98-4.43 (m, 2H), 4.20-4.01 (m, 2H), 3.91 (s, 2H), 3.81-3.72 (m, 1H), 3.72-3.64 (m, 2H), 3.63-3.55 (m, 1H), 3.16-2.95 (m, 2H), 2.95-2.87 (m, 2H), 2.56-2.52 (m, 2H), 1.91-1.67 (m, 12H), 1.61-1.48 (m, 6H), 1.39 (s, 9H), 0.92-0.86 (m, 2H), −0.10 (s, 9H).

### Example 12

### 5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol hydrogen chloride salt

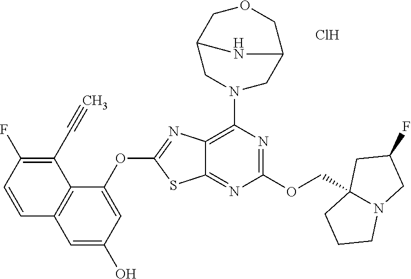 [SMILES: C#Cc1c(F)ccc2cc(O)cc(Oc3nc4c(N5CC6COCC(C5)N6)nc(OC[C@@]56CCCN5C[C@H](F)C6)nc4s3)c12 ; Cl]

To a stirred solution of tert-butyl 7-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (54.0 mg, 70.6 μmol) in dichloromethane (1.0 mL) and methanol (0.52 mL) was added HCl in dioxane (530 μl, 4.0 M, 2.1 mmol). The mixture was stirred at room temperature for 2 h. The solvent was mainly removed in vacuum and the residue was triturated with dichloromethane to give 39.0 mg (80% yield) of the title compound as a solid as a hydrogen chloride salt.

LC-MS (method A): Rₜ=0.72 min; MS (ESpos): m/z=621 [M+H]⁺

¹H-NMR (400 MHz, DMSO-d₆): δ[ppm]=11.20 (br d, 1H), 10.76-10.34 (m, 1H), 10.24-10.01 (m, 2H), 7.97 (dd, 1H), 7.50 (t, 1H), 7.39 (d, 1H), 7.32 (d, 1H), 5.70-5.42 (m, 2H), 5.35-5.10 (m, 1H), 4.61 (d, 1H), 4.48 (br d, 2H), 3.97 (br d, 3H), 3.87-3.71 (m, 4H), 3.66-3.56 (m, 2H), 3.46-3.33 (m, 1H), 3.26 (br dd, 1H), 2.61-2.52 (m, 1H), 2.48-2.41 (m, 2H), 2.31-2.23 (m, 1H), 2.23-2.08 (m, 2H), 2.07-1.97 (m, 1H).

### Intermediate 12-1

### tert-butyl 7-(5-chloro[1,3]thiazolo[5,4-d]pyrimidin-7-yl)-3-oxa-7,9-diazabicyclo[3.3.1]nonane-9-carboxylate

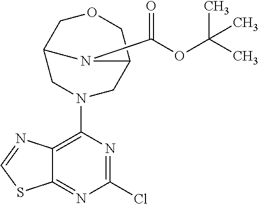 [SMILES: CC(C)(C)OC(=O)N1C2COCC1CN(c1nc(Cl)nc3scnc13)C2]

To a stirred solution of 5,7-dichloro[1,3]thiazolo[5,4-d]pyrimidine (1.00 g, 4.85 mmol) and tert-butyl 3-oxa-7,9-diazabicyclo[3.3.1]nonane-9-carboxylate (1.22 g, 5.34 mmol) in 1,4-dioxane (21 ml) was added triethylamine (1.7 ml, 12 mmol; CAS-RN: [121-44-8]) at room temperature and the mixture was stirred at 100° C. for 1 h. Water was added and the mixture was extracted with ethyl acetate. The organic phase was dried (sodium sulfate), filtered and the solvent was removed in vacuum to give 1.93 g (95% purity, 95% yield) of the title compound as a crude product that was used without further purification.

LC-MS (Method 1): Rₜ=1.32 min; MS (ESIpos): m/z=398 [M+H]⁺

¹H-NMR (400 MHz, DMSO-d₆): δ[ppm]=9.25 (s, 1H), 6.09 (br d, 1H), 5.03 (br d, 1H), 4.08 (br d, 2H), 3.90-3.78 (m, 2H), 3.73-3.58 (m, 3H), 3.31-3.24 (m, 1H), 1.44 (s, 9H).

### Intermediate 12-2

### tert-butyl 7-(5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

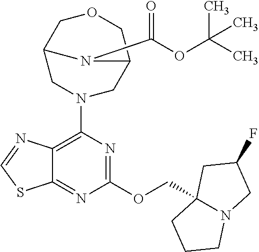 [SMILES: CC(C)(C)OC(=O)N1C2COCC1CN(c1nc(OC[C@@]34CCCN3C[C@H](F)C4)nc3scnc13)C2]

To a stirred solution of [(2R,7aS)-2-fluorotetrahydro-1H-pyrrolizin-7a(5H)-yl]methanol (260 mg, 1.63 mmol) in THF (5.0 ml) in a microwave tube, was added sodium hydride (70.4 mg, 60% purity, 1.76 mmol; CAS-RN: [7646-69-7]) at room temperature. After stirring for 10 minutes, tert-butyl 7-(5-chloro[1,3]thiazolo[5,4-d]pyrimidin-7-yl)-3-oxa-7,9-diazabicyclo[3.3.1]nonane-9-carboxylate (500 mg, 1.26 mmol) dissolved in N,N-dimethylacetamide (2.5 ml) was added and the reaction mixture was stirred at 75° C. for 1 h. The reaction mixture was combined with a second identical reaction starting with 100 mg (0.25 mM) of tert-butyl 7-(5-chloro[1,3]thiazolo[5,4-d]pyrimidin-7-yl)-3-oxa-7,9-diazabicyclo[3.3.1]nonane-9-carboxylate, water was added and the mixture was extracted with ethyl acetate and methanol (10:1 mixture). The organic phase was dried (sodium sulfate), filtered and the solvent was removed in vacuum. Aminophase-silicagel chromatography (Gradient: dichloromethane/ethanol 0-40%) gave 560 mg (90% purity, 64% yield) of the title compound.

LC-MS (Method 1): Rₜ=0.90 min; MS (ESIpos): m/z=521 [M+H]⁺

¹H-NMR (400 MHz, DMSO-d₆): δ[ppm]=8.98 (s, 1H), 6.09 (br dd, 1H), 5.38-5.17 (m, 1H), 5.16-5.04 (m, 1H), 4.09-4.01 (m, 3H), 3.94 (br d, 1H), 3.83 (br s, 2H), 3.61 (br d, 3H), 3.29-3.17 (m, 1H), 3.11-3.05 (m, 2H), 3.00 (s, 1H), 2.87-2.79 (m, 1H), 2.13-2.08 (m, 1H), 2.02 (d, 1H), 1.98-1.96 (m, 1H), 1.87-1.71 (m, 3H), 1.44 (s, 9H).

### Intermediate 12-3

### tert-butyl 7-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

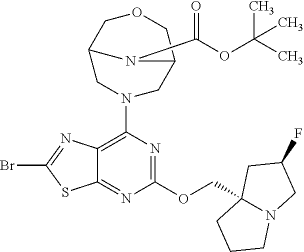 [SMILES: CC(C)(C)OC(=O)N1C2COCC1CN(c1nc(OC[C@@]34CCCN3C[C@H](F)C4)nc3sc(Br)nc13)C2]

To a stirred solution of tert-butyl 7-(5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (470 mg, 903 μmol) in tetrahydrofurane (8 mL) was added a solution of LiHMDS (1.8 ml, 1.0 M in THF, 1.8 mmol) at 0° C. The solution was stirred at 0° C. for 15 min. The solution was cooled to −78° C. and Bromine-1,4-dioxane complex (269 mg, 1.08 mmol) (CAS-RN: [15481-39-7]), dissolved in tetrahydrofurane (2 mL) was added. The solution was allowed to warm up to room temperature and was stirred at room temperature for 1 h. An aqueous solution of disodium sulfurothioate and a saturated solution of sodium bicarbonate were added and the mixture was extracted with ethyl acetate. The organic phase was dried (sodium sulfate), filtered and the solvent was removed in vacuum. Aminophase-silicagel chromatography (Gradient: hexane/ethyl acetate 10-50%) gave 272 mg (50% yield) of the title compound.

LC-MS (Method 1): Rₜ=1.05 min; MS (ESIpos): m/z=599 [M+H]⁺

¹H-NMR (400 MHz, DMSO-d₆): δ[ppm]=5.76-5.63 (m, 1H), 5.36-5.16 (m, 1H), 5.12-4.98 (m, 1H), 4.06 (br d, 2H), 4.03-3.90 (m, 2H), 3.84 (br s, 2H), 3.69-3.54 (m, 3H), 3.29-3.15 (m, 1H), 3.12-3.01 (m, 2H), 2.99 (s, 1H), 2.86-2.77 (m, 1H), 2.17-2.04 (m, 1H), 2.01 (d, 1H), 1.98-1.93 (m, 1H), 1.86-1.72 (m, 3H), 1.44 (s, 9H).

### Intermediate 12-4

### tert-butyl 7-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

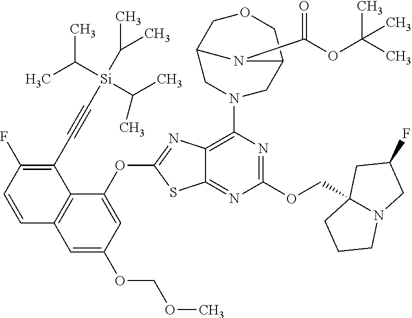 [SMILES: COCOc1cc(Oc2nc3c(N4CC5COCC(C4)N5C(=O)OC(C)(C)C)nc(OC[C@@]45CCCN4C[C@H](F)C5)nc3s2)c2c(C#C[Si](C(C)C)(C(C)C)C(C)C)c(F)ccc2c1]

To a stirred solution of tert-butyl 7-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (100 mg, 167 μmol) and 7-fluoro-3-(methoxymethoxy)-8-{[tri(propan-2-yl)silyl]ethynyl}naphthalen-1-ol (134 mg, 334 μmol) in N,N-dimethylacetamide (1.8 ml) was added potassium carbonate (115 mg, 834 μmol; CAS-RN: [584-08-7]) and the mixture was stirred at 110° C. for 2 h. Water was added and the mixture was extracted with dichloromethane. The organic phase was dried (sodium sulfate), filtered and the solvent was removed in vacuum. Silicagel chromatography (Gradient: dichloromethane/ethanol 10-30%) gave 86.0 mg (56% yield) of the title compound.

LC-MS (Method 1): Rₜ=1.57 min; MS (ESIpos): m/z=921 [M+H]⁺

¹H-NMR (400 MHz, DMSO-d6): δ[ppm]=8.08 (dd, 1H), 7.63 (d, 1H), 7.58 (t, 1H), 7.52 (d, 1H), 5.53-5.14 (m, 4H), 5.11-4.90 (m, 1H), 4.08-3.88 (m, 3H), 3.86-3.75 (m, 1H), 3.74-3.65 (m, 1H), 3.64-3.48 (m, 3H), 3.41 (s, 3H), 3.29-3.20 (m, 1H), 3.15-2.96 (m, 4H), 2.86-2.79 (m, 1H), 2.12-2.05 (m, 1H), 2.00 (br s, 1H), 1.95-1.92 (m, 1H), 1.87-1.69 (m, 3H), 1.42 (s, 9H), 1.10-1.02 (m, 3H), 1.02-0.93 (m, 18H).

### Intermediate 12-5

### tert-butyl 7-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

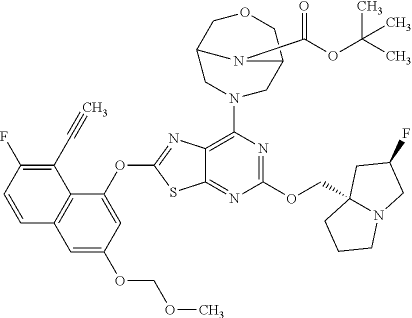 [SMILES: C#Cc1c(F)ccc2cc(OCOC)cc(Oc3nc4c(N5CC6COCC(C5)N6C(=O)OC(C)(C)C)nc(OC[C@@]56CCCN5C[C@H](F)C6)nc4s3)c12]

To a stirred solution of tert-butyl 7-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (80.0 mg, 86.8 μmol) in THF (500 μL], was added N,N,N-tributylbutan-1-aminium fluoride (170 μl, 1.0 M, 170 μmol) and the mixture was stirred at rt for 1 h. Aqueous ammonium chloride solution was added, the mixture was stirred for 3 min, then aqueous sodium bicarbonate solution was added until pH 10 was reached and the mixture was extracted with ethyl acetate. The organic phase was dried (sodium sulfate), filtered and the solvent was removed in vacuum. Aminophase-silicagel chromatography (Gradient: hexane/ethyl acetate 20-100%) gave 57.0 mg (86% yield) of the title compound.

LC-MS (Method 1): Rₜ=1.19 min; MS (ESIpos): m/z=765 [M+H]⁺

¹H-NMR (400 MHz, DMSO-d6): δ[ppm]=8.09 (dd, 1H), 7.65 (s, 2H), 7.58 (t, 1H), 5.36 (s, 2H), 5.34-5.13 (m, 2H), 5.05-4.89 (m, 1H), 4.68 (s, 1H), 4.01-3.84 (m, 3H), 3.83-3.69 (m, 1H), 3.62-3.43 (m, 4H), 3.42 (s, 3H), 3.24 (br s, 1H), 3.15-3.03 (m, 3H), 2.99 (s, 1H), 2.85-2.79 (m, 1H), 2.10-2.06 (m, 1H), 2.00 (br d, 1H), 1.97-1.93 (m, 1H), 1.86-1.70 (m, 3H), 1.40 (s, 9H).

### Example 13

### 5-ethynyl-6-fluoro-4-({7-(3-oxa-7,9-diazabicyclo[3.3.1]nonan-7-yl)-5-[(tetrahydro-1H-pyrrolizin-7a(5H)-yl)methoxy][1,3]thiazolo[5,4-d]pyrimidin-2-yl}oxy)naphthalen-2-ol, Hydrogen Chloride Salt

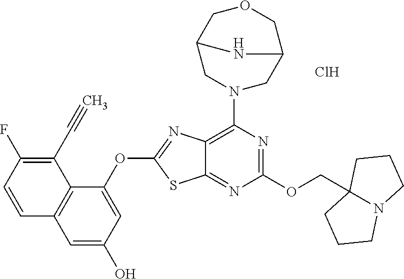 [SMILES: C#Cc1c(F)ccc2cc(O)cc(Oc3nc4c(N5CC6COCC(C5)N6)nc(OCC56CCCN5CCC6)nc4s3)c12 ; Cl]

To a stirred solution of tert-butyl 7-(2-{[8-ethynyl-7-fluoro-3-(methoxymethoxy)naphthalen-1-yl]oxy}-5-[(tetrahydro-1H-pyrrolizin-7a(5H)-yl)methoxy][1,3]thiazolo[5,4-d]pyrimidin-7-yl)-3-oxa-7,9-diazabicyclo[3.3.1]nonane-9-carboxylate (55.0 mg, 73.6 μmol) in dichloromethane (1.1 mL) and methanol (0.54 mL) was added HCl in dioxane (550 μl, 4.0 M, 2.2 mmol). The mixture was stirred at room temperature for 1.5 h. The solvent was mainly removed in vacuum and the residue was triturated with dichloromethane to give 39.0 mg (74% yield) of the title compound as a solid as a hydrogen chloride salt.

LC-MS (Method 1): Rₜ=0.75 min; MS (ESIpos): m/z=603 [M+H]⁺

¹H-NMR (400 MHz, DMSO-d₆): δ[ppm]=10.57 (br s, 2H), 10.29-10.09 (m, 2H), 7.97 (dd, 1H), 7.50 (t, 1H), 7.39 (d, 1H), 7.32 (d, 1H), 5.71-5.51 (m, 1H), 5.29-5.12 (m, 1H), 4.61 (d, 1H), 4.43 (br d, 2H), 4.04-3.88 (m, 3H), 3.78 (br s, 1H), 3.69-3.53 (m, 3H), 3.46 (dq, 2H), 3.41-3.33 (m, 1H), 3.25-3.13 (m, 2H), 2.21-2.03 (m, 4H), 2.02-1.88 (m, 4H).

### Intermediate 13-1

### tert-butyl 7-{5-[(tetrahydro-1H-pyrrolizin-7a(5H)-yl)methoxy][1,3]thiazolo[5,4-d]pyrimidin-7-yl}-3-oxa-7,9-diazabicyclo[3.3.1]nonane-9-carboxylate

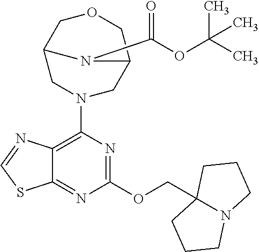 [SMILES: CC(C)(C)OC(=O)N1C2COCC1CN(c1nc(OCC34CCCN3CCC4)nc3scnc13)C2]

To a stirred solution of (tetrahydro-1H-pyrrolizin-7a(5H)-yl)methanol (231 mg, 1.63 mmol) in THF (5.0 ml) in a microwave tube, was added sodium hydride (70.4 mg, 60% purity, 1.76 mmol; CAS-RN: [7646-69-7]) at room temperature. After stirring for 10 minutes, tert-butyl 7-(5-chloro[1,3]thiazolo[5,4-d]pyrimidin-7-yl)-3-oxa-7,9-diazabicyclo[3.3.1]nonane-9-carboxylate (500 mg, 1.26 mmol) dissolved in N,N-dimethylacetamide (2.5 ml) was added and the reaction mixture was stirred at 75° C. for 1 h. Water was added and the mixture was extracted with a mixture of ethyl acetate and methanol. The organic phase was dried (sodium sulfate), filtered and the solvent was removed in vacuum. Aminophase-silicagel chromatography (Gradient: hexane/ethyl acetate 10-40%) gave 465 mg (74% yield) of the title compound.

LC-MS (Method 1): Rₜ=0.90 min; MS (ESIpos): m/z=503 [M+H]⁺

¹H-NMR (400 MHz, DMSO-d₆): δ[ppm]=8.97 (s, 1H), 6.09 (br d, 1H), 5.20-4.99 (m, 1H), 4.05 (br dd, 2H), 3.94 (br d, 2H), 3.83 (br s, 2H), 3.61 (br d, 3H), 3.24 (br s, 1H), 2.94-2.87 (m, 2H), 2.57-2.52 (m, 2H), 1.90-1.67 (m, 6H), 1.61-1.49 (m, 2H), 1.44 (s, 9H).

### Intermediate 13-2

### tert-butyl 7-{2-bromo-5-[(tetrahydro-1H-pyrrolizin-7a(5H)-yl)methoxy][1,3]thiazolo[5,4-d]pyrimidin-7-yl}-3-oxa-7,9-diazabicyclo[3.3.1]nonane-9-carboxylate

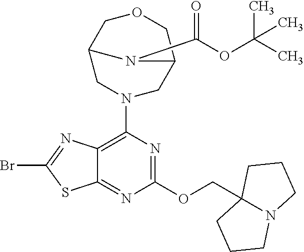 [SMILES: CC(C)(C)OC(=O)N1C2COCC1CN(c1nc(OCC34CCCN3CCC4)nc3sc(Br)nc13)C2]

To a stirred solution of tert-butyl 7-{5-[(tetrahydro-1H-pyrrolizin-7a(5H)-yl)methoxy][1,3]thiazolo[5,4-d]pyrimidin-7-yl}-3-oxa-7,9-diazabicyclo[3.3.1]nonane-9-carboxylate (450 mg, 895 μmol) in tetrahydrofurane (8 mL) was added a solution of LiHMDS (1.8 ml, 1.0 M, 1.8 mmol) at 0° C. The solution was stirred at 0° C. for 15 min. The solution was cooled to −78° C. and Bromine-1,4-dioxane complex (266 mg, 1.07 mmol) (CAS-RN: [15481-39-7]), dissolved in tetrahydrofurane (2 mL) was added. The solution was allowed to warm up to 0° C. and was stirred at 0° C. for 1 h. An aqueous solution of disodium sulfurothioate and a saturated solution of sodium bicarbonate were added and the mixture was extracted with ethyl acetate. The organic phase was dried (sodium sulfate), filtered and the solvent was removed in vacuum. Aminophase-silicagel chromatography (Gradient: hexane/ethyl acetate 10-35%) gave 235 mg (45% yield) of the title compound.

LC-MS (Method 1): Rₜ=1.00 min; MS (ESIpos): m/z=581 [M+H]⁺

¹H-NMR (400 MHz, DMSO-d₆): δ[ppm]=5.70 (br d, 1H), 5.12-4.97 (m, 1H), 4.12-4.04 (m, 2H), 3.93 (br d, 2H), 3.84 (br d, 2H), 3.69-3.55 (m, 3H), 3.29-3.17 (m, 1H), 2.95-2.85 (m, 2H), 2.57-2.53 (m, 1H), 1.90-1.66 (m, 7H), 1.59-1.50 (m, 2H), 1.44 (s, 9H).

### Intermediate 13-3

### tert-butyl 7-(2-{[7-fluoro-3-(methoxymethoxy)-8-{[tri(propan-2-yl)silyl]ethynyl}naphthalen-1-yl]oxy}-5-[(tetrahydro-1H-pyrrolizin-7a(5H)-yl)methoxy][1,3]thiazolo[5,4-d]pyrimidin-7-yl)-3-oxa-7,9-diazabicyclo[3.3.1]nonane-9-carboxylate

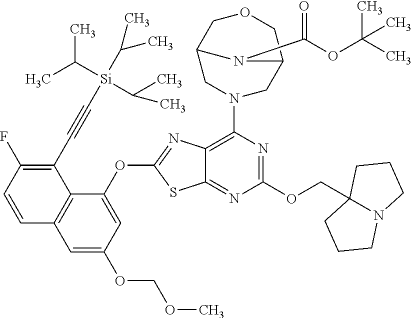 [SMILES: COCOc1cc(Oc2nc3c(N4CC5COCC(C4)N5C(=O)OC(C)(C)C)nc(OCC45CCCN4CCC5)nc3s2)c2c(C#C[Si](C(C)C)(C(C)C)C(C)C)c(F)ccc2c1]

To a stirred solution of tert-butyl 7-{2-bromo-5-[(tetrahydro-1H-pyrrolizin-7a(5H)-yl)methoxy][1,3]thiazolo[5,4-d]pyrimidin-7-yl}-3-oxa-7,9-diazabicyclo[3.3.1]nonane-9-carboxylate (100 mg, 172 μmol) and 7-fluoro-3-(methoxymethoxy)-8-{[tri(propan-2-yl)silyl]ethynyl}naphthalen-1-ol (138 mg, 344 μmol) in N,N-dimethylacetamide (1.8 ml) was added potassium carbonate (119 mg, 860 μmol) and the mixture was stirred at 110° C. for 2 h. Water was added and the mixture was extracted with methylene chloride. The organic phase was washed with half-saturated sodium chloride solution, dried (sodium sulfate), filtered and the solvent was removed in vacuum. Aminophase-silicagel chromatography (Gradient: hexanes/ethyl acetate 10-30%) gave 101 mg (90% purity, 59% yield) of the title compound.

LC-MS (Method 1): Rₜ=1.58 min; MS (ESIpos): m/z=903 [M+H]⁺

¹H-NMR (400 MHz, DMSO-d₆): δ[ppm]=8.08 (dd, 1H), 7.63 (d, 1H), 7.58 (t, 1H), 7.52 (d, 1H), 5.50-5.36 (m, 1H), 5.34 (s, 2H), 5.11-4.90 (m, 1H), 3.92 (br s, 1H), 4.16-3.45 (m, 9H), 3.41 (s, 3H), 3.06 (s, 2H), 2.93-2.86 (m, 2H), 1.87-1.61 (m, 7H), 1.56-1.47 (m, 2H), 1.42 (s, 9H).

### Intermediate 13-4

### tert-butyl 7-(2-{[8-ethynyl-7-fluoro-3-(methoxymethoxy)naphthalen-1-yl]oxy}-5-[(tetrahydro-1H-pyrrolizin-7a(5H)-yl)methoxy][1,3]thiazolo[5,4-d]pyrimidin-7-yl)-3-oxa-7,9-diazabicyclo[3.3.1]nonane-9-carboxylate

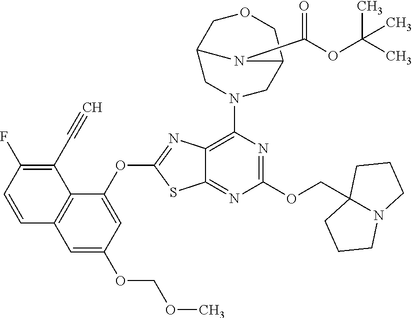 [SMILES: C#Cc1c(F)ccc2cc(OCOC)cc(Oc3nc4c(N5CC6COCC(C5)N6C(=O)OC(C)(C)C)nc(OCC56CCCN5CCC6)nc4s3)c12]

To a stirred solution of tert-butyl 7-(2-{[7-fluoro-3-(methoxymethoxy)-8-{[tri(propan-2-yl)silyl]ethynyl}naphthalen-1-yl]oxy}-5-[(tetrahydro-1H-pyrrolizin-7a(5H)-yl)methoxy][1,3]thiazolo[5,4-d]pyrimidin-7-yl)-3-oxa-7,9-diazabicyclo[3.3.1]nonane-9-carboxylate (95.0 mg, 105 μmol) in THF (590 μl), was added N,N,N-tributylbutan-1-aminium fluoride (210 μl, 1.0 M, 210 μmol) and the mixture was stirred at rt for 1 h. Aqueous sodium bicarbonate solution was added and the mixture was extracted with ethyl acetate. The organic phase was dried (sodium sulfate), filtered and the solvent was removed in vacuum. Aminophase-silicagel chromatography (Gradient: hexane/ethyl acetate 20-65%) gave 58.0 mg (90% purity, 66% yield) of the title compound.

LC-MS (Method 1): Rₜ=1.23 min; MS (ESIpos): m/z=747.6 [M+H]⁺

¹H-NMR (400 MHz, DMSO-d₆): δ[ppm]=8.09 (dd, 1H), 7.68-7.61 (m, 2H), 7.58 (t, 1H), 5.36 (s, 2H), 5.33-5.20 (m, 1H), 5.06-4.86 (m, 1H), 4.68 (s, 1H), 3.99-3.84 (m, 3H), 3.83-3.68 (m, 1H), 3.50 (br d, 4H), 3.42 (s, 3H), 3.29-3.04 (m, 2H), 2.93-2.86 (m, 2H), 2.53-2.51 (m, 2H), 1.89-1.64 (m, 6H), 1.58-1.48 (m, 2H), 1.40 (s, 9H).

### Example 14

### 4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}methyl)-5-fluoronaphthalen-2-ol

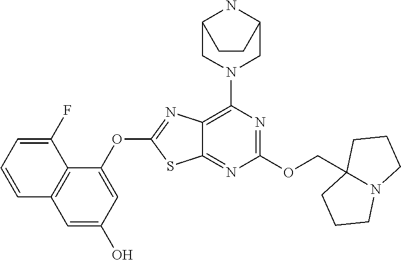 [SMILES: Oc1cc(Oc2nc3c(N4CC5CCC(C4)N5)nc(OCC45CCCN4CCC5)nc3s2)c2c(F)cccc2c1]

To a solution of tert-butyl 3-{2-[(R)-[8-fluoro-3-(methoxymethoxy)naphthalen-1-yl](hydroxy)methyl]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (50.0 mg, 69.4 μmol) in dichloromethane (450 μL) was added trifluoroacetic acid (160 μl, 2.1 mmol; CAS-RN: [76-05-1]) and triethylsilane: (130 μl, 830 μmol; CAS-RN: [617-86-7]) and the mixture was stirred at 70° C. for 1 hour in a microwave reactor. Afterwards, the mixture was concentrated. Dichloromethane (450 μL), trifluoroacetic acid (160 μl, 2.1 mmol; CAS-RN: [76-05-1]), and triethylsilane: (130 μl, 830 μmol; CAS-RN: [617-86-7]) were added again and the mixture was heated in a microwave reactor at 80° C. for 2 hours. Then, the mixture was diluted with a mixture of dichloromethane/isopropanole (7:3) and basified using saturated, aqueous NaHCO₃-solution. The layers were separated and the aqueous layer was extracted once again. The combined organic layers were dried using a water-resistant filter. The clear filtrate was concentrated under reduced pressure. The crude product was purified by prep. HPLC, basic gradient to give the title compound (1.50 mg (91% purity, 4% yield).

LC-MS (method 2): Rₜ=1.11 min; MS (ESIneg): m/z=559 [M−H]⁻

### Intermediate 14-1

### tert-butyl 3-{2-[(R)-[8-fluoro-3-(methoxymethoxy)naphthalen-1-yl](hydroxy)methyl]-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

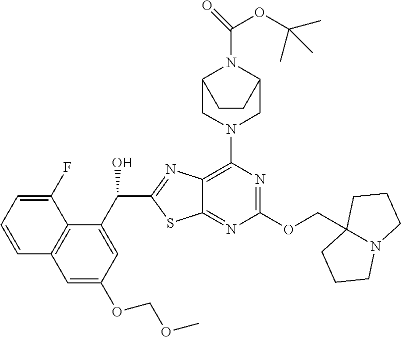 [SMILES: COCOc1cc([C@H](O)c2nc3c(N4CC5CCC(C4)N5C(=O)OC(C)(C)C)nc(OCC45CCCN4CCC5)nc3s2)c2c(F)cccc2c1]

tert-butyl 3-{5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (126 mg, 260 μmol, example 1, intermediate 2)) was dissolved in THF (1.1 ml) and cooled down to −78° C. The mixture was evacuated and flushed with Argon (3×). Then the n-BuLi (220 μl, 1.6 M in hexane, 340 μmol; CAS-RN: [109-72-8]) was added drop wise->exotherm. After 30 min, 8-fluoro-3-(methoxymethoxy)naphthalene-1-carbaldehyde (70.0 mg, 299 μmol) dissolved in THF (1.1 ml) was added drop wise and it was stirred at −78° C. for 2 hours. Afterwards, the reaction mixture was carefully quenched with water and diluted with dichloromethane. It was extracted three times with dichloromethane, washed once with water and brine, filtered through a silicone coated filter and concentrated under reduced pressure. The crude product was purified by flash chromatography to yield the title compound (100 mg, 77% purity, 41% yield).

LC-MS (method 2): Rt=1.61 min; MS (ESIpos): m/z=722 [M+H]⁺

¹H-NMR (400 MHz, DMSO-d6) delta [ppm]: 0.798 (0.81), 0.814 (0.91), 0.821 (0.91), 0.841 (0.65), 0.843 (0.57), 0.850 (0.61), 0.857 (0.65), 0.872 (0.49), 0.886 (0.53), 0.904 (1.10), 0.922 (0.55), 1.022 (0.43), 1.118 (0.55), 1.133 (0.55), 1.231 (0.65), 1.414 (4.97), 1.437 (16.00), 1.498 (0.71), 1.510 (0.59), 1.516 (0.73), 1.531 (0.89), 1.545 (0.77), 1.563 (0.55), 1.582 (0.41), 1.613 (0.51), 1.632 (0.57), 1.674 (0.51), 1.690 (0.85), 1.706 (1.05), 1.722 (1.01), 1.737 (0.95), 1.749 (1.03), 1.764 (1.12), 1.779 (1.30), 1.790 (1.28), 1.799 (0.89), 1.812 (0.99), 1.826 (1.22), 1.843 (0.95), 1.856 (0.81), 1.870 (0.49), 1.905 (0.55), 2.332 (0.81), 2.518 (5.50), 2.523 (4.22), 2.866 (0.47), 2.880 (0.83), 2.893 (0.97), 2.906 (1.10), 2.919 (0.81), 2.931 (0.53), 3.408 (9.81), 3.884 (1.85), 3.946 (1.48), 4.277 (0.51), 5.328 (3.41), 5.758 (2.19), 7.468 (0.39), 7.490 (1.05), 7.621 (1.14), 7.627 (1.03), 7.704 (0.65), 7.724 (0.55), 8.990 (3.43).

### Intermediate 14-1 Building Block 1, 1. Step

### 8-fluoro-3-(methoxymethoxy)naphthalene-1-carbonitrile

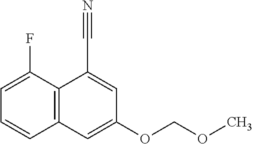 [SMILES: COCOc1cc(C#N)c2c(F)cccc2c1]

To a solution of 1-bromo-8-fluoro-3-(methoxymethoxy)naphthalene (4.50 g, 15.8 mmol, see WO 2021/041671, example 282, step F, page 524) in N,N-Dimethylformamide (24 ml) Zn(CN)₂ (2.04 g, 17.4 mmol; CAS-RN: [557-21-1]) and Tetrakis(triphenylphosphine)palladium(0) (2.28 g, 1.97 mmol; CAS-RN: [14221-01-3]) were added. N₂ was bubbled through the mixture for 5 minutes and afterwards it was stirred at 100° C. for 1 hour in the microwave reactor. Then, a formed precipitate was filtered off and the filtrate was concentrated under reduced pressure to remove the DMF. The remaining residue was dissolved in ethyl acetate and diluted with water. It was extracted three times with water, washed once with half concentrated NaCl solution and concentrated under reduced pressure. The crude product was purified by flash chromatography, 100 g column, silica gel, hexane/ethyl acetate 0%-70%, to yield the title compound (3.41 g (96% purity, 90% yield).

1H-NMR (400 MHz, DMSO-d6) delta [ppm]: 2.518 (0.74), 2.523 (0.53), 3.431 (16.00), 5.404 (8.37), 7.378 (0.50), 7.381 (0.52), 7.397 (0.65), 7.400 (0.62), 7.409 (0.53), 7.411 (0.56), 7.428 (0.66), 7.431 (0.64), 7.594 (0.47), 7.607 (0.50), 7.614 (0.75), 7.627 (0.78), 7.634 (0.54), 7.648 (0.49), 7.834 (1.07), 7.853 (0.90), 7.923 (0.78), 7.929 (1.46), 7.934 (0.92), 7.996 (2.27), 8.002 (1.93).

### Intermediate 14-1 Building Block 1, 2. Step

### 8-fluoro-3-(methoxymethoxy)naphthalene-1-carbaldehyde

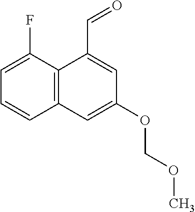 [SMILES: COCOc1cc(C=O)c2c(F)cccc2c1]

To a stirred solution of 8-fluoro-3-(methoxymethoxy)naphthalene-1-carbonitrile (1.03 g, 4.45 mmol) in dichloromethane (13 ml) was added dropwise DIBALH (8.9 ml, 1.0 M in hexane, 8.9 mmol; CAS-RN:[1191-15-7]) under N₂ atmosphere at −78° C. After 1 h, the reaction was allowed to warm up to room temperature and quenched with methanol. Then saturated aqueous NH₄Cl and ethyl acetate were added and the mixture was stirred at room temperature for 10 minutes. Afterwards, it was extracted three times with ethyl acetate. The organic layer was filtered through silicone paper and solvents were removed in vacuo. The residue was redissolved in 15 ml toluene to which 15 ml saturated aqueous NH₄Cl was added and the mixture was stirred vigorously for 30 min. Afterwards, 2 ml of 2M HCl were added and the reaction mixture was stirred for 15 min. Then, it was extracted with ethyl acetate twice and the organic layers were filtered through silicone paper. Solvents were removed in vacuo and the crude product was purified by chromatography over SiO2 using a gradient of EtOAc in hexane (0 to 50%) to afford the title compound (585 mg, 99% purity, 55% yield). 1H-NMR (400 MHz, CHLOROFORM-d) delta [ppm]: 1.558 (16.00), 3.532 (8.77), 5.342 (5.25), 7.256 (0.42), 7.457 (0.44), 7.470 (0.44), 7.646 (0.60), 7.648 (0.60), 7.667 (0.55), 7.669 (0.51), 7.674 (0.49), 7.679 (0.78), 7.684 (0.48), 7.971 (1.18), 7.978 (1.14), 10.972 (1.67), 10.977 (1.36).

### Example 15

### Formic acid-5-Chlor-4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol (2:1)

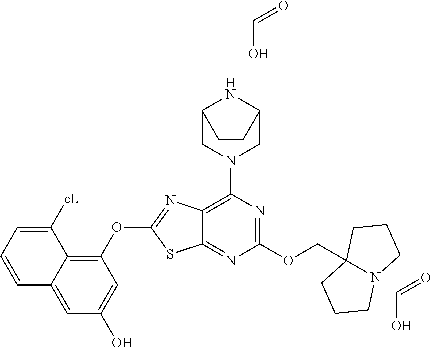

Tert-Butyl-3-[2-({8-chlor-3-[(2-methoxyethoxy)methoxy]-1-naphthyl}oxy)-5-(tetrahydro-1H-pyrrolizin-7a(5H)-ylmethoxy)[1,3]thiazolo[5,4-d]pyrimidin-7-yl]-3,8-diazabicyclo[3.2.1]octan-8-carboxylat (33.0 mg, 43.0 μmol) was dissolved in acetonitrile (340 μl), cooled to 0° C. and HCl in 1,4-dioxane (110 μl, 4.0 M, 430 μmol; CAS-RN: [7647-01-0]) was added. The reaction mixture was stirred for 30 min under cooling condition and at rt for 30 min. HCl in 1,4-dioxane (220 μl, 4.0 M, 860 μmol; CAS-RN: [7647-01-0]) was added and the mixture was stirred for 30 min at rt. HCl in 1,4-dioxane (220 μl, 4.0 M, 860 μmol; CAS-RN: [7647-01-0]) was added again and the reaction was stirred for 30 min rt. The reaction was diluted with sat. aq. NaHCO₃ solution and extracted with DCM/isopropanole (7:3). The combined organic layers were dried using a water-resistant silicone filter and the filtrate was concentrated under reduced pressure. The crude material was purified by preparative HPLC (Method A, 10-50% ACN) to afford 5.9 mg of the title compound (20% yield).

LC-MS (Method 1): Rₜ=0.79 min; MS (ESIpos): m/z=579 [M+H]⁺

¹H-NMR (400 MHz, DMSO-d₆) δ[ppm]: 1.229 (1.40), 1.474 (3.22), 1.491 (3.68), 1.523 (1.28), 1.540 (3.85), 1.552 (2.88), 1.558 (3.30), 1.571 (3.81), 1.589 (4.00), 1.600 (2.70), 1.670 (0.60), 1.688 (1.24), 1.703 (1.84), 1.718 (3.09), 1.735 (3.70), 1.751 (2.65), 1.768 (2.68), 1.784 (3.73), 1.798 (3.88), 1.813 (3.24), 1.825 (4.76), 1.837 (3.39), 1.850 (4.12), 1.864 (2.96), 1.880 (1.49), 1.955 (1.82), 2.332 (1.80), 2.336 (0.79), 2.518 (8.88), 2.523 (6.20), 2.565 (2.99), 2.571 (2.53), 2.580 (2.77), 2.586 (3.13), 2.604 (2.09), 2.678 (0.79), 2.781 (1.73), 2.920 (2.04), 2.935 (4.10), 2.942 (4.52), 2.948 (3.54), 2.959 (3.70), 2.974 (2.10), 3.031 (2.25), 3.062 (2.32), 3.385 (1.44), 3.918 (15.33), 4.681 (0.77), 7.255 (7.04), 7.261 (10.87), 7.281 (8.50), 7.287 (5.45), 7.391 (1.56), 7.400 (16.00), 7.410 (7.95), 7.415 (7.52), 7.434 (1.09), 7.793 (0.72), 7.803 (3.57), 7.813 (3.14), 7.818 (3.14), 7.827 (3.16), 7.837 (0.64), 8.087 (0.69), 8.243 (10.29).

### Intermediate 15-1

### tert-butyl 3-[5-(tetrahydro-1H-pyrrolizin-7a(5H)-ylmethoxy)[1,3]thiazolo[5,4-d]pyrimidin-7-yl]-3,8-diazabicyclo[3.2.1]octane-8-carboxylate

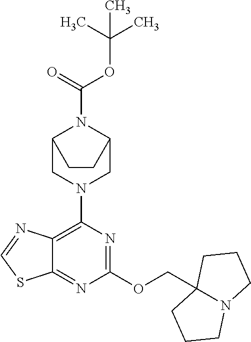 [SMILES: CC(C)(C)OC(=O)N1C2CCC1CN(c1nc(OCC34CCCN3CCC4)nc3scnc13)C2]

tert-butyl 3-(5-chloro[1,3]thiazolo[5,4-d]pyrimidin-7-yl)-3,8-diazabicyclo[3.2.1]octane-8-carboxylate (308 mg, 807 μmol) and tetrahydro-1H-pyrrolizin-7a(5H)-ylmethanol (342 mg, 2.42 mmol) were dissolved in THF (5.2 ml) and treated portionwise with NaH (96.8 mg, 60% purity, 2.42 mmol; CAS-RN: [7646-69-7]). The mixture was stirred under Ar atmosphere for 5 min. DMAc (1.5 ml) was added and the reaction was stirred under Ar atmosphere for 1 h and after that for 1 h at 75° C. The reaction mixture was quenched with water and diluted with ethyl acetate and stirred for a few minutes. The organic layer was separated and filtered through a silicone coated filter and concentrated under reduced pressure. The crude product was purified by flash chromatography (aminophase, using a gradient of hexane/ethyl acetate 25-75%) to afford 283 mg of the title compound (97% purity, 70% yield).

LC-MS (Method 2): Rt=1.48 min; MS (ESIpos): m/z=487 [M+H]⁺

¹H-NMR (400 MHz, DMSO-d6) δ[ppm]: 1.437 (16.00), 1.525 (0.45), 1.555 (0.51), 1.613 (0.44), 1.632 (0.50), 1.734 (0.52), 1.749 (0.43), 1.771 (0.50), 1.786 (0.49), 1.822 (0.66), 1.838 (0.68), 1.851 (0.84), 1.863 (0.50), 1.955 (0.84), 2.518 (1.11), 2.523 (0.90), 2.782 (0.78), 2.897 (0.55), 2.910 (0.44), 2.921 (0.52), 2.942 (1.38), 3.936 (1.79), 4.277 (0.52), 5.758 (7.73), 8.988 (3.75).

### Intermediate 15-2

### tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

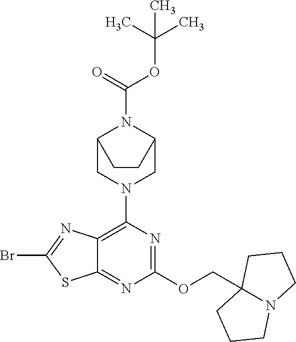 [SMILES: CC(C)(C)OC(=O)N1C2CCC1CN(c1nc(OCC34CCCN3CCC4)nc3sc(Br)nc13)C2]

tert-butyl 3-{5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (1.00 g, 2.05 mmol) was dissolved in THF (8.3 ml) and cooled to −78° C. Lithium bis(trimethylsilyl)amide (2.8 ml, 1.0 M, 2.8 mmol; CAS-RN: [4039-32-1]) was added and the reaction was stirred at −78° C. for 30 min. Bromine: (150 μl, 2.9 mmol; CAS-RN:[7726-95-6]) was dissolved in THF (2.5 ml) and the solution was then added to the reaction mixture. It was stirred at −78° C. for 25 min. The reaction mixture was quenched by addition of water and aq. Sodium thiosulfate (10% aq. solution) and it was extracted with DCM. The combined organic layers were dried using a silicone coated filter and concentrated under reduced pressure. The crude product was purified by flash chromatography (aminophase, using a gradient of hexane/ethyl acetate 5-50%) to afford 398 mg of the title compound (97% purity, 33% yield).

LC-MS (Method 2): Rₜ=1.71 min; MS (ESIpos): m/z=567 [M+H]+

### Intermediate 15-3

### tert-butyl 3-{2-({8-chloro-3-[(2-methoxyethoxy)methoxy]naphthalen-1-yl}oxy)-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate

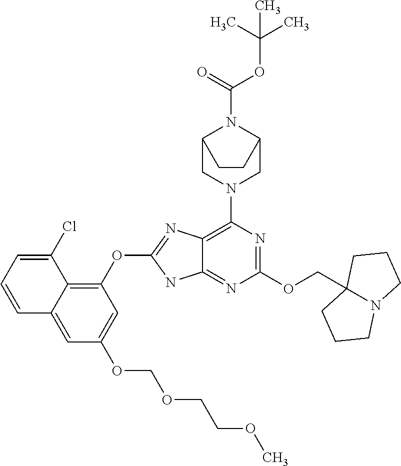 [SMILES: COCCOCOc1cc(Oc2nc3c(N4CC5CCC(C4)N5C(=O)OC(C)(C)C)nc(OCC45CCCN4CCC5)nc3[nH]2)c2c(Cl)cccc2c1]

tert-butyl 3-{2-bromo-5-[(tetrahydro-1H-pyrrolizin-7a(5H)-yl)methoxy][1,3]thiazolo[5,4-d]pyrimidin-7-yl}-3,8-diazabicyclo[3.2.1]octane-8-carboxylate (24.0 mg, 42.4 μmol) and 8-chloro-3-[(2-methoxyethoxy)methoxy]naphthalen-1-ol (24.0 mg, 84.9 μmol) were dissolved in DMAc (450 μl) and potassium carbonate (29.3 mg, 212 μmol; CAS-RN: [584-08-7]) was added. The reaction mixture was stirred in a microwave reactor at 110° C. for 2 h. The reaction was diluted with water and methylene chloride. The separated aqueous layer was extracted with methylene chloride and the combined organic layers were dried through a silicone coated filter. The filtrate was concentrated under reduced pressure to afford 33 mg of the title compound (91% purity, 92% yield), which was used without further purification.

LC-MS (Method 1): Rₜ=1.34 min; MS (ESIpos): m/z=768 [M+H]⁺

### Intermediate 15-3 Building Block Step 1

### (4-bromo-5-chloronaphthalen-2-yl)boronic acid

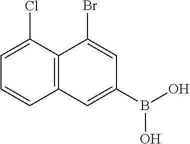 [SMILES: OB(O)c1cc(Br)c2c(Cl)cccc2c1]

1-bromo-8-chloronaphthalene (1.02 g, 4.22 mmol) and 4,4,4′,4′,5,5,5′,5′-octamethyl-2,2′-bi-1,3,2-dioxaborolane (1.18 g, 4.65 mmol; CAS-RN: [73183-34-3]) were dissolved in cyclohexane (16 ml) and treated with bis(1,5-cyclooctadiene)dimethoxydiiridium (140 mg, 211 μmol; CAS-RN: [12148-71-9]) and 4,4′-di-tert-butyl-2,2′-bipyridine (113 mg, 422 μmol; CAS-RN: [72914-19-3]). The mixture was purged with N₂ and stirred in a microwave reactor at 80° C. for 2 h. The solvent was removed under reduced pressure to afford 1.21 g of the title compound (50% purity, 50% yield), which was used without further purification.

LC-MS (Method 2): Rₜ=0.75 min; MS (ESIneg): m/z=285 [M−H]+

### Intermediate 15-3 Building Block Step 2

### 4-bromo-5-chloronaphthalen-2-ol

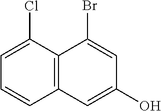 [SMILES: Oc1cc(Br)c2c(Cl)cccc2c1]

4-bromo-5-chloronaphthalen-2-yl)boronic acid (1.21 g, 50% purity, 2.12 mmol) was dissolved in THF (86 ml) and NaOH (254 mg, 6.36 mmol; CAS-RN: [1310-73-2]) was added. After 5 min of stirring an aqueous solution of H₂O₂ (650 μl, 30% purity, 6.4 mmol; CAS-RN: [7722-84-1]) was added to the mixture. The reaction was stirred at rt for 4 h. The mixture was acidified to pH 2 using aqueous 1 M HCl. It was diluted with some water and extracted with methylene chloride. The combined organic layers were filtered through a silicone coated filter and concentrated under reduced pressure. The crude material was purified by flash chromatography (silica column, using a gradient of hexane/ethyl acetate 0-50%) to afford 438 mg of the title compound (60% purity, 50% yield).

LC-MS (Method 2): Rt=0.89 min; MS (ESIneg): m/z=257 [M−H]⁺

### Intermediate 15-3 Building Block Step 3

### 1-bromo-8-chloro-3-[(2-methoxyethoxy)methoxy]naphthalene

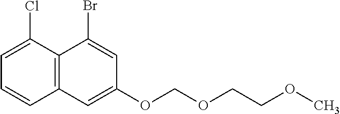 [SMILES: COCCOCOc1cc(Br)c2c(Cl)cccc2c1]

4-Bromo-5-chloronaphthalen-2-ol (425 mg, 1.65 mmol) was dissolved in DCM (8.0 ml) and N,N-diisopropylethylamine (720 μl, 4.1 mmol; CAS-RN: [7087-68-5]) was added. Under N₂ atmosphere the mixture was cooled down to 0° C. and carefully treated with 1-(chloromethoxy)-2-methoxyethane (340 μl, 2.9 mmol). It was stirred at 0° C. for 30 min and at rt for 72 h. The reaction mixture was diluted with water and extracted with methylene chloride. The combined organic layers were dried using a silicone coated filter and concentrated under reduced pressure to afford 650 mg of the title compound (100% yield) which was used without further purification.

### Intermediate 15-4 Building Block 1, 4. Step

### 8-chloro-3-[(2-methoxyethoxy)methoxy]naphthalen-1-ol

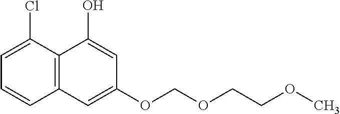 [SMILES: COCCOCOc1cc(O)c2c(Cl)cccc2c1]

1-bromo-8-chloro-3-[(2-methoxyethoxy)methoxy]naphthalene (575 mg, 1.66 mmol), potassium hydroxide (467 mg, 8.32 mmol; CAS-RN: [1310-58-3]), Pd₂(dba)₃ (107 mg, 116 μmol; CAS-RN:[52409-22-0]) and 2-di-tert-butylphosphino-2′,4′,6′-triisopropylbiphenyl (106 mg, 250 μmol; CAS-RN: [564483-19-8]) were suspended in 1,4-dioxane (4.3 ml) and water (1.8 ml) and degased with N₂ for 5 min. The mixture was stirred in a microwave reactor at 95° C. for 75 min. The mixture was diluted with DCM and rests of catalysts were filtered off over celite. The filtrate was diluted with water and the layers were separated. The aqueous layer was extracted with DCM and the combined organic layers were dried using a silicone coated filter and concentrated under reduced pressure. The crude product was purified by flash column chromatography (silica column, using a gradient of hexane/ethyl acetate 0-75%) to afford 293 mg product (22% purity). The product was purified by prep. HPLC (Method B, using a gradient of water/acetonitrile 10-100%) to afford 28.9 mg of the title compound (94% purity, 6% yield).

LC-MS (Method 2): Rt=1.08 min; MS (ESIneg): m/z=281 [M−H]⁻

¹H NMR (400 MHz, DMSO-d₆) δ[ppm]=3.22 (s, 3H) 3.44-3.50 (m, 2H) 3.69-3.79 (m, 2H) 5.32 (s, 2H) 6.68 (d, 1H) 6.95 (d, 1H) 7.27 (s, 1H) 7.29 (d, 1H) 7.58-7.67 (m, 1H) 10.25 (br s, 1H).

### Example 16

### 4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol, Hydrogen Chloride Salt (Mixture of 2 Diastereomers)

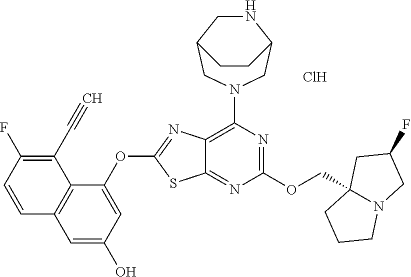 [SMILES: C#Cc1c(F)ccc2cc(O)cc(Oc3nc4c(N5CC6CCC(C5)NC6)nc(OC[C@@]56CCCN5C[C@H](F)C6)nc4s3)c12 ; Cl]

tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 diastereomers) (35.0 mg, 45.9 μmol) was dissolved in dichloromethane (680 μl) and methanol (340 μmol). HCl in 1,4-dioxane (340 μl, 4.0 M, 1.4 mmol) was added and the reaction mixture was stirred in a microwave vessel at rt for 1 h. The reaction mixture was concentrated to the half of the volume under reduced pressure. Dichloromethane was added and the solid material was centrifuged. The solvent was decanted and the solid was dried under reduced pressure to afford 8.2 mg of the title compound (90% purity, 65% yield).

LC-MS (Method 1): Rₜ=0.82 min; MS (ESIpos): m/z=620 [M+H]⁺

¹H-NMR (400 MHz, DMSO-d6) δ[ppm]: 1.440 (0.97), 1.466 (1.14), 1.487 (0.61), 1.534 (0.43), 1.803 (0.42), 2.021 (0.55), 2.043 (0.53), 2.080 (0.52), 2.095 (0.58), 2.111 (0.62), 2.127 (0.70), 2.144 (0.89), 2.168 (0.59), 2.183 (0.48), 2.248 (0.48), 2.277 (0.63), 2.293 (0.45), 2.322 (0.64), 2.326 (0.83), 2.332 (0.59), 2.433 (0.74), 2.453 (0.79), 2.518 (4.23), 2.522 (2.64), 2.575 (0.54), 2.664 (0.58), 2.669 (0.80), 2.673 (0.59), 3.162 (0.44), 3.228 (1.22), 3.244 (1.04), 3.514 (1.00), 3.552 (0.98), 3.564 (1.29), 3.732 (0.70), 3.745 (0.68), 3.776 (0.93), 3.820 (0.69), 4.289 (2.28), 4.469 (4.56), 4.591 (3.51), 4.593 (3.57), 5.486 (0.60), 5.618 (0.60), 5.759 (16.00), 7.313 (2.16), 7.319 (3.20), 7.343 (2.79), 7.348 (2.01), 7.465 (1.26), 7.488 (2.50), 7.511 (1.30), 7.949 (1.07), 7.964 (1.11), 7.972 (1.16), 7.986 (1.08), 9.088 (0.49), 9.422 (0.53), 11.256 (0.59).

### Intermediate 16-1

### tert-butyl (1S*,5R*)-3-(5-chloro[1,3]thiazolo[5,4-d]pyrimidin-7-yl)-3,6-diazabicyclo[3.2.2]nonane-6-carboxylate (Mixture of 2 Enantiomers)

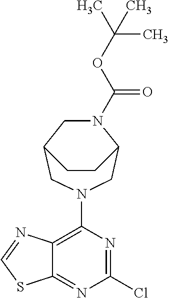 [SMILES: CC(C)(C)OC(=O)N1CC2CCC1CN(c1nc(Cl)nc3scnc13)C2]

5,7-dichloro[1,3]thiazolo[5,4-d]pyrimidine (191 mg, 929 μmol; CAS-RN: [13479-88-4]) and tert-butyl (1S*,5R*)-3,6-diazabicyclo[3.2.2]nonane-6-carboxylate (210 mg, 929 μmol; CAS-RN: [1214743-62-0) were dissolved in 1,4-dioxane (4.0 ml) and TEA (320 μl, 2.3 mmol; CAS-RN: [121-44-8]) was added. The reaction mixture was stirred under Ar atmosphere at rt for 90 min. The solvent was removed under reduced pressure. Water was added and the mixture was extracted with dichloromethane. The combined organic layers were washed with water and dried using a silicone coated filter. The filtrate was concentrated under reduced pressure to afford 393 mg of the title compound (99% purity, 99% yield).

LC-MS (Method 1): Rₜ=1.47 min; MS (ESIpos): m/z=396 [M+H]⁺

### Intermediate 16-2

### tert-butyl (1S*,5R*)-3-(5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 Diastereomers)

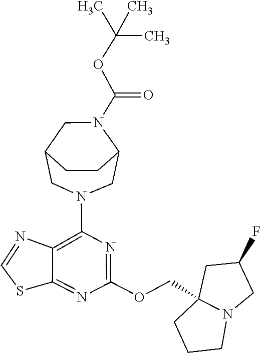 [SMILES: CC(C)(C)OC(=O)N1CC2CCC1CN(c1nc(OC[C@@]34CCCN3C[C@H](F)C4)nc3scnc13)C2]

[(2R,7aS)-2-fluorotetrahydro-1H-pyrrolizin-7a(5H)-yl]methanol (204 mg, 1.28 mmol) was dissolved in THF (4.0 ml) and sodium hydride (55.2 mg, 60% purity, 1.38 mmol; CAS-RN: [7646-69-7]) was added at rt. The mixture was stirred for 10 min. tert-butyl (1S*,5R*)-3-(5-chloro[1,3]thiazolo[5,4-d]pyrimidin-7-yl)-3,6-diazabicyclo[3.2.2]nonane-6-carboxylate (390 mg, 985 μmol) was dissolved in N,N-dimethylacetamide (2.0 ml) and added to the first mixture. The reaction mixture was stirred at 75° C. for 1 h. The mixture was quenched with water and extracted with ethyl acetate/methanol (10:1). The combined organic layers were dried with sodium sulfate. After filtration the filtrate was concentrated under reduced pressure. The crude product was purified by flash chromatography (amino phase, using a gradient of hexane/ethyl acetate 10-40%) to afford 373.9 mg of the title compound (95% purity, 70% yield).

LC-MS (Method 1): Rₜ=1.05 min; MS (ESIpos): m/z=520 [M+H]⁺

### Intermediate 16-3

### tert-butyl (1S*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 Diastereomers)

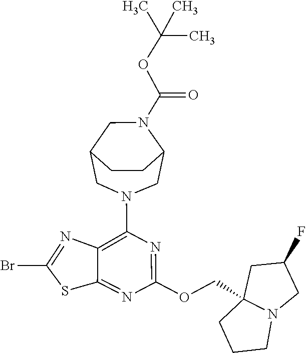 [SMILES: CC(C)(C)OC(=O)N1CC2CCC1CN(c1nc(OC[C@@]34CCCN3C[C@H](F)C4)nc3sc(Br)nc13)C2]

tert-butyl (1S*,5R*)-3-(5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (370 mg, 713 μmol) was dissolved in THF (6.0 ml) and at 0° C. Lithium bis(trimethylsilyl)amide (1.4 ml, 1.0 M in THF, 1.4 mmol; CAS-RN: [4039-32-1]) was added and the reaction was stirred for 15 min at this temperature. bromine-1,4-dioxane complex (1:1) (212 mg, 856 μmol; CAS-RN: [15481-39-7]) was dissolved in THF (2.0 ml) and added at −78° C. to the first reaction mixture. The mixture was allowed to get to 0° C. and was stirred for 1 h at this temperature. The reaction was quenched with aq. sodium thiosulfate and sat. aq. NaHCO₃ solution and extracted with ethyl acetate. The combined organic layers were dried with sodium sulfate and after filtration the filtrate was concentrated under reduced pressure. The crude product was purified by flash chromatography (amino phase, using a gradient of hexane/ethyl acetate 10-35%) to afford 324 mg of the title compound (95% purity, 76% yield).

LC-MS (Method 1): Rₜ=1.15 min; MS (ESIpos): m/z=599 [M+H]⁺

¹H-NMR (400 MHz, DMSO-d₆): δ[ppm]=5.37-5.16 (m, 1H), 4.89-4.75 (m, 1H), 4.39-4.19 (m, 1H), 4.02-3.89 (m, 2H), 3.81-3.38 (m, 3H), 3.26-2.94 (m, 4H), 2.86-2.76 (m, 1H), 2.47-2.37 (m, 1H), 2.12-1.99 (m, 2H), 1.98-1.46 (m, 9H), 1.37 (s, 9H).

### Intermediate 16-4

### tert-butyl (1S*,5R*)-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 Diastereomers)

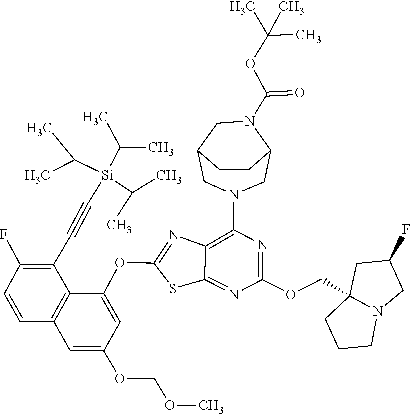 [SMILES: COCOc1cc(Oc2nc3c(N4CC5CCC(C4)N(C(=O)OC(C)(C)C)C5)nc(OC[C@@]45CCCN4C[C@H](F)C5)nc3s2)c2c(C#C[Si](C(C)C)(C(C)C)C(C)C)c(F)ccc2c1]

tert-butyl (1S*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (220 mg, 368 μmol) and 7-fluoro-3-(methoxymethoxy)-8-{[tri(propan-2-yl)silyl]ethynyl}naphthalen-1-ol (296 mg, 736 μmol) were suspended in N,N-dimethylacetamide (3.9 ml) and potassium carbonate (254 mg, 1.84 mmol; CAS-RN: [584-08-7]) was added. The reaction mixture was stirred at 110° C. for 2 h. The mixture was quenched with water and extracted with methylene chlorid. The combined organic layers were dried using a silicon coated filter and concentrated under reduced pressure. The crude product was purified by flash chromatography (amino phase, using a gradient of hexane/ethyl acetate 10-30%) to afford 273 mg of the title compound (90% purity, 73% yield).

LC-MS (Method 1): Rₜ=1.61 min; MS (ESIpos): m/z=920 [M+H]⁺

¹H-NMR (400 MHz, DMSO-d₆): δ[ppm]=8.07 (ddd, 1H), 7.62 (t, 1H), 7.57 (td, 1H), 7.49 (t, 1H), 5.34 (d, 2H), 5.32-5.15 (m, 1H), 5.13-4.72 (m, 2H), 4.18-3.77 (m, 3H), 3.41 (s, 4H), 3.26-2.94 (m, 7H), 2.84-2.79 (m, 1H), 2.12-2.04 (m, 1H), 1.99 (br d, 1H), 1.95-1.39 (m, 9H), 1.37 (d, 9H), 1.05-0.93 (m, 20H).

### Intermediate 16-5

### tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 Diastereomers)

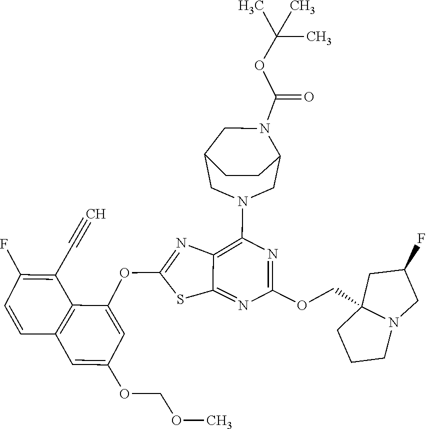 [SMILES: C#Cc1c(F)ccc2cc(OCOC)cc(Oc3nc4c(N5CC6CCC(C5)N(C(=O)OC(C)(C)C)C6)nc(OC[C@@]56CCCN5C[C@H](F)C6)nc4s3)c12]

tert-butyl (1S*,5R*)-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (270 mg, 294 μmol) was dissolved in THF (2.0 ml) and N,N,N-tributylbutan-1-aminium fluoride (590 μl, 1.0 M in THF, 590 μmol; CAS-RN: [429-41-4]) was added. The reaction was stirred at rt for 1 h. The reaction mixture was quenched with aq. NaHCO₃ solution and extracted with ethyl acetate. The combined organic layers were dried with sodium sulfate. After filtration the filtrate was concentrated under reduced pressure and the crude product was purified by flash chromatography (amino phase, using a gradient of hexane/ethyl acetate 20-100%) to afford 162 mg of the title compound (95% purity, 69% yield).

LC-MS (Method 1): Rₜ=1.29 min; MS (ESIpos): m/z=764 [M+H]⁺

¹H-NMR (400 MHz, DMSO-d6) δ[ppm]: 1.154 (1.33), 1.172 (2.66), 1.189 (1.23), 1.335 (16.00), 1.365 (1.20), 1.382 (0.82), 1.726 (0.83), 1.739 (0.73), 1.753 (0.67), 1.826 (0.50), 1.954 (0.56), 1.987 (5.01), 2.003 (1.02), 2.078 (0.66), 2.088 (0.52), 2.518 (1.92), 2.523 (1.34), 2.797 (0.45), 2.813 (0.53), 2.985 (1.18), 3.054 (1.17), 3.059 (1.29), 3.068 (1.15), 3.413 (0.66), 3.419 (8.00), 3.423 (9.32), 3.853 (0.46), 3.870 (0.42), 3.878 (0.67), 3.895 (0.56), 3.963 (0.64), 3.973 (0.66), 3.989 (0.42), 3.999 (0.64), 4.017 (1.04), 4.035 (1.05), 4.053 (0.42), 4.645 (2.06), 5.184 (0.46), 5.318 (0.46), 5.345 (2.58), 5.358 (3.42), 7.542 (0.52), 7.545 (0.49), 7.564 (1.75), 7.589 (1.17), 7.608 (0.74), 7.614 (0.62), 7.629 (1.13), 7.636 (0.86), 8.060 (0.72), 8.070 (0.42), 8.075 (0.51), 8.083 (0.73), 8.097 (0.41).

### Example 17

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethenyl-6-fluoronaphthalen-2-ol, Hydrogen Chloride Salt

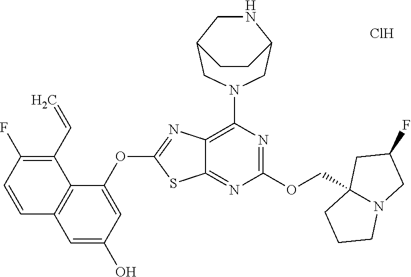 [SMILES: C=Cc1c(F)ccc2cc(O)cc(Oc3nc4c(N5CC6CCC(C5)NC6)nc(OC[C@@]56CCCN5C[C@H](F)C6)nc4s3)c12 ; Cl]

[(E)-2-{8-[(7-[8-(tert-butoxycarbonyl)-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-2-fluoro-6-(methoxymethoxy)-1-naphthyl}vinyl]boronic acid (5.00 mg, 6.29 μmol) was dissolved in dichloromethane (46 μl) and methanol (93 μl). HCl in 1,4-dioxane (47.5 μl, 4.0 M, 190 μmol) was added and the mixture was stirred at rt for 1 h. HCl in 1,4-dioxane (47.5 μl, 4.0 M, 190 μmol) was added and the mixture was stirred at 60° C. for 1 h. HCl in 1,4-dioxane (47.5 μl, 4.0 M, 190 μmol) was added and the mixture was stirred at 90° C. for 30 min. HCl in 1,4-dioxane (47.5 μl, 4.0 M, 190 μmol) was added and the mixture was stirred at 90° C. for 1 h. HCl in 1,4-dioxane (47.5 μl, 4.0 M, 190 μmol) was added and the mixture was stirred at 75° C. for 1 h. The reaction mixture was concentrated to the half of the volume under reduced pressure. Dichloromethane was added and the solid material was centrifuged. The solvent was decanted and the solid was dried under reduced pressure to afford 2.5 mg of the title compound (60% purity, 35% yield).

LC-MS (Method 1): Rₜ=0.77 min; MS (ESIpos): m/z=608 [M+H]⁺

¹H-NMR (400 MHz, DMSO-d6) δ[ppm]: 1.228 (0.58), 1.470 (0.50), 1.486 (0.50), 1.695 (1.28), 1.714 (1.70), 1.729 (1.13), 1.860 (0.57), 1.877 (0.62), 1.945 (2.37), 1.966 (1.89), 2.082 (0.75), 2.135 (1.47), 2.237 (0.83), 2.258 (0.98), 2.439 (2.06), 2.924 (1.62), 3.250 (0.90), 3.540 (1.26), 3.571 (1.00), 3.661 (0.53), 3.733 (1.25), 3.809 (1.13), 3.846 (0.44), 4.076 (1.95), 4.453 (5.64), 4.923 (0.46), 5.225 (0.92), 5.269 (0.98), 5.370 (1.02), 5.402 (1.10), 5.479 (0.94), 5.609 (0.89), 5.758 (16.00), 6.949 (0.79), 6.977 (0.78), 6.993 (0.79), 7.022 (0.72), 7.231 (0.42), 7.291 (6.78), 7.333 (0.96), 7.358 (0.51), 7.390 (0.55), 7.409 (0.84), 7.433 (1.29), 7.457 (0.80), 7.826 (0.79), 7.840 (0.82), 7.849 (0.83), 7.862 (0.70), 9.553 (0.80), 9.762 (0.56), 9.786 (0.73), 11.274 (0.79).

### Intermediate 17-1

### tert-butyl 3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

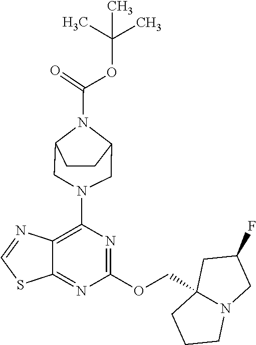 [SMILES: CC(C)(C)OC(=O)N1C2CCC1CN(c1nc(OC[C@@]34CCCN3C[C@H](F)C4)nc3scnc13)C2]

[(2R,7aS)-2-fluorotetrahydro-1H-pyrrolizin-7a(5H)-yl]methanol (1.93 g, 12.1 mmol) was dissolved in THF (37 ml) and sodium hydride (521 mg, 60% purity, 13.0 mmol; CAS-RN: [7646-69-7]) was added. The mixture was stirred at rt for 10 min. tert-butyl 3-(5-chloro[1,3]thiazolo[5,4-d]pyrimidin-7-yl)-3,8-diazabicyclo[3.2.1]octane-8-carboxylate (3.56 g, 9.31 mmol) was dissolved in N,N-dimethylacetamide (19 ml) and was added to the first mixture. The reaction mixture was stirred at 75° C. for 1 h. It was quenched with water and extracted with ethyl acetate/methanol. The combined organic layers were dried using a silicon coated filter and concentrated under reduced pressure. The crude product was purified by flash chromatography (amino phase, using a gradient of hexane/ethyl acetate 10-40%) to afford 4.07 g of the title compound (95% purity, 82% yield).

LC-MS (Method 1): Rt=1.01 min; MS (ESIpos): m/z=506 [M+H]⁺

### Intermediate 17-2

### tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

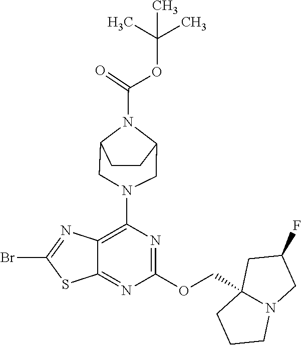 [SMILES: CC(C)(C)OC(=O)N1C2CCC1CN(c1nc(OC[C@@]34CCCN3C[C@H](F)C4)nc3sc(Br)nc13)C2]

tert-butyl 3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (500 mg, 991 μmol) was dissolved in THF (2.2 ml) and at 0° C. Lithium bis(trimethylsilyl)amide (2.0 ml, 1.0 M in THF, 2.0 mmol; CAS-RN:[4039-32-1]) was added and the reaction was stirred for 15 min at this temperature. bromine-1,4-dioxane complex (1:1) (310 mg, 1.19 mmol; CAS-RN: [15481-39-7]) was dissolved in THF and added at −78° C. to the first reaction mixture. The mixture was allowed to get to rt and was stirred for 1 h at this temperature. The reaction was quenched with aq. sodium thiosulfate and sat. aq. NaHCO₃ solution and extracted with ethyl acetate. The combined organic layers were dried with sodium sulfate and after filtration the filtrate was concentrated under reduced pressure. The crude product was purified by flash chromatography (amino phase, using a gradient of hexane/ethyl acetate 10-50%) to afford 931 mg of the title compound (60% purity, 97% yield).

LC-MS (Method 1): Rt=1.16 min; MS (ESIpos): m/z=585 [M+H]⁺

### Intermediate 17-3

### [(E)-2-{8-[(7-[8-(tert-butoxycarbonyl)-3,8-diazabicyclo[3.2.1]octan-3-yl]-5-{1[(2R,7aS)-2-fluorotetrahydro-1H-pyrrolizin-7a(5H)-yl]methoxy}[1,3]thiazolo[5,4-d]pyrimidin-2-yl)oxy]-2-fluoro-6-(methoxymethoxy)-1-naphthyl}vinyl]boronic acid

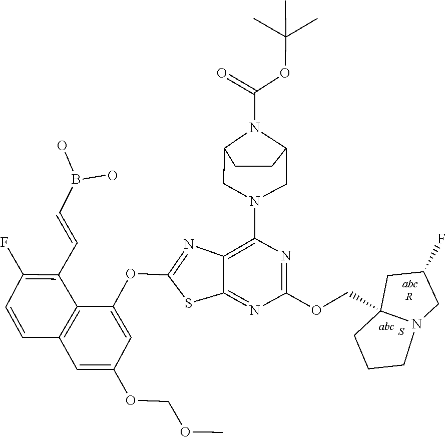

tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (29.6 mg, 50.8 μmol) and 7-fluoro-3-(methoxymethoxy)-8-[(E)-2-(4,4,5,5-tetramethyl-1,3,2-dioxaborolan-2-yl)ethenyl]naphthalen-1-ol (19.0 mg, 50.8 μmol) were suspended in N,N-dimethylacetamide (540 μl). Potassium carbonate (35.1 mg, 254 μmol; CAS-RN: [584-08-7]) was added and the mixture was stirred at 110° C. for 2 h. The reaction was diluted with water and extracted with methylene chloride. The combined organic layers were dried using a silicone coated filter and was concentrated under reduced pressure. The crude product was purified by flash chromatography (amino phase, using a gradient of hexane/ethyl acetate 10-100% and methylene chlorid/ethanol 0-50%) to afford 6.4 mg of the title compound (96% purity, 16% yield).

LC-MS (Method 1): Rt=1.14 min; MS (ESIpos): m/z=796 [M+H]⁺

¹H-NMR (400 MHz, DMSO-d6): δ[ppm]=7.94 (dd, 1H), 7.70 (s, 2H), 7.59 (d, 1H), 7.57-7.46 (m, 3H), 5.55 (dd, 1H), 5.35 (s, 2H), 5.33-5.14 (m, 1H), 4.87-4.53 (m, 2H), 4.11-4.01 (m, 1H), 3.96-3.81 (m, 2H), 3.43 (s, 2H), 3.11-2.93 (m, 5H), 2.85-2.75 (m, 1H), 2.52 (d, 2H), 2.10-2.03 (m, 1H), 2.02-1.89 (m, 2H), 1.86-1.65 (m, 5H), 1.56-1.47 (m, 2H), 1.41 (s, 9H).

### Intermediate 17-3 Building Block 1, 1. Step

### 8-ethynyl-7-fluoro-3-(methoxymethoxy)naphthalen-1-ol

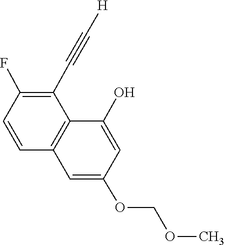 [SMILES: C#Cc1c(F)ccc2cc(OCOC)cc(O)c12]

7-fluoro-3-(methoxymethoxy)-8-{[tri(propan-2-yl)silyl]ethynyl}naphthalen-1-ol (500 mg, 1.24 mmol) was dissolved in THF (10 ml) and at 0° C. N,N,N-Tributylbutan-1-aminium fluoride (1.9 ml, 1.0 M, 1.9 mmol; CAS-RN: [429-41-4]) was added. The reaction mixture was stirred at 0° C. for 30 min. The reaction was quenched with water and extracted with ethyl acetate. The combined organic layers were filtered using a silicon coated filter and the filtrate was concentrated under reduced pressure. The crude product was purified by flash chromatography (silica phase, using a gradient of hexane/ethyl acetate 10-20%) to afford 216 mg (71% yield) of the title compound.

LC-MS (Method 1): Rₜ=1.23 min; MS (ESIpos): m/z=245 [M+H]⁺

¹H-NMR (400 MHz, DMSO-d6) δ[ppm]: 1.986 (0.47), 2.518 (0.68), 2.523 (0.47), 3.339 (7.84), 4.515 (4.58), 4.518 (4.69), 5.236 (16.00), 6.671 (2.86), 6.678 (3.01), 6.965 (3.79), 6.970 (3.67), 7.346 (2.01), 7.369 (4.06), 7.391 (2.11), 7.768 (1.72), 7.782 (1.78), 7.790 (1.70), 7.805 (1.59), 10.307 (2.99).

### Intermediate 17-3 Building Block 1, 2. Step

### tert-butyl{[8-ethynyl-7-fluoro-3-(methoxymethoxy)naphthalen-1-yl]oxy}dimethylsilane

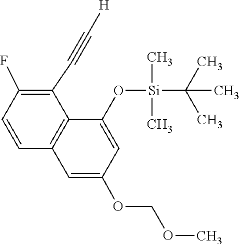 [SMILES: C#Cc1c(F)ccc2cc(OCOC)cc(O[Si](C)(C)C(C)(C)C)c12]

8-ethynyl-7-fluoro-3-(methoxymethoxy)naphthalen-1-ol (208 mg, 845 μmol) was dissolved in DMF (4.0 ml). tert-Butyl(chloro)di(methyl)silane (153 mg, 1.01 mmol) and 1H-imidazole (144 mg, 2.11 mmol; CAS-RN: [288-32-4]) were added. The reaction mixture was stirred at rt for 2 h. The reaction was diluted with water and extracted with ethyl acetate. The combined organic layers were washed for three times with half sat. aq. NaCl solution and dried using a silicon coated filter and the filtrate was concentrated under reduced pressure. The crude product was purified by flash chromatography (silica phase, using a gradient of hexane/ethyl acetate 10-20%) to afford 280 mg (95% purity, 71% yield) of the title compound.

LC-MS (Method 1): Rₜ=1.73 min; MS (ESIpos): m/z=361 [M+H]⁺

¹H-NMR (400 MHz, DMSO-d₆): δ[ppm]=7.86 (dd, 1H), 7.42 (t, 1H), 7.15 (d, 1H), 6.73 (d, 1H), 5.26 (s, 2H), 4.63 (d, 1H), 3.40 (s, 3H), 0.95 (s, 9H), 0.37 (s, 6H).

### Intermediate 17-3 Building Block 1, 3. Step

### Dicyclohexylborane

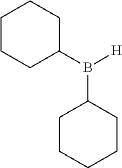 [SMILES: B(C1CCCCC1)C1CCCCC1]

Borane-tetrahydrofuran complex (2.0 ml, 1.0 M, 2.0 mmol; CAS-RN: [14044-65-6]) was diluted with THF (2.6 ml) to a 0.4 M solution. At 0° C. cyclohexene (410 μl, 4.0 mmol; CAS-RN: [110-83-8]) was added and stirred at this temperature for 1.5 h. The suspension was diluted with THF (15 ml) to a 0.1 M solution and was used without further work up.

### Intermediate 17-3 Building Block 1, 4. Step

### tert-butyl({7-fluoro-3-(methoxymethoxy)-8-[(E)-2-(4,4,5,5-tetramethyl-1,3,2-dioxaborolan-2-yl)ethenyl]naphthalen-1-yl}oxy)dimethylsilane

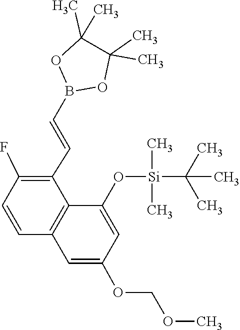 [SMILES: COCOc1cc(O[Si](C)(C)C(C)(C)C)c2c(/C=C/B3OC(C)(C)C(C)(C)O3)c(F)ccc2c1]

tert-butyl{[8-ethynyl-7-fluoro-3-(methoxymethoxy)naphthalen-1-yl]oxy}dimethylsilane (310 mg, 860 μmol) was dissolved in THF (2.0 ml) and the solution was cooled to 0° C. dicyclohexylborane (3.4 ml, 0.10 M, 340 μmol) was added and the mixture was stirred at rt for 2 h. 4,4,5,5-tetramethyl-1,3,2-dioxaborolane (480 μl, 97% purity, 3.2 mmol) was added at 0° C. and the reaction was stirred at rt overnight. The solvent was removed under reduced pressure. Dicyclohexylborane (0.85 ml, 0.10 M, 85 μmol) was added and the solvent removed under reduced pressure. 4,4,5,5-tetramethyl-1,3,2-dioxaborolane (143 μl, 97% purity, 0.95 mmol) was added and the mixture was stirred at rt overnight. Further dicyclohexylborane (1.7 ml, 0.10 M, 170 μmol) was added and the solvent removed under reduced pressure. 4,4,5,5-tetramethyl-1,3,2-dioxaborolane (143 μl, 97% purity, 0.95 mmol) was added and the mixture was stirred at rt overnight. The reaction mixture was diluted with aq. NaHCO₃ solution and extracted with ethyl acetate. The combined organic layers were dried using a silicon coated filter and the filtrate was concentrated under reduced pressure. The crude product was purified by flash chromatography (silica phase, using a gradient of hexane/ethyl acetate 5-20%) to afford 32 mg (8% yield) of the title compound.

LC-MS (Method 1): Rₜ=1.89 min; MS (ESIpos): m/z=489 [M+H]⁺

¹H-NMR (400 MHz, DMSO-d₆): δ[ppm]=7.82 (d, 1H), 7.76 (dd, 1H), 7.36 (dd, 1H), 7.11 (d, 1H), 6.71 (d, 1H), 5.81-5.70 (m, 1H), 5.25 (s, 2H), 3.39 (s, 3H), 1.26 (s, 12H), 0.91 (s, 9H), 0.34 (s, 6H)

### Intermediate 17-3 building block 1, 5. Step

### 7-fluoro-3-(methoxymethoxy)-8-[(E)-2-(4,4,5,5-tetramethyl-1,3,2-dioxaborolan-2-yl)ethenyl]naphthalen-1-ol

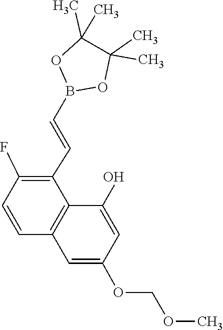 [SMILES: COCOc1cc(O)c2c(/C=C/B3OC(C)(C)C(C)(C)O3)c(F)ccc2c1]

tert-butyl({7-fluoro-3-(methoxymethoxy)-8-[(E)-2-(4,4,5,5-tetramethyl-1,3,2-dioxaborolan-2-yl)ethenyl]naphthalen-1-yl}oxy)dimethylsilane (30.0 mg, 61.4 μmol) was dissolved in THF (420 μl). N,N,N-Tributylbutan-1-aminium fluoride (120 μl, 1.0 M, 120 μmol; CAS-RN: [429-41-4]) was added and the reaction was stirred at rt for 1 h. 2,3-Dimethylbutane-2,3-diol (36.2 mg, 307 μmol) was added and the reaction was stirred at rt for 2 h. The reaction mixture was diluted with water and extracted with ethyl acetate. The combined organic layers were washed with sat. aq. NaCl solution and dried with sodium sulfate. After filtration the filtrate was concentrated under reduced pressure to afford 20 mg (87% yield) of the title compound which was used without further purification.

LC-MS (Method 1): Rₜ=1.34 min; MS (ESIneg): m/z=373 [M−H]⁻

¹H-NMR (400 MHz, DMSO-d₆): δ[ppm]=10.42 (s, 1H), 8.10 (d, 1H), 7.69 (dd, 1H), 7.37-7.27 (m, 1H), 6.93 (d, 1H), 6.61 (d, 1H), 5.79-5.68 (m, 1H), 5.22 (s, 2H), 3.39 (s, 3H), 1.26 (s, 12H)

### Example 18

### 4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)naphthalen-2-ol

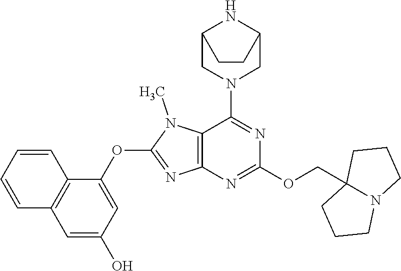 [SMILES: Cn1c(Oc2cc(O)cc3ccccc23)nc2nc(OCC34CCCN3CCC4)nc(N3CC4CCC(C3)N4)c21]

tert-butyl 3-{8-[(3-methoxynaphthalen-1-yl)oxy]-7-methyl-2-[(tetrahydro-1H-pyrrolizin-7a(5H)-yl)methoxy]-7H-purin-6-yl}-3,8-diazabicyclo[3.2.1]octane-8-carboxylate (35.9 mg, 100% purity, 54.7 μmol) was dissolved in dichloromethane (5.0 ml), boron tribromide (550 μl, 1.0 M in DCM, 550 μmol) was added and the reaction mixture was stirred at rt for 4 h. The reaction was quenched with water and extracted with DCM/propanol 7:3. The combined organic layers were dried using a silicon coated filter and concentrated under reduced pressure. The crude product was purified by preparative HPLC (Method: column: Chromatorex C18; 125 mm×30 mm; 10 μm; flow=50 ml/min; using a gradient of water (+0.05% formic acid)/acetonitrile 15-50%) to afford 11.8 mg (100% purity, 40% yield) of the title compound.

LC-MS (Method 1): Rₜ=0.70 min; MS (ESIneg): m/z=540 [M−H]⁻

¹H-NMR (600 MHz, DMSO-d6) δ[ppm]: 1.586 (0.77), 1.597 (2.10), 1.605 (1.47), 1.609 (1.65), 1.617 (1.99), 1.629 (1.46), 1.726 (0.99), 1.737 (1.49), 1.747 (2.12), 1.758 (3.01), 1.769 (2.90), 1.778 (1.92), 1.798 (0.73), 1.808 (1.71), 1.818 (2.49), 1.828 (2.74), 1.838 (2.82), 1.848 (3.02), 1.855 (3.64), 1.862 (2.59), 1.871 (2.60), 1.882 (1.90), 1.892 (0.90), 2.633 (0.93), 2.645 (1.71), 2.650 (1.50), 2.655 (1.62), 2.661 (1.92), 2.672 (1.09), 3.000 (1.09), 3.010 (2.29), 3.019 (1.91), 3.027 (2.18), 3.037 (1.09), 3.353 (2.49), 3.373 (2.58), 3.839 (2.65), 3.859 (2.49), 3.902 (16.00), 3.977 (8.79), 4.494 (0.52), 7.130 (3.22), 7.133 (3.35), 7.250 (4.07), 7.254 (3.86), 7.297 (1.23), 7.308 (2.03), 7.322 (1.40), 7.460 (1.29), 7.471 (2.10), 7.485 (1.19), 7.786 (2.59), 7.799 (2.36), 7.830 (2.24), 7.844 (2.11), 8.275 (3.50).

### Intermediate 18-1

### tert-butyl 3-(2-chloro-7-methyl-7H-purin-6-yl)-3,8-diazabicyclo[3.2.1]octane-8-carboxylate

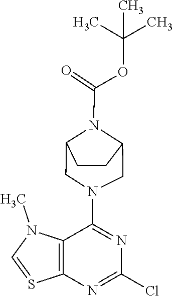

2,6-dichloro-7-methyl-7H-purine (100 mg, 493 μmol) and tert-butyl 3,8-diazabicyclo[3.2.1]octane-8-carboxylate (110 mg, 517 μmol) were suspended in ethanol (2.0 ml), triethylamine (69 μl, 490 μmol; CAS-RN: [121-44-8]) was added and the reaction mixture was stirred at 60° C. for 6 h. The solvent was removed under reduced pressure and the residue was dissolved in ethyl acetate and washed with water. The organic layer was dried using a silicon coated filter and concentrated under reduced pressure to afford 144 mg (98% purity, 76% yield) of the title compound which was used without further purification.

LC-MS (Method 1): Rₜ=1.64 min; MS (ESIpos): m/z=379 [M+H]⁺

¹H-NMR (600 MHz, DMSO-d6) δ[ppm]: 1.440 (16.00), 1.696 (0.41), 1.709 (0.43), 1.987 (0.71), 3.347 (0.54), 3.367 (0.55), 3.944 (4.98), 4.025 (0.68), 4.045 (0.67), 4.224 (0.48), 8.379 (1.64).

### Intermediate 18-2

### tert-butyl 3-{7-methyl-2-[(tetrahydro-1H-pyrrolizin-7a(5H)-yl)methoxy]-7H-purin-6-yl}-3,8-diazabicyclo[3.2.1]octane-8-carboxylate

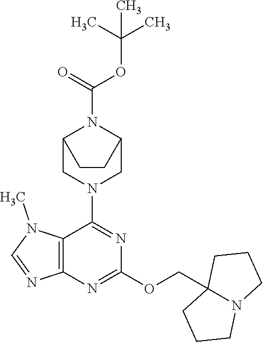 [SMILES: Cn1cnc2nc(OCC34CCCN3CCC4)nc(N3CC4CCC(C3)N4C(=O)OC(C)(C)C)c21]

Sodium hydride (29.9 mg, 60% purity, 746 μmol; CAS-RN: [7646-69-7]) was suspended in THF (3.0 ml). (tetrahydro-1H-pyrrolizin-7a(5H)-yl)methanol (105 mg, 746 μmol) was dissolved in THF (3.0 ml) and added to the sodium hydride mixture and stirred at rt for 15 min. tert-butyl 3-(2-chloro-7-methyl-7H-purin-6-yl)-3,8-diazabicyclo[3.2.1]octane-8-carboxylate (144 mg, 98% purity, 373 μmol) was dissolved in THF (3.0 ml) and added to the stirring mixture and it was stirred at 70° C. for 2 h. The reaction was quenched with aq. NaHCO₃ solution and ethyl acetate. The organic layer was washed with water and dried using a silicon coated filter and concentrated under reduced pressure to afford 177 mg (88% purity, 86% yield) of the title compound which was used without further purification.

LC-MS (Method 1): Rₜ=1.07 min; MS (ESIpos): m/z=484 [M+H]⁺

### Intermediate 18-3

### tert-butyl 3-{8-bromo-7-methyl-2-[(tetrahydro-1H-pyrrolizin-7a(5H)-yl)methoxy]-7H-purin-6-yl}-3,8-diazabicyclo[3.2.1]octane-8-carboxylate

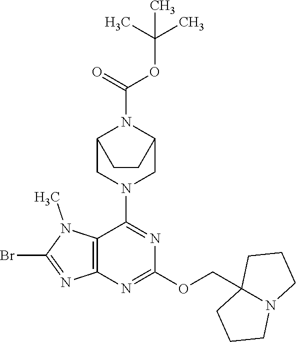 [SMILES: Cn1c(Br)nc2nc(OCC34CCCN3CCC4)nc(N3CC4CCC(C3)N4C(=O)OC(C)(C)C)c21]

tert-butyl 3-{7-methyl-2-[(tetrahydro-1H-pyrrolizin-7a(5H)-yl)methoxy]-7H-purin-6-yl}-3,8-diazabicyclo[3.2.1]octane-8-carboxylate (174 mg, 92% purity, 330 μmol) was dissolved in THF (3.0 ml) and cooled to −78° C. Lithium bis(trimethylsilyl)amide (890 μl, 1.0 M, 890 μmol; CAS-RN: [4039-32-1]) was added and the mixture was stirred at −78° C. for 45 min. Lithium bis(trimethylsilyl)amide (890 μl, 1.0 M, 890 μmol; CAS-RN: [4039-32-1]) was added again and in THF (3.0 ml) dissolved bromine (48 μl, 920 μmol; CAS-RN: [7726-95-6]) was added and the reaction mixture was stirred at −78° C. for 30 min. The reaction was quenched with water and aq. sodium thiosulfate solution (w=10%) and extracted with methylene chloride. The organic layer was washed with sat. aq. NaCl solution, dried using a silicon coated filter and concentrated under reduced pressure. The crude product was purified by flash chromatography (silica phase, using a gradient of methylene chloride/methanol 2-75%) to afford 69.4 mg (100% purity, 37% yield) of the title compound.

LC-MS (Method 1): Rₜ=1.20 min; MS (ESIpos): m/z=562 [M+H]⁺

¹H-NMR (600 MHz, DMSO-d6) δ[ppm]: 1.436 (16.00), 1.530 (0.61), 1.538 (0.48), 1.542 (0.48), 1.550 (0.64), 1.709 (0.54), 1.719 (0.74), 1.730 (0.92), 1.741 (0.71), 1.753 (0.52), 1.759 (0.51), 1.768 (0.76), 1.778 (0.98), 1.788 (0.96), 1.797 (0.68), 1.830 (0.57), 1.841 (0.58), 1.850 (0.72), 1.860 (0.50), 2.520 (0.74), 2.897 (0.41), 2.907 (0.68), 2.915 (0.59), 2.923 (0.60), 3.798 (5.50), 3.917 (2.64), 4.195 (0.67).

### Intermediate 18-4

### tert-butyl 3-{8-[(3-methoxynaphthalen-1-yl)oxy]-7-methyl-2-[(tetrahydro-1H-pyrrolizin-7a(5H)-yl)methoxy]-7H-purin-6-yl}-3,8-diazabicyclo[3.2.1]octane-8-carboxylate

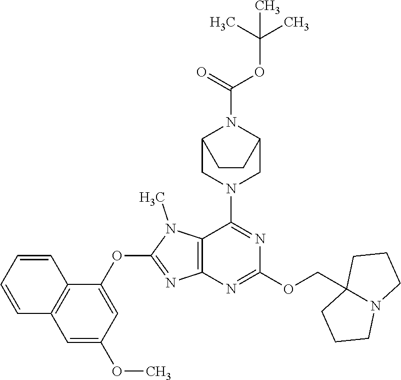 [SMILES: COc1cc(Oc2nc3nc(OCC45CCCN4CCC5)nc(N4CC5CCC(C4)N5C(=O)OC(C)(C)C)c3n2C)c2ccccc2c1]

Under argon sodium hydride (2.83 mg, 60% purity, 118 μmol; CAS-RN: [7646-69-7]) was suspended in THF (2.0 ml). 3-methoxynaphthalen-1-ol (20.5 mg, 118 μmol) was dissolved in THF (2.0 ml) and added to the sodium hydride mixture and stirred at rt for 15 min. tert-butyl 3-{8-bromo-7-methyl-2-[(tetrahydro-1H-pyrrolizin-7a(5H)-yl)methoxy]-7H-purin-6-yl}-3,8-diazabicyclo[3.2.1]octane-8-carboxylate (48.0 mg, 92% purity, 78.5 μmol) was dissolved in THF (2.0 ml) and added to the stirring mixture and it was stirred at rt overnight. The reaction was quenched with aq. NaHCO₃ solution and ethyl acetate. The organic layer was washed with water and dried using a silicon coated filter and concentrated under reduced pressure. The crude product was purified by preparative HPLC (Method: column: Chromatorex C18; 125 mm×30 mm; 10 μm; flow=50 ml/min; using a gradient of water (+0.05% formic acid)/acetonitrile 50-70%) to afford 35.9 mg (100% purity, 70% yield) of the title compound.

LC-MS (Method 6): Rₜ=3.78 min; MS (ESIpos): m/z=656 [M+H]⁺

### Example 19

### 5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol

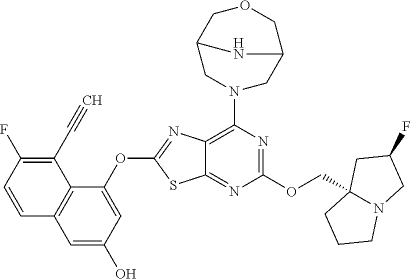 [SMILES: C#Cc1c(F)ccc2cc(O)cc(Oc3nc4c(N5CC6COCC(C5)N6)nc(OC[C@@]56CCCN5C[C@H](F)C6)nc4s3)c12]

5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl) [1,3]thiazolo[5,4-d]pyrimidin-2-yl]oxy}naphthalen-2-ol hydrogen chloride 1:2 (540 mg, 779 μmol) was dissolved in methanol. Ammonia (330 μl, 33% purity, 7.8 mmol; CAS-RN: [7664-41-7]) was added and the mixture was concentrated under reduced pressure. The crude product was purified by flash chromatography (amino phase, using a gradient of methylene chloride/methanol 0-15%). The isolated product was stirred in methylene chloride. The solid was filtrated and dried to afford 331 mg (95% purity, 65% yield) of the title compound.

LC-MS (Method 1): Rₜ=0.69 min; MS (ESIpos): m/z=622 [M+H]⁺

¹H-NMR (400 MHz, DMSO-d₆): δ[ppm]=10.47-10.12 (m, 1H), 7.95 (dd, 1H), 7.48 (t, 1H), 7.32-7.19 (m, 2H), 5.36-5.14 (m, 2H), 4.97-4.79 (m, 1H), 4.58 (d, 1H), 4.01-3.81 (m, 2H), 3.75-3.52 (m, 3H), 3.48-3.37 (m, 1H), 3.31-3.21 (m, 1H), 3.19-3.10 (m, 1H), 3.09-3.02 (m, 2H), 2.98 (s, 1H), 2.85-2.76 (m, 2H), 2.69-2.58 (m, 1H), 2.46 (dt, 1H), 2.10-2.04 (m, 1H), 2.02-1.92 (m, 2H), 1.86-1.68 (m, 3H).

### Intermediate 19-1

### tert-butyl 7-(5-chloro[1,3]thiazolo[5,4-d]pyrimidin-7-yl)-3-oxa-7,9-diazabicyclo[3.3.1]nonane-9-carboxylate

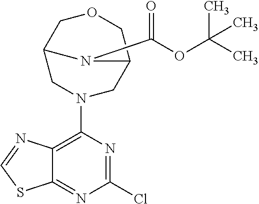 [SMILES: CC(C)(C)OC(=O)N1C2COCC1CN(c1nc(Cl)nc3scnc13)C2]

5,7-dichloro[1,3]thiazolo[5,4-d]pyrimidine (1.27 g, 6.16 mmol) and tert-butyl 3-oxa-7,9-diazabicyclo[3.3.1]nonane-9-carboxylate (1.55 g, 6.78 mmol) were dissolved in 1,4-dioxane (26 ml), triethylamine (2.1 ml, 15 mmol; CAS-RN: [121-44-8]) was added and the reaction mixture was stirred at 50° C. for 30 min. The mixture was quenched with water and extracted with ethyl acetate. The organic layer was dried using a silicon coated filter and concentrated under reduced pressure to afford 2.42 g (99% yield) of the title compound which was used without further purification.

LC-MS (Method 1): Rₜ=1.39 min; MS (ESIpos): m/z=398 [M+H]⁺

¹H-NMR (400 MHz, DMSO-d6) δ[ppm]: 1.406 (1.79), 1.443 (16.00), 2.518 (0.46), 3.329 (10.04), 3.607 (0.62), 3.635 (0.74), 3.836 (0.67), 4.067 (0.54), 4.098 (0.67), 5.758 (0.64), 9.244 (2.77).

### Intermediate 19-2

### tert-butyl 7-(5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

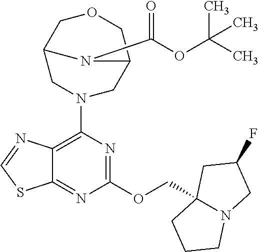 [SMILES: CC(C)(C)OC(=O)N1C2COCC1CN(c1nc(OC[C@@]34CCCN3C[C@H](F)C4)nc3scnc13)C2]

[(2R,7aS)-2-fluorotetrahydro-1H-pyrrolizin-7a(5H)-yl]methanol (1.25 g, 7.87 mmol) was dissolved in THF (24 ml), sodium hydride (339 mg, 60% purity, 8.48 mmol; CAS-RN: [7646-69-7]) was added and the mixture was stirred at rt for 10 min. tert-butyl 7-(5-chloro[1,3]thiazolo[5,4-d]pyrimidin-7-yl)-3-oxa-7,9-diazabicyclo[3.3.1]nonane-9-carboxylate (2.41 g, 6.06 mmol) was dissolved in N,N-dimethylacetamide (12 ml) and added to the stirring mixture. It was stirred at 75° C. for 1 h. The reaction was quenched with water and extracted with ethyl acetate. The organic layer was washed with aq. half sat. NaCl solution, dried using a silicon coated filter and was concentrated under reduced pressure. The crude product was purified by flash chromatography (amino phase, using a gradient of hexane/ethyl acetate 10-40%) to afford 2.48 g (79% yield) of the title compound.

LC-MS (Method 1): Rₜ=0.87 min; MS (ESIpos): m/z=522 [M+H]⁺

¹H-NMR (400 MHz, DMSO-d6) δ[ppm]: 1.173 (0.48), 1.412 (1.97), 1.440 (12.33), 1.763 (0.43), 1.776 (0.44), 1.955 (10.55), 1.988 (1.20), 2.018 (0.61), 2.025 (0.59), 2.518 (0.55), 2.523 (0.42), 2.781 (9.95), 2.942 (16.00), 3.004 (0.79), 3.067 (0.58), 3.085 (0.51), 3.092 (0.50), 3.600 (0.60), 3.628 (0.77), 4.017 (0.43), 4.035 (0.75), 4.053 (0.55), 4.062 (0.51), 5.759 (1.16), 8.978 (4.36).

### Intermediate 19-3

### tert-butyl 7-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

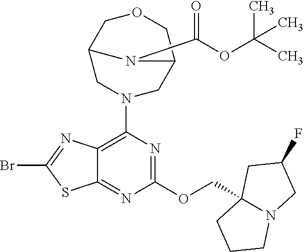 [SMILES: CC(C)(C)OC(=O)N1C2COCC1CN(c1nc(OC[C@@]34CCCN3C[C@H](F)C4)nc3sc(Br)nc13)C2]

tert-butyl 7-(5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (2.48 g, 4.75 mmol) was dissolved in THF (72 ml) and at 0° C. Lithium bis(trimethylsilyl)amide (9.5 ml, 1.0 M, 9.5 mmol; CAS-RN: [4039-32-1]) was added and the reaction was stirred for 15 min at this temperature. bromine-1,4-dioxane complex (1.41 g, 5.70 mmol; CAS-RN: [15481-39-7]) was dissolvedin THF (12 ml) and added at −78° C. to the first reaction mixture. The mixture was allowed to get to 0° C. and was stirred for 1 h at this temperature. The reaction was quenched with aq. sodium thiosulfate and sat. aq. NaHCO3 solution and extracted with ethyl acetate. The combined organic layers were dried with sodium sulfate and after filtration the filtrate was concentrated under reduced pressure. The crude product was purified by flash chromatography (amino phase, using a gradient of hexane/ethyl acetate 10-35%) to afford 935 mg (33% yield) of the title compound.

LC-MS (Method 1): Rₜ=1.06 min; MS (ESIpos): m/z=599 [M+H]⁺

¹H-NMR (400 MHz, DMSO-d6) δ[ppm]: 1.439 (16.00), 1.742 (0.42), 1.756 (0.44), 1.769 (0.44), 2.006 (0.60), 2.013 (0.63), 2.518 (0.85), 2.523 (0.67), 2.895 (0.79), 2.994 (0.75), 3.054 (1.20), 3.060 (0.64), 3.073 (0.59), 3.079 (0.54), 3.596 (0.59), 3.624 (0.76), 4.071 (0.73), 5.758 (0.78).

### Intermediate 19-4

### tert-butyl 7-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

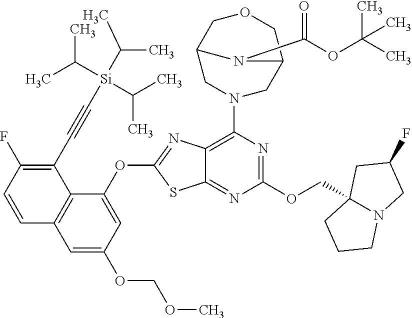 [SMILES: COCOc1cc(Oc2nc3c(N4CC5COCC(C4)N5C(=O)OC(C)(C)C)nc(OC[C@@]45CCCN4C[C@H](F)C5)nc3s2)c2c(C#C[Si](C(C)C)(C(C)C)C(C)C)c(F)ccc2c1]

tert-butyl 7-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (1.08 g, 1.79 mmol) and 7-fluoro-3-(methoxymethoxy)-8-{[tri(propan-2-yl)silyl]ethynyl}naphthalen-1-ol (1.44 g, 3.59 mmol) were dissolved in N,N-dimethylacetamide (19 ml) and potassium carbonate (1.24 g, 8.97 mmol; CAS-RN: [584-08-7]) was added. The suspension was stirred at 110° C. for 2 h. The reaction mixture was quenched with water and extracted with methylene chloride. The combined organic layers were dried using a silicon coated filter and were concentrated under reduced pressure. The crude product was purified by flash chromatography (amino phase, using a gradient of hexane/ethyl acetate 10-30%) to afford 1.16 g (70% yield) of the title compound.

LC-MS (Method 1): Rₜ=1.62 min; MS (ESIpos): m/z=922 [M+H]⁺

¹H-NMR (400 MHz, DMSO-d6) δ[ppm]: 0.982 (3.91), 1.012 (1.62), 1.024 (1.17), 1.033 (0.51), 1.043 (0.87), 1.061 (0.41), 1.404 (0.60), 1.419 (16.00), 1.431 (0.64), 1.438 (1.23), 1.736 (0.41), 1.749 (0.43), 1.955 (5.49), 1.986 (0.62), 1.992 (0.53), 2.061 (0.40), 2.518 (0.67), 2.523 (0.49), 2.781 (5.21), 2.941 (8.69), 2.980 (0.65), 3.044 (0.51), 3.051 (0.47), 3.061 (0.56), 3.069 (0.56), 3.408 (10.75), 3.569 (0.41), 5.342 (3.83), 7.518 (0.92), 7.523 (1.02), 7.574 (0.70), 7.629 (1.25), 7.635 (1.09), 8.060 (0.46), 8.074 (0.48), 8.083 (0.49), 8.098 (0.46).

### Intermediate 19-5

### tert-butyl 7-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate

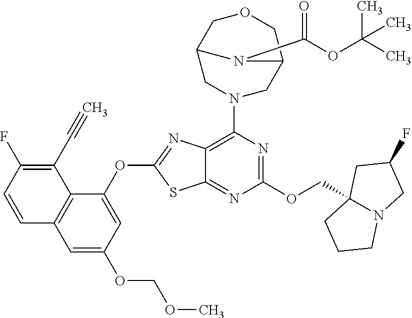 [SMILES: C#Cc1c(F)ccc2cc(OCOC)cc(Oc3nc4c(N5CC6COCC(C5)N6C(=O)OC(C)(C)C)nc(OC[C@@]56CCCN5C[C@H](F)C6)nc4s3)c12]

tert-butyl 7-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (1.16 g, 1.25 mmol) was dissolved in THF (7.2 ml), N,N,N-tributylbutan-1-aminium fluoride (2.5 ml, 1.0 M, 2.5 mmol) was added and the reaction was stirred at rt for 1 h. The mixture was diluted with ethyl acetate and quenched with NaHCO₃ solution. It was extracted with ethyl acetate and the combined organic layers were dried (sodium sulfate) and after filtration concentrated under reduced pressure. The crude product was purified by flash chromatography (amino phase, using a gradient of hexane/ethyl acetate 20-40%) and the isolated product was purified by flash chromatography again (amino phase, using a gradient of hexane/ethyl acetate 10-35%) to afford 673 mg (70% yield) of the title compound.

LC-MS (Method 1): Rₜ=1.14 min; MS (ESIpos): m/z=766 [M+H]⁺

¹H-NMR (400 MHz, DMSO-d6) δ[ppm]: 1.154 (1.01), 1.172 (2.31), 1.190 (1.21), 1.403 (16.00), 1.440 (0.44), 1.955 (0.54), 1.987 (4.42), 1.997 (0.50), 2.004 (0.52), 2.518 (0.58), 2.523 (0.40), 2.941 (0.46), 2.988 (0.60), 3.055 (0.52), 3.067 (0.55), 3.073 (0.53), 3.422 (8.25), 3.999 (0.43), 4.017 (0.96), 4.034 (0.93), 4.681 (0.62), 5.360 (1.85), 5.758 (1.68), 7.552 (0.45), 7.575 (0.90), 7.597 (0.47), 7.644 (1.54), 7.649 (0.81), 8.081 (0.41), 8.090 (0.41).

### Intermediate 19-6

### 5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol, Hydrogen Chloride Salt

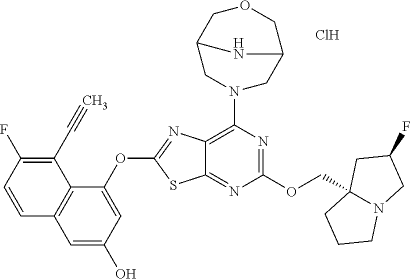 [SMILES: C#Cc1c(F)ccc2cc(O)cc(Oc3nc4c(N5CC6COCC(C5)N6)nc(OC[C@@]56CCCN5C[C@H](F)C6)nc4s3)c12 ; Cl]

tert-butyl 7-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (667 mg, 872 μmol) was dissolved in methylene chloride (13 ml) and methanol (6.4 ml), HCl in 1,4-dioxane (6.5 ml, 4.0 M, 26 mmol) was added and the reaction was stirred at 40° C. for 2 h. The reaction mixture was concentrated to the half of the volume under reduced pressure. Dichloromethane was added and the solid material was centrifuged. The solvent was decanted and the solid was dried under reduced pressure to afford 595 mg (98% yield) of the title compound.

LC-MS (Method 1): Rₜ=0.70 min; MS (ESIpos): m/z=621 [M+H]⁺

¹H-NMR (400 MHz, DMSO-d₆): δ[ppm]=11.20 (br d, 1H), 10.76-10.34 (m, 1H), 10.24-10.01 (m, 2H), 7.97 (dd, 1H), 7.50 (t, 1H), 7.39 (d, 1H), 7.32 (d, 1H), 5.70-5.42 (m, 2H), 5.35-5.10 (m, 1H), 4.61 (d, 1H), 4.48 (br d, 2H), 3.97 (br d, 3H), 3.87-3.71 (m, 4H), 3.66-3.56 (m, 2H), 3.46-3.33 (m, 1H), 3.26 (br dd, 1H), 2.61-2.52 (m, 1H), 2.48-2.41 (m, 2H), 2.31-2.23 (m, 1H), 2.23-2.08 (m, 2H), 2.07-1.97 (m, 1H).

### Example 20

### 4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol, hydrogen chloride salt (Single Enantiomer 1)

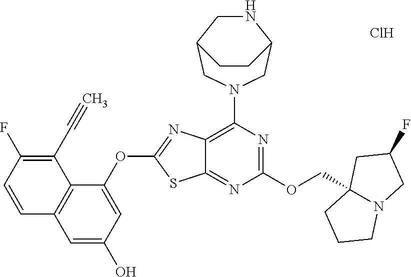 [SMILES: C#Cc1c(F)ccc2cc(O)cc(Oc3nc4c(N5CC6CCC(C5)NC6)nc(OC[C@@]56CCCN5C[C@H](F)C6)nc4s3)c12 ; Cl]

tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (single enantiomer 1) (35.0 mg, 45.9 μmol) was dissolved in methylene chloride (680 μl) and methanol (340 μl), HCl in 1,4-dioxane (340 μl, 4.0 M, 1.4 mmol) was added and the reaction was stirred at rt for 1 h. The reaction mixture was concentrated to the half of the volume under reduced pressure. Dichloromethane was added and the solid material was centrifuged. The solvent was decanted and the solid was dried under reduced pressure to afford 23 mg (95% purity, 69% yield) of the title compound.

LC-MS (Method 1): Rₜ=0.72 min; MS (ESIpos): m/z=620 [M+H]⁺

Specific optical rotation: [α]_{D}=−15.55 (from solution in DMSO, c=2.8 mg/mL)

¹H-NMR (400 MHz, DMSO-d6) δ[ppm]: 1.441 (1.00), 1.466 (1.15), 1.537 (0.46), 2.020 (0.56), 2.043 (0.54), 2.080 (0.56), 2.095 (0.60), 2.113 (0.68), 2.128 (0.72), 2.143 (0.93), 2.168 (0.60), 2.182 (0.50), 2.247 (0.51), 2.276 (0.68), 2.292 (0.46), 2.434 (0.81), 2.454 (1.07), 2.518 (3.19), 2.523 (2.24), 2.575 (0.55), 3.163 (0.49), 3.228 (1.36), 3.244 (1.09), 3.259 (1.37), 3.516 (1.05), 3.555 (1.04), 3.564 (0.52), 3.732 (0.74), 3.746 (0.69), 3.821 (0.72), 4.293 (1.73), 4.470 (4.82), 4.592 (3.66), 4.594 (3.81), 5.487 (0.64), 5.618 (0.62), 5.759 (16.00), 7.314 (2.39), 7.319 (3.39), 7.345 (2.93), 7.350 (2.09), 7.465 (1.39), 7.488 (2.64), 7.510 (1.34), 7.949 (1.15), 7.963 (1.19), 7.972 (1.19), 7.986 (1.12), 9.432 (0.55), 11.264 (0.61), 11.277 (0.59).

### Intermediate 20-1

### tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Single Enantiomer 1)

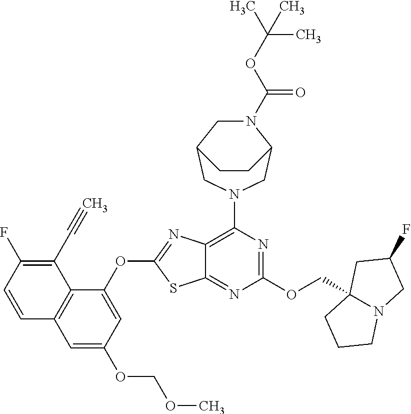 [SMILES: C#Cc1c(F)ccc2cc(OCOC)cc(Oc3nc4c(N5CC6CCC(C5)N(C(=O)OC(C)(C)C)C6)nc(OC[C@@]56CCCN5C[C@H](F)C6)nc4s3)c12]

The isomers of tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 diastereomers)

(120 mg, 157 μmol) were separated (using the following conditions: Instrument: PrepCon Labomatic HPLC-2; Column: YMC Amylose SA 5, 250×50; eluent A: methyl tert-butyl ether+0.1 vol % diethylamine; eluent B: acetonitrile; isocratic: 95% A+5% B; flow: 100 ml/min; temperature: 25° C.; UV: 280 nm) to afford 41.0 mg (95% purity, 32% yield) of the title compound. The second isomer is described as intermediate 21-1.

LC-MS (Method 1): Rₜ=1.21 min; MS (ESIpos): m/z=764 [M+H]⁺

Specific optical rotation: [α]_{D}=−11.24 (from solution in DMSO, c=2.5 mg/mL)

¹H-NMR (400 MHz, DMSO-d6) δ[ppm]: 0.794 (0.50), 0.836 (0.41), 0.849 (1.05), 0.866 (2.41), 0.885 (2.10), 0.904 (3.87), 0.923 (1.74), 1.083 (0.86), 1.231 (1.48), 1.259 (1.70), 1.279 (0.98), 1.288 (1.14), 1.298 (1.28), 1.315 (1.30), 1.335 (16.00), 1.367 (1.23), 1.391 (1.23), 1.410 (1.12), 1.425 (0.69), 1.430 (0.70), 1.683 (0.51), 1.698 (0.58), 1.713 (0.52), 1.726 (0.78), 1.743 (0.59), 1.754 (0.58), 1.826 (0.44), 1.953 (0.43), 2.003 (0.81), 2.078 (0.51), 2.327 (0.72), 2.331 (0.51), 2.518 (2.60), 2.523 (1.84), 2.669 (0.73), 2.673 (0.51), 2.813 (0.43), 2.986 (0.90), 3.054 (0.94), 3.061 (0.97), 3.068 (0.90), 3.419 (6.09), 3.423 (7.06), 3.870 (0.53), 3.895 (0.84), 3.948 (0.54), 3.963 (0.73), 3.989 (0.47), 4.227 (0.93), 4.233 (1.04), 4.241 (1.03), 4.247 (0.95), 4.645 (1.39), 5.345 (2.00), 5.358 (2.66), 7.543 (0.42), 7.565 (1.37), 7.591 (0.96), 7.609 (0.58), 7.615 (0.49), 7.630 (0.87), 7.636 (0.67), 8.061 (0.57), 8.075 (0.47), 8.087 (7.30).

### Example 21

### 4-[(7-[(1S*,5S*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol, Hydrogen Chloride Salt (Single Enantiomer 2)

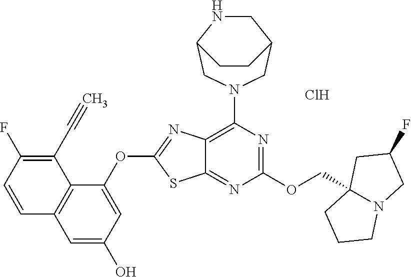 [SMILES: C#Cc1c(F)ccc2cc(O)cc(Oc3nc4c(N5CC6CCC(C5)NC6)nc(OC[C@@]56CCCN5C[C@H](F)C6)nc4s3)c12 ; Cl]

tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (single enantiomer 2) (34.0 mg, 44.6 μmol) was dissolved in methylene chloride (660 μl) and methanol (330 μl), HCl in 1,4-dioxane (330 μl, 4.0 M, 1.3 mmol) was added and the reaction was stirred at rt for 2 h. The reaction mixture was concentrated to the half of the volume under reduced pressure. Dichloromethane was added and the solid material was centrifuged. The solvent was decanted and the solid was dried under reduced pressure to afford 26 mg (95% purity, 80% yield) of the title compound.

LC-MS (Method 1): Rₜ=0.72 min; MS (ESIpos): m/z=620 [M+H]⁺

Specific optical rotation: [α]_{D}=+17.48 (from solution in DMSO, c=3.2 mg/mL)

¹H-NMR (400 MHz, DMSO-d6) δ[ppm]: 1.440 (0.96), 1.466 (1.13), 1.489 (0.59), 1.536 (0.41), 2.023 (0.52), 2.045 (0.51), 2.080 (0.50), 2.096 (0.54), 2.112 (0.60), 2.128 (0.65), 2.144 (0.84), 2.169 (0.54), 2.184 (0.45), 2.248 (0.45), 2.276 (0.61), 2.295 (0.42), 2.434 (0.77), 2.444 (0.73), 2.453 (0.95), 2.518 (4.66), 2.523 (2.92), 2.575 (0.41), 3.163 (0.43), 3.226 (1.12), 3.245 (1.00), 3.513 (1.00), 3.552 (0.96), 3.565 (0.43), 3.717 (0.44), 3.731 (0.64), 3.744 (0.64), 3.762 (0.61), 3.776 (0.87), 3.821 (0.65), 4.362 (1.55), 4.468 (4.62), 4.591 (3.32), 4.593 (3.44), 5.486 (0.57), 5.617 (0.56), 5.759 (16.00), 7.313 (2.08), 7.319 (3.05), 7.343 (2.66), 7.348 (1.91), 7.466 (1.16), 7.488 (2.32), 7.511 (1.20), 7.950 (1.03), 7.964 (1.06), 7.972 (1.07), 7.987 (1.03), 9.418 (0.50), 11.234 (0.55), 11.249 (0.55).

### Intermediate 21-1

### tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Single Enantiomer 2)

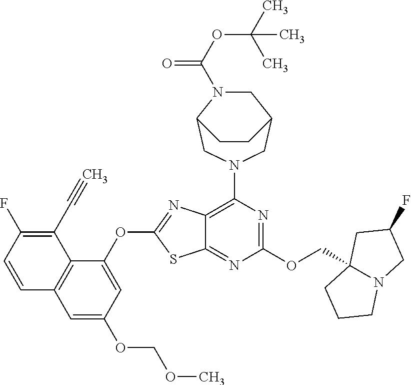 [SMILES: C#Cc1c(F)ccc2cc(OCOC)cc(Oc3nc4c(N5CC6CCC(C5)N(C(=O)OC(C)(C)C)C6)nc(OC[C@@]56CCCN5C[C@H](F)C6)nc4s3)c12]

The isomers of tert-butyl (1S*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,6-diazabicyclo[3.2.2]nonane-6-carboxylate (Mixture of 2 diastereomers) (120 mg, 157 μmol) were separated (using the following conditions: Instrument: PrepCon Labomatic HPLC-2; Column: YMC Amylose SA 5, 250×50; eluent A: methyl tert-butyl ether+0.1 vol % diethylamine; eluent B: acetonitrile; isocratic: 95% A+5% B; flow: 100 ml/min; temperature: 25° C.; UV: 280 nm) to afford 47.0 mg (39% yield) of the title compound. The second isomer is described as intermediate 20-1.

LC-MS (Method 1): Rₜ=1.19 min; MS (ESIpos): m/z=764 [M+H]⁺

Specific optical rotation: [α]_{D}=+19.62 (from solution in DMSO, c=2.8 mg/mL)

### Example 22

### 4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)-5-ethynyl-6-fluoronaphthalen-2-ol

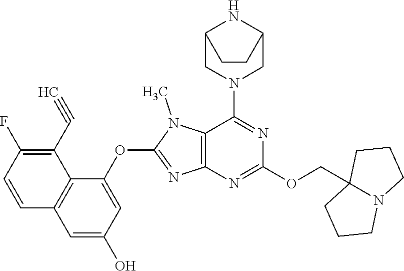 [SMILES: C#Cc1c(F)ccc2cc(O)cc(Oc3nc4nc(OCC56CCCN5CCC6)nc(N5CC6CCC(C5)N6)c4n3C)c12]

4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)-6-fluoro-5-{[tri(propan-2-yl)silyl]ethynyl}naphthalen-2-ol (13.9 mg, 18.8 μmol) was dissolved in THF (1.0 ml) and N,N,N-tributylbutan-1-aminium fluoride (19 μl, 1.0 M, 19 μmol; CAS-RN: [429-41-4]) was added. The reaction mixture was stirred at rt for 1 h. It was stirred at 55° C. overnight. Water and acetonitrile were added und the mixture was purified by prep HPLC (Method: column: Chromatorex C18; 125 mm×30 mm; 10 μm; flow=50 ml/min; using a gradient of water (+0.05% formic acid)/acetonitrile 15-50%) to afford 5.8 mg (95% purity, 50% yield) of the title compound.

LC-MS (Method 6): Rₜ=1.85 min; MS (ESIpos): m/z=583 [M]⁺

¹H-NMR (500 MHz, DMSO-d6) δ[ppm]: 1.924 (3.13), 1.940 (3.13), 1.950 (3.28), 1.963 (2.17), 1.984 (1.47), 2.013 (8.24), 2.034 (2.66), 2.043 (3.72), 2.056 (4.13), 2.065 (3.75), 2.072 (3.01), 3.153 (1.38), 3.163 (1.86), 3.174 (1.94), 3.184 (1.67), 3.373 (1.34), 3.645 (2.73), 3.672 (3.02), 3.906 (16.00), 3.944 (2.26), 4.140 (3.46), 4.361 (7.51), 4.627 (4.86), 7.214 (3.20), 7.218 (3.15), 7.307 (3.97), 7.311 (3.57), 7.461 (1.59), 7.479 (3.11), 7.497 (1.59), 7.960 (1.56), 7.972 (1.68), 7.979 (1.63), 7.990 (1.47), 9.473 (0.79), 9.794 (0.93), 9.813 (0.77), 10.416 (1.18).

### Intermediate 22-1

### tert-butyl 3-(2-chloro-7-methyl-7H-purin-6-yl)-3,8-diazabicyclo[3.2.1]octane-8-carboxylate

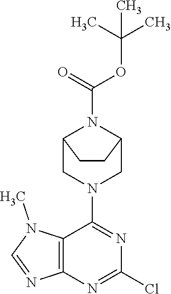 [SMILES: Cn1cnc2nc(Cl)nc(N3CC4CCC(C3)N4C(=O)OC(C)(C)C)c21]

2,6-dichloro-7-methyl-7H-purine (400 mg, 1.97 mmol) and tert-butyl 3,8-diazabicyclo[3.2.1]octane-8-carboxylate (439 mg, 2.07 mmol) were suspended in ethanol (8.0 ml) and triethylamine (270 μl, 2.0 mmol; CAS-RN: [121-44-8]) was added. The reaction mixture was stirred at 60° C. for 6 h. The solvent was removed under reduced pressure and the residue was diluted with ethyl acetate, washed with water and the organic layer was dried using a silicon coated filter. The filtrate was concentrated under reduced pressure to afford 700 mg (100% purity, 94% yield) of the title compound which was used without further purification.

LC-MS (Method 1): Rt=1.64 min; MS (ESIpos): m/z=379 [M+H]⁺

¹H-NMR (600 MHz, DMSO-d6) δ[ppm]: −0.017 (0.66), −0.010 (0.78), 1.426 (11.16), 1.433 (13.87), 1.441 (13.94), 1.443 (14.68), 1.698 (1.58), 1.781 (1.25), 1.973 (0.40), 1.979 (0.46), 1.989 (0.50), 2.486 (7.33), 2.489 (8.56), 2.525 (4.28), 3.306 (12.82), 3.313 (16.00), 3.320 (14.42), 3.324 (15.93), 3.326 (11.22), 3.348 (1.47), 3.930 (3.58), 3.936 (4.54), 3.946 (4.94), 4.026 (1.74), 4.046 (1.22), 4.219 (1.61), 8.365 (1.14), 8.372 (1.44), 8.381 (1.63).

### Intermediate 22-2

### tert-butyl 3-{7-methyl-2-[(tetrahydro-1H-pyrrolizin-7a(5H)-yl)methoxy]-7H-purin-6-yl}-3,8-diazabicyclo[3.2.1]octane-8-carboxylate

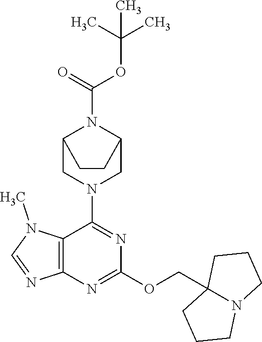 [SMILES: Cn1cnc2nc(OCC34CCCN3CCC4)nc(N3CC4CCC(C3)N4C(=O)OC(C)(C)C)c21]

Sodium hydride (55.2 mg, 60% purity, 1.38 mmol; CAS-RN: [7646-69-7]) was suspended in THF (25 ml) and (tetrahydro-1H-pyrrolizin-7a(5H)-yl)methanol (453 mg, 3.21 mmol), dissolved in THF (5.0 ml), was added. The mixture was stirred at rt for 15 min. tert-butyl 3-(2-chloro-7-methyl-7H-purin-6-yl)-3,8-diazabicyclo[3.2.1]octane-8-carboxylate (617 mg, 98% purity, 1.60 mmol) was dissolved in THF (5.0 ml) and added to the stirring mixture. It was stirred at 70° C. for 2 h. The reaction was quenched with NaHCO₃ solution and extracted with ethyl acetate. The combined organic layers were washed with water and dried using a silicon coated filter and concentrated under reduced pressure to afford 780 mg (87% purity, 88% yield) of the title compound which was used without further purification.

LC-MS (Method 1): Rt=1.03 min; MS (ESIpos): m/z=484 [M+H]⁺

### Intermediate 22-3

### tert-butyl 3-{8-bromo-7-methyl-2-[(tetrahydro-1H-pyrrolizin-7a(5H)-yl)methoxy]-7H-purin-6-yl}-3,8-diazabicyclo[3.2.1]octane-8-carboxylate

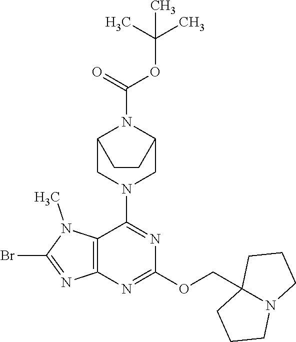 [SMILES: Cn1c(Br)nc2nc(OCC34CCCN3CCC4)nc(N3CC4CCC(C3)N4C(=O)OC(C)(C)C)c21]

tert-butyl 3-{7-methyl-2-[(tetrahydro-1H-pyrrolizin-7a(5H)-yl)methoxy]-7H-purin-6-yl}-3,8-diazabicyclo[3.2.1]octane-8-carboxylate (780 mg, 87% purity, 1.40 mmol) was dissolved in THF (15 ml) and at −78° C. Lithium bis(trimethylsilyl)amide (3.8 ml, 1.0 M, 3.8 mmol; CAS-RN: [4039-32-1]) was added and the reaction was stirred for 45 min at this temperature. Lithium bis(trimethylsilyl)amide (3.8 ml, 1.0 M, 3.8 mmol; CAS-RN: [4039-32-1]) was added again and bromine (200 μl, 3.9 mmol; CAS-RN: [7726-95-6]), dissolved in THF (10 ml) was added too. The reaction mixture was stirred at −78° C. for 30 min. The reaction was quenched with water and aq. sodium thiosulfate (w=10%) and extracted with methylene chloride. The combined organic layers were washed with sat. NaCl− solution and dried using a silicon coated filter and concentrated under reduced pressure. The crude product was purified by flash chromatography (silica phase, using a gradient of methylene chloride/methanol 5-80%) to afford 37.1 mg (95% purity, 4% yield) and 122 mg (100% purity, 16% yield) of the title compound.

LC-MS (Method 1): Rₜ=1.21 min; MS (ESIpos): m/z=562 [M+H]⁺

### Intermediate 22-4

### tert-butyl 3-(8-{[7-fluoro-3-(methoxymethoxy)-8-{[tri(propan-2-yl)silyl]ethynyl}naphthalen-1-yl]oxy}-7-methyl-2-[(tetrahydro-1H-pyrrolizin-7a(5H)-yl)methoxy]-7H-purin-6-yl)-3,8-diazabicyclo[3.2.1]octane-8-carboxylate

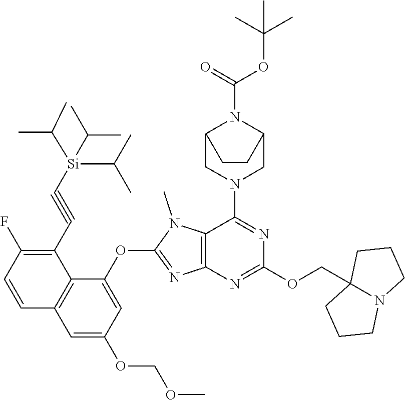 [SMILES: COCOc1cc(Oc2nc3nc(OCC45CCCN4CCC5)nc(N4CC5CCC(C4)N5C(=O)OC(C)(C)C)c3n2C)c2c(C#C[Si](C(C)C)(C(C)C)C(C)C)c(F)ccc2c1]

Sodium hydride (2.02 mg, 60% purity, 50.6 μmol; CAS-RN: [7646-69-7]) was suspended in THF (2.0 ml). 7-fluoro-3-(methoxymethoxy)-8-{[tri(propan-2-yl)silyl]ethynyl}naphthalen-1-ol (20.4 mg, 50.6 μmol) was dissolved in THF (2.0 ml) und added to the stirring sodium hydride mixture. It was stirred at rt for 15 min. tert-butyl 3-{8-bromo-7-methyl-2-[(tetrahydro-1H-pyrrolizin-7a(5H)-yl)methoxy]-7H-purin-6-yl}-3,8-diazabicyclo[3.2.1]octane-8-carboxylate (20.0 mg, 95% purity, 33.7 μmol) was dissolved in THF (2.0 ml) and added to the stirring mixture. It was stirred at rt for 4 h. The reaction mixture was diluted with water/acetonitrile and purified by prep. HPLC (Method: column: Chromatorex C18; 125 mm×30 mm; 10 μm; flow=50 ml/min; using a gradient of water (+0.05% formic acid)/acetonitrile 80-100%) to afford 13.1 mg (97% purity, 42% yield) of the title compound.

LC-MS (Method 1): Rₜ=2.34 min; MS (ESIpos): m/z=884 [M+H]⁺

### Intermediate 22-5

### tert-butyl 3-(8-{[7-fluoro-3-(methoxymethoxy)-8-{[tri(propan-2-yl)silyl]ethynyl}naphthalen-1-yl]oxy}-7-methyl-2-[(tetrahydro-1H-pyrrolizin-7a(5H)-yl)methoxy]-7H-purin-6-yl)-3,8-diazabicyclo[3.2.1]octane-8-carboxylate

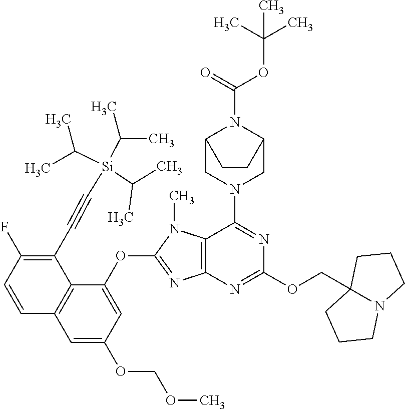 [SMILES: COCOc1cc(Oc2nc3nc(OCC45CCCN4CCC5)nc(N4CC5CCC(C4)N5C(=O)OC(C)(C)C)c3n2C)c2c(C#C[Si](C(C)C)(C(C)C)C(C)C)c(F)ccc2c1]

tert-butyl 3-{8-bromo-7-methyl-2-[(tetrahydro-1H-pyrrolizin-7a(5H)-yl)methoxy]-7H-purin-6-yl}-3,8-diazabicyclo[3.2.1]octane-8-carboxylate (20.0 mg, 100% purity, 35.6 μmol), 7-fluoro-3-(methoxymethoxy)-8-{[tri(propan-2-yl)silyl]ethynyl}naphthalen-1-ol (28.6 mg, 71.1 μmol) and potassium carbonate were suspended in N,N-dimethylacetamide (1.0 ml) and the mixture was stirred at 110° C. for 2 h. Water was added to the reaction mixture and it was extracted with ethyl acetate. The combined organic layers were washed with sat. NaCl solution, dried using a silicon coated filter and concentrated under reduced pressure. The crude product was purified by prep. HPLC (Method: column: Chromatorex C18; 125 mm×30 mm; 10 μm; flow=50 ml/min; using a gradient of water (+0.05% formic acid)/acetonitrile 70-90%) to afford 8.6 mg (90% purity, 25% yield) of the title compound.

LC-MS (Method 6): Rₜ=5.29 min; MS (ESIpos): m/z=884 [M]⁺

### Intermediate 22-6

### 4-{[6-(3,8-diazabicyclo[3.2.1]octan-3-yl)-7-methyl-2-(tetrahydro-1H-pyrrolizin-7a(5H)-ylmethoxy)-7H-purin-8-yl]oxy}-6-fluoro-5-[(triisopropylsilyl)ethynyl]-2-naphthol

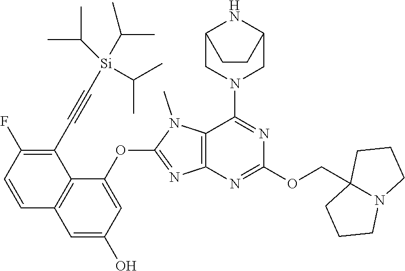 [SMILES: CC(C)[Si](C#Cc1c(F)ccc2cc(O)cc(Oc3nc4nc(OCC56CCCN5CCC6)nc(N5CC6CCC(C5)N6)c4n3C)c12)(C(C)C)C(C)C]

tert-butyl 3-(8-{[7-fluoro-3-(methoxymethoxy)-8-{[tri(propan-2-yl)silyl]ethynyl}naphthalen-1-yl]oxy}-7-methyl-2-[(tetrahydro-1H-pyrrolizin-7a(5H)-yl)methoxy]-7H-purin-6-yl)-3,8-diazabicyclo[3.2.1]octane-8-carboxylate (13.1 mg, 14.8 μmol) was dissolved in methylene chloride (2.0 ml), tribromborane (150 μl, 1.0 M, 150 μmol) was added and the reaction mixture was stirred at rt for 2 h. It was quenched with water and methylene chloride was removed under reduced pressure. The liquid residue was diluted with acetonitrile and purified by prep. HPLC (Method: column: Chromatorex C18; 125 mm×30 mm; 10 μm; flow=50 ml/min; using a gradient of water (+0.05% formic acid)/acetonitrile 50-70%) to afford 5.7 mg (100% purity, 52% yield) of the title compound.

LC-MS (Method 4): Rt=1.36 min; MS (ESIneg): m/z=738 [M−H]⁻

### Intermediate 22-7

### 4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)-6-fluoro-5-{[tri(propan-2-yl)silyl]ethynyl}naphthalen-2-ol

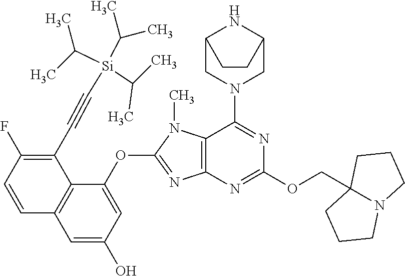 [SMILES: CC(C)[Si](C#Cc1c(F)ccc2cc(O)cc(Oc3nc4nc(OCC56CCCN5CCC6)nc(N5CC6CCC(C5)N6)c4n3C)c12)(C(C)C)C(C)C]

tert-butyl 3-(8-{[7-fluoro-3-(methoxymethoxy)-8-{[tri(propan-2-yl)silyl]ethynyl}naphthalen-1-yl]oxy}-7-methyl-2-[(tetrahydro-1H-pyrrolizin-7a(5H)-yl)methoxy]-7H-purin-6-yl)-3,8-diazabicyclo[3.2.1]octane-8-carboxylate (8.60 mg, 90% purity, 8.76 μmol) was dissolved in HCl/1,4-dioxane (1.0 ml, 4.0 M, 4.0 mmol) and stirred at rt for 45 min. The solvent was removed under reduced pressure. Methylene chloride was added and removed under reduced pressure and this process was repeated twice more to afford 8.2 mg (79% purity, 100% yield) of the title compound which was used without further purification.

LC-MS (Method 1): Rₜ=1.34 min; MS (ESIneg): m/z=738 [M−H]⁻

### Example 23

### 6-chloro-4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynylnaphthalen-2-ol

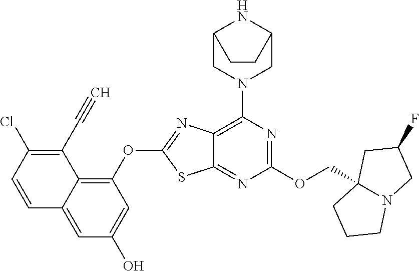 [SMILES: C#Cc1c(Cl)ccc2cc(O)cc(Oc3nc4c(N5CC6CCC(C5)N6)nc(OC[C@@]56CCCN5C[C@H](F)C6)nc4s3)c12]

tert-butyl 3-[2-({7-chloro-8-ethynyl-3-[(2-methoxyethoxy)methoxy]naphthalen-1-yl}oxy)-5-{[(2R,7aS)-2-fluorotetrahydro-1H-pyrrolizin-7a(5H)-yl]methoxy}[1,3]thiazolo[5,4-d]pyrimidin-7-yl]-3,8-diazabicyclo[3.2.1]octane-8-carboxylate (36.0 mg, 44.5 μmol) was dissolved in acetonitrile (350 μl) and cooled down with an ice bath. HCl in 1,4-dioxane (360 μl, 4.0 M, 1.4 mmol; CAS-RN:[7647-01-0]) was added and it was stirred at 0° C. for 30 min under Ar atmosphere. The reaction mixture was concentrated under reduced pressure and the residue was diluted with ethyl acetate and quenched with saturated NaHCO₃ solution. It was stirred for a few minutes and the organic layer was dried using a silicone coated filter and concentrated under reduced pressure. The crude product was treated with methylene chloride and a few drops ethanol and this mixture was sonicated. The unsolved precipitate was filtered off and dried under reduced pressure at 50° C. to afford 31 mg (97% purity, 109% yield) of the title compound which was used without further purification.

LC-MS (Method 1): Rₜ=0.79 min; MS (ESIpos): m/z=621 [M+H]⁺

¹H-NMR (400 MHz, DMSO-d6) δ[ppm]: 1.052 (0.64), 1.172 (0.80), 1.190 (0.80), 1.224 (1.20), 1.352 (0.42), 1.463 (2.45), 1.539 (2.41), 1.614 (1.63), 1.683 (0.73), 1.711 (2.97), 1.726 (3.18), 1.738 (3.00), 1.761 (1.71), 1.783 (2.19), 1.813 (2.00), 1.947 (1.65), 1.987 (4.23), 2.021 (0.92), 2.059 (2.40), 2.332 (2.77), 2.336 (1.27), 2.518 (16.00), 2.523 (10.95), 2.673 (2.76), 2.678 (1.25), 2.765 (0.94), 2.787 (2.09), 2.803 (2.43), 2.824 (1.21), 2.975 (6.28), 3.007 (1.94), 3.043 (4.61), 3.054 (4.83), 3.060 (4.76), 3.820 (2.04), 3.845 (2.79), 3.920 (4.32), 3.945 (2.69), 3.988 (0.45), 4.509 (0.49), 4.562 (7.83), 5.174 (2.01), 5.309 (1.74), 5.758 (6.85), 6.991 (1.85), 7.078 (2.02), 7.406 (1.73), 7.427 (2.05), 7.660 (1.54), 7.682 (1.46), 8.943 (0.97).

### Intermediate 23-1

### tert-butyl 3-(2-[(7-chloro-3-[(2-methoxyethoxy)methoxy]-8-{[tri(propan-2-yl)silyl]ethynyl}naphthalen-1-yl)oxy]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

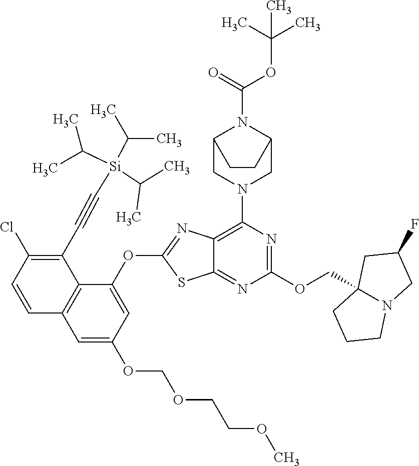 [SMILES: COCCOCOc1cc(Oc2nc3c(N4CC5CCC(C4)N5C(=O)OC(C)(C)C)nc(OC[C@@]45CCCN4C[C@H](F)C5)nc3s2)c2c(C#C[Si](C(C)C)(C(C)C)C(C)C)c(Cl)ccc2c1]

tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (111 mg, 190 μmol), 7-chloro-3-[(2-methoxyethoxy)methoxy]-8-{[tri(propan-2-yl)silyl]ethynyl}naphthalen-1-ol (176 mg, 380 μmol) and potassium carbonate (131 mg, 951 μmol; CAS-RN: [584-08-7]) were suspended in N,N-dimethylacetamide (2.0 ml) and the reaction mixture was stirred in a microwave reactor at 110° C. for 2 h. The reaction mixture was diluted with methylene chloride and a lot of water. It was extracted three times with methylene chloride, the combined organic layers were washed twice with half sat. and once with sat. NaCl solution, dried using a silicone coated filter and concentrated under reduced pressure. The crude product was purified by flash chromatography (aminophase, using a gradient of hexane/ethyl acetate 0-50%) to afford 91 mg (84% purity, 42% yield) of the title compound.

LC-MS (Method 1): Rₜ=1.61 min; MS (ESIpos): m/z=966 [M+H]⁺

### Intermediate 23-2

### tert-butyl 3-[2-({7-chloro-8-ethynyl-3-[(2-methoxyethoxy)methoxy]naphthalen-1-yl}oxy)-5-{[(2R,7aS)-2-fluorotetrahydro-1H-pyrrolizin-7a(5H)-yl]methoxy}[1,3]thiazolo[5,4-d]pyrimidin-7-yl]-3,8-diazabicyclo[3.2.1]octane-8-carboxylate

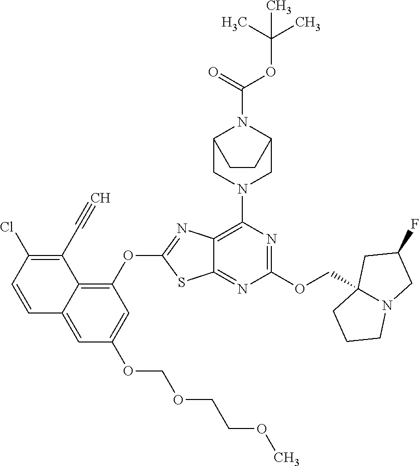 [SMILES: C#Cc1c(Cl)ccc2cc(OCOCCOC)cc(Oc3nc4c(N5CC6CCC(C5)N6C(=O)OC(C)(C)C)nc(OC[C@@]56CCCN5C[C@H](F)C6)nc4s3)c12]

tert-butyl 3-(2-[(7-chloro-3-[(2-methoxyethoxy)methoxy]-8-{1[tri(propan-2-yl)silyl]ethynyl}naphthalen-1-yl)oxy]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (45.0 mg, 46.6 μmol) was dissolved in THF (260 μl) and treated with tetrabutylammonium fluoride (93 μl, 1.0 M, 93 μmol; CAS-RN: [429-41-4]). It was stirred at rt under Ar atmosphere for 30 min. The reaction mixture was quenched with sat. NaHCO₃ solution and diluted with ethyl acetate. It was stirred for a few minutes and the organic layer was filtered through a silicone coated filter and concentrated under reduced pressure to afford 40 mg (90% purity, 95% yield) of the title compound which was used without further purification.

LC-MS (Method 2): Rₜ=1.70 min; MS (ESIneg): m/z=809 [M−H]⁻

### Intermediate 23-1 building block 1, 1. Step

### 5-[(4-chlorophenyl)acetyl]-2,2-dimethyl-1,3-dioxane-4,6-dione

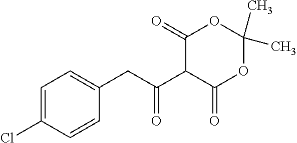 [SMILES: CC1(C)OC(=O)C(C(=O)Cc2ccc(Cl)cc2)C(=O)O1]

(4-chlorophenyl)acetic acid (5.00 g, 29.3 mmol), 2,2-dimethyl-1,3-dioxane-4,6-dione (4.22 g, 29.3 mmol) and N,N-dimethylpyridin-4-amine (304 mg, 2.49 mmol; CAS-RN: [1122-58-3]) were treated with acetonitrile (15 ml) and triethylamine (8.2 ml, 59 mmol; CAS-RN: [121-44-8]) was added and the temperature was kept at a maximum of 45° C. during this and the following slowly addition of 2,2-dimethylpropanoyl chloride. The reaction mixture was then stirred at 45° C. for 2 h. The reaction mixture was cooled down to 0° C. and 1M HCl solution was added slowly. The precipitated solid was filtered off and washed with acetonitrile and water. The solid was dried under reduced pressure to afford 6.01 g (95% purity, 66% yield) of the title compound.

LC-MS (Method 1): Rₜ=1.85 min; MS (ESIneg): m/z=295 [M−H]⁻

¹H-NMR (600 MHz, DMSO-d6) δ[ppm]: 1.681 (16.00), 4.345 (4.34), 7.328 (1.41), 7.342 (2.46), 7.379 (2.68), 7.393 (1.53).

### Intermediate 23-1 Building Block 1, 2. Step

### tert-butyl 4-(4-chlorophenyl)-3-oxobutanoate

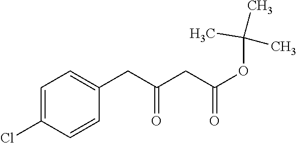 [SMILES: CC(C)(C)OC(=O)CC(=O)Cc1ccc(Cl)cc1]

5-[(4-chlorophenyl)acetyl]-2,2-dimethyl-1,3-dioxane-4,6-dione (6.00 g, 20.2 mmol) was dissolved in tert. butyl alcohol (19 ml) and the solution was stirred at 90° C. for 2 h. The solvent was removed under reduced pressure and the residue was diluted with methylene chloride. The organic layer was washed with water and sat. NaCl solution and was dried with magnesium sulfate. The filtrate was concentrated under reduced pressure to afford 4.57 g (91% purity, 76% yield) of the title compound.

LC-MS (Method 1): Rₜ=2.06 min; MS (ESIneg): m/z=267 [M−H]⁻

### Intermediate 23-1 Building Block 1, 3. Step

### 4-(4-chlorophenyl)-3-oxobutanoic acid

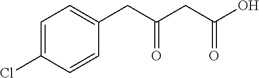 [SMILES: O=C(O)CC(=O)Cc1ccc(Cl)cc1]

tert-butyl 4-(4-chlorophenyl)-3-oxobutanoate (4.57 g, 17.0 mmol) was dissolved in methylene chloride (9.1 ml), trifluoroacetic acid (8.9 ml, 120 mmol; CAS-RN: [76-05-1]) was added and the mixture was stirred at rt for 1 h. The solvent was removed under reduced pressure. The residue was treated with cyclohexane and stirred at rt. The solid was filtered off and dried under reduced pressure to afford 2.83 g (96% purity, 75% yield) of the title compound.

LC-MS (Method 1): Rₜ=1.30 min; MS (ESIneg): m/z=211 [M−H]⁻

¹H-NMR (600 MHz, DMSO-d6) δ[ppm]: 2.137 (0.61), 3.536 (0.99), 3.553 (16.00), 3.894 (12.31), 4.994 (0.50), 7.193 (5.11), 7.207 (5.98), 7.211 (0.75), 7.306 (0.46), 7.362 (1.02), 7.366 (6.70), 7.370 (2.17), 7.377 (2.07), 7.380 (5.76), 7.384 (0.80).

### Intermediate 23-1 Building Block 1, 4. Step

### 7-chloronaphthalene-1,3-diol

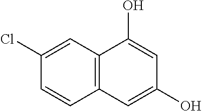 [SMILES: Oc1cc(O)c2cc(Cl)ccc2c1]

Under icebath cooling 4-(4-chlorophenyl)-3-oxobutanoic acid (2.83 g, 13.3 mmol) was treated with trifluoromethanesulfonic acid (29 ml, 330 mmol; CAS-RN: [1493-13-6]) and the solution was allowed to warm up to room temperature and stirred at rt overnight. The mixture was given on iced water and the precipitate was filtered off and dried under reduced pressure to afford 2.52 g (93% purity, 90% yield) of the title compound.

LC-MS (Method 1): Rₜ=1.45 min; MS (ESIpos): m/z=195 [M+H]⁺

¹H-NMR (600 MHz, DMSO-d6) δ[ppm]: 2.518 (1.07), 2.521 (0.96), 6.546 (15.26), 6.550 (16.00), 6.615 (13.63), 6.619 (12.12), 7.310 (7.74), 7.314 (7.51), 7.324 (8.50), 7.328 (8.10), 7.598 (13.80), 7.612 (12.46), 7.895 (12.02), 7.898 (11.47), 10.287 (1.18).

### Intermediate 23-1 building block 1, 5. Step

### 7-chloro-8-{[tri(propan-2-yl)silyl]ethynyl}naphthalene-1,3-diol

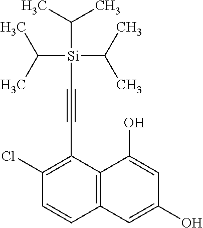 [SMILES: CC(C)[Si](C#Cc1c(Cl)ccc2cc(O)cc(O)c12)(C(C)C)C(C)C]

7-chloronaphthalene-1,3-diol (2.00 g, 93% purity, 9.53 mmol), (bromoethynyl)tri(propan-2-yl)silane (2.99 g, 11.4 mmol), dichloro(p-cymene)ruthenium(II) dimer (583 mg, 953 μmol; CAS-RN: [52462-29-0]) and potassium acetate (1.87 g, 19.1 mmol; CAS-RN: [127-08-2]) were suspended in 1,4-dioxane (22 ml) and it was stirred at 110° C. overnight. The reaction mixture was allowed to cool down to room temperature and was filtered over silica phase and washed with ethyl acetate. The organic layer was washed three times with water, dried over magnesium sulfate and the filtrate was concentrated under reduced pressure. The crude product was purified by flash chromatography (silica phase, using a gradient of cyclohexane/ethyl acetate 2:1) to afford 1.53 g (100% purity, 43% yield) of the title compound.

LC-MS (Method 1): Rₜ=2.97 min; MS (ESIpos): m/z=375 [M+H]⁺

¹H-NMR (500 MHz, DMSO-d6) δ[ppm]: 1.146 (16.00), 1.397 (0.68), 6.600 (0.45), 6.604 (0.60), 6.628 (0.55), 6.633 (0.40), 7.385 (0.52), 7.402 (0.59), 7.578 (0.51), 7.596 (0.44), 9.734 (0.99), 10.118 (1.06).

### Intermediate 23-1 Building Block 1, 6. Step

### 7-chloro-3-[(2-methoxyethoxy)methoxy]-8-{[tri(propan-2-yl)silyl]ethynyl}naphthalen-1-ol

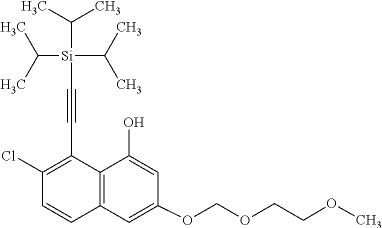 [SMILES: COCCOCOc1cc(O)c2c(C#C[Si](C(C)C)(C(C)C)C(C)C)c(Cl)ccc2c1]

7-chloro-8-{[tri(propan-2-yl)silyl]ethynyl}naphthalene-1,3-diol (1.43 g, 3.81 mmol) was dissolved in methylene chloride (18 ml) treated with DIPEA (1.9 ml, 11 mmol; CAS-RN: [7087-68-5]). The mixture was cooled down with an ice bath and carefully treated with 1-(chloromethoxy)-2-methoxyethane (780 μl, 6.8 mmol). It was stirred for 30 min at 0° C. under Ar atmosphere and at rt overnight. The reaction mixture was diluted with water and methylene chloride. It was extracted three times, washed once with water and brine, filtered through a silicone coated filter and concentrated under reduced pressure. The crude product was purified by flash chromatography (silica phase, using a gradient of hexane/ethyl acetate 5-30%) to afford 1.01 g (99% purity, 57% yield) of the title compound.

LC-MS (Method 2): Rt=1.91 min; MS (ESIneg): m/z=463 [M−H]⁻

¹H-NMR (400 MHz, DMSO-d6) δ[ppm]: 1.145 (16.00), 3.216 (4.30), 5.310 (1.10), 6.714 (0.40), 7.476 (0.42), 7.498 (0.49), 7.703 (0.40), 10.352 (0.95). (1.21), 8.078 (1.27), 8.092 (1.14).

### Example 24

### 5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol (Diastereomer 1, Single Enantiomer 1)

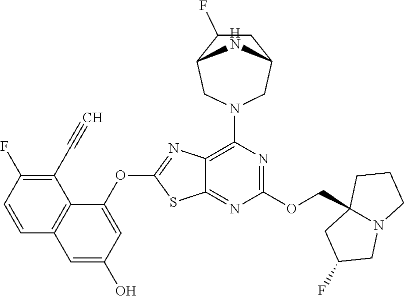 [SMILES: C#Cc1c(F)ccc2cc(O)cc(Oc3nc4c(N5C[C@H]6CC(F)[C@@H](C5)N6)nc(OC[C@@]56CCCN5C[C@H](F)C6)nc4s3)c12]

Tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 1, single enantiomer 1) (30.0 mg, 39.1 μmol) was dissolved in acetonitrile (1.0 ml), at 0° C. HCl (500 μl, 4.0 M in 1,4-dioxane, 2.0 mmol) was added and the mixture was stirred for 30 minutes at room temperature. Afterwards, the reaction mixture was concentrated under reduced pressure and the crude material was purified by preparative HPLC (Method B) to afford 13.6 mg (56% yield) of the title compound.

LC-MS (Method 4): Rₜ=0.91 min; MS (ESIneg): m/z=621 [M−H]⁻

¹H-NMR (600 MHz, CHLOROFORM-d) δ[ppm]: 1.198 (1.72), 1.209 (3.42), 1.221 (1.77), 1.811 (0.44), 1.825 (0.51), 1.837 (0.51), 1.902 (0.99), 1.931 (0.82), 1.973 (1.28), 2.051 (0.61), 2.060 (0.67), 2.084 (0.95), 2.094 (1.34), 2.107 (1.24), 2.118 (1.18), 2.212 (1.60), 2.261 (1.29), 2.626 (16.00), 3.001 (0.76), 3.011 (0.80), 3.086 (1.15), 3.216 (0.82), 3.248 (0.88), 3.356 (0.59), 3.464 (1.17), 3.476 (2.13), 3.488 (2.23), 3.499 (4.61), 3.676 (0.87), 3.706 (0.49), 4.095 (0.77), 4.113 (1.49), 4.129 (0.65), 5.039 (0.72), 5.049 (0.69), 5.131 (0.68), 5.141 (0.72), 5.227 (0.78), 5.300 (1.28), 5.315 (0.76), 7.124 (2.37), 7.127 (2.51), 7.162 (2.40), 7.224 (1.06), 7.239 (2.02), 7.640 (1.00), 7.649 (1.08), 7.655 (1.03), 7.664 (0.91).

### Example 24, Intermediate 1

### tert-Butyl (1R*,5R*)-3-(5-chloro[1,3]thiazolo[5,4-d]pyrimidin-7-yl)-6-fluoro-3,8-diazabicyclo[3.2.1]octane-8-carboxylate (Single Diastereomer 1, Racemate)

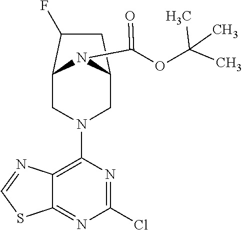 [SMILES: CC(C)(C)OC(=O)N1[C@@H]2CC(F)[C@H]1CN(c1nc(Cl)nc3scnc13)C2]

Tert-butyl (1R*,5R*,6S*)-6-fluoro-3,8-diazabicyclo[3.2.1]octane-8-carboxylate (single diastereomer 1, racemate) (250 mg, 1.09 mmol) and 5,7-dichloro[1,3]thiazolo[5,4-d]pyrimidine (224 mg, 1.09 mmol; CAS-RN: [13479-88-4]) were dissolved in 1,4-dioxane (4.6 ml), then triethylamine (610 μl, 4.3 mmol; CAS-RN: [121-44-8]) was added and the mixture was stirred for 2 h at room temperature. Afterwards, the reaction mixture was concentrated under reduced pressure. The residue was dissolved in dichloromethane and the organic layer was washed three times with water. The combined organic layer was dried using a water-resistant filter and concentrated under reduced pressure to afford 418 mg (93% yield) of the title compound.

LC-MS (Method 4): Rt=2.15 min; MS (ESIpos): m/z=400 [M+H]⁺

¹H-NMR (600 MHz, DMSO-d6) δ[ppm]: 1.439 (5.09), 3.566 (16.00), 9.271 (1.60).

### Example 24, Intermediate 2

### tert-Butyl (1R*,5R*)-6-fluoro-3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (Mixture of Diastereomers 1)

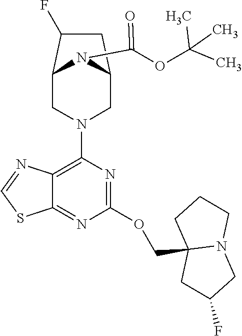 [SMILES: CC(C)(C)OC(=O)N1[C@@H]2CC(F)[C@H]1CN(c1nc(OC[C@@]34CCCN3C[C@H](F)C4)nc3scnc13)C2]

Tert-Butyl (1R*,5R*)-3-(5-chloro[1,3]thiazolo[5,4-d]pyrimidin-7-yl)-6-fluoro-3,8-diazabicyclo[3.2.1]octane-8-carboxylate (single diastereomer 1, racemate) (415 mg, 1.04 mmol) and [(2R,7aS)-2-fluorotetrahydro-1H-pyrrolizin-7a(5H)-yl]methanol (248 mg, 1.56 mmol) were dissolved in THF (6.7 ml), then NaH (37.4 mg, 1.56 mmol; CAS-RN: [7646-69-7]) was added and the mixture was stirred over night at room temperature. Afterwards, the reaction mixture was quenched with water and was extracted with ethyl acetate. The combined organic layers were dried using a water-resistant filter and concentrated under reduced pressure to afford 554 mg (100% yield) of the title compound.

LC-MS (Method 4): Rₜ=1.25 min; MS (ESIpos): m/z=523 [M+H]⁺

¹H-NMR (400 MHz, DMSO-d6) δ[ppm]: 0.834 (0.54), 0.854 (0.66), 1.157 (0.84), 1.169 (0.58), 1.175 (1.54), 1.193 (1.20), 1.235 (1.85), 1.440 (16.00), 1.611 (1.53), 1.625 (2.17), 1.638 (2.27), 1.666 (0.91), 1.751 (1.77), 1.770 (2.47), 1.779 (2.27), 1.796 (2.23), 1.825 (2.15), 1.835 (3.39), 1.846 (2.02), 1.858 (1.74), 1.895 (1.53), 1.935 (1.89), 1.946 (1.92), 1.971 (2.06), 1.983 (1.93), 1.989 (3.28), 2.021 (2.29), 2.029 (2.14), 2.041 (1.68), 2.066 (1.27), 2.078 (1.42), 2.097 (1.46), 2.109 (1.38), 2.367 (0.42), 2.711 (0.45), 2.740 (0.52), 2.762 (1.40), 2.777 (1.43), 2.785 (1.08), 2.802 (0.97), 2.815 (0.92), 2.832 (1.00), 2.925 (2.82), 2.931 (4.09), 2.963 (0.54), 3.007 (4.95), 3.014 (4.00), 3.039 (2.68), 3.050 (2.36), 3.069 (1.88), 3.092 (3.26), 3.106 (2.23), 3.119 (1.32), 3.132 (1.20), 3.210 (0.64), 3.568 (0.86), 3.922 (0.64), 3.949 (1.02), 4.020 (1.52), 4.038 (1.26), 4.352 (1.00), 4.391 (1.06), 4.515 (1.94), 5.131 (1.46), 5.137 (1.73), 5.201 (1.03), 5.268 (1.58), 5.273 (1.69), 5.335 (0.92), 9.019 (6.88).

### Example 24, Intermediate 3

### tert-Butyl (1R*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (Mixture of Diastereomers 1)

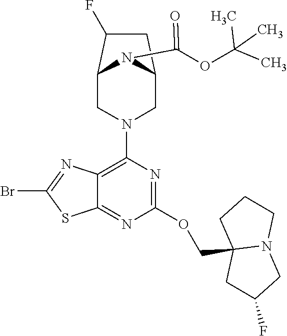 [SMILES: CC(C)(C)OC(=O)N1[C@@H]2CC(F)[C@H]1CN(c1nc(OC[C@@]34CCCN3C[C@H](F)C4)nc3sc(Br)nc13)C2]

Tert-Butyl (1R*,5R*)-6-fluoro-3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (mixture of diastereomers 1) (553 mg, 1.06 mmol) was dissolved in THF (7.8 ml) and at −78° C. lithium bis(trimethylsilyl)amide (2.5 ml, 1.0 M in THF, 2.5 mmol; CAS-RN: [4039-32-1]) was added dropwise. The mixture was stirred for 30 minutes at this temperature. Then, bromine (76 μl, 1.5 mmol; CAS-RN: [7726-95-6]) was added dropwise and the mixture was stirred for 1 h at −78° C. Afterwards, the reaction mixture was quenched at −78° C. with sodium thiosulfate (10% aq. solution). Then, it was allowed to warm up to room temperature and was extracted three times with ethyl acetate. The combined organic layers were dried using a water-resistant filter and concentrated under reduced pressure to afford 552 mg (87% yield) of the title compound.

LC-MS (Method 4): Rₜ=1.40 min; MS (ESIpos): m/z=601 [M+H]⁺

¹H-NMR (400 MHz, DMSO-d6) δ[ppm]: −0.069 (5.57), −0.057 (2.13), 1.371 (2.13), 2.472 (16.00), 2.996 (0.50).

### Example 24, Intermediate 4

### tert-Butyl (1R*,5R*)-6-fluoro-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (Mixture of Diastereomers 1)

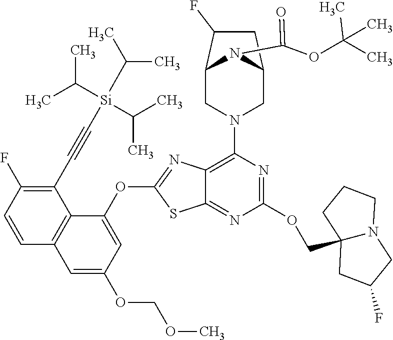 [SMILES: COCOc1cc(Oc2nc3c(N4C[C@H]5CC(F)[C@@H](C4)N5C(=O)OC(C)(C)C)nc(OC[C@@]45CCCN4C[C@H](F)C5)nc3s2)c2c(C#C[Si](C(C)C)(C(C)C)C(C)C)c(F)ccc2c1]

Tert-Butyl (1R*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (mixture of diastereomers 1) (250 mg, 416 μmol) and 7-fluoro-3-(methoxymethoxy)-8-{[tri(propan-2-yl)silyl]ethynyl}naphthalen-1-ol (335 mg, 831 μmol) were dissolved in N,N-dimethylacetamide (4.4 ml), then K₂CO₃ (287 mg, 2.08 mmol; CAS-RN: [584-08-7]) was added and the mixture was stirred for 2 h at room temperature. Afterwards, the reaction mixture was diluted with water and was extracted with water. The combined organic layers were dried using a water-resistant filter and concentrated under reduced pressure. The crude product was purified by preparative HPLC (Method: Column: Reprosil C18; 10 μm; 125×30 mm/Flow: 75 ml/min/Eluent: A=H2O (0.01% HCOOH), B=Acetonitril/Gradient: 0.00-2.50 min=10% B, 17.65-19.50 min=95% B, 19.65-20.65 min=10% B) to afford 119 mg (31% yield) of the title compound.

LC-MS (Method 5): Rₜ=0.73 min; MS (ESIpos): m/z=923 [M+H]⁺

¹H-NMR (400 MHz, DMSO-d6) δ[ppm]: 0.969 (7.93), 0.978 (16.00), 1.002 (1.98), 1.021 (0.87), 1.038 (0.49), 1.423 (6.69), 1.736 (0.62), 1.836 (0.43), 1.941 (0.52), 2.003 (0.87), 2.077 (0.65), 2.526 (0.42), 3.000 (0.96), 3.039 (0.76), 3.075 (0.64), 3.393 (0.90), 3.418 (12.69), 5.229 (0.49), 5.347 (5.44), 7.515 (1.41), 7.520 (1.61), 7.569 (0.60), 7.591 (1.21), 7.614 (0.64), 7.649 (1.66), 7.655 (1.56), 8.076 (0.67), 8.090 (0.71), 8.099 (0.71), 8.113 (0.66).

### Example 24, Intermediate 5

### tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (mixture of diastereomers 1)

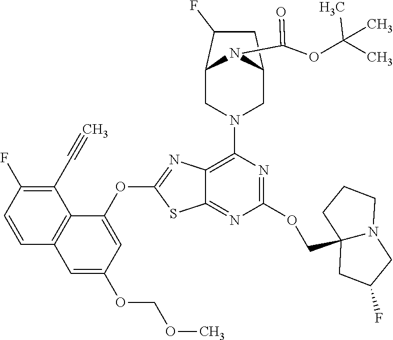 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

Tert-Butyl (1R*,5R*)-6-fluoro-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (mixture of diastereomers 1) (110 mg, 119 μmol) was dissolved in THF (1.7 ml) and at 0° C. tetra-n-butylammoniumfluorid (130 μl, 1.0 M in THF, 130 μmol; CAS-RN: [429-41-4]) was added and the mixture was stirred over night at room temperature. Afterwards, the reaction mixture was diluted with water and was extracted with dichloromethane. The combined organic layers were dried using a water-resistant filter and concentrated under reduced pressure to afford 90 mg (96% yield) of the title compound.

LC-MS (Method 4): Rₜ=1.90 min; MS (ESIpos): m/z=767 [M+H]⁺

### Example 24, Intermediate 6

### tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 1, single enantiomer 1)

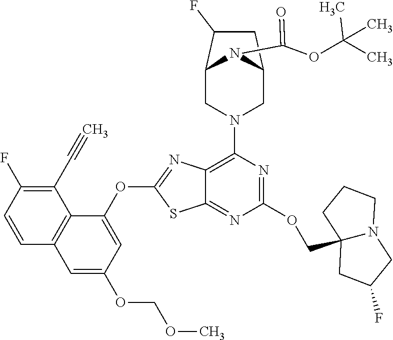 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

The mixture of diastereomers 1 of tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (90 mg) were separated (Method: Column: 250×20 mm ReproSil Chiral NR, 5 m, flow: 15.00 ml/min, eluent: TBME/n-Heptan 75:25) to afford 30.0 mg (31% yield) of the title compound.

LC-MS (Method4): Rₜ=1.87 min; MS (ESIpos): m/z=767 [M+H]⁺

### Example 25

### 5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol (diastereomer 1, single enantiomer 2)

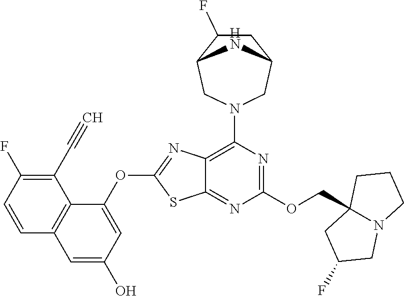 [SMILES: C#Cc1c(F)ccc2cc(O)cc(Oc3nc4c(N5C[C@H]6CC(F)[C@@H](C5)N6)nc(OC[C@@]56CCCN5C[C@H](F)C6)nc4s3)c12]

Tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 1, single enantiomer 2) (32.0 mg, 41.7 μmol) was dissolved in acetonitrile (1.0 ml), at 0° C. HCl (500 μl, 4.0 M in 1,4-dioxane, 2.0 mmol) was added and the mixture was stirred for 30 minutes at room temperature. Afterwards, the reaction mixture was concentrated under reduced pressure and the crude material was purified by preparative HPLC (Method B) to afford 17.2 mg (63% yield) of the title compound.

LC-MS (Method 4): Rₜ=0.92 min; MS (ESIneg): m/z=621 [M−H]⁻

¹H-NMR (600 MHz, CHLOROFORM-d) δ[ppm]: 1.255 (0.46), 1.809 (0.70), 1.822 (0.80), 1.833 (0.74), 1.889 (1.73), 1.901 (1.80), 1.932 (1.50), 1.941 (1.38), 1.974 (2.45), 2.030 (0.86), 2.045 (0.88), 2.054 (0.99), 2.078 (1.40), 2.103 (2.20), 2.116 (1.92), 2.212 (3.33), 2.258 (2.36), 2.628 (7.82), 3.003 (1.73), 3.012 (1.74), 3.083 (2.60), 3.192 (0.94), 3.216 (1.81), 3.248 (2.16), 3.316 (1.01), 3.357 (1.48), 3.473 (1.34), 3.497 (7.18), 3.573 (0.77), 3.632 (0.89), 3.641 (1.82), 3.652 (1.38), 3.674 (1.62), 3.696 (1.36), 3.741 (1.11), 3.748 (0.74), 3.755 (1.16), 3.765 (1.66), 3.774 (0.80), 4.070 (1.21), 4.087 (2.08), 4.115 (3.43), 4.132 (1.68), 4.698 (0.54), 4.763 (1.74), 4.918 (0.53), 5.039 (1.36), 5.050 (1.21), 5.131 (1.31), 5.141 (1.23), 5.225 (1.65), 5.300 (16.00), 5.314 (1.67), 7.116 (4.16), 7.159 (4.01), 7.217 (1.67), 7.232 (3.12), 7.247 (2.12), 7.355 (0.61), 7.631 (1.69), 7.640 (1.84), 7.646 (1.74), 7.654 (1.47).

### Example 25, Intermediate 1

### tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 1, single enantiomer 2)

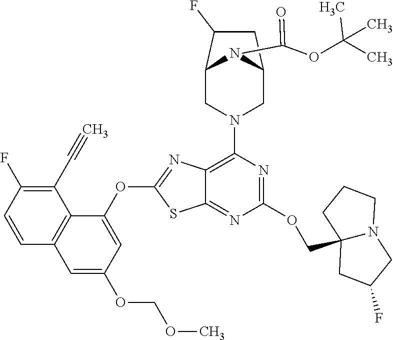 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

The mixture of diastereomers 1 of tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (90 mg) were separated (Method: Column: 250×20 mm ReproSil Chiral NR, 5 m, flow: 15.00 ml/min, eluent: TBME/n-Heptan 75:25) to afford 32.0 mg (34% yield) of the title compound.

LC-MS (Method 1): Rₜ=1.86 min; MS (ESIpos): m/z=767 [M+H]⁺

### Example 26

### 5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol (Diastereomer 2, Single Enantiomer 1)

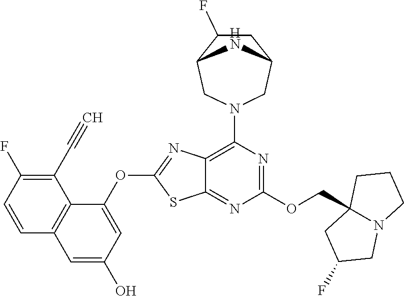 [SMILES: C#Cc1c(F)ccc2cc(O)cc(Oc3nc4c(N5C[C@H]6CC(F)[C@@H](C5)N6)nc(OC[C@@]56CCCN5C[C@H](F)C6)nc4s3)c12]

tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 2, single enantiomer 1) (25.0 mg, 32.6 μmol) was dissolved in acetonitrile (1.0 ml) and at 0° C. HCl (500 μl, 4.0 M in 1,4-dioxane, 2.0 mmol) was added and the mixture was stirred for 30 minutes at room temperature. Afterwards, the reaction mixture was concentrated under reduced pressure and the crude material was purified by preparative HPLC (Method B) to afford 11.9 mg (50% yield) of the title compound.

LC-MS (Method 4): Rₜ=0.92 min; MS (ESIneg): m/z=621 [M−H]⁻

### Example 26, Intermediate 1

### tert-Butyl (1R*,5R*)-3-(5-chloro[1,3]thiazolo[5,4-d]pyrimidin-7-yl)-6-fluoro-3,8-diazabicyclo[3.2.1]octane-8-carboxylate (Single Diastereomer 2, Racemate)

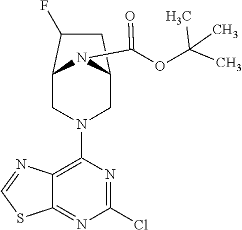 [SMILES: CC(C)(C)OC(=O)N1[C@@H]2CC(F)[C@H]1CN(c1nc(Cl)nc3scnc13)C2]

Tert-butyl (1R*,5R*,6R*)-6-fluoro-3,8-diazabicyclo[3.2.1]octane-8-carboxylate (250 mg, 1.09 mmol) and 5,7-dichloro[1,3]thiazolo[5,4-d]pyrimidine (single diastereomer 2, racemate) (224 mg, 1.09 mmol; CAS-RN: [13479-88-4]) were dissolved in 1,4-dioxane (4.6 ml), then triethylamine (610 μl, 4.3 mmol; CAS-RN: [121-44-8]) was added and the mixture was stirred for 2 h at room temperature. Afterwards, the reaction mixture was concentrated under reduced pressure. The residue was dissolved in dichloromethane and the organic layer was washed three times with water. The combined organic layer was dried using a water-resistant filter and concentrated under reduced pressure to afford 380 mg (88% yield) of the title compound.

LC-MS (Method 4): Rₜ=2.15 min; MS (ESIpos): m/z=400 [M+H]⁺

¹H-NMR (600 MHz, DMSO-d6) δ[ppm]: 1.440 (5.42), 3.566 (16.00), 9.273 (1.77).

### Example 26, Intermediate 2

### tert-Butyl (1R*,5R*)-6-fluoro-3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (Mixture of Diastereomers 2)

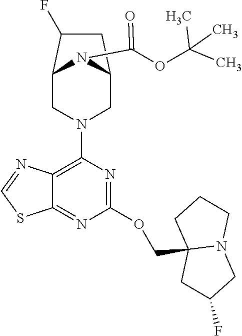 [SMILES: CC(C)(C)OC(=O)N1[C@@H]2CC(F)[C@H]1CN(c1nc(OC[C@@]34CCCN3C[C@H](F)C4)nc3scnc13)C2]

Tert-Butyl (1R*,5R*)-3-(5-chloro[1,3]thiazolo[5,4-d]pyrimidin-7-yl)-6-fluoro-3,8-diazabicyclo[3.2.1]octane-8-carboxylate (single diastereomer 2, racemate) (375 mg, 938 μmol, racemate) and [(2R,7aS)-2-fluorotetrahydro-1H-pyrrolizin-7a(5H)-yl]methanol (224 mg, 1.41 mmol) were dissolved in THF (6.1 ml), then NaH (33.8 mg, 1.41 mmol; CAS-RN: [7646-69-7]) was added and the mixture was stirred over night at room temperature. Afterwards, the reaction mixture was quenched with water and was extracted with ethyl acetate. The combined organic layers were dried using a water-resistant filter and concentrated under reduced pressure to afford 489 mg (100% yield) of the title compound.

LC-MS (Method 4): Rₜ=1.25 min; MS (ESIpos): m/z=523 [M+H]⁺

¹H-NMR (400 MHz, DMSO-d6) δ[ppm]: 0.835 (0.46), 0.854 (0.55), 1.157 (0.63), 1.170 (0.52), 1.176 (1.25), 1.194 (1.10), 1.214 (0.51), 1.235 (1.64), 1.398 (0.82), 1.441 (16.00), 1.611 (1.23), 1.625 (1.80), 1.635 (1.90), 1.652 (0.83), 1.667 (0.74), 1.751 (1.70), 1.770 (2.27), 1.780 (2.08), 1.796 (1.90), 1.826 (1.85), 1.836 (2.82), 1.846 (1.84), 1.858 (1.49), 1.895 (1.26), 1.935 (1.59), 1.947 (1.57), 1.971 (1.85), 1.984 (1.70), 1.989 (2.71), 2.023 (2.23), 2.029 (1.86), 2.041 (1.41), 2.066 (1.06), 2.078 (1.20), 2.090 (1.44), 2.097 (1.44), 2.109 (1.38), 2.741 (0.43), 2.761 (1.14), 2.776 (1.18), 2.785 (0.91), 2.802 (0.87), 2.814 (0.89), 2.831 (1.01), 2.852 (0.45), 2.925 (2.45), 2.930 (3.33), 2.962 (0.46), 3.007 (4.49), 3.014 (3.36), 3.039 (2.19), 3.050 (1.89), 3.070 (1.87), 3.093 (2.85), 3.105 (1.83), 3.119 (1.05), 3.130 (0.97), 3.201 (0.61), 3.568 (0.76), 3.922 (0.61), 3.950 (1.04), 4.021 (1.38), 4.039 (1.11), 4.352 (1.00), 4.391 (1.04), 4.513 (1.54), 5.132 (1.25), 5.137 (1.49), 5.201 (1.01), 5.269 (1.35), 5.273 (1.45), 5.336 (0.87), 9.019 (6.27).

### Example 26, Intermediate 3

### tert-Butyl (1R*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (Mixture of Diastereomers 2)

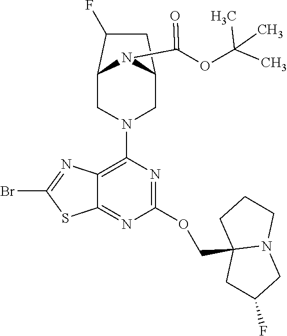 [SMILES: CC(C)(C)OC(=O)N1[C@@H]2CC(F)[C@H]1CN(c1nc(OC[C@@]34CCCN3C[C@H](F)C4)nc3sc(Br)nc13)C2]

Tert-Butyl (1R*,5R*)-6-fluoro-3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (mixture of diastereomers 2) (485 mg, 928 μmol) was dissolved in THF (6.9 ml) and at −78° C. Lithium bis(trimethylsilyl)amide (2.2 ml, 1.0 M in THF, 2.2 mmol; CAS-RN: [4039-32-1]) was added dropwise. The mixture was stirred for 30 minutes. Then, bromine (67 μl, 1.3 mmol; CAS-RN: [7726-95-6]) was added dropwise and the mixture was stirred for 1 h at −78° C. Afterwards, the reaction mixture was quenched at −78° C. with sodium thiosulfate (10% aq. solution). Then, it was allowed to warm up to room temperature and was extracted three times with ethyl acetate. The combined organic layers were dried using a water-resistant filter and concentrated under reduced pressure to afford 443 mg (79% yield) of the title compound.

LC-MS (Method 4): Rₜ=1.39 min; MS (ESIpos): m/z=601 [M+H]⁺

¹H-NMR (400 MHz, DMSO-d6) δ[ppm]: −0.010 (0.94), 0.010 (11.28), 0.057 (1.18), 0.066 (16.00), 0.833 (0.42), 0.851 (0.52), 1.192 (0.47), 1.234 (1.42), 1.353 (0.52), 1.437 (9.25), 1.645 (0.76), 1.743 (0.99), 1.760 (0.99), 1.771 (0.99), 1.793 (0.94), 1.808 (0.85), 1.850 (0.94), 1.870 (0.57), 1.914 (0.66), 1.960 (0.94), 2.009 (1.37), 2.041 (0.42), 2.081 (1.13), 2.093 (0.80), 2.327 (0.52), 2.800 (0.61), 2.823 (0.66), 2.931 (0.47), 2.954 (0.47), 2.996 (1.60), 3.030 (1.04), 3.061 (1.46), 3.109 (0.57), 3.136 (0.85), 3.160 (1.04), 3.215 (0.61), 3.240 (1.23), 3.264 (1.09), 3.939 (0.52), 4.001 (0.57), 4.357 (0.52), 4.394 (0.57), 5.141 (0.52), 5.192 (0.52), 5.284 (1.04), 5.325 (0.52).

### Example 26, Intermediate 4

### tert-Butyl (1R*,5R*)-6-fluoro-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (Mixture of Diastereomers 2)

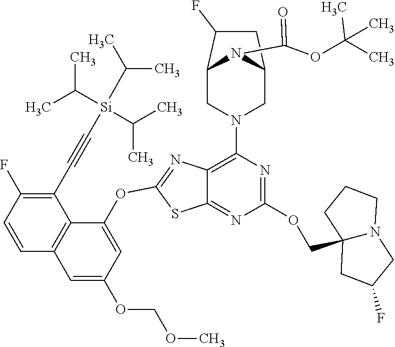 [SMILES: COCOc1cc(Oc2nc3c(N4C[C@H]5CC(F)[C@@H](C4)N5C(=O)OC(C)(C)C)nc(OC[C@@]45CCCN4C[C@H](F)C5)nc3s2)c2c(C#C[Si](C(C)C)(C(C)C)C(C)C)c(F)ccc2c1]

Tert-Butyl (1R*,5R*)-3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (mixture of diastereomers 2) (250 mg, 416 μmol) and 7-fluoro-3-(methoxymethoxy)-8-{[tri(propan-2-yl)silyl]ethynyl}naphthalen-1-ol (335 mg, 831 μmol, racemate) were dissolved in N,N-dimethylacetamide (4.4 ml), then K₂CO₃ (287 mg, 2.08 mmol; CAS-RN: [584-08-7]) was added and the mixture was stirred for 2 h at room temperature. Afterwards, the reaction mixture was diluted with water and was extracted with water. The combined organic layers were dried using a water-resistant filter and concentrated under reduced pressure. The crude product was purified by preparative HPLC (Method: Column: Reprosil C18; 10 μm; 125×30 mm/Flow: 75 ml/min/Eluent: A=H2O (0.01% HCOOH), B=Acetonitril/Gradient: 0.00-2.50 min=10% B, 17.65-19.50 min=95% B, 19.65-20.65 min=10% B) to afford 114 mg (30% yield) of the title compound.

LC-MS (Method 4): Rₜ=2.15 min; MS (ESIpos): m/z=923 [M+H]⁺

¹H-NMR (400 MHz, DMSO-d6) δ[ppm]: 0.969 (7.14), 0.978 (16.00), 0.992 (3.58), 1.002 (1.93), 1.021 (0.78), 1.038 (0.46), 1.423 (6.18), 1.726 (0.61), 1.743 (0.53), 1.825 (0.43), 1.933 (0.47), 1.993 (0.93), 2.066 (0.79), 2.525 (0.64), 2.811 (0.41), 2.980 (1.00), 3.006 (0.62), 3.042 (1.06), 3.060 (0.75), 3.393 (1.12), 3.418 (11.34), 5.229 (0.56), 5.347 (4.86), 7.514 (1.26), 7.520 (1.45), 7.569 (0.54), 7.591 (1.10), 7.614 (0.58), 7.648 (1.49), 7.654 (1.38), 8.076 (0.59), 8.090 (0.64), 8.099 (0.63), 8.113 (0.60).

### Example 26, Intermediate 5

### tert-Butyl (1R*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (mixture of diastereomers 2)

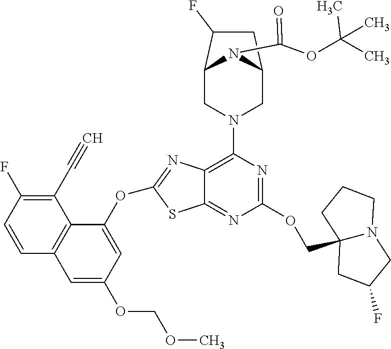 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

Tert-Butyl (1R*,5R*)-6-fluoro-3-(2-{[7-fluoro-3-(methoxymethoxy)-8-{[tri(propan-2-yl)silyl]ethynyl}naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (mixture of diastereomers 2) (100 mg, 108 μmol) was dissolved in THF (1.5 ml) and at 0° C. tetra-n-butylammoniumfluorid (120 μl, 1.0 M in THF, 120 μmol; CAS-RN: [429-41-4]) was added and the mixture was stirred over night at room temperature. Afterwards, the reaction mixture was diluted with water and was extracted with dichloromethane. The combined organic layers were dried using a water-resistant filter and concentrated under reduced pressure to afford 78 mg (90% yield) of the title compound.

LC-MS (Method 4): Rₜ=1.86 min; MS (ESIpos): m/z=767 [M+H]⁺

### Example 26, Intermediate 6

### tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (Diastereomer 2, Single Enantiomer 1)

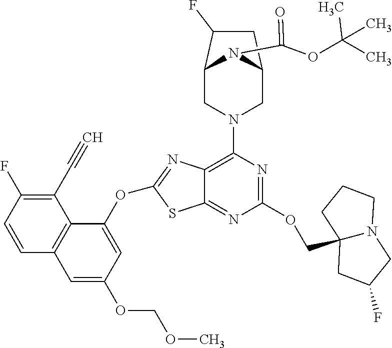 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

The mixture of diastereomers 2 of tert-Butyl (1R*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (78 mg, 0.10 mmol) were separated (Method: Column: 250×20 mm ReproSil Chiral NR, m, flow: 15.00 ml/min, eluent: TBME/n-Heptan 75:25) to afford 25 mg (83% yield) of the title compound.

LC-MS (Method 4): Rₜ=1.86 min; MS (ESIpos): m/z=767 [M+H]⁺

### Example 27

### 5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol (Diastereomer 2, Single Enantiomer 2)

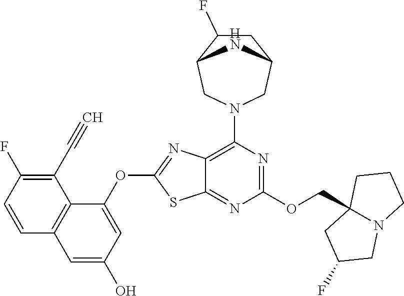 [SMILES: C#Cc1c(F)ccc2cc(O)cc(Oc3nc4c(N5C[C@H]6CC(F)[C@@H](C5)N6)nc(OC[C@@]56CCCN5C[C@H](F)C6)nc4s3)c12]

Tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (diastereomer 2, single enantiomer 2) (25.0 mg, 32.6 μmol) was dissolved in acetonitrile (1.0 ml) and at 0° C. HCl (500 μl, 4.0 M in 1,4-dioxane, 2.0 mmol) was added and the mixture was stirred for 30 minutes at room temperature. Afterwards, the reaction mixture was concentrated under reduced pressure and the crude material was purified by preparative HPLC (Method B) to afford 8.9 mg (44% yield) of the title compound.

LC-MS (Method 4): Rₜ=0.92 min; MS (ESIneg): m/z=621 [M−H]⁻

¹H-NMR (500 MHz, CHLOROFORM-d) δ[ppm]: 0.073 (0.49), 0.834 (1.15), 1.256 (4.01), 1.817 (4.48), 1.899 (7.82), 1.939 (7.48), 1.967 (8.38), 2.035 (3.30), 2.077 (4.64), 2.106 (6.28), 2.176 (4.71), 2.211 (7.95), 2.267 (4.11), 2.633 (1.60), 3.008 (7.46), 3.076 (9.23), 3.223 (6.36), 3.260 (6.74), 3.366 (7.02), 3.498 (13.70), 3.645 (9.22), 3.699 (8.48), 3.737 (7.87), 3.767 (5.39), 4.103 (8.12), 4.121 (8.11), 4.699 (2.24), 4.766 (5.96), 4.933 (2.06), 5.031 (3.67), 5.141 (3.41), 5.221 (4.43), 5.303 (16.00), 5.327 (4.24), 7.113 (9.51), 7.164 (9.43), 7.228 (6.58), 7.638 (5.77), 8.450 (0.89).

### Example 27, Intermediate 1

### tert-Butyl (1R*,5R*)-3-(2-{[8-ethynyl-7-fluoro-3-(methoxymethoxy)-1-naphthyl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (Diastereomer 2, Single Enantiomer 2)

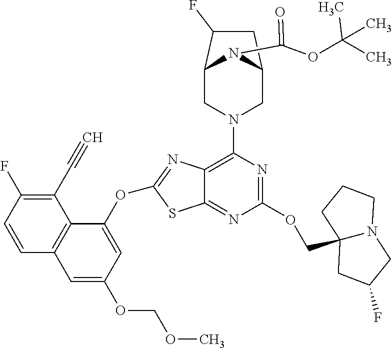 [SMILES: C#Cc1c(F)ccc2cc(OCOC)cc(Oc3nc4c(N5C[C@H]6CC(F)[C@@H](C5)N6C(=O)OC(C)(C)C)nc(OC[C@@]56CCCN5C[C@H](F)C6)nc4s3)c12]

The mixture of diastereomers 2 of tert-Butyl (1R*,5R*)-3-(2-{[8-ethynyl-7-fluoro-3-(methoxymethoxy)naphthalen-1-yl]oxy}-5-{[(2R,7aS)-2-fluorotetrahydro-1H-pyrrolizin-7a(5H)-yl]methoxy}[1,3]thiazolo[5,4-d]pyrimidin-7-yl)-6-fluoro-3,8-diazabicyclo[3.2.1]octane-8-carboxylate (78 mg, 0.10 mmol) were separated (Method: Column: 250×20 mm ReproSil Chiral NR, m, flow: 15.00 ml/min, eluent: TBME/n-Heptan 75:25) to afford 25 mg (83% yield) of the title compound.

LC-MS (Method 4): Rₜ=1.86 min; MS (ESIpos): m/z=767 [M+H]⁺

### Example 28

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]amino}naphthalen-2-ol

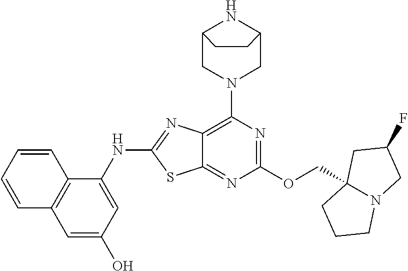 [SMILES: Oc1cc(Nc2nc3c(N4CC5CCC(C4)N5)nc(OC[C@@]45CCCN4C[C@H](F)C5)nc3s2)c2ccccc2c1]

Tert-butyl 3-(5-{[(2R,7aS)-2-fluorotetrahydro-1H-pyrrolizin-7a(5H)-yl]methoxy}-2-[(3-methoxynaphthalen-1-yl)amino][1,3]thiazolo[5,4-d]pyrimidin-7-yl)-3,8-diazabicyclo[3.2.1]octane-8-carboxylate (65.0 mg, 96.2 μmol) was dissolved in dichloromethane (620 μl), at 0° C. tribromborane (1.4 ml, 1.0 M in dichloromethane, 1.4 mmol; CAS-RN: [10294-33-4]) was added and the mixture was stirred for 75 minutes under Ar atmosphere at 0° C. Afterwards, the reaction mixture was diluted with K₂CO₃ (2 M aq. solution) and was extracted with a mixture of dichloromethane and methanol (8:2). The combined organic layers were dried using a water-resistant filter and concentrated under reduced pressure. The crude product was purified by flash chromatography (silica phase, using a gradient of dichloromethane/methanol+2% ammonia: 0-60%). The combined fractions were concentrated under reduced pressure and the residue was stirred in a mixture of dichloromethane and methanol (95:5). The mixture was filtered and the filtrate was concentrated under reduced pressure to afford 14.0 mg (25% yield) of the title compound.

LC-MS (Method 1): Rₜ=0.53 min; MS (ESIpos): m/z=562 [M+H]⁺

¹H-NMR (400 MHz, DMSO-d6) δ[ppm]: 0.850 (0.46), 1.230 (1.46), 1.600 (2.86), 1.624 (11.73), 1.655 (2.64), 1.693 (0.85), 1.707 (1.04), 1.734 (3.56), 1.748 (3.61), 1.760 (3.48), 1.783 (1.99), 1.794 (0.97), 1.805 (1.08), 1.832 (2.05), 1.924 (0.97), 1.957 (2.29), 1.966 (2.36), 1.978 (1.65), 2.005 (4.42), 2.011 (4.33), 2.040 (0.69), 2.050 (0.99), 2.076 (2.59), 2.083 (2.35), 2.090 (2.21), 2.101 (2.49), 2.128 (0.51), 2.138 (0.56), 2.350 (0.83), 2.365 (0.67), 2.383 (0.62), 2.518 (16.00), 2.523 (11.18), 2.562 (1.29), 2.577 (1.06), 2.585 (0.73), 2.600 (0.57), 2.679 (1.31), 2.784 (0.86), 2.807 (2.00), 2.823 (2.28), 2.845 (0.91), 2.962 (0.63), 2.997 (5.80), 3.010 (1.69), 3.019 (1.47), 3.027 (1.00), 3.039 (1.54), 3.062 (4.31), 3.075 (4.53), 3.082 (4.52), 3.100 (3.49), 3.140 (3.30), 3.159 (9.37), 3.171 (8.81), 3.184 (0.84), 3.447 (8.12), 3.835 (4.78), 3.860 (6.88), 3.886 (1.19), 3.937 (7.77), 3.962 (5.10), 3.993 (1.27), 4.018 (2.51), 4.053 (2.12), 4.079 (1.02), 4.087 (0.65), 4.100 (1.62), 4.113 (1.63), 4.126 (0.55), 4.485 (0.62), 4.501 (0.81), 4.518 (0.64), 4.901 (0.71), 5.194 (2.23), 5.328 (1.84), 6.898 (8.77), 6.903 (8.81), 7.278 (2.79), 7.281 (2.95), 7.295 (3.91), 7.298 (5.32), 7.302 (3.40), 7.316 (3.78), 7.319 (3.68), 7.394 (3.93), 7.397 (4.00), 7.414 (5.95), 7.432 (3.26), 7.677 (6.55), 7.697 (5.58), 7.816 (10.81), 7.822 (10.68), 8.117 (5.78), 8.139 (5.40), 9.726 (9.11), 10.206 (6.71).

### Example 28, Intermediate 1

### 3-methoxynaphthalen-1-amine

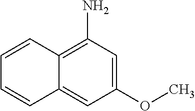

1-Bromo-3-methoxynaphthalene (1.00 g, 4.22 mmol) and benzophenone imine (803 mg, 4.43 mmol; CAS-RN: [1013-88-3]) were dissolved in toluene (18 ml), (R)-(+)-2,2′-Bis-(diphenylphosphino)-1,1′-binaphthalin (131 mg, 211 μmol, CAS-RN: [76189-55-4]) was added and N₂ was bubbled through this mixture for 5 minutes. Then tris(dibenzylideneacetone)di-palladium(0) (96.6 mg, 105 μmol; CAS-RN: [52409-22-0]) and sodium tert-butylate (608 mg, 6.33 mmol; CAS-RN: [865-48-5]) were added and the mixture was stirred at 90° C. under N₂ atmosphere for 2 hours. The reaction mixture was quenched with NH₂Cl (sat. aq. solution) and diluted with water. It was extracted three times with ethyl acetate, the combined organic layers were washed once with water and brine, filtered through a silicone coated filter and concentrated under reduced pressure. The crude was dissolved in ethyl acetate (50 ml) and treated with HCl (50 ml, 2 M aq. solution). The mixture was stirred at room temperature for 4 h. The mixture was filtered and the pH value of the filtrate was adjusted to 8 by addition of NaOH (2 M aq. solution). The aq. mixture was extracted three times with ethyl acetate. The combines organic layers were filtered through a silicone coated filter and concentrated under reduced pressure. The crude product was purified by flash chromatographie (amino phase, using a gradient of hexane/ethyl acetate 0-25%) to afford 488 mg (67% yield) of the title compound.

LC-MS (Method 2): Rₜ=0.97 min; MS (ESIpos): m/z=174 [M+H]⁺

¹H-NMR (400 MHz, DMSO-d6) δ[ppm]: 2.518 (0.80), 2.523 (0.53), 3.334 (16.00), 5.737 (3.65), 6.305 (3.75), 6.311 (4.16), 6.520 (2.53), 6.525 (2.40), 7.153 (0.97), 7.157 (1.03), 7.170 (1.29), 7.174 (1.83), 7.177 (1.12), 7.191 (1.30), 7.195 (1.27), 7.310 (1.09), 7.313 (1.13), 7.327 (0.99), 7.331 (1.92), 7.334 (1.37), 7.348 (1.05), 7.351 (1.00), 7.596 (1.75), 7.617 (1.52), 7.619 (1.47), 7.925 (1.63), 7.946 (1.54).

### Example 28, Intermediate 2

### tert-butyl 3-(5-{[(2R,7aS)-2-fluorotetrahydro-1H-pyrrolizin-7a(5H)-yl]methoxy}-2-[(3-methoxynaphthalen-1-yl)amino][1,3]thiazolo[5,4-d]pyrimidin-7-yl)-3,8-diazabicyclo[3.2.1]octane-8-carboxylate

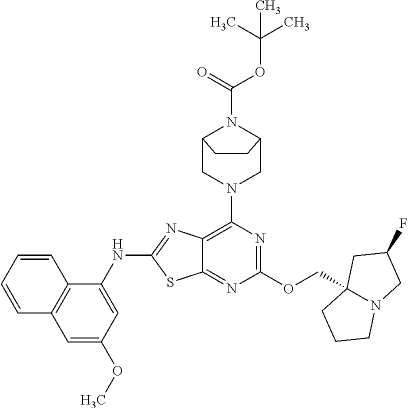 [SMILES: COc1cc(Nc2nc3c(N4CC5CCC(C4)N5C(=O)OC(C)(C)C)nc(OC[C@@]45CCCN4C[C@H](F)C5)nc3s2)c2ccccc2c1]

Tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (200 mg, 343 μmol) and 3-methoxynaphthalen-1-amine (59.4 mg, 343 μmol) were dissolved in 1,4-dioxane (730 μl), then Cs₂CO₃ (55.8 mg, 171 μmol; CAS-RN: [534-17-8]), palladium(II) acetate (7.69 mg, 34.3 μmol; CAS-RN: [3375-31-3]) and 4,5-bis(diphenylphosphino)-9,9-dimethylxanthene (19.8 mg, 34.3 μmol; CAS-RN: [161265-03-8]) were added and the mixture was stirred over night at 100° C. Afterwards, the reaction mixture was diluted with water and was extracted with ethyl acetate. The combined organic layers were washed with NaCl (sat., aq. solution), dried using a water-resistant filter and concentrated under reduced pressure. The crude product was purified twice by flash chromatography (amino phase, using a gradient of hexane/ethyl acetate 50-100% and silica phase, using a gradient of dichloromethane/ethanol 0-30%) to afford 86.2 mg (35% yield) of the title compound.

LC-MS (Method 2): Rₜ=1.64 min; MS (ESIpos): m/z=677 [M+H]⁺

¹H-NMR (400 MHz, DMSO-d6) δ[ppm]: 1.154 (0.84), 1.172 (1.87), 1.190 (0.93), 1.429 (0.46), 1.448 (16.00), 1.702 (0.42), 1.847 (0.49), 1.907 (0.66), 1.988 (2.98), 2.518 (1.49), 2.523 (1.14), 3.893 (6.07), 4.017 (0.67), 4.034 (0.65), 4.215 (0.54), 5.760 (4.72), 7.097 (0.82), 7.103 (0.81), 7.401 (0.40), 7.404 (0.54), 7.501 (0.57), 7.826 (0.59), 7.846 (0.52), 8.050 (0.90), 8.057 (0.88), 8.222 (0.54), 8.243 (0.50), 10.361 (1.14).

### Example 29

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}naphthalen-2-ol

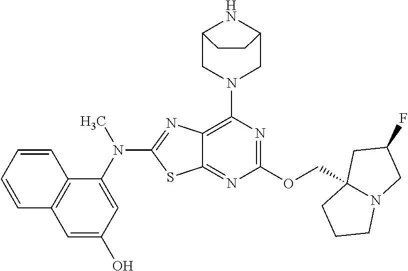 [SMILES: CN(c1nc2c(N3CC4CCC(C3)N4)nc(OC[C@@]34CCCN3C[C@H](F)C4)nc2s1)c1cc(O)cc2ccccc12]

Tert-butyl 3-(5-{[(2R,7aS)-2-fluorotetrahydro-1H-pyrrolizin-7a(5H)-yl]methoxy}-2-[(3-methoxynaphthalen-1-yl)(methyl)amino][1,3]thiazolo[5,4-d]pyrimidin-7-yl)-3,8-diazabicyclo[3.2.1]octane-8-carboxylate (62.0 mg, 89.9 μmol) was dissolved in dichloromethane (580 μl), at 0° C. tribromborane (1.3 ml, 1.0 M in dichloromethane, 1.3 mmol; CAS-RN: [10294-33-4]) was added and the mixture was stirred for 75 minutes under Ar atmosphere at 0° C. Afterwards, the reaction mixture was diluted with K₂CO₃ (2 M aq. solution) and was extracted with a mixture of dichloromethane and methanol (8:2). The combined organic layers were dried using a water-resistant filter and concentrated under reduced pressure. The crude product was purified by flash chromatography (amino phase, using a gradient of dichloromethane/methanol 0-20%) to afford 26.0 mg (48% yield) of the title compound.

LC-MS (Method 1): Rₜ=0.59 min; MS (ESIpos): m/z=576 [M+H]⁺

¹H-NMR (400 MHz, DMSO-d6) δ[ppm]: 1.631 (0.90), 1.652 (3.11), 1.684 (0.82), 1.700 (0.67), 1.707 (0.64), 1.715 (0.60), 1.723 (0.43), 1.896 (0.46), 1.903 (0.42), 1.997 (0.62), 2.018 (0.61), 2.030 (0.86), 2.051 (0.76), 2.287 (0.49), 2.304 (0.53), 2.322 (0.67), 2.326 (0.60), 2.332 (0.48), 2.336 (0.55), 2.518 (2.20), 2.523 (1.52), 2.664 (0.43), 2.669 (0.56), 2.673 (0.43), 2.931 (0.41), 2.945 (1.35), 2.955 (0.73), 2.966 (0.87), 2.974 (0.95), 2.996 (0.87), 3.105 (1.07), 3.119 (1.25), 3.133 (0.89), 3.148 (0.55), 3.480 (1.91), 3.545 (12.33), 3.788 (0.52), 3.865 (0.59), 3.890 (0.40), 3.921 (0.96), 3.947 (1.68), 3.985 (1.41), 4.011 (0.69), 4.466 (0.53), 4.483 (0.41), 5.759 (16.00), 7.256 (7.11), 7.302 (0.74), 7.304 (0.73), 7.319 (0.98), 7.322 (1.34), 7.340 (0.98), 7.342 (0.92), 7.449 (0.90), 7.452 (0.93), 7.469 (1.44), 7.472 (1.08), 7.487 (0.77), 7.489 (0.74), 7.633 (1.49), 7.654 (1.27), 7.813 (1.65), 7.833 (1.45).

### Example 29, Intermediate 1

### tert-butyl 3-(5-{[(2R,7aS)-2-fluorotetrahydro-1H-pyrrolizin-7a(5H)-yl]methoxy}-2-[(3-methoxynaphthalen-1-yl)(methyl)amino][1,3]thiazolo[5,4-d]pyrimidin-7-yl)-3,8-diazabicyclo[3.2.1]octane-8-carboxylate

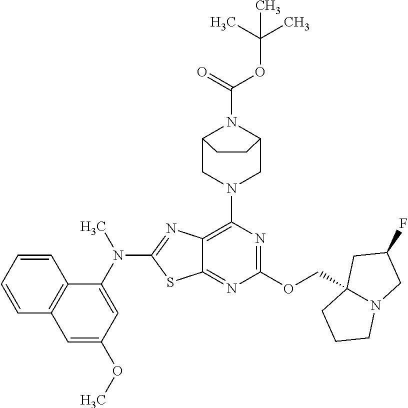 [SMILES: COc1cc(N(C)c2nc3c(N4CC5CCC(C4)N5C(=O)OC(C)(C)C)nc(OC[C@@]45CCCN4C[C@H](F)C5)nc3s2)c2ccccc2c1]

Tert-butyl 3-(5-{[(2R,7aS)-2-fluorotetrahydro-1H-pyrrolizin-7a(5H)-yl]methoxy}-2-[(3-methoxynaphthalen-1-yl)amino][1,3]thiazolo[5,4-d]pyrimidin-7-yl)-3,8-diazabicyclo[3.2.1]octane-8-carboxylate (47.0 mg, 69.5 μmol) was dissolved in THF (690 μl), at 0° C. NaH (4.55 mg, 55% purity, 104 μmol; CAS-RN: [7646-69-7]) was added and the mixture was stirred 30 minutes at 0° C. Then iodomethane (17 μl, 280 μmol; CAS-RN: [74-88-4] was added and the mixture was stirred for 1 h at room temperature. Afterwards, the reaction mixture was diluted with NH₄Cl (sat., aq. solution) and was extracted with ethyl acetate. The combined organic layers were, dried using a water-resistant filter and concentrated under reduced pressure. The crude product was purified by preparative HPLC (Method B, using a gradient of water/acetonitrile 65-100%) to afford 9.60 mg (19% yield) of the title compound.

LC-MS (Method 2): Rₜ=1.74 min; MS (ESIpos): m/z=691 [M+H]⁺

¹H-NMR (400 MHz, DMSO-d6) δ[ppm]: 0.851 (0.70), 0.867 (1.22), 0.885 (1.03), 0.904 (1.81), 0.923 (0.83), 1.231 (2.92), 1.279 (0.48), 1.288 (0.58), 1.299 (0.57), 1.315 (0.44), 1.411 (0.44), 1.447 (16.00), 1.684 (0.61), 1.698 (0.70), 2.518 (3.26), 2.523 (2.17), 3.578 (5.85), 3.922 (6.05), 4.227 (1.04), 4.233 (1.08), 4.241 (0.90), 4.247 (0.67), 5.758 (5.40), 7.404 (0.44), 7.407 (0.58), 7.426 (1.54), 7.432 (1.45), 7.490 (0.93), 7.496 (0.77), 7.550 (0.60), 7.554 (0.44), 7.686 (0.63), 7.707 (0.53), 7.949 (0.68), 7.969 (0.60), 8.088 (3.20).

### Example 30

### 4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl) [1,3]thiazolo[5,4-d]pyrimidin-2-yl]amino}-2-naphthol

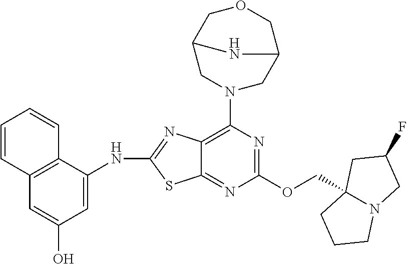 [SMILES: Oc1cc(Nc2nc3c(N4CC5COCC(C4)N5)nc(OC[C@@]45CCCN4C[C@H](F)C5)nc3s2)c2ccccc2c1]

Tert-butyl 7-(5-{[(2R,7aS)-2-fluorotetrahydro-1H-pyrrolizin-7a(5H)-yl]methoxy}-2-[(3-methoxy-1-naphthyl)amino][1,3]thiazolo[5,4-d]pyrimidin-7-yl)-3-oxa-7,9-diazabicyclo[3.3.1]nonane-9-carboxylate (38.0 mg, 54.9 μmol) was dissolved in dichloromethane (350 μl), at 0° C. tribromborane (820 μl, 1.0 M, 820 μmol; CAS-RN: [10294-33-4]) was added and the mixture was stirred for 30 minutes at 0° C. Afterwards, the reaction mixture was diluted with water and was extracted with ethyl acetate. The combined organic layers were, dried using a water-resistant filter and concentrated under reduced pressure. The crude product was purified by preparative HPLC (Method: Column: Reprosil C18; 10 μm; 125×30 mm/Flow: 75 ml/min/Eluent: A=H2O (0.01% HCOOH), B=Acetonitril/Gradient: 0.00-5.00 min=10% B, 6.50 min=20% B, 17.0-19.75 min=100% B, 19.75.00-23.00 min=90% B) to afford 7.20 mg (22% yield) of the title compound.

LC-MS (Method 4): Rₜ=0.75 min; MS (ESIneg): m/z=576 [M−H]⁻

¹H-NMR (500 MHz, DMSO-d6) δ[ppm]: 0.995 (16.00), 1.008 (15.93), 1.755 (0.41), 2.036 (0.47), 3.027 (0.67), 3.095 (0.49), 3.100 (0.49), 3.612 (0.64), 3.625 (0.85), 3.627 (0.73), 3.638 (0.70), 3.641 (0.85), 3.654 (0.63), 3.745 (0.46), 3.767 (0.76), 3.875 (0.51), 3.895 (0.67), 3.974 (0.65), 3.994 (0.46), 5.476 (0.55), 5.491 (0.55), 6.886 (0.76), 6.890 (0.77), 7.300 (0.47), 7.412 (0.50), 7.674 (0.56), 7.690 (0.49), 7.852 (1.03), 7.856 (0.98), 8.138 (0.51), 8.154 (0.50), 8.174 (1.46), 10.185 (0.97).

### Example 30, Intermediate 1

### tert-butyl 7-(5-{[(2R,7aS)-2-fluorotetrahydro-1H-pyrrolizin-7a(5H)-yl]methoxy}-2-[(3-methoxy-1-naphthyl)amino][1,3]thiazolo[5,4-d]pyrimidin-7-yl)-3-oxa-7,9-diazabicyclo[3.3.1]nonane-9-carboxylate

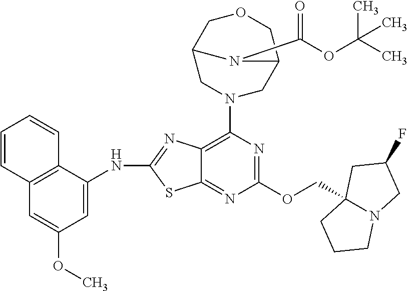 [SMILES: COc1cc(Nc2nc3c(N4CC5COCC(C4)N5C(=O)OC(C)(C)C)nc(OC[C@@]45CCCN4C[C@H](F)C5)nc3s2)c2ccccc2c1]

Tert-butyl 7-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3-oxa-7,9-diazabicyclo[3.3.1]nonane-9-carboxylate (46.0 mg, 76.7 μmol) was dissolved in 1,4-dioxane (160 μl), then 3-methoxynaphthalen-1-amine (14.6 mg, 84.4 μmol), palladium(II) acetate (1.72 mg, 7.67 μmol; CAS-RN: [3375-31-3]), 4,5-bis(diphenylphosphino)-9,9-dimethylxanthene (4.44 mg, 7.67 μmol; CAS-RN: [161265-03-8]) and Cs2CO3 (30.0 mg, 92.1 μmol; CAS-RN: [534-17-8]) were added and the mixture was stirred overnight under Ar atmosphere at 100° C. Afterwards, the reaction mixture was filtered and purified preparative HPLC (Method: Column: Reprosil C18; 10 μm; 125×30 mm/Flow: 75 ml/min/Eluent: A=H2O (0.01% HCOOH), B=Acetonitril/Gradient: 0.00-5.00 min=10% B, 6.50 min=20% B, 17.0-19.75 min=100% B, 19.75.00-23.00 min=90% B) to afford 34.8 mg (61% yield) of the title compound.

LC-MS (Method 4): Rt=1.61 min; MS (ESIpos): m/z=692 [M+H]⁺

¹H-NMR (600 MHz, DMSO-d6) δ[ppm]: 1.454 (16.00), 1.764 (0.61), 1.773 (0.55), 2.045 (0.53), 2.515 (0.44), 2.518 (0.41), 3.040 (0.77), 3.102 (0.61), 3.863 (0.67), 3.881 (5.34), 3.904 (0.57), 3.920 (0.56), 3.994 (0.42), 7.094 (0.84), 7.407 (0.60), 7.421 (0.42), 7.487 (0.49), 7.500 (0.68), 7.830 (0.77), 7.843 (0.72), 8.140 (0.83), 8.236 (0.48), 8.250 (0.47), 10.332 (1.01).

### Example 31

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}-5-ethynylnaphthalen-2-ol

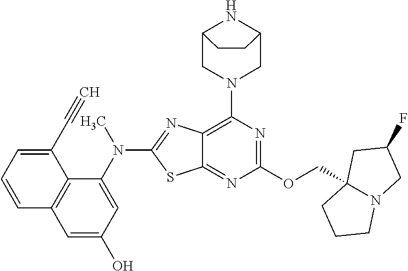 [SMILES: C#Cc1cccc2cc(O)cc(N(C)c3nc4c(N5CC6CCC(C5)N6)nc(OC[C@@]56CCCN5C[C@H](F)C6)nc4s3)c12]

Tert-butyl 3-[(2Z)-2-{[8-ethynyl-3-(methoxymethoxy)naphthalen-1-yl]imino}-5-{[(2R,7aS)-2-fluorotetrahydro-1H-pyrrolizin-7a(5H)-yl]methoxy}-1-methyl-,2-dihydro[1,3]thiazolo[5,4-d]pyrimidin-7-yl]-3,8-diazabicyclo[3.2.1]octane-8-carboxylate (90.0 mg, 121 μmol) was dissolved in a mixture of dichloromethane (2.2 ml) and methanol (1.2 ml), at 0° C. HCl (1.2 ml, 4.0 M in 1,4-dioxane, 4.8 mmol) was added and the mixture was stirred for 1.5 h at room temperature. Afterwards, the reaction mixture was concentrated under reduced pressure. The residue was dissolved in methanol, NaHCO₃ (sat., aq. solution) was added and the mixture was purified by flash chromatography (amino phase, using a gradient of dichloromethane/methanol 5-40%) to afford 44.0 mg (58% yield) of the title compound.

LC-MS (Method 2): Rₜ=1.05 min; MS (ESIpos): m/z=601 [M+H]⁺

¹H-NMR (400 MHz, DMSO-d6) δ[ppm]: 1.621 (1.18), 1.686 (0.76), 1.700 (0.67), 1.714 (0.60), 1.798 (0.46), 1.907 (0.41), 1.918 (0.47), 1.957 (0.84), 1.964 (0.95), 2.038 (0.68), 2.049 (0.50), 2.518 (1.46), 2.523 (0.97), 2.773 (0.41), 2.788 (0.48), 2.957 (0.79), 3.030 (1.27), 3.388 (8.76), 3.447 (0.57), 3.751 (0.80), 3.776 (1.17), 3.860 (1.14), 3.885 (0.75), 4.072 (2.33), 5.155 (0.41), 5.759 (16.00), 7.272 (1.78), 7.278 (2.13), 7.332 (1.61), 7.339 (1.35), 7.426 (0.75), 7.444 (1.14), 7.464 (1.11), 7.526 (1.11), 7.530 (1.17), 7.544 (0.80), 7.547 (0.75), 7.869 (0.97), 7.871 (1.01), 7.890 (0.94), 7.892 (0.87).

### Example 31, Intermediate 1

### 3-(methoxymethoxy)-8-((triisopropylsilyl)ethynyl)naphthalen-1-ol

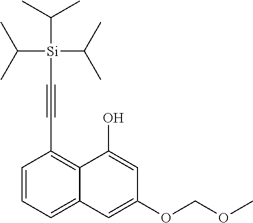 [SMILES: COCOc1cc(O)c2c(C#C[Si](C(C)C)(C(C)C)C(C)C)cccc2c1]

To a solution of 8-(2-triisopropylsilylethynyl)naphthalene-1,3-diol (28.5 g, 83.69 mmol) and DIEA (32.45 g, 251.08 mmol, 43.73 mL) in DCM (350 mL), bromo(methoxy)methane (15.69 g, 125.54 mmol, 10.25 mL) was added dropwise at 0° C. The mixture was stirred at 0° C. for 2 h. The reaction mixture was diluted with DCM (500 mL) and washed with H₂O, sat. brine, dried over Na₂SO₄, filtered and concentrated to afford the residue. The residue was purified by column chromatography (silica phase, using a gradient of petroleum ether/ethyl acetate 1-10%). The pure fraction was concentrated to afford 3-(methoxymethoxy)-8-(2-triisopropylsilylethynyl)naphthalen-1-ol (22.1 g, 54.99 mmol, 65.70% yield, 95.7% purity) as brown oil.

¹H NMR (CDCl3-d6 400 MHz) δ=9.26 (s, 1H), 7.70 (d, J=8.25 Hz, 1H), 7.50 (dd, J=7.13, 1.13 Hz, 1H), 7.27-7.38 (m, 1H), 6.98 (d, J=2.38 Hz, 1H), 6.78 (d, J=2.50 Hz, 1H), 5.27 (s, 2H), 3.52 (s, 3H), 1.14-1.28 (m, 21H)

### Example 31, Intermediate 2

### 3-(methoxymethoxy)-8-((triisopropylsilyl)ethynyl)naphthalen-1-yl trifluoromethanesulfonate

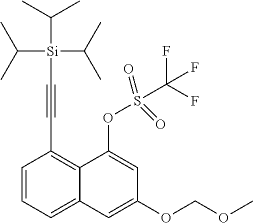 [SMILES: COCOc1cc(OS(=O)(=O)C(F)(F)F)c2c(C#C[Si](C(C)C)(C(C)C)C(C)C)cccc2c1]

To a solution of 3-(methoxymethoxy)-8-(2-triisopropylsilylethynyl)naphthalen-1-ol (9.8 g, 25.48 mmol) and DIEA (9.88 g, 76.45 mmol, 13.32 mL) in DCM (150 mL) was added Tf₂O (10.78 g, 38.22 mmol) at −40° C. The mixture was stirred at −40° C. for 1 h. The reaction mixture was quenched with H₂O (100 mL) and extracted with DCM (100 mL×3). The combined organic phase was washed with sat. brine, dried over Na₂SO₄, filtered and concentrated to afford the residue. The residue was purified by column chromatography (silica phase, using a gradient of petroleum ether/ethyl acetate 1-7%). The pure fraction was concentrated to afford the desired product (10.5 g, 19.92 mmol, 78.16% yield, 98% purity) as yellow oil.

¹H NMR (CDCl3-d6 400 MHz) δ=7.75 (t, J=8.31 Hz, 2H), 7.41-7.52 (m, 2H), 7.32 (s, 1H), 5.30 (s, 2H), 3.53 (d, J=1.59 Hz, 3H), 1.13-1.30 (m, 21H)

### Example 31, Intermediate 3

### N-(diphenylmethylene)-3-(methoxymethoxy)-8-((triisopropylsilyl)ethynyl)naphthalen-1-amine

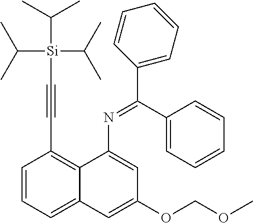 [SMILES: COCOc1cc(N=C(c2ccccc2)c2ccccc2)c2c(C#C[Si](C(C)C)(C(C)C)C(C)C)cccc2c1]

To a solution of [3-(methoxymethoxy)-8-(2-triisopropylsilylethynyl)-1-naphthyl]trifluoromethanesulfonate (8 g, 15.48 mmol), diphenylmethanimine (8.42 g, 46.45 mmol) and Cs₂CO₃ (12.61 g, 38.71 mmol) in toluene (100 mL) was added Pd(OAc)₂ (521.47 mg, 2.32 mmol) and BINAP (1.45 g, 2.32 mmol) under N₂ atmosphere. The mixture was stirred at 100° C. for 17 h. The mixture was combined with another batch (1.5 g of rare material was charged), diluted with H₂O (200 mL) and extracted with ethyl acetate (300 mL×3). The combined organic phase was washed with sat. brine, dried over Na₂SO₄, filtered and concentrated to afford the residue. The residue was purified by column chromatography (silica phase, using a gradient of petroleum ether/ethyl acetate 1-5%). The pure fraction was concentrated to afford N-[3-(methoxymethoxy)-8-(2-triisopropylsilylethynyl)-1-naphthyl]-1,1-diphenyl-methanimine (9.4 g, 73% purity) as yellow oil.

¹H NMR (CDCl3-d6 400 MHz) δ=7.82 (d, J=8.19 Hz, 4H), 7.69 (d, J=7.09 Hz, 1H), 7.47-7.53 (m, 4H), 7.41 (br d, J=7.58 Hz, 2H), 7.33 (t, J=7.70 Hz, 1H), 7.19-7.23 (m, 1H), 6.93 (d, J=2.32 Hz, 1H), 6.10 (d, J=2.08 Hz, 1H), 5.06 (s, 2H), 3.34 (s, 3H), 0.92 (d, J=7.34 Hz, 18H), 0.55-0.71 (m, 3H)

### Example 31, Intermediate 4

### 3-(methoxymethoxy)-8-{[tri(propan-2-yl)silyl]ethynyl}naphthalen-1-amine

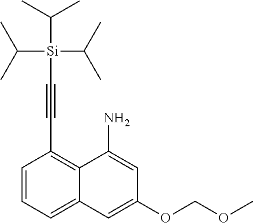 [SMILES: COCOc1cc(N)c2c(C#C[Si](C(C)C)(C(C)C)C(C)C)cccc2c1]

To a solution of N-[3-(methoxymethoxy)-8-(2-triisopropylsilylethynyl)-1-naphthyl]-1,1-diphenyl-methanimine (9.4 g, 73% purity from example 4) in ethanol (100 mL) was added sodium acetate (2.17 g, 26.43 mmol) and hydroxylamine hydrochloride (1.84 g, 26.43 mmol). The mixture was stirred at 60° C. for 2 h. The solid was filtered out and washed with ethanol. The solution was concentrated to dryness. The residue was dissolved in ethyl acetate (300 mL), washed with sat. NaHCO₃, sat. brine, dried over Na₂SO₄, filtered and concentrated to afford the residue. The residue was triturated with petroleum ether and the solid was filtered out. The filtrate was concentrated and purified by column chromatography (silica phase, using a gradient of petroleum ether/ethyl acetate 1-7%). The pure fraction was concentrated to afford 3-(methoxymethoxy)-8-(2-triisopropylsilylethynyl)naphthalen-1-amine (2.54 g, 6.62 mmol) as light-yellow oil.

¹H NMR (DMSO-d6 400 MHz) δ=7.66 (d, J=7.75 Hz, 1H), 7.38 (d, J=6.38 Hz, 1H), 7.19-7.32 (m, 1H), 6.69 (d, J=2.25 Hz, 1H), 6.57 (s, 2H), 6.43 (d, J=2.38 Hz, 1H), 5.21 (s, 2H), 3.39 (s, 3H), 1.09-1.19 (m, 21H)

### Example 31, Intermediate 5

### tert-butyl 3-(5-{[(2R,7aS)-2-fluorotetrahydro-1H-pyrrolizin-7a(5H)-yl]methoxy}-2-{[3-(methoxymethoxy)-8-{[tri(propan-2-yl)silyl]ethynyl}naphthalen-1-yl]amino}[1,3]thiazolo[5,4-d]pyrimidin-7-yl)-3,8-diazabicyclo[3.2.1]octane-8-carboxylate

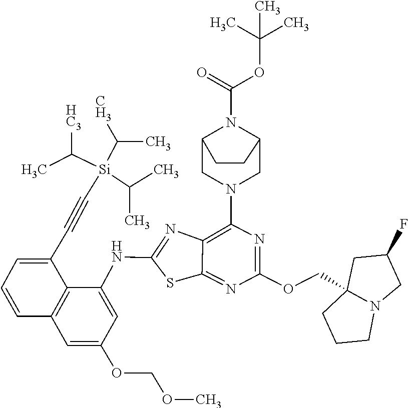

Tert-butyl 3-(2-bromo-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (300 mg, 514 μmol) and 3-(methoxymethoxy)-8-{[tri(propan-2-yl)silyl]ethynyl}naphthalen-1-amine (197 mg, 514 μmol) were dissolved in 1,4-dioxane (1.1 ml), then (tris-(dibenzylidenaceton)-dipalladium(0) (47.1 mg, 51.4 μmol; CAS-RN: [51364-51-3]), 4,5-bis(diphenylphosphino)-9,9-dimethylxanthene (29.7 mg, 51.4 μmol; CAS-RN: [161265-03-8]) and Cs₂CO₃ (201 mg, 617 μmol; CAS-RN: [534-17-8]) were added and the mixture was stirred for 1 h at 80° C. Afterwards, the reaction mixture was diluted with water and was extracted with ethyl acetate. The combined organic layers were washed with NaCl (sat., aq. solution), dried using a water-resistant filter and concentrated under reduced pressure. The crude product was purified by flash chromatography (amino phase, using a gradient of hexane/ethyl acetate 10-40%) to afford 252 mg (50% yield) of the title compound.

LC-MS (Method 1): Rₜ=1.70 min; MS (ESIpos): m/z=886 [M+H]⁺

¹H-NMR (400 MHz, DMSO-d6) δ[ppm]: 10.04 (s, 1H), 7.90 (dd, 1H), 7.57 (dd, 1H), 7.50-7.45 (m, 2H), 7.42 (d, 1H), 5.38-5.14 (m, 3H), 5.12-4.56 (m, 2H), 4.12-3.98 (m, 2H), 3.97-3.80 (m, 2H), 3.43 (s, 3H), 3.11-3.02 (m, 2H), 3.01-2.91 (m, 3H), 2.85-2.75 (m, 1H), 2.07 (br d, 1H), 1.99 (s, 1H), 1.97-1.90 (m, 1H), 1.87-1.67 (m, 5H), 1.64-1.55 (m, 2H), 1.41 (s, 9H), 1.07-0.94 (m, 21H)

### Example 31, Intermediate 6

### tert-butyl 3-(5-{[(2R,7aS)-2-fluorotetrahydro-1H-pyrrolizin-7a(5H)-yl]methoxy}-2-{[3-(methoxymethoxy)-8-{[tri(propan-2-yl)silyl]ethynyl}naphthalen-1-yl](methyl)amino}[1,3]thiazolo[5,4-d]pyrimidin-7-yl)-3,8-diazabicyclo[3.2.1]octane-8-carboxylate

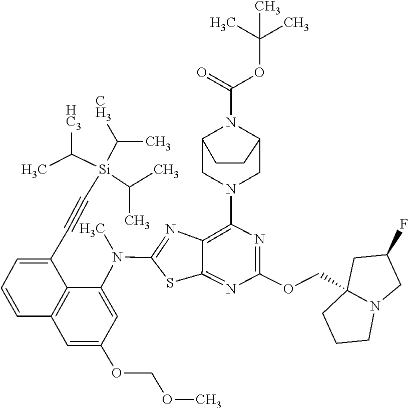

Tert-butyl 3-(5-{[(2R,7aS)-2-fluorotetrahydro-1H-pyrrolizin-7a(5H)-yl]methoxy}-2-{[3-(methoxymethoxy)-8-{[tri(propan-2-yl)silyl]ethynyl}naphthalen-1-yl]amino}[1,3]thiazolo[5,4-d]pyrimidin-7-yl)-3,8-diazabicyclo[3.2.1]octane-8-carboxylate (255 mg, 288 μmol) was dissolved in THF (8.0 ml), at 0° C. NaH (18.8 mg, 55% purity, 432 μmol; CAS-RN: [7646-69-7]) was added and the mixture was stirred for 40 minutes at 0° C. Then iodomethane (45 μl, 720 μmol; CAS-RN: [74-88-4]) was added and the mixture was stirred for 30 minutes at room temperature. Afterwards, the reaction mixture was diluted with NaHCO₃ (half concentrated sat., aq. solution) and was extracted with ethyl acetate. The combined organic layers were dried using a water-resistant filter and concentrated under reduced pressure. The crude product was purified by flash chromatography (silica phase, using a gradient of dichloromethane/methanol+2% ammonia: 5-30%) to afford 130 mg (48% yield) of the title compound.

LC-MS (Method 1): Rₜ=1.57 min; MS (ESIpos): m/z=901 [M+H]⁺

¹H-NMR (400 MHz, DMSO-d6) δ[ppm]: 0.854 (4.58), 0.864 (4.51), 0.871 (5.95), 0.972 (0.63), 0.989 (1.16), 1.009 (10.13), 1.154 (1.48), 1.172 (3.28), 1.190 (1.73), 1.235 (0.47), 1.445 (16.00), 1.691 (0.92), 1.705 (0.91), 1.718 (0.76), 1.797 (0.41), 1.898 (0.71), 1.962 (0.84), 1.987 (6.18), 2.035 (0.63), 2.518 (0.83), 2.523 (0.60), 2.787 (0.42), 2.955 (0.98), 3.019 (0.88), 3.034 (0.91), 3.431 (15.05), 3.461 (6.85), 3.792 (0.40), 3.818 (0.60), 3.891 (0.56), 3.906 (0.51), 3.999 (0.45), 4.017 (1.34), 4.035 (1.36), 4.053 (0.44), 5.157 (0.40), 5.333 (1.20), 5.350 (2.00), 5.385 (2.30), 5.402 (1.28), 5.759 (5.43), 7.535 (0.75), 7.547 (1.16), 7.554 (2.15), 7.574 (0.90), 7.659 (0.96), 7.676 (1.90), 7.682 (1.22), 7.998 (0.92), 8.001 (0.95), 8.019 (0.92), 8.022 (0.84).

### Example 31, Intermediate 7

### tert-butyl 3-(2-{[8-ethynyl-3-(methoxymethoxy)naphthalen-1-yl](methyl)amino}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

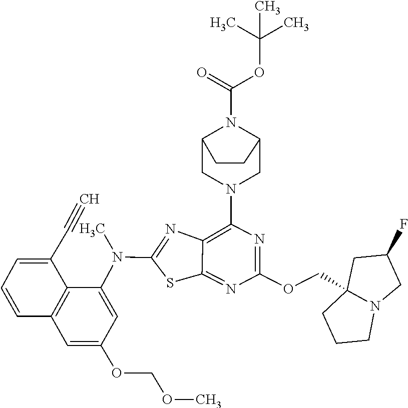 [SMILES: C#Cc1cccc2cc(OCOC)cc(N(C)c3nc4c(N5CC6CCC(C5)N6C(=O)OC(C)(C)C)nc(OC[C@@]56CCCN5C[C@H](F)C6)nc4s3)c12]

Tert-butyl 3-(5-{[(2R,7aS)-2-fluorotetrahydro-1H-pyrrolizin-7a(5H)-yl]methoxy}-2-{[3-(methoxymethoxy)-8-{[tri(propan-2-yl)silyl]ethynyl}naphthalen-1-yl](methyl)amino}[1,3]thiazolo[5,4-d]pyrimidin-7-yl)-3,8-diazabicyclo[3.2.1]octane-8-carboxylate (128 mg, 142 μmol) was dissolved in THF (5.0 ml), then N,N,N-tributylbutan-1-aminium fluoride (280 μl, 1.0 M in THF, 280 μmol) was added and the mixture was stirred for 1 h at room temperature. Afterwards, the reaction mixture was diluted with NaHCO₃ (sat., aq. solution) and was extracted with ethyl acetate. The combined organic layers were dried using a water-resistant filter and concentrated under reduced pressure. The crude product was purified by flash chromatography (amino phase, using a gradient of hexane/ethyl acetate 20-100%) to afford 100 mg (90% yield) of the title compound.

LC-MS (Method 2): Rt=1.65 min; MS (ESIpos): m/z=744 [M+H]⁺

¹H-NMR (400 MHz, DMSO-d6) δ[ppm]: 1.154 (0.45), 1.172 (0.99), 1.190 (0.49), 1.439 (16.00), 1.692 (0.57), 1.706 (0.48), 1.720 (0.44), 1.801 (0.59), 1.959 (0.56), 1.966 (0.67), 1.988 (1.59), 2.040 (0.53), 2.518 (0.85), 2.523 (0.58), 2.957 (0.51), 3.033 (0.85), 3.433 (6.06), 3.440 (12.90), 3.796 (0.53), 3.872 (0.87), 3.898 (0.57), 4.173 (1.30), 5.347 (1.03), 5.363 (1.72), 5.395 (1.74), 5.412 (0.89), 5.760 (3.07), 7.508 (0.54), 7.527 (0.80), 7.547 (0.82), 7.552 (1.15), 7.559 (1.19), 7.629 (0.72), 7.631 (0.74), 7.646 (0.54), 7.649 (0.52), 7.675 (1.04), 7.682 (0.93), 7.995 (0.71), 7.998 (0.73), 8.015 (0.69), 8.019 (0.64).

### Example 32

### 4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]methyl}-5-ethynyl-6-fluoronaphthalen-2-ol formic acid (1/1)

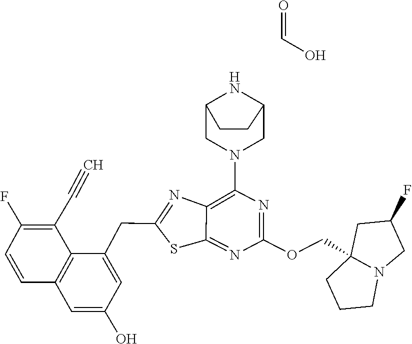 [SMILES: O=CO ; C#Cc1c(F)ccc2cc(O)cc(Cc3nc4c(N5CC6CCC(C5)N6)nc(OC[C@@]56CCCN5C[C@H](F)C6)nc4s3)c12]

Tert-butyl 3-(2-{[8-ethynyl-7-fluoro-3-(methoxymethoxy)naphthalen-1-yl]methyl}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (216 mg, 289 μmol) was dissolved in acetonitrile (3.0 ml), at 0° C. HCl (1.1 ml, 4.0 M in 1,4-dioxane, 4.3 mmol; CAS-RN: [7647-01-0]) was added and the mixture was stirred at 0° for 60 minutes under Ar atmosphere. The reaction mixture was quenched with NaHCO₃ (sat. aq. Solution) and was extracted with a mixture of ethyl acetate and ethanol. The combined organic layers were dried using a water-resistant filter and concentrated under reduced pressure. The crude product was purified by preparative HPLC (Method A, using a gradient of water/acetonitrile 10-30%) to afford 31 mg (17% yield) of the title compound.

LC-MS (Method 1): Rt=0.72 min; MS (ESIpos): m/z=603 [M+H]⁺

¹H-NMR (400 MHz, DMSO-d6) δ[ppm]: 1.46 (m, 2H) 1.60-1.68 (m, 2H) 1.72 (m, 2H) 1.77-1.85 (m, 1H) 1.91-1.96 (m, 1H) 1.98 (m, 1H) 2.06 (m, 1H) 2.75-2.83 (m, 1H) 2.95-3.12 (m, 6H) 3.83-3.98 (m, 3H) 4.64 (d, 1H) 5.15-5.33 (m, 3H) 7.21 (m, 2H) 7.41 (t, 1H) 7.89 (dd, 1H) 8.20 (s, 2H)

### Example 32, Intermediate 1

### 7-fluoro-3-(methoxymethoxy)-8-{[tri(propan-2-yl)silyl]ethynyl}naphthalen-1-yl trifluoromethanesulfonate

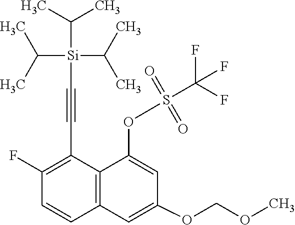 [SMILES: COCOc1cc(OS(=O)(=O)C(F)(F)F)c2c(C#C[Si](C(C)C)(C(C)C)C(C)C)c(F)ccc2c1]

7-Fluoro-3-(methoxymethoxy)-8-{[tri(propan-2-yl)silyl]ethynyl}naphthalen-1-ol (3.00 g, 7.45 mmol) was dissolved in dichloromethane (60 ml), N,N-diisopropylethylamine (3.9 ml, 22 mmol; CAS-RN:[7087-68-5]) was added and at 0° C. trifluoromethanesulfonic anhydride (11 ml, 1.0 M in dichloromethane, 11 mmol; CAS-RN: [358-23-6]) was added dropwise. The mixture was stirred for 1 h at 0° C. and then for 1 h at room temperature under Ar atmosphere. Afterwards, the reaction mixture was diluted with water and was extracted with ethyl acetate. The combined organic layers were washed with NaCl (half concentrated sat., aq. solution), dried using a water-resistant filter and concentrated under reduced pressure. The crude product was purified by flash chromatography (silica phase, using a gradient of hexane/ethyl acetate 20-80%) to afford 3.63 g (91% yield) of the title compound.

LC-MS (Method 2): Rt=1.98 min; MS (ESIpos): m/z=535 [M+H]⁺

### Example 32, Intermediate 2

### 7-fluoro-3-(methoxymethoxy)-8-{[tri(propan-2-yl)silyl]ethynyl}naphthalene-1-carbonitrile

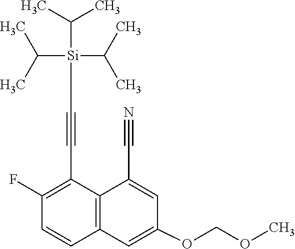 [SMILES: COCOc1cc(C#N)c2c(C#C[Si](C(C)C)(C(C)C)C(C)C)c(F)ccc2c1]

7-Fluoro-3-(methoxymethoxy)-8-{[tri(propan-2-yl)silyl]ethynyl}naphthalen-1-yl trifluoromethanesulfonate (3.63 g, 6.79 mmol) was dissolved in DMF (48 ml), then zinc cyanide (1.20 g, 10.2 mmol; CAS-RN: [557-21-1]), dichloro[1,1′-bis(diphenylphosphino)ferrocene]palladium(II), complex with dichloromethane (1:1) (829 mg, 1.02 mmol; CAS-RN: [95464-05-4]) and tris(dibenzylideneacetone)di-palladium(0) (933 mg, 1.02 mmol; CAS-RN: [52409-22-0]) were added. Ar was bubbled through the mixture for 10 minutes and then the mixture was stirred over night 110° C. Afterwards, the reaction mixture was filtered over celite and washed with ethyl acetate. The filtrate was diluted with water and was extracted with ethyl acetate. The combined organic layers were washed with NaCl (half concentrated sat., aq. solution), dried using a water-resistant filter and concentrated under reduced pressure. The crude product was purified by flash chromatography (silica phase, using a gradient of hexane/ethyl acetate 0-80%) to afford 2.47 g (88% yield) of the title compound.

¹H-NMR (400 MHz, DMSO-d6) δ[ppm]: 0.971 (1.96), 0.986 (0.94), 1.122 (1.63), 1.133 (7.30), 1.146 (16.00), 1.161 (0.95), 1.164 (0.74), 1.171 (2.38), 1.184 (1.63), 1.189 (0.83), 1.199 (0.90), 1.206 (0.78), 1.221 (0.60), 1.987 (1.74), 2.518 (0.88), 2.523 (0.65), 3.415 (0.44), 3.424 (10.15), 4.016 (0.40), 4.034 (0.41), 5.385 (4.19), 5.760 (1.34), 7.625 (0.50), 7.648 (0.96), 7.670 (0.55), 7.961 (0.84), 7.968 (1.22), 7.991 (1.18), 7.998 (0.79), 8.106 (0.46), 8.121 (0.49), 8.129 (0.47), 8.144 (0.44).

### Example 32, Intermediate 3

### 7-fluoro-3-(methoxymethoxy)-8-{[tri(propan-2-yl)silyl]ethynyl}naphthalene-1-carbaldehyde

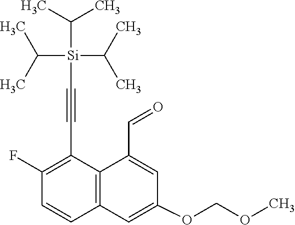 [SMILES: COCOc1cc(C=O)c2c(C#C[Si](C(C)C)(C(C)C)C(C)C)c(F)ccc2c1]

7-Fluoro-3-(methoxymethoxy)-8-{[tri(propan-2-yl)silyl]ethynyl}naphthalene-1-carbonitrile (770 mg, 1.87 mmol) was dissolved in toluene (20 ml) and the mixture was flushed with Ar. At −40° C. diisobutylaluminum hydride (5.6 ml, 1.0 M in toluene, 5.6 mmol; CAS-RN: [1191-15-7]) was added dropwise druing 30 minutes and the mixture was stirred at this temperature under Ar atmosphere. The reaction mixture was quenched at −40° C. with methanol and allowed to reach room temperature. Then the pH was adjusted to 3-4 by addition of citric acid (1 M. aq. solution) and the mixture was stirred at room temperature for 1 hour. Afterwards, the reaction mixture was diluted with water and was extracted with ethyl acetate. The combined organic layers were dried using a water-resistant filter and concentrated under reduced pressure to afford 843 mg (100% yield) of the title compound.

¹H-NMR (400 MHz, DMSO-d6) δ[ppm]: 0.971 (0.73), 0.986 (0.40), 1.106 (7.76), 1.119 (16.00), 1.137 (2.13), 1.149 (1.95), 1.164 (0.97), 1.170 (0.88), 1.184 (0.62), 2.295 (0.66), 3.420 (5.84), 5.369 (3.12), 5.758 (2.11), 7.593 (0.45), 7.616 (0.86), 7.634 (1.03), 7.640 (1.24), 7.872 (0.93), 7.879 (0.87), 8.124 (0.41), 8.139 (0.45), 8.147 (0.43), 11.498 (1.89).

### Example 32, Intermediate 4

### tert-butyl 3-(2-[(RS)-[7-fluoro-3-(methoxymethoxy)-8-{[tri(propan-2-yl)silyl]ethynyl}naphthalen-1-yl](hydroxy)methyl]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

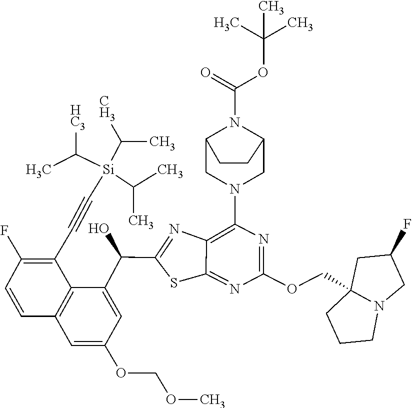

Tert-butyl 3-(5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (406 mg, 804 μmol) was placed in an Ar flushed sealed flask and dissolved in dry THF (3.3 ml) under Ar atmosphere. At −78° C. n-butyllithium (650 μl, 1.6 M in hexane, 1.0 mmol; CAS-RN: [109-72-8]) was added dropwise and the mixture was stirred at −78° C. for 30 minutes.

7-Fluoro-3-(methoxymethoxy)-8-{[tri(propan-2-yl)silyl]ethynyl}naphthalene-1-carbaldehyde (500 mg, 1.21 mmol) was placed in another Ar flushed sealed vial and dissolved in dry THF (3.3 ml). This solution was added dropwise to the first reaction mixture and then it was stirred for 1 h at −78° C. Afterwards, the reaction mixture was diluted with water and was extracted with dichloromethane. The combined organic layers were washed with NaCl (sat., aq. solution), dried using a water-resistant filter and concentrated under reduced pressure. The crude product was purified by flash chromatography (silica phase, using a gradient of hexane/ethyl acetate 20-100% and ethyl acetate/ethanol 0-20%) to afford 352 mg (47% yield) of the title compound.

LC-MS (Method 1): Rt=1.35 min; MS (ESIpos): m/z=920 [M+H]⁺

### Example 32, Intermediate 5

### tert-butyl 3-(2-[(RS)-[8-ethynyl-7-fluoro-3-(methoxymethoxy)naphthalen-1-yl](hydroxy)methyl]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

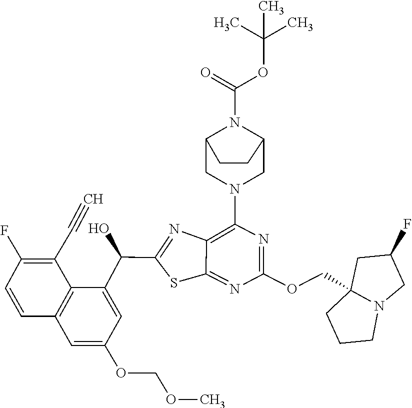 [SMILES: C#Cc1c(F)ccc2cc(OCOC)cc([C@@H](O)c3nc4c(N5CC6CCC(C5)N6C(=O)OC(C)(C)C)nc(OC[C@@]56CCCN5C[C@H](F)C6)nc4s3)c12]

Tert-butyl 3-(2-[(RS)-[7-fluoro-3-(methoxymethoxy)-8-{[tri(propan-2-yl)silyl]ethynyl}naphthalen-1-yl](hydroxy)methyl]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (359 mg, 391 μmol) was dissolved in THF (2.2 ml), then N,N,N-tributylbutan-1-aminium fluoride (780 μl, 1.0 M in THF, 780 μmol; CAS-RN: [429-41-4]) was added and the mixture was stirred for 30 minutes at room temperature. Afterwards, the reaction mixture was diluted with NaHCO₃ (half concentrated sat., aq. solution) and was extracted with ethyl acetate. The combined organic layers were dried using a water-resistant filter and concentrated under reduced pressure to afford 321 mg (100% yield) of the title compound.

LC-MS (Method 2): Rt=1.53 min; MS (ESIpos): m/z=763 [M+H]⁺

¹H-NMR (400 MHz, DMSO-d6) δ[ppm]: 0.879 (0.43), 0.886 (0.71), 0.903 (0.90), 0.907 (0.74), 0.912 (0.60), 0.924 (1.61), 0.933 (0.78), 0.938 (1.50), 0.950 (0.74), 0.974 (16.00), 0.989 (7.52), 0.997 (1.95), 1.003 (0.48), 1.154 (0.72), 1.171 (1.37), 1.189 (0.68), 1.352 (0.49), 1.414 (2.80), 1.436 (0.98), 1.708 (0.42), 1.976 (0.42), 1.987 (2.37), 2.518 (1.95), 2.523 (1.29), 2.968 (0.49), 3.036 (0.47), 3.399 (4.90), 3.870 (0.40), 4.017 (0.49), 4.034 (0.50), 5.113 (2.87), 5.308 (1.73), 7.534 (0.55), 7.733 (0.61), 7.740 (0.56).

### Example 32, Intermediate 6

### tert-butyl 3-(2-{(RS)-[8-ethynyl-7-fluoro-3-(methoxymethoxy)naphthalen-1-yl][(methanesulfonyl)oxy]methyl}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

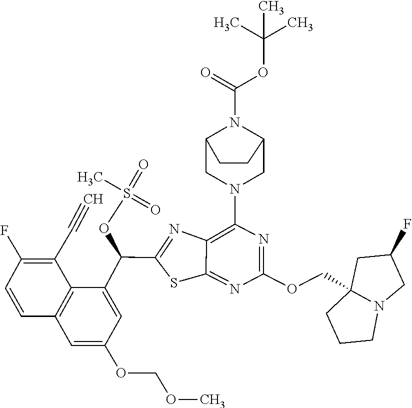 [SMILES: C#Cc1c(F)ccc2cc(OCOC)cc([C@@H](OS(C)(=O)=O)c3nc4c(N5CC6CCC(C5)N6C(=O)OC(C)(C)C)nc(OC[C@@]56CCCN5C[C@H](F)C6)nc4s3)c12]

Tert-butyl 3-(2-[(RS)-[8-ethynyl-7-fluoro-3-(methoxymethoxy)naphthalen-1-yl](hydroxy)methyl]-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (297 mg, 389 μmol) was dissolved in dichloromethane (1.5 ml), N-diisopropylethylamine (140 μl, 780 μmol; CAS-RN: [7087-68-5]) was added and at 0° C. methanesulfonyl chloride (60 μl, 780 μmol) was added and the mixture was stirred for 30 minutes at 0° C. Afterwards, the reaction mixture was diluted with NaHCO₃ (half concentrated sat., aq. solution) and was extracted with dichloromethane. The combined organic layers were washed with NaCl (sat., aq. solution), dried using a water-resistant filter and concentrated under reduced pressure to afford 334 mg (100% yield) of the title compound.

### Example 32, Intermediate 7

### tert-butyl 3-(2-{[8-ethynyl-7-fluoro-3-(methoxymethoxy)naphthalen-1-yl]methyl}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate

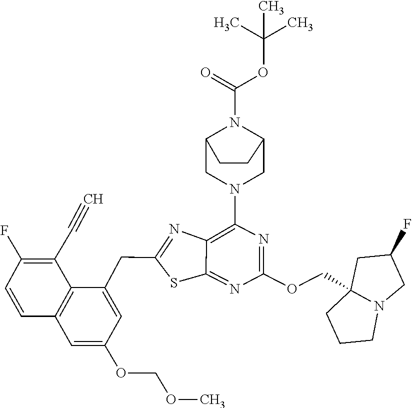 [SMILES: C#Cc1c(F)ccc2cc(OCOC)cc(Cc3nc4c(N5CC6CCC(C5)N6C(=O)OC(C)(C)C)nc(OC[C@@]56CCCN5C[C@H](F)C6)nc4s3)c12]

Tert-butyl 3-(2-{(RS)-[8-ethynyl-7-fluoro-3-(methoxymethoxy)naphthalen-1-yl][(methanesulfonyl)oxy]methyl}-5-{[(2R,7aS)-2-fluorotetrahydro-1H-pyrrolizin-7a(5H)-yl]methoxy}[1,3]thiazolo[5,4-d]pyrimidin-7-yl)-3,8-diazabicyclo[3.2.1]octane-8-carboxylate (327 mg, 389 μmol) was dissolved in a mixture of DMF (3.0 ml) and ethanol (2.3 ml), then Pd/C (12.4 mg, 10% purity, 11.7 μmol; CAS-RN: [7440-05-3]) was added and the mixture was stirred for 2 h under hydrogen atmosphere at room temperature. Afterwards, the catalyst was filtered off over celite and washed with ethanol. The filtrate was concentrated under reduced pressure to afford 316 mg (88% yield) of the title compound.

LC-MS (Method 2): Rₜ=1.68 min; MS (ESIpos): m/z=748 [M+H]⁺

¹H-NMR (400 MHz, DMSO-d6) δ[ppm]: 0.887 (0.74), 0.894 (0.61), 0.903 (0.95), 0.908 (0.81), 0.912 (1.26), 0.923 (1.71), 0.930 (0.59), 0.934 (1.09), 0.938 (1.45), 0.951 (0.77), 0.974 (16.00), 0.989 (7.63), 0.997 (2.12), 1.004 (0.63), 1.096 (0.61), 1.165 (0.46), 1.183 (0.55), 1.230 (0.55), 1.303 (0.41), 1.315 (0.40), 1.352 (0.67), 1.420 (13.56), 1.440 (1.86), 1.468 (0.57), 2.518 (2.45), 2.523 (1.77), 2.727 (2.85), 2.729 (2.83), 2.824 (0.53), 2.888 (3.43), 2.932 (0.46), 2.983 (0.87), 3.401 (0.40), 3.417 (10.49), 4.414 (0.48), 4.658 (1.69), 4.660 (1.74), 5.113 (1.30), 5.332 (3.37), 5.760 (8.85), 7.436 (0.88), 7.443 (0.94), 7.481 (0.47), 7.503 (0.92), 7.526 (0.51), 7.573 (1.15), 7.580 (1.04), 7.950 (0.42), 8.013 (0.45), 8.028 (0.49), 8.035 (0.48), 8.051 (0.44).

### Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds of the present invention can be demonstrated in the following assays:

### Biochemical KRAS/SOS1 Activation Assays

Preparation of test compound dilutions. A 100-fold concentrated solution of the test compound (50 nL) in DMSO was transferred to microtiter test plates (384 or 1,536 wells, Greiner Bio-One, Germany) using either a Hummingbird liquid handler (Digilab, MA, USA) or an Echo acoustic system (Labcyte, CA, USA). Plates were sealed with adhesive foil or heat-sealed and stored at −20 C until use. Serial dilutions of test compounds were prepared in 100% DMSO using a Precision Pipetting System (BioTek, USA).

Measurement and evaluation of inhibition data, calculation of IC₅₀ values. Homogeneous time-resolved fluorescence (HTRF) was measured with a PHERAstar reader (BMG, Germany) using the HTRF module (excitation: 337 nm; emission 1: 620 nm, emission 2: 665 nm). The ratio of the emissions at 665 and 620 nm was used as the specific signal for further evaluation. The data were normalized using the controls: DMSO=0% inhibition, inhibition control wells with inhibitor control solution=100% inhibition. Compounds were tested in duplicates at up to 11 concentrations (e.g. 20 μM, 5.7 μM, 1.6 μM, 0.47 μM, 0.13 μM, 38 nM, 11 nM, 3.1 nM, 0.89 nM, 0.25 nM and 0.073 nM). IC₅₀ values were calculated using a four-parameter fit, with a commercial software package (Genedata Screener, Switzerland).

KRAS^{G12C} activation by SOS1^{cat} assay (“On-assay”). This assay quantifies SOS1^{cat} mediated loading of KRAS^{G12C}-GDP with a fluorescent GTP analogue. Detection of successful loading was achieved by measuring resonance energy transfer from anti-GST-terbium (FRET donor) bound to GST-KRAS^{G12C} to the loaded fluorescent GTP analogue (FRET acceptor). The fluorescent GTP analogue EDA-GTP-DY-647P1 [2′/3′-O-(2-aminoethyl-carbamoyl)guanosine-5′-triphosphate labelled with DY-647P1 (Dyomics GmbH, Germany)] was synthesized by Jena Bioscience (Germany) and supplied as a 1 mM aqueous solution. A KRAS^{G12C} working solution was prepared in assay buffer [10 mM HEPES pH 7.4 (AppliChem), 150 mM NaCl (Sigma), 5 mM MgCl₂ (Sigma), 1 mM DTT (Thermo Fisher), 0.05% BSA Fraction V pH 7.0 (ICN Biomedicals), 0.0025% (v/v) Igepal (Sigma)] containing GST-KRAS^{G12C} (final concentration in assay 50 nM) and anti-GST-terbium (Cisbio, France) (final concentration in assay 1 nM). A SOS1^{cat} working solution was prepared in assay buffer containing SOS1^{cat} (final concentration 10 nM) and EDA-GTP-DY-647P1 (final concentration 100 nM). An inhibitor control solution was prepared in assay buffer containing EDA-GTP-DY-647P1 (final concentration 100 nM) without SOS1^{cat}. All steps of the assay were performed at 20° C. A volume of 3 μL of the KRAS^{G12C} working solution was added to all wells of the test plate using a Multidrop dispenser (Thermo LabSystems). After 15 min, 2 μL of the SOS1^{c}t working solution was added to all wells, except for the inhibitor control solution wells. After 30 min incubation, HTRF was measured.

G12D and Wild-type KRAS activation by SOS1cat assays. These assays quantify human SOS1^{cat} mediated loading of wild-type GST-KRAS^{WT}-GDP or GST-KRAS^{G12D}-GDP with a fluorescent GTP analogue. The assay was performed similarly to the KRAS^{G12C} activation by SOS1^{cat} assay with the following differences: final concentration of SOS1^{cat} of 60 nM and final incubation time before measurement of 10 min for KRAS^{G12D} assay, final concentration of SOS1^{cat} of 20 nM and final incubation time before measurement of 15 min for wild-type KRAS assay, respectively. GST-KRAS^{G12C} was replaced by GST-KRAS^{WT} or GST-KRAS^{G12D}, respectively, also at 50 nM final concentration.

### K-Ras Surface Plasmon Resonance Assay

The surface plasmon resonance experiments were performed at 20° C. using a Biacore 8K system (Cytiva Europe GmbH).

The biotinylated recombinant K-Ras G12D at 50 μg/mL was immobilized on SA-Chip (Cytiva Europe GmbH) in 10 mM Hepes pH7.5, 150 mM NaCl, 5 mM MgCl₂, 0.05% BSA, 1 mM DTT, 0.0025% Igepal (NP40) with a flow rate of 5 μL/min to reach ligand density between 1000 and 4000 RU. KD titrations were performed in multi-cycle mode using running buffer of 10 mM Hepes pH7.5, 150 mM NaCl, 5 mM MgCl₂, 1 mM DTT, 0.05% BSA, 0.0025% Igepal (NP40), 2% DMSO in a flow rate of 30 μL/min with a contact time of 90 s and a dissociation time of 150 s. For selected high-affinity compounds, KD titration were performed in single-cycle mode in the same running buffer in a flow rate of 100 μL/min with a contact time of 60 s and dissociation time of 3500 s.

All SPR sensograms were analysed using the Biacore Insight Evaluation Software (Version: 3.0.12.15655 GE. Healthcare Bio-Sciences Corp.). Multi cycle experiments were analysed by steady state affinity, while single cycle data was analysed by 1:1 binding kinetics model.

The result table was exported as excel files. All measurements were performed at least as duplicates. Final K_{D}-value were prepared in Excel by taking the mean value of the independently determined K_{D}-values.

### KRas Protein Production for SPR

### Cloning and In Vivo Biotinylating Expression

cDNA fragments of human K-Ras (Acc. No P01116-2) with G12D mutant (K-Ras G12D: Aa 1-169; G12D; C118S) were cloned into N-terminal His-StrepII-Avi-tag vector using Gateway technologies. This vector was co-transfected with pBirAcm and expressed into E. coli BL21(DE3) using LB 184 medium in the presence of 200 μg/mL Ampicillin and 34 μg/mL Chloramphenicol. The cells were grown at 37° C. until the OD₅₅₀ reached 1, at which point 0.1 mM IPTG and 50 μM Biotin were added and the temperature was lowered to 27° C. The cells were harvested after 24 hours.

### Purification

E. coli cell pellet was resuspended in 3.5 mL buffer (50 mM Tris-HCl pH 7.5, 300 mM NaCl, 10 mM Imidazole, 0.5% CHAPS, Complete-EDTAfree protease inhibitor, 2 μg Benzonase) per gram wet weight and lysed by sonication. The soluble protein was separated by centrifugation at 24000×g for an hour at 4° C. The protein was purified via Ni-NTA affinity chromatography using buffer (50 mM Tris HCl pH 7.5, 300 mM NaCl) with 10 mM Imidazole for washing and 300 mM Imidazole for elution. The eluted protein was then concentrated and further purified by size exclusion chromatography (Superdex 200) in 20 mM Tris-HCl pH 7.5, 100 mM NaCl, 5 mM MgCl₂.

KRAS^{G12D} activation by SOS1^{cat} assay (“On-assay”). This assay quantifies SOS1^{cat} mediated loading of KRAS^{G12D}-GDP with a fluorescent GTP analogue. Detection of successful loading was achieved by measuring resonance energy transfer from anti-GST-terbium (FRET donor) bound to GST-KRAS^{G12D} to the loaded fluorescent GTP analogue (FRET acceptor). The fluorescent GTP analogue EDA-GTP-DY-647P1 [2′/3′-(2-aminoethyl-carbamoyl)guanosine-5′-triphosphate labelled with DY-647P1 (Dyomics GmbH, Germany)] was synthesized by Jena Bioscience (Germany) and supplied as a 1 mM aqueous solution. A KRAS^{G12D} working solution was prepared in assay buffer [10 mM HEPES pH 7.4 (AppliChem), 150 mM NaCl (Sigma), 5 mM MgCl₂ (Sigma), 1 mM DTT (Thermo Fisher), 0.05% BSA Fraction V pH 7.0 (ICN Biomedicals), 0.0025% (v/v) Igepal (Sigma)]containing GST-KRAS^{G12D} (final concentration in assay 2 nM) and anti-GST-terbium (Cisbio, France) (final concentration in assay 1 nM). A SOS1^{cat} working solution was prepared in assay buffer containing SOS1^{cat} (final concentration 5 nM) and EDA-GTP-DY-647P1 (final concentration 100 nM). An inhibitor control solution was prepared in assay buffer containing EDA-GTP-DY-647P1 (final concentration 100 nM) without SOS1^{cat} but with addition of GDP (final concentration 20 μM, Jena Bioscience, prepared from 100 mM stock solution). All steps of the assay were performed at 20° C. A volume of 3 μL of the KRAS^{G12D} working solution was added to all wells of the test plate using a Multidrop dispenser (Thermo LabSystems). After 15 min, 2 μL of the SOS1^{cat} working solution was added to all wells, except for the inhibitor control solution wells. After 10 min incubation, HTRF was measured.

Wild-type KRAS activation by SOS1^{cat} assay. This assay quantifies human SOS1^{cat} mediated loading of wild-type GST-KRAS-GDP with a fluorescent GTP analogue. The assay was performed similarly to the KRAS^{G12D} activation by SOS1^{cat} assay just by replacement of GST-KRAS^{G12D} by wild-type GST-KRAS while concentrations of all components and incubation times were the same.

### Caco-2 Permeability Assay

Caco-2 cells [purchased from the German Collection of Microorganisms and Cell Cultures (DSMZ), Braunschweig, Germany] were seeded at a density of 4.5×10⁴ cells/well on 24-well insert plates, 0.4 μm pore size (Costar) and grown for 13-15 days in DMEM supplemented with 10% FCS, 1% GlutaMAX (100×, Gibco), 100 U/mL penicillin, 100 g/mL streptomycin (Gibco), and 1% nonessential amino acids (100×, Thermo Fischer Scientific). Cells were maintained at 37° C. in a humidified 5% CO₂ atmosphere. Medium was changed every 2-3 days. Before the assay was run, the culture medium was replaced by FCS-free transport buffer. For the assessment of monolayer integrity, the transepithelial electrical resistance (TEER) was measured. Test compounds were pre-dissolved in DMSO and added either to the apical or basolateral compartment at a final concentration of 2 μM. The organic solvent in the incubations was limited to ≤1% dimethylsulfoxide (DMSO). Before and after incubation for 2 h at 37° C., samples were taken from both compartments and analyzed by LC-MS/MS after precipitation with MeOH. The apparent permeability coefficient (Pₐₚₚ) was calculated both for the apical to basolateral (A→B) and the basolateral to apical (B→A) direction using the following equation: Pₐₚₚ=(Vᵣ/P₀)(1/S)(P₂/t), where Vᵣ is the volume of medium in the receiver chamber, P₀ is the measured peak area of the test compound in the donor chamber at t=0, S is the surface area of the monolayer, P₂ is the measured peak area of the test compound in the acceptor chamber after incubation for 2 h, and t is the incubation time. The efflux ratio (ER) basolateral (B) to apical (A) was calculated by dividing Papp B-A by Papp A-B. In addition, the compound recovery was calculated. As assay control, reference compounds were analyzed in parallel.

### Results

| | K-Ras G12D activation | K-Ras-wildtype activation |
|---|---|---|
| | by SOS1 | by SOS1 |
| Ex. | IC₅₀ - [mol/l] (mean) | IC₅₀ - [mol/l] (mean) |
| 1 | 1.00E−6 | 2.00E−5 |
| 2 | 5.55E−9 | 1.26E−7 |
| 3 | 3.25E−7 | 8.14E−6 |
| 4 | 7.98E−8 | 1.55E−6 |
| 5 | 3.79E−9 | 6.91E−8 |
| 6 | 2.18E−9 | 3.31E−8 |
| 6* | 1.86E−9 | 3.28E−8 |
| 7 | 3.36E−6 | 2.00E−5 |
| 8 | >2.00E−5 | >2.00E−5 |
| 9 | >2.00E−5 | >2.00E−5 |
| 10 | >2.00E−5 | >2.00E−5 |
| 11 | >2.00E−5 | >2.00E−5 |
| 12 | 2.37E−8 | 1.58E−8 |
| 13 | 2.56E−8 | 1.53E−8 |
| 14 | 6.35E−7 | >2.00E−5 |
| 15 | 3.53E−8 | 1.07E−6 |
| 16 | 1.52E−8 | 2.12E−7 |
| 17 | 1.34E−8 | 4.25E−7 |
| 18 | 1.53E−5 | >2.00E−5 |
| 19 | 1.43E−8 | 1.07E−8 |
| 20 | 8.11E−9 | 4.05E−7 |
| 21 | 2.19E−7 | 1.82E−7 |
| 22 | 8.95E−8 | >2.00E−5 |
| 23 | 4.90E−9 | 9.50E−8 |
| 24 | 2.67E−8 | 2.41E−8 |
| 25 | 1.87E−8 | 6.99E−8 |
| 26 | 2.63E−8 | 2.59E−8 |
| 27 | 2.37E−8 | 9.12E−8 |
| 28 | 1.41E−6 | >2.00E−5 |
| 29 | 2.84E−7 | 1.19E−5 |
| 30 | 1.06E−5 | 7.61E−6 |
| 31 | 4.42E−9 | 6.15E−8 |
| 32 | 3.95E−9 | 9.98E−8 |

6*: The data mentioned for example 6* (same compound as example 6) differ a little from the data mentioned for example 6. This is the result of 2 independent measurements. However, this deviation seems to be within the normal tolerance in such assays.

1. A compound of general formula (I) 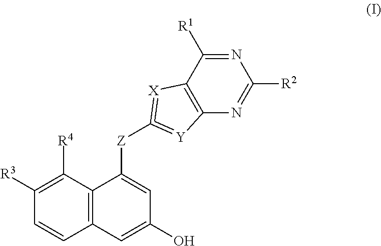
in which:
X is selected from the group consisting of ═N—, —NH—, —N(CH₃)—, —CH═, —C(CH₃)═ and —S—;
Y is selected from the group consisting of ═N—, —NH—, —N(CH₃)—, —CH═, —C(CH₃)═ and —S—,
with the condition, that X and Y are not simultaneous —CH═ or simultaneous —S—;
It is understood that the fragment —X═C(—Z)═Y— in general formula (I) has the meaning of either —X—C(—Z)═Y— or —X═C(—Z)—Y—;
Z is selected from the group consisting of —NH—, —N(CH₃)—, —CH₂—, —CH(CH₃)—, —CH(OH)— and —O—;
R¹ is selected from the group consisting of 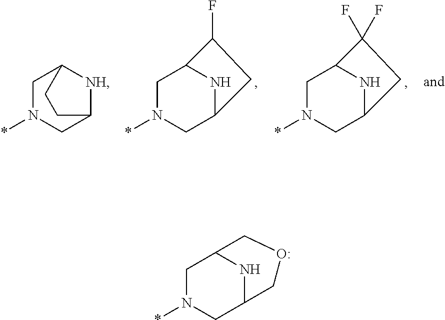
R² is selected from the group consisting of 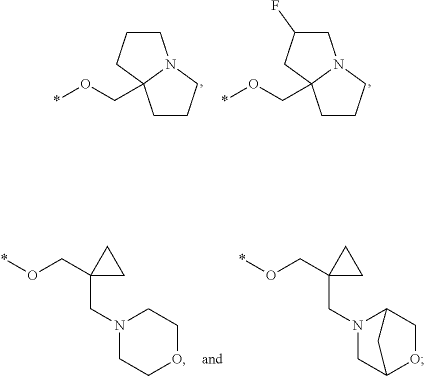
R³ is selected from the group consisting of H, F, Cl, and —CH₃;
R⁴ is selected from the group consisting of H, F, Cl, —CH₃, and —C≡CH;
or a stereoisomer, a tautomer, an N-oxide, a hydrate, a solvate, or a salt thereof, or a mixture of same.

2. The compound according to claim 1 which is selected from the group consisting of:
4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol,
4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-5-ethynyl-2-naphthol,
4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-fluoronaphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}oxy)-5-methylnaphthalen-2-ol,
4-({7-[(1R,5S)-3,8-diazabicyclo[3.2.1]octan-3-yl]-5-[(tetrahydro-1H-pyrrolizin-7a(5H)-yl)methoxy][1,3]thiazolo[5,4-d]pyrimidin-2-yl}oxy)-5-ethynyl-6-fluoronaphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynyl-6-fluoronaphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}amino)naphthalen-2-ol,
4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)-5-fluoronaphthalen-2-ol,
4-({6-(3,8-diazabicyclo[3.2.1]octan-3-yl)-9-methyl-2-[(tetrahydro-1H-pyrrolizin-7a(5H)-yl)methoxy]-9H-purin-8-yl}oxy)naphthalen-2-ol,
4-({6-(3,8-diazabicyclo[3.2.1]octan-3-yl)-2-[(tetrahydro-1H-pyrrolizin-7a(5H)-yl)methoxy]-9H-purin-8-yl}oxy)naphthalen-2-ol,
4-({6-(3,8-diazabicyclo[3.2.1]octan-3-yl)-2-[(tetrahydro-1H-pyrrolizin-7a(5H)-yl)methoxy]-9H-purin-8-yl}oxy)-5-fluoronaphthalen-2-ol,
5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol,
5-ethynyl-6-fluoro-4-({7-(3-oxa-7,9-diazabicyclo[3.3.1]nonan-7-yl)-5-[(tetrahydro-1H-pyrrolizin-7a(5H)-yl)methoxy][1,3]thiazolo[5,4-d]pyrimidin-2-yl}oxy)naphthalen-2-ol,
4-({7-(3,8-diazabicyclo[3.2.1]octan-3-yl)-5-[(tetrahydro-1H-pyrrolizin-7a(5H)-yl)methoxy][1,3]thiazolo[5,4-d]pyrimidin-2-yl}methyl)-5-fluoronaphthalen-2-ol,
5-Chlor-4-{[7-(3,8-diazabicyclo[3.2.1]octan-3-yl)-5-(tetrahydro-1H-pyrrolizin-7a(5H)-ylmethoxy)[1,3]thiazolo[5,4-d]pyrimidin-2-yl]oxy}-2-naphthol,
4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethenyl-6-fluoronaphthalen-2-ol,
4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)naphthalen-2-ol,
5-ethynyl-6-fluoro-4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]oxy}naphthalen-2-ol,
4-[(7-[(1R*,5R*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-[(7-[(1S*,5S*)-3,6-diazabicyclo[3.2.2]nonan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]-5-ethynyl-6-fluoronaphthalen-2-ol,
4-({6-(3,8-diazabicyclo[3.2.1]octan-3-yl)-7-methyl-2-[(tetrahydro-1H-pyrrolizin-7a(5H)-yl)methoxy]-7H-purin-8-yl}oxy)-5-ethynyl-6-fluoronaphthalen-2-ol
6-chloro-4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]oxy}-5-ethynylnaphthalen-2-ol
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
5-Ethynyl-6-fluoro-4-[(7-[(1R*,5R*)-6-fluoro-3,8-diazabicyclo[3.2.1]octan-3-yl]-5-{[(2R,7aS)-2-fluorotetrahydro-1H-pyrrolizin-7a(5H)-yl]methoxy}[1,3]thiazolo[5,4-d]pyrimidin-2-yl)oxy]naphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]amino}naphthalen-2-ol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}naphthalen-2-ol,
4-{[5-{[(2R,7aS)-2-fluorotetrahydro-1H-pyrrolizin-7a(5H)-yl]methoxy}-7-(3-oxa-7,9-diazabicyclo[3.3.1]nonan-7-yl)[1,3]thiazolo[5,4-d]pyrimidin-2-yl]amino}-2-naphthol,
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl](methyl)amino}-5-ethynylnaphthalen-2-ol, and
4-{[7-(3,8-diazabicyclo[3.2.1]octan-3-yl)-5-{[(2R,7aS)-2-fluorotetrahydro-1H-pyrrolizin-7a(5H)-yl]methoxy}[1,3]thiazolo[5,4-d]pyrimidin-2-yl]methyl}-5-ethynyl-6-fluoronaphthalen-2-ol formic acid (1/1)
or a stereoisomer, a tautomer, an N-oxide, a hydrate, a solvate, or a salt thereof, or a mixture of same.

3. The compound of general formula (I) according to claim 1 for use in the treatment of a disease.

4. A pharmaceutical composition comprising a compound of general formula (I) according to claim 1 and one or more pharmaceutically acceptable excipients.

5. A pharmaceutical combination comprising:
one or more first active ingredients, in particular compounds of general formula (I) according to claim 1, and
one or more active ingredients selected from the group consisting of:
131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, darolutamide, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, and zorubicin.

6. A method of treating a disease, the method comprising use of a compound of general formula (I) according to claim 1.

7. A method for preparing a medicament for treating a disease, the method comprising use of a compound of general formula (I) according to claim 1.

8. The method according to claim 7, wherein the disease is a neoplastic disorder, such as cancer or conditions with dysregulated immune responses or other disorders associated with aberrant KRAS signaling.

9. A method for controlling neoplastic diseases in humans and animals by administering an anti-neoplastic effective amount of at least one compound as defined in claim 1.

10. The method of claim 1, wherein the neoplastic disease is selected from the group consisting of lung adenocarcinoma, colorectal cancer, pancreatic cancer, cholangial cancer, ovarian cancer, and endometrial cancer.
